(19) United States
(12) Patent Application Publication
(10) Pub. No.: US 2025/0281465 A1
(43) Pub. Date: Sep. 11, 2025

(54) MOLECULES THAT ENHANCE EXTRACELLULAR VESICLE RELEASE

(71) Applicants: Dennis A. CARSON, La Jolla, CA (US); Masiel BELSAZURRI, Panorama City, CA (US); Michael CHAN, San Diego, CA (US); Mary Patricia CORR, San Diego, CA (US); Howard B. COTTAM, Escondido, CA (US); Tomoko HAYASHI, San Diego, CA (US); Fumi SATO-KANEKO, San Diego, CA (US); Nikunj SHUKLA, San Diego, CA (US); Yukiya SAKO, San Diego, CA (US); The Regents of the University of California, Oakland, CA (US)

(72) Inventors: Dennis A. Carson, La Jolla, CA (US); Masiel Belsazurri, Panorama City, CA (US); Michael Chan, San Diego, CA (US); Mary Patricia Corr, San Diego, CA (US); Howard B. Cottam, Escondido, CA (US); Tomoko Hayashi, San Diego, CA (US); Fumi Sato-Kaneko, San Diego, CA (US); Nikunj Shukla, San Diego, CA (US); Yukiya Sako, San Diego, CA (US)

(21) Appl. No.: 18/861,136

(22) PCT Filed: Apr. 26, 2023
(86) PCT No.: PCT/US2023/020041

§ 371 Date: Oct. 28, 2024

Related U.S. Application Data
(60) Provisional application No. 63/335,556, filed on Apr. 27, 2022.
(60) Provisional application No. 63/442,987, filed on Feb. 2, 2023.

(51) Int. Cl. A61K 31/433; A61K 31/4439; A61P 37/04

(52) U.S. Cl. CPC A61K 31/433; A61K 31/4439; A61P 37/04

(57) ABSTRACT

Provided herein are compounds that alter calcium mobilization and/or enhance extracellular vesicle production, compositions having the compounds, and methods of making and using the compounds.

### CROSS-REFERENCE TO RELATED APPLICATIONS

This application claims the benefit of the filing date of U.S. application No. 63/335,556, filed on Apr. 27, 2022, and U.S. application No. 63/442,987, filed on Feb. 2, 2023, the disclosures of which are incorporated by reference herein.

### STATEMENT OF GOVERNMENT RIGHTS

This invention was made with government support under grant numbers HHSN272201800048 and 75N93019000042 awarded by the National Institutes of Health. The government has certain rights in the invention.

### BACKGROUND

Extracellular vesicles (EVs) act as carriers of cell-type-specific molecules, including those involved in innate immune responses, such as cytokines, chemokines, adhesion molecules, lipids, nucleic acids, coding and non-coding RNAs (including microRNAs), and DNA fragments (Cossetti et al., 2014; Yanez-Mo et al., 2015; Skog et al., 2008; Valadi et al., 2007). EV cargo can convey specific intercellular communications and mediate immune responses to microbial pathogens and tumors (Wang et al., 2017; Campos et al., 2015; Kalluri & LeBleu, 2020). Thus, EVs are a potential tool for vaccine adjuvant strategies (Santos & Almeida, 2021; Sabanovic et al., 2021).

EVs derived from dendritic cells (DCs) may present on their surface the major histocompatibility (MHC) class I and II molecules, as well as B7 costimulatory molecules, such as B7.1 (CD80) and B7.2 (CD86), which directly engage and activate CD4⁺ or CD8⁺ T cells (Admyre et al., 2006; Schorey et al., 2015; Lindenbergh, 2018; Anand, 2014). EVs also act as antigen-transferring/delivering tools. EVs from tumor cells can contribute to immunotherapy via delivering the tumor antigens to antigen-presenting cells (Moroishi et al., 2016). Circulating EVs from individuals who received mRNA-based vaccination for severe acute respiratory syndrome coronavirus 2 (SARS-CoV-2) were loaded with SARS-CoV-2 spike protein and induced spike protein-specific T cell response and antibodies (Bansal et al., 2021). EVs released from antigen-pulsed DCs or engineered EVs equipped with antigens can serve as artificial antigen-presenting particles (Sabanovic et al., 2021; Nakayama, 2015; Montecalvo et al., 2008). Thus, EVs are recognized as a next-generation vaccine platform because they function as cargo that transfers antigens and adjuvants and could be a promising strategy for enhancing vaccine efficacy (Santos & Almeida, 2021; Sabanovic et al., 2021; Jesus et al., 2018).

Intracellular Ca²⁺ influx is associated with both EV secretion and immune responses (Rao & Hogan, 2009; Vig & Kinet, 2009; Savina et al., 2003; Ambattui et al., 2020; Taylor et al., 2020; Messenger et al., 2018; Kramer-Albers et al., 2007; Czerniecki et al., 1997). Calcium signaling plays multiple roles in the activation, migration, and maturation of DCs that contribute to T cell priming and activation (Rao & Hogan, 2009; Vig & Kinet, 2009). Intracellular Ca²⁺ increment leads to plasma membrane EV biogenesis (Savina et al., 2003; Ambattu et al., 2020; Taylor et al., 2020; Messenger et al., 2018). Recent reports indicate that the calcium ionophores ionomycin (ION) and A23187 enhance EV release (Savina et al., 2003; Messenger et al., 2018; Kramer-Albers et al., 2007) and induce maturation and activation of antigen-presenting cells (APCs) (Czerniecki et al., 1997). However, these compounds are often toxic for in vivo utilization (Smith & Hall, 1994).

### SUMMARY

The successful use of suitable adjuvant formulations in vaccines against globally important infectious agents or disease, such as cancer, represents a major step forward in medicine to broaden and enhance the protection of individuals from the ever-changing threats of infectious pathogens and disease.

Compounds having various chemical scaffolds were identified in a HTS screen of a commercial compound library of compounds, to induce calcium mobilization and/or extracellular vesicles (EVs) that have immuno-enhancing activity. In one embodiment, the derivatives are TLR4 derivatives of pyrimidoindoles. These derivatives contain hydrophilic substituents, such as phosphate and sulfate. In one embodiment, the scaffolds include 1) benzothiadiazole sulfonamide derivatives (#634 series); 2) thienylsulfonamides (#504 series); and 3) diaryl derivatives of oxadiazoles (#645 series). The compounds may be useful as vaccine adjuvants, calcium inducers and/or EV inducers with immune activity

In one embodiment, the disclosure provides for a method to enhance an immune response in a mammal, comprising administering to a mammal in need thereof a compound disclosed herein and optionally an immunogen in an effective amount. In one embodiment, the compound and an immunogen are administered simultaneously. In one embodiment, the compound and an immunogen are administered separately. In one embodiment, the immunogen is a microbial immunogen. In one embodiment, the microbe is a virus, such as influenza or varicella, or a bacteria. In one embodiment, the mammal is a human. In one embodiment, the immunogen is a cancer antigen.

Specifically, to identify small molecules that increase EV release from APCs, three independent high-throughput screenings (HTS) were performed on a 28K compound library from Maybridge (Leeds, United Kingdom) (Shukla et al., 2022). A human monocytic leukemia THP-1 reporter cell line engineered with a fusion construct of EV-associated tetraspanin (CD63)-linked Turbo-luciferase (Tluc) (CD63Tluc-CD9EmGFP THP-1 reporter cells) (Shpigelman et al., 2021), and two additional THP-1 reporter cell lines for NF-κB and interferon-stimulated response element (ISRE) activation, were used. Eighty hit compounds were identified after the validation using murine bone marrow-derived dendritic cells (mBMDCs) and assessment from a medicinal chemistry perspective (Shukla et al., 2022).

The 80 hit compounds were screened for the ability to induce Ca²⁺ influx and a hit compound was identified, e.g., ethyl 2-(benzo[c][1,2,5]thiadiazole-4-sulfonamido)-4,5-dimethylthiophene-3-carboxylate (hereafter designated compound 634), that triggers Ca²⁺ influx in mBMDCs. 634 enhanced the number of EVs released and also costimulatory molecule expression on EVs. Purified EVs from 634-pulsed mBMDCs promoted antigen-specific T cell proliferation. Moreover, focused structure-activity relationship (SAR) studies on 634 suggested that an increase in intracellular Ca²⁺ is closely associated with the immunostimulatory potency of EVs released by 634-treated mBMDCs.

Thus, small molecule compounds were identified that increase immunostimulatory extracellular vesicles (EVs) released by antigen presenting dendritic cells (DCs) which is useful for enhancement of vaccine immunogenicity. These compounds may induce calcium influx. These compounds are useful tools for the development of effective EV-based vaccines, e.g., small molecule compounds that enhance immunostimulatory EV release via induction of Ca2+ influx.

Therefore, the disclosure provides EV-based vaccines, e.g., adjuvanted vaccines.

Also provided are compositions having the disclosed compounds, which compounds increase immunostimulatory extracellular vesicles (EVs) release by antigen-presenting dendritic cells (DCs) which in turn enhances vaccine immunogenicity, and methods of using the EVs.

In one embodiment, a method is provided comprising: administering to a mammal an effective amount of a composition comprising an agent that promotes the release of extracellular vesicles, e.g., from dendritic cells. In one embodiment, the agent enhances calcium flux. In one embodiment, the composition is orally, intramuscularly or parenterally administered or by intrapulmonary routes, e.g., intranasally. In one embodiment, the composition is administered to a tumor by direct injection. In one embodiment, the composition is administered systemically using liposomes, antibodies or other targeting mechanisms.

In one embodiment, a method to induce extracellular vesicle production in a mammal is provided comprising: administering to the mammal a compound disclosed herein. In one embodiment, a method to induce extracellular vesicle production in mammalian cells is provided comprising: contacting the cells with a compound disclosed herein. In one embodiment, the method further comprises administering to the mammal or contacting the mammalian cells with an antigen. In one embodiment, a method to enhance the immunogenicity of an antigen is provided, comprising administering to a mammal an amount of the antigen and the composition of EVs effective to enhance the immune response in the mammal. In one embodiment, the mammal is a human.

### BRIEF DESCRIPTION OF THE FIGURES

FIGS. 1A-1D. Intracellular Ca²⁺ influx screening of 84 HTS hit candidate compounds. (A) THP-1 cells were loaded with the ratiometric Ca²⁺ indicator, Fura-2, and treated with lonomycin (ION, 1 μM), Thapsigargin (1 μM), or 84 hit candidate compounds (10 μM) for 25 minutes. Data are presented as the area under the curve (AUG) of OD_{340/380} ratios corresponding to the intracellular Ca²⁺ kinetics. The baseline-subtracted AUG was calculated using GraphPad Prism (GraphPad Software, San Diego, CA). (B) Ca²⁺ mobilization by compound #634. mBMDCs were loaded with the ratiometric Ca²⁺ indicator, Fura-8, and treated with ION (1 μM), or #634 (2 and 10 μM) for 25 minutes. The dashed line indicates the timing of compounds added. The data shown are representative of three independent experiments showing similar results. (C) Ca²⁺ add-back assay. Fura-8-loaded mBMDC were treated with ION (1 μM), or compound #634 (2 μM, 10 μM) for 15 minutes in the absence of extracellular Ca²⁺, and then Ca²⁺ (final 1.8 mM) was added at 15 minutes. Data shown are representative of three independent experiments showing similar results. (D) C57BL/6 mice (n=5/group) were intramuscularly immunized with ovalbumin (OVA, 20 μg/mouse) adjuvanted with compound #634 (200 nmol/mouse), or Veh (10% DMSO) on days 0 and 21 and bled on day 28. OVA-specific IgGI and IgG2c levels in sera were determined by ELISA. **p<0.01, ***p<0.001, ns (not significant) by KruskalWallis tests with Dunn's post hoc test vs. Veh.

FIGS. 2A-2C. RNA sequencing analysis of mBMDC treated with compound #634, (A) mBMDCs were treated with compound #634 (5 μM) or Veh (0.1% DMSO) or in triplicate for 5 hours. RNA was isolated, and RNA-seq analysis was performed by UC San Diego IGM Genomic Center. Data were analyzed by UC San Diego Moore Cancer Center Biostatistics and Bioinformatics Shared Resources. The volcano plot shows all 11759 significantly differentially expressed genes induced by #634 exposed cells compared to Veh-treated cells. The 15 genes with the top p-value are displayed in blue. (B) Kyoto Encyclopedia of Genes and Genomes (KEGG) pathway analysis. Normalized enrichment scores (NES) of the top 10 positively and negatively enriched genes sets (FDR≤0.05 and fold change >2). (C) Two down-regulated and 5 up-regulated genes related to Ca²⁺ influx and signaling were identified by Gene Summaries from NCBI RNA reference sequence collection (RefSeq) Database, Gene Ontology (GO) Biological Process, GO Molecular Function, or KEGG pathway.

FIGS. 3A-3D. Compound #634 enhances EV release. (A) mBMDC (7.5×10⁶/ml in 40 ml) were incubated with compound #634 or Veh (0.01% DMSO) for 48 hours and the culture supernatants were collected. EVs were isolated from the culture supernatant by differential ultracentrifugation, and final pellets were re-suspended in 50 μl filtered PBS (designated as EV₆₃₄ and EVᵥₑₕ). An aliquot of EV suspension was diluted 100-fold with 1% Tween 20 in filtered PBS, and particle numbers were measured by MRPS using the nCS1 instrument with C-400 cartridges (Spectradyne, Signal Hill, CA). A number of particles was calculated to the number isolated from 1 ml culture supernatant. The results were analyzed using the nCS1 Data Analyzer software. Data shown are means±SD of 3 to 4 different mBMDC batches. *p<0.05 by Student's t-test. (B) Concentration spectral density (CSD) shows the size range/distribution of EV₆₃₄ and EVᵥₑₕ. Data shown are mean of 3 to 4 different mBMDC batches. Dashed line represents 75 nm. (C) Immunoblot of EVs and parental cell lysates. The proteins in EV₆₃₄ and EVᵥₑₕ and their parent cells (C) were separated by 4-12% gel electrophoresis using NuPage 4 to 12% Bis-Tris Gel (Thermo Fisher Scientific) and transferred onto Immobilon-P PVDF membranes (Millipore). Blots were probed with anti-CD81, Tsg101, and Calnexin. The images shown are representative of two independent experiments. (D) Total RNA yield from 30 μl EVs suspension was determined by the RNeasy Mini Kit using Nanodrop. Data shown are means±SD of two different mBMDC batches.

FIG. 4. T cell proliferation assay protocol.

FIGS. 5A-5D. EV₆₃₄ enhances T cell proliferation in the presence of Antigenic peptides mBMDCs (7.5×10⁶/mL in 40 mL) were incubated with MPLA (1 μg/mL), ION (1 μM), #634 (10 μM), or Veh (0.01% DMSO) for 48 hours. EVs were isolated from the culture supernatant by differential ultracentrifugation and resuspended in particle-free PBS. (A) CFSE-labeled CD4⁺ T cells isolated from OVA TCR transgenic strain, DO.11.10, were treated with an equal amount (7 μL) of EVs in the presence of OVA₃₂₃₋₃₃₉ peptide for 5 days. T cell proliferation was determined by CFSE dilution using flow cytometry. (B) Percent T cells that divided relative to the original population are shown. Data presented are means±SD of triplicates. *p<0.05, **p<0.01 by one-way ANOVA with Dunnett's post hoc test vs. Veh. (C) EVs isolated from an increased number of mBMDC was cultured with total CD4⁺ T cells in the presence of OVA₃₂₃₋₃₃₉ peptide for 5 days. Data shown are means±SD of duplicates of one experiment. (D) EV₆₃₄ or EVᵥₑₕ from 3.2×10⁶ BMDCs were cultured with total CD4⁺ T cells in the presence or the absence of OVA₃₂₃₋₃₃₉ peptide for 5 days. T cell proliferation was determined b CFSE dilution using flow cytometry.

FIGS. 6A-6D. Intracellular Ca²⁺ influx and costimulatory molecules expression induced by SAR derivatives of compound #634. (A) Intracellular Ca²⁺ levels of mBMDCs were analyzed following the SAR compound treatment by Fura-2 assay. mBMDC were loaded with and treated with Veh (0.5% DMSO), ION (1 μM), compound #634 (10 μM), or twelve 634 analogs (10 μM) {all are in house derivatives) for 30 minutes. Data were presented as AUC of OD_{340/380} ratios. The baseline-subtracted AUC was calculated using GraphPad Prism. AUC was normalized to Veh (=1). Data are presented as means±SD of duplicates of two independent experiments showing similar trends. *p<0.05, ***p<0.001 by one-way ANOVA with Dunnet's post hoc test vs #634-treated cells. (B) Cytotoxic effects of the SAR derivatives of #634. mBMDCs after 20 hour treatment with Veh (0.5% DMSO), ION (1 μM), #634 (1 μM), or twelve 634 analogs (10 μM), and cell viability was determined by MTT assay. The relative viability was normalized to Veh-treated cells. (C) Costimulatory molecule expression induced by #634 analogs. mBMDCs were incubated with Veh (0.5% DMSO), ION (1 μM), 634 (1 μM), or 12 analogs (10 μM) for 20 hours and subjected to flow cytometry analysis with anti-CD86 and anti-MHC class II antibodies. Mean fluorescence intensities (MFI) for CD86 and MHC class II relative to Veh are shown. Data shown are means±SD of duplicates of two independent experiments. *p<0.05, ***p<0.001 by one-way ANOVA with Dunnet's post hoc test vs Veh-treated cells. (D) Correlation between CD86 expression and intracellular Ca²⁺ increase following compound treatment. MFI for CD86 and AUC in Fura-2 assay normalized to Veh treated cells are shown in the scatter plot. The linear regression was analyzed by Spearman's rank correlation coefficient using Graphpad Prism. Each dot corresponds to the mean of two independent experiments. The dashed line indicates each Veh value.

FIGS. 7A-7B. Induction of intracellular Ca²⁺ influx in mBMDGs by #504 analogs. (A) mBMDCs were loaded with Fura-2 and treated with Veh (0.5% DMSO), ION (1 μM), Thapsigargin (1 μM), #504 (10 μM), or thirty-seven #504 analogs (10 μM) (All are repurchased derivatives) for 30 minutes. Data were presented as AUC of OD_{340/380} fluorescence ratios corresponding to changes in the intracellular Ca²⁺ level. The baseline-subtracted AUC was calculated using GraphPad Prism. Data shown are means±SD of duplicates and are representative of three independent experiments. (B) Fluorescent properties of #504 analogs. #504 analogs were mixed with Fura-2 buffer and a ratio of OD₃₄₀ and OD₃₈₀ was obtained as measured by the TECAN plate reader. Data shown are means±SD of duplicates and are representative of two independent studies showing similar results.

FIG. 8. Structures of purchased 3-pyridyl-oxadiazole analogs of #645.

FIG. 9. Structures of synthesized analogs of compound #645.

FIGS. 10A and 10B. (A) Bioactivity data for selected analogs of compound #645. (B) IL-12 induction in BMDCs and THP-1 CD63 reporter assay for triazole analogs of compound #645.

FIGS. 11A-11B. HTS for immunostimulatory compounds that enhance EV release and the HTS workflow. (A) A cartoon depicting the rationale for identification of compounds that enhance EV release as well as induction of cytokines and chemokines. (B) Three independent THP-1 cell-based high throughput screenings were performed using NF-κB-beta-lactamase (bla), ISRE-bla and CD63-Tluc-CD9-EmGFP reporter cells. These assays evaluated 27,895 compounds in duplicate and 644 compounds were identified as hits using two different statistical methods. These compounds were cherry-picked and were subjected to screening for immune stimulating activity, including induction of cytokine IL-12 and evaluation of cytotoxicity using MTT assay in mBMDCs which identified 130 compounds. Further medicinal chemistry approaches eliminated 50 compounds and the remaining 80 compounds were subjected to in vivo adjuvanticity screening, followed by co-stimulatory molecule expression screening and quantitation of EVs released from BMDCs. Eight distinct compounds were identified that belonged to 7 different chemotypes. The number in parentheses corresponds to the number of compounds.

FIGS. 12A-12F. Hit selection methods from the NF-κB and ISRE HTS. The hit selection process for NF-κB HTS (A,B,C) and ISRE HTS (D,E,F) are depicted: (A,D) MA plots of log₁₀ transformed % activation for all compounds identified as hits in one experiment (orange spheres) or in both experiments as Top X hits (red spheres). The positive (LPS for NF-κB HTS; IFN-α for ISRE HTS) and negative (Veh, 0.5% DMSO) controls used in the assay are shown as blue and green spheres, respectively. Panels (B,E) represent first steps towards the mixture model method and involved the identification of a null cluster and elimination of compounds to the left side of the red vertical dotted line. (C,F) The next step involved identification of linear boundaries based on the apparent false-positive clusters (black symbols) to identify GMM hits (red symbols) that included all compounds within these linear boundaries.

FIGS. 13A-13D. Hit selection from HTS. Scatter plot of activation data for test compounds (evaluated in duplicates) and controls from (A) NF-κB HTS, (B) ISRE HTS and, (C) CD63 HTS. The activation data were calculated as “% activation” based on 2-point normalization between the controls in each plate of the HTS assay. These controls included Veh (0.5% DMSO, negative control, 0%) and LPS (100 ng/ml, 100%) for NF-κB HTS, IFN-α (50 nM, 100%) for ISRE HTS and PMA (10 ng/ml, 100%) for CD63 HTS. Two different statistical methods including Top X and GMM, were utilized for hit identification. All test compounds are shown by grey dots, while the compounds that were identified as hits by Top X only or GMM only methods are shown as green and blue spheres, respectively. The compounds identified as hits by both of these statistical techniques are shown as red spheres. Controls (purple stars) are shown as mean±standard deviation calculated by intra-assay statistics of the % activation values. (D) Venn diagram showing the number of compounds identified as hits in each assay after eliminating toxic compound (<40% viability) identified by PrestoBlue viability assay in CD63 HTS. Compounds confirmed as hits in at least 2 different assays as shown by numbers in the intersections of the Venn diagram were selected for the further bioactivity analysis (total 644 compounds).

FIG. 14. Evaluation of immunostimulatory activity and toxicity in murine BMDCs. Selected hits candidates (644 compounds) were cherry-picked and evaluated in triplicates for induction of IL-12 release and cell viability by MTT assay in mBMDCs. IL-12 induced by test compounds was normalized to IL-12 induced by Veh (0.5% DMSO, 1 Arbitrary unit) in each plate. The cell viability following treatment with each compound was normalized to the viability following treatment of cells with Veh (% viability of Veh=100%). A scatter plot for all compounds showing normalized IL-12 induction on the X-axis vs. cellular viability on the Y-axis helped for selecting immunostimulatory compounds that were relatively less toxic. All tested compounds are shown in grey, while the compounds that induced IL-12 above mean+3SD (standard deviation within each assay plate) of the vehicle are shown in color, of which compounds that led to cellular viabilities less than 60% are shown in red spheres (191 compounds) while relatively non-toxic (% viability ≥60%) and potent IL-12 inducing compounds are shown by blue spheres (229 compounds).

FIGS. 15A-15C. In vivo adjuvanticity screening of test compounds with ovalbumin as a model antigen in mice. (A) C57BU6 mice (n=3/group) were intramuscularly immunized with OVA (20 μg/mouse) as antigen and adjuvanted with test compounds (200 nmoles/mouse), or MPLA (1 μg/mouse), or Veh (10% DMSO) on days O and day 21 and bled on day 28 to measure OVA-specific IgG2c and IgG1 levels in sera by ELISA. A scatter plot of the geomean (n=3) values of the IgG2c titers (Y-axis) and IgG1 (X-axis) for each compound was generated to show the adjuvant potency distribution.

Compounds were represented by different symbol types based on the type of hit identified, namely, triple hits (red symbols) and dual hits including CD63 and NF-κB dual hits (magenta symbols), CD63 and ISRE dual hits (green symbols) and NF-κB and ISRE dual hits (blue symbols). MPLA was the positive control while Veh was used as the negative control shown as grey stars. Based on the potency of compound to enhance the induction of combined IgG1 and IgG2c titers, they were divided into three tiers, Tier 1 (circles), Tier 2 (squares) and Tier 3 (triangles). These tiers were obtained by first calculating log 10 transformed values of the IgG1 and IgG2c titers and normalizing these values in between 0 and 10 (10 for MPLA and 0 for Veh). This was followed by averaging these values (for each compound) for IgG1 and IgG2c to obtain a combination value, where Tier 1 compounds had a combination value ≥8, Tier 2 compounds had a combination value ≥6 and ≤8, while Tier 3 compounds had a combination value <6. Correlation of antigen-specific IgG1 (B) and IgG2c (C) antibody titers with primary HTS (CD63, NF-κB, and ISRE) data. The data were analyzed by two-tailed nonparametric correlation (Spearman) with calculated p-values shown.

FIG. 16. Heat map depicting a summary of biological activities of selected 18 hits. A heat map was generated for the selected 18 compounds based on all the biological data, including adjuvanticity (IgG1 and IgG2c), cell viability (MTT and PrestoBlue (PB)), primary HTS (CD63, NF-κB, and ISRE), cytokine IL-12 induction (5 and 10 μM compound concentration), and costimulatory molecules expression (CD40, CD80, CD83, CD86, and MHC class II). The absolute values from these assays were standardized and clustered for compounds presenting similar biological outcomes, as shown using a hierarchical plot on the left. This allowed us to identify 4 different clusters of compounds with similar activity profiles shown within a black box on the heat map.

FIGS. 17A-17F. EV characterization and spider plots for selected hits depicting similar biological activities. (A) Number of particles per ml of starting culture medium volume assessed by MRPS using the nCS1 instruments with C-400 cartridges. mBMDCs were incubated with compound (10 μM), Veh, or bafilomycin A1 (Baf, positive control) for 48 hours and EVs in the supernatant were isolated. The EVs were diluted 100-fold in 1% Tween 20-PBS and quantitated using the nCS1 system. All results were analyzed using the nCS1 Data Analyzer software. Bars indicate means±SEM of 3-4 replicates of mBMDC batches. *p<0.05,***p<0.0001, One-way ANOVA with Dunnett's post hoc test. (B) Co-culture of mBMDCs with isolated EVs. mBMDCs were cultured with 10 μM #645, #504 or Veh for 48 hours and EVs in the culture supernatant were isolated and resuspended in 50 μL PBS. Freshly prepared mBMDCs (10⁵/100 μL) were mixed with 7 μL of the EVs and co-cultured for 18 hours. IL-12 levels in the supernatant were evaluated by ELISA. Bars indicate means±SD of duplicate wells. *p<0.05, One-way ANOVA with Dunnett's post hoc test vs. Veh. Spider plots show selected activity profiles' overlay for compounds within the cluster. These activity profiles include 1. Adjuvanticity as a composite of IgG1 and IgG2c titers, 2. EVs particle concentration, 3. IL-12 induction, 4. Composite value for the expression of co-stimulatory molecules including CD40, CD80, CD83, CD86 and MHC class II, 5. NF-κB HTS data, 6. CD63 HTS data, and 7. Cell viability by MTT. The figures are shown as overlays of activity profiles of Veh with (CJ Cluster 1 compounds #645 and #339; (DJ Cluster 2 compounds #298 and #455; (E) Cluster 3 compounds #456 and #504; (F) Cluster 4 compounds #311 and #336. These clustered compounds show similar activity profiles’, thus validating the HTS.

FIGS. 18A-18C. Compound 634 induces intracellular Ca²⁺ levels in mBMDCs. (A) Intracellular Ca²⁺ influx levels of the top eight compounds. THP-1 cells were loaded with the ratiometric Ca²⁺ indicator, Fura-2, and treated with ION (1 μM), TG (1 μM), or test compounds (5 μM). The time-response pattern of intracellular Ca²⁺ levels was recorded for 25 minutes. AUC of OD_{340/380} ratios corresponds to the intracellular Ca²⁺ kinetics, and the baseline-subtracted AUC was calculated by GraphPad Prism. Data presented are relative AUC to Veh (1.74 at 1st exp., and 1.06±0.05 at 2nd exp were set as 1, respectively), and mean±SD of pooled data from three experiments showing similar results. **p<0.01, ***p<0.001 by one-way ANOVA with Dunnett's post hoc test compared to Veh. (B) Ca²⁺ mobilization by compounds 456 and 634 in mBMDCs. mBMDCs were loaded with Fura-8 and treated with ION (1 μM), or 634 and 456 (10 μM) for 25 minutes. The dashed line indicates the timing of compounds added. The data shown are representative of three independent experiments showing similar results. (C) Ca²⁺ add-back assay. Fura-8-loaded mBMDC were treated with ION (1 μM) or compound 634 (2 μM, 10 μM) for 15 minutes in the absence of extracellular Ca²⁺, and then Ca²⁺ (final 1.8 mM) was added at 15 minutes. The data shown are representative of three independent experiments showing similar results.

FIGS. 19A-19E. 634 enhances EV release. mBMDCs were incubated with compound 634 (10 μM), ION (1 μM), or Veh (0.5% DMSO) for 48 hours. EVs were isolated from the culture supernatant by differential ultracentrifugation, and final pellets were re-suspended with 50 μL PBS (designated as EV₆₃₄, EV_{ION}, and EVᵥₑₕ). (A) The results were analyzed by MRPS, and EV number was calculated per mL. Data shown are means±SD of EVs from six experiments using different mBMDC batches. *p<0.05 by one-way ANOVA with Dunnett's post hoc test vs. Veh. (B) Size distributions of EV₆₃₄, EV_{ION}, and EVᵥₑₕ were measured by MRPS. Data shown are means±SEM of EVs from six different mBMDC batches. (C) Immunoblots of EVs and parental cell lysates. The proteins (2 μg/well) were separated by 4-12% NuPAGE gel. Blots were probed with anti-CD81, anti-Alix, or anti-Calnexin antibodies. The images shown are representative of two independent experiments showing similar results. (D) Morphological examination of EV₆₃₄, EV_{ION}, and EVᵥₑₕ by TEM. Scale bars represent 200 nm. (E) The total protein contents of EV₆₃₄, EV_{ION}, and EVᵥₑₕ were calculated per 10¹⁰ EV particles. The results were measured using a Micro BCA assay kit. Experiments were repeated 6 times using individual mBMDC batches. Data shown are means±SD of data from 6 measurements. n.s., not significant by one-way ANOVA with Dunnett's post hoc test vs. Veh.

FIGS. 20A-20C. 634 induces costimulatory molecule expression on EV₆₃₄. (A and B) EVᵥₑₕ, EV₆₃₄, EV_{ION}, and EV_{MPLA}, were stained with a cocktail of vFRed™, anti-CD86 APC (A), and anti-MHC class II AF488 (B) and analyzed by flow cytometry. MFI for CD86 and MHC class II relative to EVᵥₑₕ are shown. (CD86; 58.4±0.4 at 1st batch, 49.2±0.2 at 2nd exp, 56.0±0.5 at 3rd exp., and 63.1±3.7 at 4th exp. and MHC class II; 416.5±57.3 at 1st exp., 556.5±44.5 at 2nd exp, 711.5±16.3 at 3rd exp. and 559.0±14.1 at 4th were set as 1, respectively). Each dot represents a dataset from individual mBMDC batches. Data shown are means±SD of EVs from four different mBMDC batches. *p<0.05 by one-way ANOVA with Dunnett's post hoc test vs. EVᵥₑₕ. (C) Immunoblot of EVs. The protein (2 μg/well) was separated and probed with anti-CD86, anti-CD80, anti-MHC class II, anti-CD40, or anti-CD81 antibodies. The images shown are representative of two independent experiments with similar results.

FIGS. 21A-21D. EV₆₃₄ enhance T cell proliferation in the presence of antigenic peptides. (A and B) CFSE-labeled CD4⁺ T cells isolated from splenocyte of OVA TCR transgenic strain, DO11.10, splenocytes were treated with an equal volume (7 μL out of 50 μL) of the suspensions of EVVeh, EV₆₃₄, EV_{ION}, or EV_{MPLA}, in the presence or absence of OVA₃₂₃₋₃₃₉ peptide for 5 days. EV_{No cells} were used as a negative control. (C and D) T cell proliferation was determined by CFSE dilution using flow cytometry. Percentages of divided T cells induced by EVs from same number of parent cells) and equal number (3.13×10⁹) of EV particles (D) are shown. Data shown are means±SD of triplicates representative of two independent experiments. **p<0.01, ***p<0.001 by one-way ANOVA with Dunnett's post hoc test vs. EVᵥₑₕ.

FIG. 22. Focused structure-activity relationship (SAR) studies in 634. Syntheses of twelve 634 analogs using a modification of the ester site of 634.

FIGS. 23A-23D. Correlation between intracellular Ca²⁺ influx and T cell proliferation by EV from mBMDCs treated with 634 analogs. (A) Intracellular Ca²⁺ levels in mBMDCs were monitored following 634 analogs treatment. mBMDCs were loaded with Fura-2 and treated with 634, its SAR analogs (10 μM), Veh (0.5% DMSO), ION (1 μM), or TG (1 μM). Data are presented as the normalized AUC (Veh was 1.00±0.15). Data presented are mean±SD of pooled two independent experiments performed in triplicate, showing similar results. *p<0.05 by one-way ANOVA with Dunnett's post hoc test compared to Veh. (B) Correlation analysis between intracellular Ca²⁺ induction (AUC) and the CD63 Tluc-CD9EmGFP THP-1 reporter responses. Relative Luminescence activity to Veh (792.7±84.58 at 1st exp., and 353.3±29.14 at 2nd exp. were set as 1, respectively) is shown. (C) Correlation analysis between intracellular Ca²⁺ induction (AUC) and CD86 expression (MFI). mBMDCs were incubated with Veh, ION (1 μM), 634, and its analogs (10 μM) for 24 hours. MFI was normalized to Veh (1.00±0.02). The fitted regression line is shown. Pearson correlation analysis for 634 and its SAR analogs was performed by Graphpad Prism 9. (D) CFSE-labeled DO11.10 CD4⁺ T cells were cultured with an equal volume of EV suspension (7 μL out of 50 μL) in the presence of OVA₃₂₃₋₃₃₉ peptide for 5 days. Percentages of divided T cells relative to the original population are calculated. Each dot shown is the average of three independent experiments performed in triplicates, and data shown are means±SEM of negative compounds (2E241, 2F186, and 2H013) or five positive compounds (634, 2G176, 2G179a, 2G179c, 2G179g), respectively. *p<0.05 by Mann-Whitney U test.

FIGS. 24A-24B. Intracellular Ca2+ screening of eighty HTS hit candidate compounds. THP-1 cells were loaded with the ratiometric Ca²⁺ indicator, Fura-2, and treated with ION (1 μM), TG (1 μM), or test compounds (5 μM). The time-response pattern of intracellular Ca²⁺ levels was recorded with a plate reader for 25 minutes. (A) The AUC of OD_{340/380} ratios corresponds to the intracellular Ca²⁺ kinetics and the baseline-subtracted AUC was calculated by GraphPad Prism (FIG. 36). Data presented are relative AUC to Veh (1.74 was set as 1). (B) Ca²⁺ mobilization by the top eight compounds identified in the previously performed HTS by us (1). Data presented are averages of duplicates and representative of two independent experiments showing similar results.

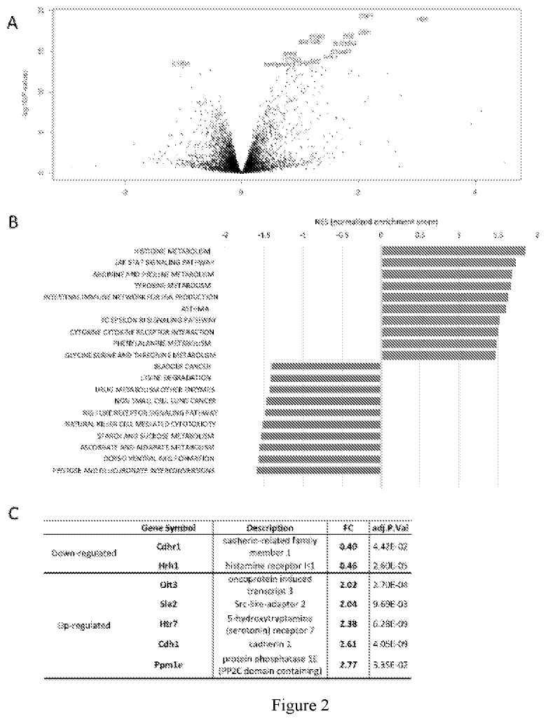

FIGS. 25A-25C. Full original immunoblots presented in FIG. 2C. The blots of parental cell lysate (labeled as C) and EVs (labeled as E) were stained with anti-CD81(A), anti-Alix (B), and anti-Calnexin (C). In staining with CD81, samples were run under non-reducing conditions. AccuRuler Prestained Protein Ladder (Lamda Biotech) was used as a molecular weight marker.

FIGS. 26A-26D. Flow cytometric analysis for costimulatory molecules on mBMDCs. For costimulatory molecules on mBMDCs, mBMDCs (10⁶ cells/mL) were incubated with 634 (10 μM), ION (1 μM), and MPLA (1 μg/mL) for 20-24 hours. 0.5% DMSO was used as a vehicle. After removing the supernatant, cells were washed with the stain buffer and incubated with anti-mouse CD16/32 antibody for blocking FcR. Cells were stained with an antibody cocktail with anti-CD86 (A), anti-CD80 (B), anti-MHC class II (C), and anti-CD40 (D) antibodies for 30 minutes at 4° C. Cells were then washed and stained with DAPI (4′,6-diamino-2phenylindole) for 10 minutes at room temperature to exclude dead cells (DAPI^{high}) from the analysis. Data were acquired using MACSQuant Analyzer 10 (Miltenyi Biotec, Germany) and analyzed with FlowJo (version 10.8.1). Mean fluorescence intensity (MFI) is shown. **p<0.01, ***p<0.001 by one-way ANOVA with Dunnett's post hoc test vs. Veh.

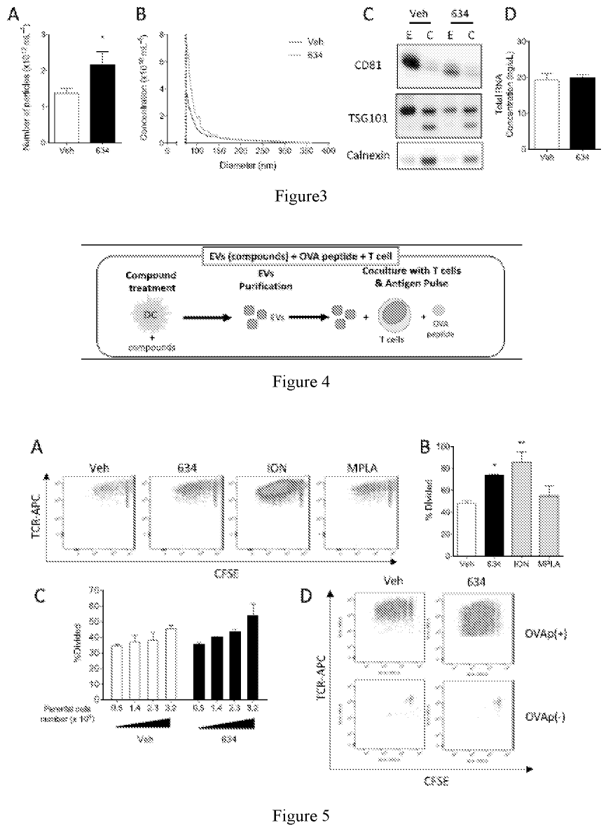

FIGS. 27A-27E. Full original immunoblots presented in FIG. 3C. The blots of EVs were stained with anti-CD86 (A), anti-CD80 (B), anti-MHC class II (C), anti-CD40 (D), and ant-CD81 (E) antibodies. AccuRuler Prestained Protein Ladder (Lamda Biotech) was used as a molecular weight marker.

FIGS. 28A-28B. IL-2 levels in the culture supernatants in the T cell proliferation studies in FIG. 4. CFSE-labeled CD4⁺ T cells isolated from OVA TCR transgenic strain, DO.11.10, splenocytes were treated with an equal volume (7 μL out of 50 μL) (A) or an equal particle number (3.13×10⁹ EV particles) (B) of EV₆₃₄, EV_{ION}, EV_{MPLA}, or EV_{No cell} in the presence of OVA₃₂₃₋₃₃₉ peptide for three days. The supernatants were assayed for IL-2. Data shown are means±SD of triplicates representative of two independent experiments. ***p<0.001 by one way ANOVA with Dunnett's post hoc test vs. Veh.

FIG. 29. Intracellular Ca²⁺ mobilization of the SAR derivatives of 634. mBMDCs were loaded with the ratiometric Ca²⁺ indicator, Fura-2, and treated with Veh (0.5% DMSO), ION (1 μM), TG (1 μM), or test compounds (10 μM). The time-response pattern of intracellular Ca²⁺ levels was recorded with a plate reader for 25 minutes. The experiment was performed in duplicate, and the data represent two independent experiments showing similar results. The dashed line indicates the timing of compounds added.

FIGS. 30A-30B. Cytotoxic effects of the SAR derivatives of 634. mBMDCs were cultured in RPMI1640 supplemented with 10% exosome depleted FBS (A) or with 10% fetal bovine serum (FBS) (B) and treated with Veh (0.5% DMSO), lonomycin (ION, 1 μM), MPLA (1 μg/mL), or test compounds (10 μM). After 48 hours incubation, cell viability was determined by MTT assay. The relative viability was normalized to Veh-treated cells [Veh was 1.00±0.03 at (A), and 1.00±0.02 at (B)]. Data shown are means±SD of triplicates of a representative experiment of two independent experiments.

FIG. 31. T cell proliferation in the presence of antigenic peptides by EVs derived from mBMDCs treated with the SAR derivatives of 634. CFSE-labeled CD4⁺ T cells isolated from OVA TCR transgenic strain, DO.11.10, splenocytes were treated with an equal volume of EV suspension (7 μL out of 50 μL) in the presence of OVA₃₂₃₋₃₃₉ peptide for 5 days. T cell proliferation was determined by CFSE dilution using flow cytometry. Percentages of divided T cells relative to the original population are calculated. Data shown are means±SEM of three independent experiments performed in triplicates.

FIG. 32. Calculation method of Area Under Curve (AUC) of Ca²⁺ influx. The baseline-subtracted AUC of 340/380 ratios was calculated as net AUC using GraphPad Prism. The baseline was calculated as the mean of 340/380 ratios without compound in each measurement.

FIGS. 33A-33E. Gating strategy for MFIs of costimulatory molecules on CD11c positive cells. Flow cytometry data were gated to distinguish (A) lymphocytes and (B) singlets based on forward and side scatter. (C) DAPI^{high} dead cells were excluded from the analysis. (D) CD11c positive cells were gated, and MFIs of costimulatory molecules on CD11c positive cells were calculated.

FIGS. 34A-34E. Vesicle flow cytometry gating strategy. (A) Data were gated on time to remove events associated with any fluidic anomalies. (B) The particles larger than four pixels on the vFRed™ object area were excluded because they would be coincident or out-of-focus events. (C) These events were further gated to exclude low-intensity background events. (D) MFI of CD86 and MHC class II on vesicles was calculated. (E) The CellStream flow cytometer configuration was used in this study. FSC and SSC lasers were turned off, and its Small Particle mode was activated. Each sample was introduced at the Slow sample flow rate (3.66 μL/minute) and analyzed for 20 seconds.

FIGS. 35A-35E. Gating strategy for T cell proliferation. Flow cytometry data were gated to distinguish (A) lymphocytes and (B) singlets based on forward and side scatter. (C) DAPI^{high} dead cells were excluded from the analysis. (D) DO11.10 TCR-positive cells were gated. Cell proliferation was monitored by CFSE dilution in DO11.10 TCR gated population. Percentages of divided cells relative to the original population were calculated.

FIG. 36. Structures of analogs of TLR4 agonist 2B182c.

FIG. 37. TLR4 agonism assays with analogs of 2B182c in human and murine TLR4 reporter cells.

FIGS. 38A-38C. Antigen-specific IgG levels induced by IN boosting with Fos47 were compatible to intramuscular boosting with Fos47. (A) IM-IM and IM-IN-IN regimen. BALB/c mice (n=6−I 2 group) were IM primed with IIAV (10 μg/injection) plus Fos47 (1 nmol 1V270+200 nmol 2B182C)/injection or Lipo-Veh (blank liposomes) in 50 μL on day 0. For IM-IM regimen, the mice were IM boosted on day 21 with a full dose (50 μL per dose). For IM-IN-IN regimen, the mice were IN administered with a half dose of the vaccine (25 μL per dose; 5 μg IIAV+0.5 nmol 1V270+100 nmol 2BI82C) twice 7 days apart. One week after the last boost, sera were collected to evaluate serum IgG and IgG2a against HA. (B) Anti-HA IgG1. (C) Anti-HA IgG2a. Bars indicate means±SEM. **P<0.01, Kruskal-Wallis with Dunn's post hoc test to compare 4 groups (IM-Lipo-Veh, IM-Fos47, IN-Lipo-Veh and IN-Fos47).

FIGS. 39A-39C. IM-IN-IN vaccination regimen with Fos47 enhanced antigen-specific IgG2a responses/or 6 months. (A) Experimental schedule. BALB/c mice (n=5-10/group) were IM injected with IIAV (10 μg/injection) plus Lipo-Veh, Fos47 (1 nmol IV270 and 200 nmol 2B182C in 50 L/injection or AS01B (10 μl/injection: 1 μg MPLA and 1 μg saponin in 50 μL) on days 0. AS01B, licensed liposomal TLR4 agonists, served as a positive control. For IM-IM regimen, the mice were IM boosted on day 21 with a full dose (50 μL per dose). For IM-IN groups, the mice were IN administered with half dose vaccine on days 21 and 28. Serum samples were collected on days 56 and 182 to test anti-HA IgG1 and IgG2a levels by ELISA. (BJ Anti-HA IgG1. (C) Anti-HA IgG2a. Bars indicate±means SEM*P<0.05, **P<0.01, Kruskal-Wallis with Dunn's post hoc test to compare 4 groups (IM-Lipo-Veh, IM-Fos47, IN Lipo-Veh and IN-Fos47).

FIGS. 40A-40C. IM-IN-IN combination regimen with Fos47 enhanced serum IgG responses against NA. (A) BALB/c mice (n=5-10/group) were IM primed with IIAV plus Lipo-Veh, Fos47 or AS01B. The mice were IN boosted with a half dose of the same agents on days 21 and 28 (IM-IN-IN). Sera were collected on day 56. IgG1 (B) and IgG2a (C) specific for NA [A/California/0412009 (H1N1)] were evaluated by ELISA. Bars indicate means±SEM. *P<0.05, **P<0.01, ***P<0.0001, Kruskal-Wallis with Dunn's post hoc test to compare three groups with Lipo-Veh, Fos47 and AB01B (IM-IN-IN).

FIGS. 41A-41C. IM-IN-IN combination regimen with Fos47 induced cross-reactive antibodies against HA and NA from heterologous influenza virus strains. BALB/c mice (n=5-10/group) were IM primed with IIAV plus Lipo-Veh, Fos47 or AS01B. The mice were IN boosted with a half dose of same agents on days 21 and 28 (IM-IN-IN). Sera were collected on day 56. (A and C) Amino acid sequences of proteins used in ELISA were aligned by the MUSCLE algorithm using the Influenza Research Database. Phylogenetic trees were constructed by the neighbor-joining method using MEGAX software. A/California/04/2009(H1N1 pdm09) was used/or vaccination. Cross-reactivity against H7 and N7 of A/Netherlands/219/2003(H7N7) were tested in this assay. A/Puerto Rico/8/1934(H1N1), A/Victoria/3/1975(H3N2), A/Thailand/1 KAN 1/2001 (H5N1) are shown as a reference. (B and D) Sera were serially diluted (1:50 to 1:204800) and evaluated for total IgG levels against H7 (D) and N7 (E) proteins by ELISA. Bars indicate means±SEM*P<0.05, **P<0.01, ***P<0.0001, Kruskal-Wallis with Dunn's post hoc test to compare three IN-Lipo-Veh, Fos47, and AS01B groups.

FIGS. 42A-42C. IgA levels in BALF and serum were enhanced by intranasal boosting with Fos47. (A) BALB/c mice (n=6/group) were IM injected with IIAV plus Lipo-Veh, Fos47 or AS01B. The mice were either IM or IN boosted with a half dose of the same vaccine on days 21 and 28. The mice were sacrificed on day 35 and sera and BALF were collected. IgA levels in BALF (B) and sera (C) were evaluated by ELISA. Arbitrary units are shown. See methods for details. Bars indicate means±SEM**P<0.01, Kruskal-Wallis with Dunn's post hoc test to compare 4 groups (IM-Lipo-Veh, IM-Fos47, IN Lipo-Veh and IN-Fos47). ††P<0.01, Two-tailed Mann Whitney U-test to com are IM-Fos47 and IN-Fos47.

FIGS. 43A-43B. IgA levels in BALF and serum induced by IN-Fos47 lasted for 56 days. BALB/c mice (n=6/group) were IM injected with IIAV plus Lipo-Veh, Fos47 or AS01B. The mice were either IM or IN boosted with a half dose of the same vaccine on days 21 and 28. The mice were sacrificed on day 35 and sera and BALF were collected. IgA levels in BALF (A) and sera (B) were evaluated by ELISA. Bars indicate means±SEM*P<0.05, Kruskal-Wallis with Dunn's post hoc test to compare 4 groups (IM-Lipo-Veh, IM-Fos47, IN Lipo-Veh and IN-Fos47. ††P<0.01, Two-tailed Mann Whitney U-test to compare IM-Fos47 and IN-Fos47.

FIGS. 44A-44D. IN boosting with Fos47 enhanced antigen-specific T cell responses in the lung. (A) BALB/c mice (n=5/group) were IM injected with IIAV plus Lipo-Veh, Fos47 or AS01B. The mice were either IM boosted on day 21 or IN boosted with a half dose of the same agents on days 21 and 28. The mice were sacrificed on day 56 and activated T cells in the lung were examined by flow cytometry. % CD44⁺CD69⁺ activated T cells in CD4⁺ and CD8⁺ T cells are shown. Bars indicate means±SEM*P<0.05, **P<0.01, Kruskal-Wallis with Dunn's post hoc test to compare 4 groups (IM-IM-IM-Lipo-Veh, IM-IN-IN-Lipo-Veh, IM-IM-IM-Fos47 and IM-IN-IN-Fos47). (B-C) BALB/c mice (n=6/group) were immunized with IIAV adjuvanted with Lipo-Veh, Fos47 or AS01B by IM-IN-IN regimen on days 0, 21 and 28 (IM-IN-IN) and were sacrificed on day 35. CD4⁺CD44⁺ and CD8⁺CD44⁺ cells in the lung were analyzed using MHC I tetramer and MHC II tetramer reagents by flow cytometry. (B) % MHC II tetramer⁺ in CD4⁺ CD44⁺ cells. (C) % MHC I tetramer⁺ in CD8⁺CD44⁺ cells. Bars indicate means±SEM*P<0.05, Mann Whitney U test to compare Lipo-Veh and Fos47. (D) Splenocytes were isolated on day 35 and cultured with 10 μg hemagglutinin for 3 days. IFNγ, IL-5 and IL-17 secretion levels in the supernatants were evaluated by ELISA. Bars indicate means±SEM*P<0.05, Mann Whitney U test to compare IN-Lipo-Veh and Fos47.

FIGS. 45A-45B. In vivo labeling with anti-CD45-PE/Cy7 antibody. On day 56, mice(n=5-6/group) immunized with IIAV adjuvanted with Lipo-Veh, Fos47 or AS01B by IM-IN-IN regimen were i.v. injected with anti-CD45-PE/Cy7 antibody prior to euthanasia and then lung cells were isolated. CD4⁺CD44⁺ cells and CD4⁺CD8⁺ memory T cell subsets were farther separated by i. v. CD45 staining. iv. CD45⁻ non-circulating CD4⁺CD44⁺ cells (A) and CD8⁺CD44⁺ cells (B) in the lungs are shown. *P<0.05, **<0.01, one-tailed Mann Whitney U test to com are IN-Lipo-Veh and IN-Fos47.

FIG. 46. Systemic reactogenicity was not induced by IN delivery of Fos47. BALB/c mice (n=5-6/group) were IN administered with 25 μL Lipo-Veh, Fos47 or AS01B. Sera were collected at 6 hours or 24 hours after administration. IL-6, TNFα and KC levels in sera were evaluated by Multiplex cytokine assay Luminex. *P<0.05, Kruskal-Wallis with Dunn's post hoc test.

### DETAILED DESCRIPTION

The use of adjuvants in vaccines is a well-established method to promote a stronger immune response to weakly immunogenic antigens. In addition, adjuvants may also enhance and potentially broaden the immune response by promoting the immunogenicity of weakly immunogenic antigens. Only a few adjuvants are currently licensed for use in vaccines (O'Hagan, et al. doi: 10.1016/j.vaccine.2015.01.088). Moreover, the majority of existing vaccines contain a single adjuvant and recent evidence suggests that it is unlikely to be sufficient for induction of a protective immune response against many emerging infectious diseases. (Underhill, doi: 10.1111/j.1600-065X.2007.00548.x).

### Definitions

A composition is comprised of “substantially all” of a particular compound, or a particular form a compound (e.g., an isomer) when a composition comprises at least about 90%, and at least about 95%, 99%, and 99.9%, of the particular composition on a weight basis. A composition comprises a “mixture” of compounds, or forms of the same compound, when each compound (e.g., isomer) represents at least about 10% of the composition on a weight basis.

The term “toll-like receptor agonist” (TLR agonist) refers to a molecule that binds to a TLR. Synthetic TLR agonists are chemical compounds that are designed to bind to a TLR and activate the receptor. A TLR agonist, or a conjugate thereof, can be prepared as an acid salt or as a base salt, as well as in free acid or free base forms. In solution, certain of the compounds of the disclosure may exist as zwitterions, wherein counter ions are provided by the solvent molecules themselves, or from other ions dissolved or suspended in the solvent.

Within the present disclosure it is to be understood that a compound as described herein or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

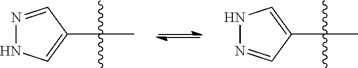 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

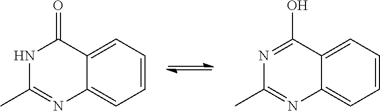

is an example of tautomerism. Accordingly, a structure depicted herein as one tautomer is intended to also include the other tautomer.

### Optical Isomerism

It will be understood that when compounds of the present invention contain one or more chiral centers, the compounds may exist in, and may be isolated as pure enantiomeric or diastereomeric forms or as racemic mixtures. The present disclosure therefore includes any possible enantiomers, diastereomers, racemates or mixtures thereof of the compounds of the disclosure.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

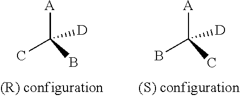

The present disclosure is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. In one embodiment, the isolated isomer is at least about 80%, e.g., at least 90%, 98% or 99% pure, by weight.

Isolated optical isomers may be purified from racemic mixtures by well-known chiral separation techniques. According to one such method, a racemic mixture of a compound of the disclosure, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, behenic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present disclosure can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be employed. Lists of suitable salts are found in Remington's Pharmaceutical Sciences, 17th ed., Mack Publishing Company, Easton, PA, p. 1418 (1985), the disclosure of which is hereby incorporated by reference.

The compounds of the formulas described herein can be solvates, and in some embodiments, hydrates. The term “solvate” refers to a solid compound that has one or more solvent molecules associated with its solid structure. Solvates can form when a compound is crystallized from a solvent. A solvate forms when one or more solvent molecules become an integral part of the solid crystalline matrix upon solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms. In one embodiment those groups have 10 or fewer carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

The term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl” or “Het” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (e.g., from 1 to 3 rings) that are fused together (i.e., a fused ring aryl) or linked covalently. A fused ring aryl refers to 15 multiple rings fused together wherein at least one of the fused rings is an aryl ring. The term “heteroaryl” refers to aryl groups (or rings) that contain at least one heteroatom selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR″C(NR′R″R′″)═NR″″, —NR—C(NR′R″)═NR″, —S(O)R′, —S(O)₂R′, —S(O)₂NR′R″, —NRSO₂R′, —CN, and —NO₂ in a number ranging from zero to (2 m′+1), where m′ is the total number of carbon atoms in such radical. R′, R″, R′″, and R″″ in one embodiment each independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the disclosure includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)z, fluoro(C₁-C₄)alkoxy, and fluoro(C₁-C₄)alkyl, in a number ranging from zero to the total number of open valences on the aromatic ring system; and where R′, R″, R′″, and R″″ are in one embodiment independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, O-(alkylene)_{0,1}-phosphate, O-sulfate, and a phospholipid as defined herein. When a compound of the disclosure includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula-A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where sand dare independently integers of from 0 to 3, and X′ is —O—, —NR′—, —S—, —S(O)—, —S(O)₂—, or —S(O)₂NR′—. The substituents R, R′, R″, and R′″ are in one embodiment independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

A “phospholipid” as the term is used herein refers to a glycerol mono- or diester bearing a phosphate group bonded to a glycerol hydroxyl group with an alkanolamine group being bonded as an ester to the phosphate group, of the general formula

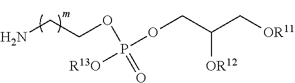

wherein R¹¹ and R¹² are each independently hydrogen or an acyl group, and R¹³ is a negative charge or a hydrogen, depending upon pH. When R13 is a negative charge, a suitable counterion, such as a sodium ion, can be present. For example, the alkanolamine moiety can be an ethanolamine moiety, such that m=1. It is also understood that the NH group can be protonated and positively charged, or unprotonated and neutral, depending upon pH. For example, the phospholipid can exist as a zwitterion with a negatively charged phosphate oxy anion and a positively charged protonated nitrogen atom. The carbon atom bearing OR¹² is a chiral carbon atom, so the molecule can exist as an R isomer, an S isomer, or any mixture thereof. When there are equal amounts of R and S isomers in a sample of the compound of formula (II-A), the sample is referred to as a “racemate.” For example, in the commercially available product 1,2-dioleoyl-sn-glycero-3-phosphoethanolamine, as used in Example I below, the R³ group is of the chiral structure

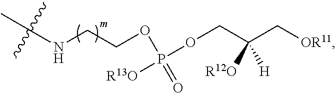

which is of the R absolute configuration.
A phospholipid can be either a free molecule, or covalently linked to another group for example as shown

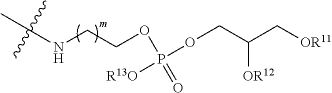

wherein a wavy line indicates a point of bonding.

Accordingly, when a substituent group, such as R³ of the compound of formula (I) herein, is stated to be a phospholipid what is meant that a phospholipid group is bonded as specified by the structure to another group, such as to an N-benzyl heterocyclic ring system as disclosed herein. The point of attachment of the phospholipid group can be at any chemically feasible position unless specified otherwise, such as by a structural depiction. For example, in the phospholipid structure shown above, the point of attachment to another chemical moiety can be via the ethanolamine nitrogen atom, for example as an amide group by bonding to a carbonyl group of the other chemical moiety, for example

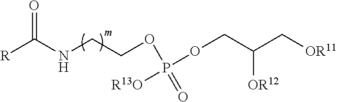

wherein R represents the other chemical moiety to which the phospholipid is bonded. In this bonded, amide derivative, the R¹³ group can be a proton or can be a negative charge associated with a counterion, such as a sodium ion. The acylated nitrogen atom of the alkanolamine group is no longer a basic amine, but a neutral amide, and as such is not protonated at physiological pH.

An “acyl” group as the term is used herein refers to an organic structure bearing a carbonyl group through which the structure is bonded, e.g., to glycerol hydroxyl groups of a phospholipid, forming a “carboxylic ester” group. Examples of acyl groups include fatty acid groups such as oleoyl groups, that thus form fatty (e.g., oleoyl) esters with the glycerol hydroxyl groups. Accordingly, when R¹¹ or R¹², but not both, are acyl groups, the phospholipid shown above is a mono-carboxylic ester, and when both R¹¹ and R¹² are acyl groups, the phospholipid shown above is a di-carboxylic ester.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —CCl₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from: oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is, for example, a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalky1.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine agonist activity using the standard tests described herein, or using other similar tests which are well known in the art. It is also understood by those of skill in the art that the compounds described herein include their various tautomers, which can exist in various states of equilibrium with each other.

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing symptoms of a condition. A candidate molecule or compound described herein may be in an amount in a formulation or medicament, which is an amount that can lead to a biological effect, or lead to ameliorating, alleviating, lessening, relieving, diminishing or removing symptoms of a condition, e.g., disease, for example. The terms also can refer to reducing or stopping a cell proliferation rate (e.g., slowing or halting tumor growth) or reducing the number of proliferating cancer cells (e.g., removing part or all of a tumor). These terms also are applicable to reducing a titre of a microorganism (microbe) in a system (e.g., cell, tissue, or subject) infected with a microbe, reducing the rate of microbial propagation, reducing the number of symptoms or an effect of a symptom associated with the microbial infection, and/or removing detectable amounts of the microbe from the system. Examples of microbe include but are not limited to virus, bacterium and fungus.

The term “therapeutically effective amount” as used herein refers to an amount of a compound, or an amount of a combination of compounds, to treat or prevent a disease or disorder, or to treat a symptom of the disease or disorder, in a subject. As used herein, the terms “subject” and “patient” generally refers to an individual who will receive or who has received treatment (e.g., administration of a compound) according to a method described herein.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present disclosure.

The terms “subject,” “patient” or “subject in need thereof” refers to a living organism suffering from or prone to a disease or condition that can be treated by administration of a compound, pharmaceutical composition, mixture or vaccine as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is a parrot. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a hamster. In some embodiments, a patient is a test animal. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

The term “effective amount” as used herein refers to an amount effective to achieve an intended purpose. Accordingly, the terms “therapeutically effective amount” and the like refer to an amount of a compound, mixture or vaccine, or an amount of a combination thereof, to treat or prevent a disease or disorder, or to treat a symptom of the disease or disorder, in a subject in need thereof.

The term “TLR” refers to Toll-like receptors which are components of the innate immune system that regulate NF_{κ}B activation.

The terms “TLR modulator,” “TLR immunomodulator” and the like as used herein refer, in the usual and customary sense, to compounds which agonize or antagonize a Toll Like Receptor. See e.g., PCT/US2010/000369, Hennessy, E. J., et al., Nature Reviews 2010, 9:283-307; PCT/US2008/001631; PCT/US2006/032371; PCT/US2011/000757. Accordingly, a “TLR agonist” is a TLR modulator which agonizes a TLR, and a “TLR antagonist” is a TLR modulator which antagonizes a TLR.

The term “TLR4” as used herein refers to the product of the TLR4 gene, and homologs, isoforms, and functional fragments thereof: Isoform 1 (NCBI Accession NP_612564.1); Isoform 2 (NCBI Accession NP_003257.1); Isoform 3 (NCBI Accession NP_612567.1). Agonists of TLR4 that may be included in the disclosed formulations include but are not limited, a compound of formula (II-A), e.g., a pyrimidoindole, aminoalkyl glucosaminide phosphates, e.g., CRX-601 and CRX-547), RC-29, monophosphoryl lipid A (MPL), glucopyranosyl lipid adjuvant (GLA and SLA), OM-174, PET Lipid A, ONO-4007, INI-2004 (a di-amine allose phosphate), and E6020.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms. In one embodiment those groups have 10 or fewer carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃. The term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (e.g., from 1 to 3 rings) that are fused together (i.e., a fused ring aryl) or linked covalently. A fused ring aryl refers to 15 multiple rings fused together wherein at least one of the fused rings is an aryl ring. The term “heteroaryl” refers to aryl groups (or rings) that contain at least one heteroatom selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR″, —S(O)R′, —S(O)₂R′, —S(O)₂NR′R″, —NRSO₂R′, —CN, and —NO₂ in a number ranging from zero to (2 m′+1), where m′ is the total number of carbon atoms in such radical. R′, R″, R′″, and R″″ in one embodiment each independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the disclosure includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)z, fluoro(C₁-C₄)alkoxy, and fluoro(C₁-C₄)alkyl, in a number ranging from zero to the total number of open valences on the aromatic ring system; and where R′, R″, R′″, and R″″ are in one embodiment independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the disclosure includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula-A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R)_{d}—, where sand dare independently integers of from 0 to 3, and X′ is —O—, —NR′—, —S—, —S(O)—, —S(O)₂—, or —S(O)₂NR′—. The substituents R, R′, R″, and R′″ are in one embodiment independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —CCl₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from: oxo, —OH, —NH₂, —SH, —CN, —CF₃, —CCl₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is, for example, a substituted or unsubstituted C₁—C alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalky1.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

### Extracellular Vesicles

Extracellular vesicles (EVs) transfer antigens and immunomodulatory molecules in immunologic synapses as a part of intracellular communication, and EVs equipped with immunostimulatory functions can be utilized for vaccine formulation. Hence, we sought small molecule compounds that increase immunostimulatory EVs released by antigen-presenting dendritic cells (DCs) for enhancement of vaccine immunogenicity.

### Compounds

The present disclosure provides in various embodiments compounds, their pharmaceutically acceptable salts, and use of the compounds in the methods that are described herein.

### 634 Compound Series

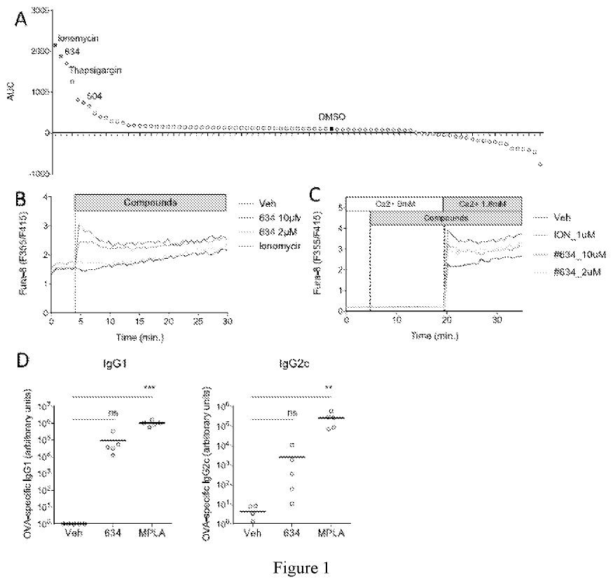

In some embodiments, the present disclosure provides compounds premised upon Compound #634. The compounds enhance exosome release as assayed by THP-1 CD63 reporter cells, but also induce calcium uptake into cells as its unique mechanism of action. Thus, SAR studies were employed to identify compounds in this series. The structure of compound #634 is described herein throughout. It is a sulfonamide analog formed by coupling of benzothiadiazole ring and substituted thiazole ring. The different sites of modification are shown in in FIG. 1B. Thus, in various embodiments, the compounds are of formula (IV):

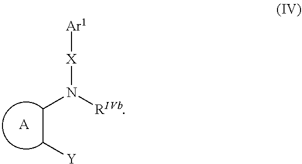

Ar¹ is selected from the group consisting of monocyclic or bicyclic C₆-C₁₀-aryl and bicyclic 9- to 10-membered heteroaryl (wherein 1-4 heteroaryl members are independently selected from N, O, S, and Se), optionally substituted with 1 to 3 R^{IVc}.

Ring A is a monocyclic or bicyclic 5- to 10-membered fully or partially saturated heterocycloalkyl (wherein 1 to 4 ring members are independently selected from N, O, and S) or 5- to 10-membered heteroaryl (wherein 1-4 heteroaryl members are independently selected from N, O, and S), optionally substituted with 1 to 3 R^{IVc}.

X is —SO₂—,—(HN)S(O)—, or —C(O)—.

In some embodiments, Y is selected from (a)

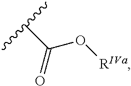

wherein R^{IVa} is selected from the group consisting of H, C₁-C₆-alkyl, C₃-C₁₀-cycloalkyl, C₆-C₁₀-aryl, —C₁-C₆-alkyl(C₆-C₁₀-aryl), and —C₁-C₆-alkyl-NRR′ (wherein R and R′ are independently selected from H, C₁-C₆-alkyl, —C(O)OC₁-C₆-alkyl, —C(O)C₀-C₆-alkyl(fluorophore or biotin)).

In other embodiments, Y is selected from (b) H, halo, CN, C(O)NRR′, N(R)C(O)(C₁-C₆-alkyl), and 5- to 10-membered heteroaryl (wherein 1-4 heteroaryl members are independently selected from N, O, and S) that is optionally substituted with 1 to 3 R^{IVc}, and —C(O)(C₁-C₆)(biotin).

R^{IVb} is H or C₁-C₆-alkyl.

R^{IVc} in each instance is independently selected from the group consisting of C₁-C₆-alkyl, OH, NH₂, halo, —C(O)C₁-C₆-alkyl, —C(O)OC₁-C₆-alkyl, C₆-C₁₀-aryl, and 5- to 10-membered heteroaryl (wherein 1-4 heteroaryl members are independently selected from N, O, and S) optionally substituted with 1 to 3 C₁-C₆-alkyl.

In some embodiments, X is —SO₂—.

In additional embodiments, Y is (a)

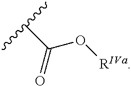

In other embodiments, Ar¹ is a bicyclic 9- to 10-membered heteroaryl (wherein 1-4 heteroaryl members are independently selected from N, O, S, and Se), optionally substituted with 1 to 3 R^{IVc}. Illustrative examples of Ar¹ include optionally substituted:

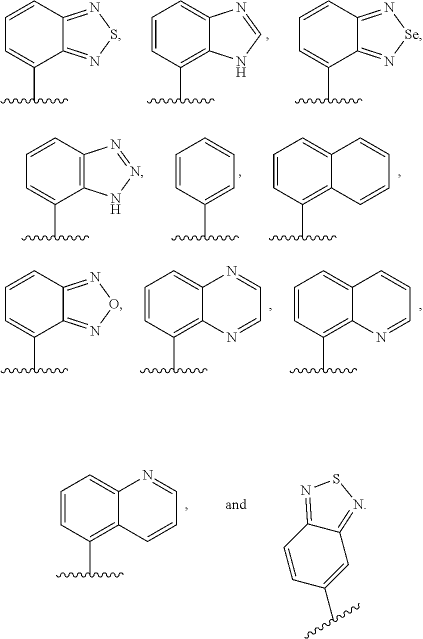

In additional embodiments, Ring A is a 5- to 10-membered heteroaryl (wherein 1-4 heteroaryl members are independently selected from N, O, and S), optionally substituted with 1 to 3 R^{IVc}. In illustrative embodiments, Ring A is a 5-membered heteroaryl (wherein 1-2 heteroaryl members are independently selected from N and S), optionally substituted with 1 to 3 R^{IVc}. Examples of Ring A, in various embodiments, include optionally substituted:

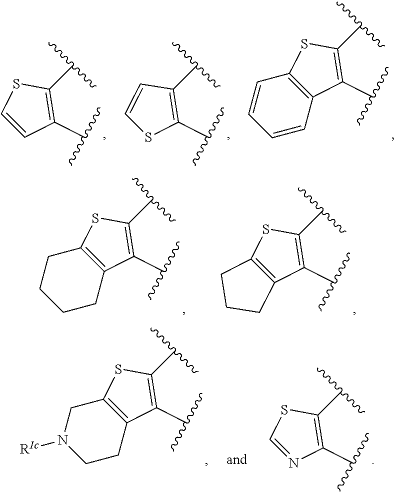

In additional embodiments, R^{IVa} is selected from the group consisting of H, C₁-C₆-alkyl, C₃-C₁₀-cycloalkyl, C₆-C₁₀-aryl, —C₁-C₆-alkyl(C₆-C₁₀-aryl), —C₁-C₆-alkyl-NRR′ (wherein R and R′ are independently selected from H, C₁-C₆-alkyl, and —C(O)OC₁-C₆-alkyl).

In still additional embodiments, the compound of formula (IV) is of formula (IVA):

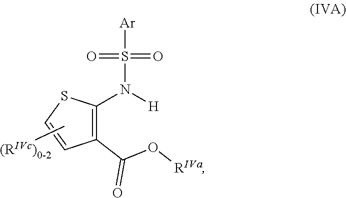

wherein
Ar¹ is a bicyclic 9- to 10-membered heteroaryl (wherein 1-4 heteroaryl members are independently selected from N, O, S, and Se), optionally substituted with 1 to 3 R^{IVc}.

### 645 Compound Series

The HTS described herein identified hit compound #645 (FIG. 9, structure shown in black box) that belongs to 3-pyridyl-oxadiazole chemotype, and it was found to be a good adjuvant (in vivo) that enhanced the upregulation of CD63 and other co-stimulatory molecules including CD80, 83, 86 and MHC class II (in vitro). Thus, on this premise, the present disclosure provides in additional embodiments a compound of formula (V) or a pharmaceutically acceptable salt thereof:

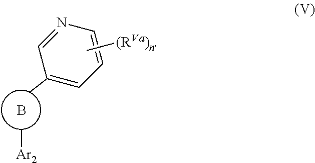

Ring B is 5-membered heteroaryl (wherein 1-4 heteroaryl members are independently selected from N, O, and S).
Ar² is selected from the group consisting of C₁-C₆-alkyl, monocyclic or bicyclic C₆-C₁₀-aryl, monocyclic or bicyclic 5- to 10-membered heteroaryl (wherein 1-4 heteroaryl members are independently selected from N, O, and S), and monocyclic or bicyclic 5- to 10-membered fully or partially saturated heterocycloalkyl (wherein 1 to 4 ring members are independently selected from N, O, and S), wherein Ar² is optionally substituted with 1 to 3 R^{IIIb}.
R^{Va} is selected from the group consisting of C₁-C₆-alkyl, C₁-C₆-haloalkyl, halo, C₆-C₁₀-aryl, —O(C₆-C₁₀-aryl), —S(C₆-C₁₀-aryl), and 5- to 10-membered heteroaryl (wherein 1-4 heteroaryl members are independently selected from N, O, and S), wherein aryl or heteroaryl is optionally substituted with 1 to 3 substituents selected from C₁-C₆-alkyl and halo.
n is 0, 1, or 2.
R^{Vb} is selected from the group consisting of C₁-C₆-alkyl, —OC₁-C₆-alkyl, C₁-C₆-haloalkyl, —OC₁-C₆-haloalkyl, halo, oxo, NO₂, CN, NRR′ (wherein R and R′ are independently selected from H and C₁-C₆-alkyl).

In some embodiments, Ring B is selected from the group consisting of:

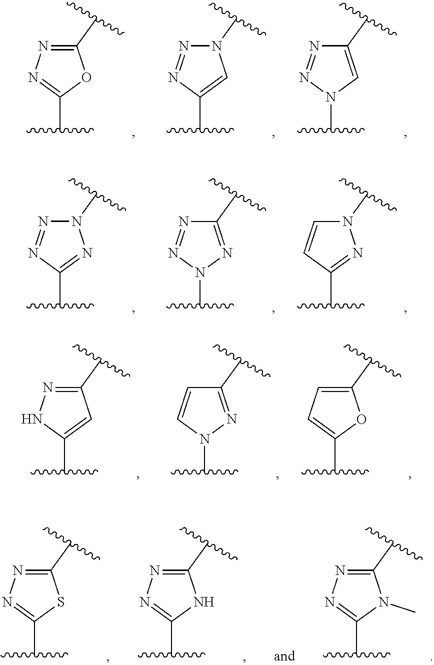

In additional embodiments, Ar² is optionally substituted monocyclic or bicyclic C₆-C₁₀-aryl. In an embodiment, Ar² is optionally substituted phenyl. In another embodiment, Ar² is optionally substituted naphthyl.

In still additional embodiments, Ar² is optionally substituted monocyclic or bicyclic 5- to 10-membered heteroaryl (wherein 1-4 heteroaryl members are independently selected from N, O, and S). In an embodiment, Ar² is optionally substituted monocyclic 5- to 6-membered heteroaryl (wherein 1-4 heteroaryl members are independently selected from N, O, and S).

In other embodiments, n is 1 and R^{IIIa} is optionally substituted thiophenyl.

Exemplary embodiments include the specific compounds in the following table:

| | |
|---|---|
| 645 | 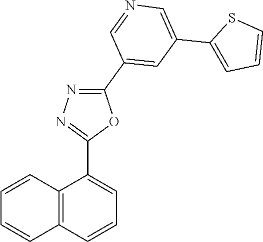 [SMILES: c1csc(-c2cncc(-c3nnc(-c4cccc5ccccc45)o3)c2)c1] |
| 1301 | 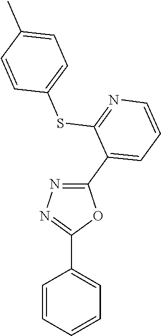 [SMILES: Cc1ccc(Sc2ncccc2-c2nnc(-c3ccccc3)o2)cc1] |
| 1302 | 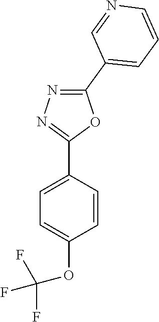 [SMILES: FC(F)(F)Oc1ccc(-c2nnc(-c3cccnc3)o2)cc1] |
| 1303 | 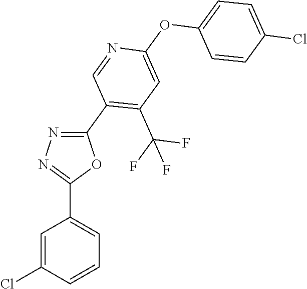 [SMILES: FC(F)(F)c1cc(Oc2ccc(Cl)cc2)ncc1-c1nnc(-c2cccc(Cl)c2)o1] |
| 1304 | 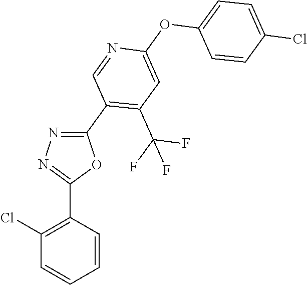 [SMILES: FC(F)(F)c1cc(Oc2ccc(Cl)cc2)ncc1-c1nnc(-c2ccccc2Cl)o1] |
| 1305 | 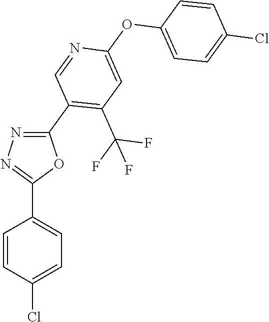 [SMILES: FC(F)(F)c1cc(Oc2ccc(Cl)cc2)ncc1-c1nnc(-c2ccc(Cl)cc2)o1] |
| 1310 | 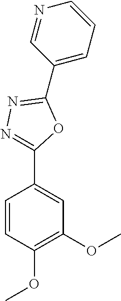 [SMILES: COc1ccc(-c2nnc(-c3cccnc3)o2)cc1OC] |
| 1306 | 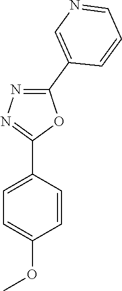 [SMILES: COc1ccc(-c2nnc(-c3cccnc3)o2)cc1] |
| 1311 | 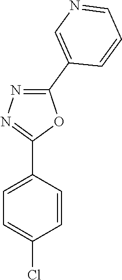 [SMILES: Clc1ccc(-c2nnc(-c3cccnc3)o2)cc1] |
| 1307 | 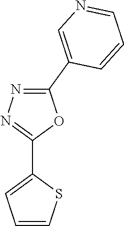 [SMILES: c1cncc(-c2nnc(-c3cccs3)o2)c1] |
| 1308 | 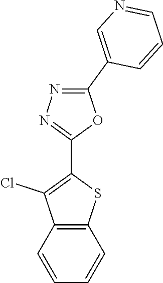 [SMILES: Clc1c(-c2nnc(-c3cccnc3)o2)sc2ccccc12] |
| 1309 | 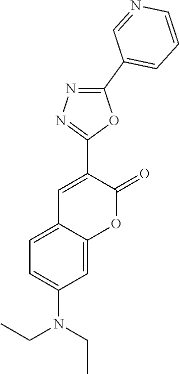 [SMILES: CCN(CC)c1ccc2cc(-c3nnc(-c4cccnc4)o3)c(=O)oc2c1] |
| 2C176 | 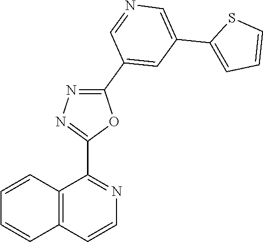 [SMILES: c1csc(-c2cncc(-c3nnc(-c4nccc5ccccc45)o3)c2)c1] |
| 2C173 | 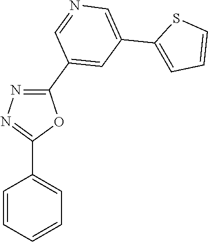 [SMILES: c1ccc(-c2nnc(-c3cncc(-c4cccs4)c3)o2)cc1] |
| 2C201 | 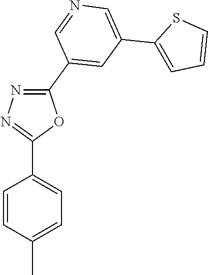 [SMILES: Cc1ccc(-c2nnc(-c3cncc(-c4cccs4)c3)o2)cc1] |
| 2H019 | 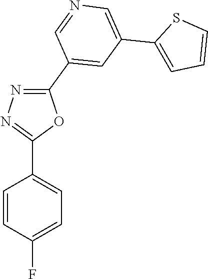 [SMILES: Fc1ccc(-c2nnc(-c3cncc(-c4cccs4)c3)o2)cc1] |
| 2H022 | 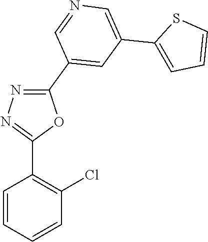 [SMILES: Clc1ccccc1-c1nnc(-c2cncc(-c3cccs3)c2)o1] |
| 2H023 | 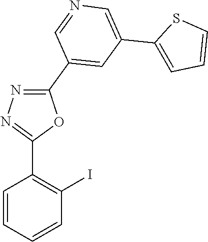 [SMILES: Ic1ccccc1-c1nnc(-c2cncc(-c3cccs3)c2)o1] |
| 2H028 | 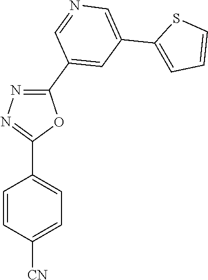 [SMILES: N#Cc1ccc(-c2nnc(-c3cncc(-c4cccs4)c3)o2)cc1] |
| 2H027 | 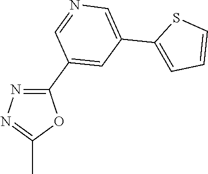 [SMILES: Cc1nnc(-c2cncc(-c3cccs3)c2)o1] |
| 2H032 | 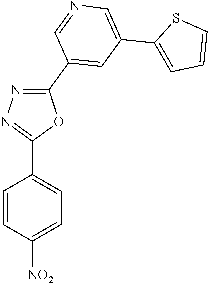 [SMILES: O=[N+]([O-])c1ccc(-c2nnc(-c3cncc(-c4cccs4)c3)o2)cc1] |
| 2H037 | 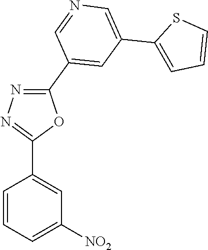 [SMILES: O=[N+]([O-])c1cccc(-c2nnc(-c3cncc(-c4cccs4)c3)o2)c1] |
| 2C211 | 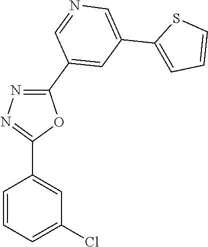 [SMILES: Clc1cccc(-c2nnc(-c3cncc(-c4cccs4)c3)o2)c1] |
| 2F188B | 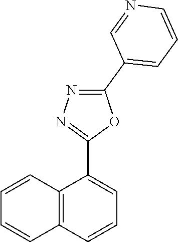 [SMILES: c1cncc(-c2nnc(-c3cccc4ccccc34)o2)c1] |
| 2F187 | 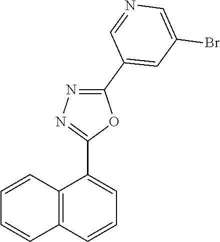 [SMILES: Brc1cncc(-c2nnc(-c3cccc4ccccc34)o2)c1] |
| 2C178 | 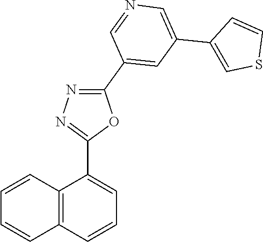 [SMILES: c1ccc2c(-c3nnc(-c4cncc(-c5ccsc5)c4)o3)cccc2c1] |
| 2H005 | 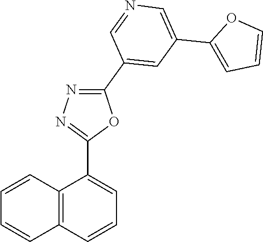 [SMILES: c1coc(-c2cncc(-c3nnc(-c4cccc5ccccc45)o3)c2)c1] |
| 2C179 | 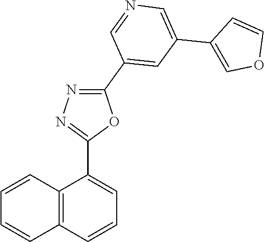 [SMILES: c1ccc2c(-c3nnc(-c4cncc(-c5ccoc5)c4)o3)cccc2c1] |
| 2C181 | 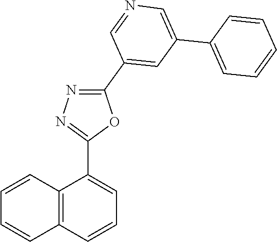 [SMILES: c1ccc(-c2cncc(-c3nnc(-c4cccc5ccccc45)o3)c2)cc1] |
| 2C183 | 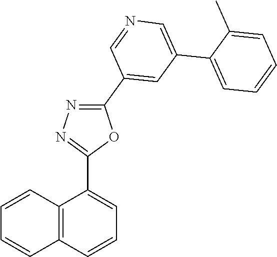 [SMILES: Cc1ccccc1-c1cncc(-c2nnc(-c3cccc4ccccc34)o2)c1] |
| 2C185 | 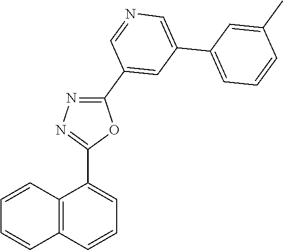 [SMILES: Cc1cccc(-c2cncc(-c3nnc(-c4cccc5ccccc45)o3)c2)c1] |
| 2C182 | 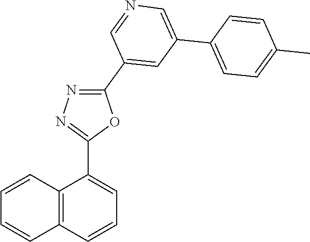 [SMILES: Cc1ccc(-c2cncc(-c3nnc(-c4cccc5ccccc45)o3)c2)cc1] |
| 2H008 | 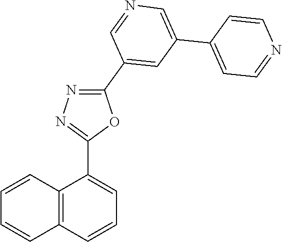 [SMILES: c1ccc2c(-c3nnc(-c4cncc(-c5ccncc5)c4)o3)cccc2c1] |
| 2C188 | 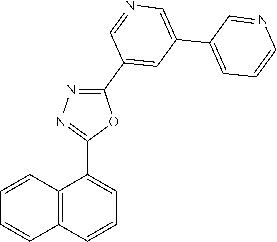 [SMILES: c1cncc(-c2cncc(-c3nnc(-c4cccc5ccccc45)o3)c2)c1] |
| 2H050 | 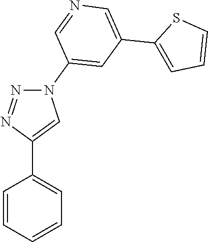 [SMILES: c1ccc(-c2cn(-c3cncc(-c4cccs4)c3)nn2)cc1] |
| 2H042 | 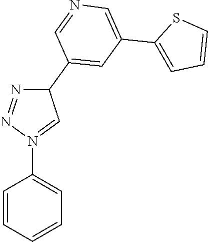 |
| 2E279 | 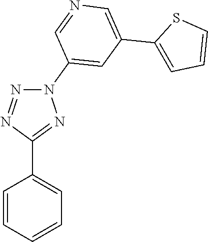 [SMILES: c1ccc(-c2nnn(-c3cncc(-c4cccs4)c3)n2)cc1] |
| 2E277 | 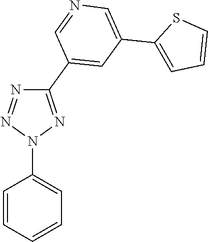 [SMILES: c1ccc(-n2nnc(-c3cncc(-c4cccs4)c3)n2)cc1] |
| 2C214 | 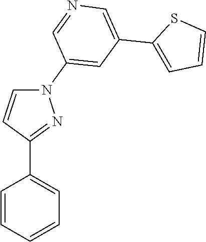 [SMILES: c1ccc(-c2ccn(-c3cncc(-c4cccs4)c3)n2)cc1] |
| 2E280 | 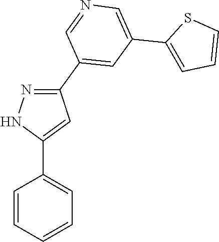 [SMILES: c1ccc(-c2cc(-c3cncc(-c4cccs4)c3)n[nH]2)cc1] |
| | 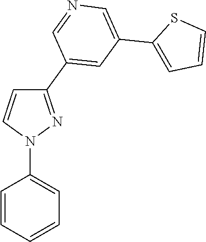 [SMILES: c1ccc(-n2ccc(-c3cncc(-c4cccs4)c3)n2)cc1] |
| | 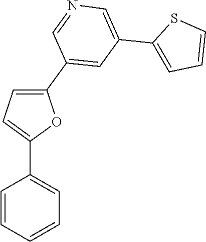 [SMILES: c1ccc(-c2ccc(-c3cncc(-c4cccs4)c3)o2)cc1] |
| | 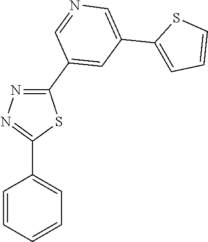 [SMILES: c1ccc(-c2nnc(-c3cncc(-c4cccs4)c3)s2)cc1] |
| | 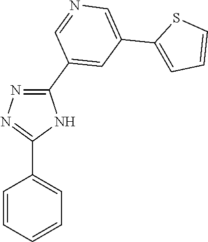 [SMILES: c1ccc(-c2nnc(-c3cncc(-c4cccs4)c3)[nH]2)cc1] |
| | 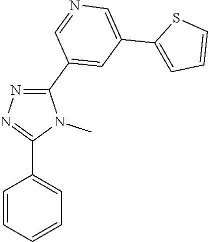 [SMILES: Cn1c(-c2ccccc2)nnc1-c1cncc(-c2cccs2)c1] |

### TLR7 Ligands

Recent advances in cancer immunotherapy have improved patient survival. However, only a minority patients with pulmonary metastatic disease responds to treatment with checkpoint inhibitors. As an alternate approach, we have tested the ability of systemically administered 1V270, a toll-like receptor 7 (TLR7) agonist conjugated to a phospholipid, to inhibit lung metastases in two variant murine 4T1 breast cancer models, as well as in B16 melanoma, and Lewis lung c models.

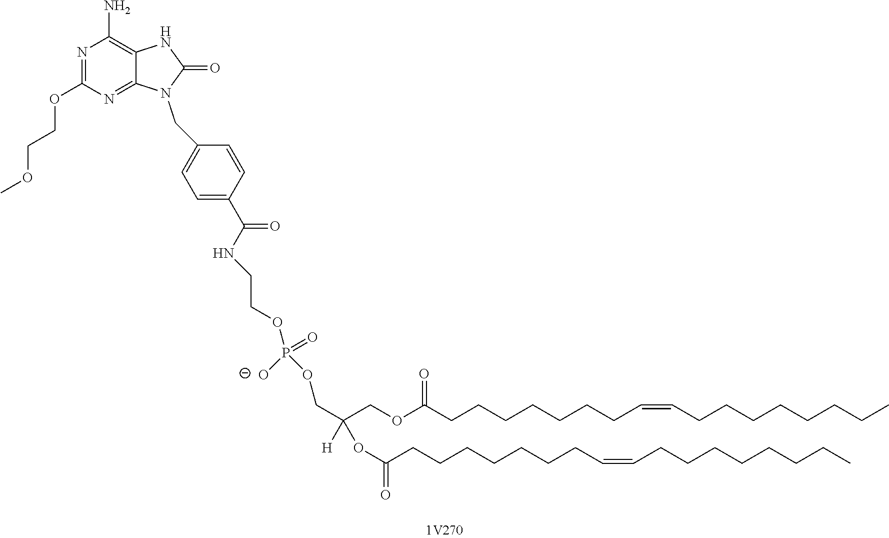 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

In the 4T1 breast cancer models, 1V270 therapy inhibited lung metastases if given up to a week after primary tumor initiation. The treatment protocol was facilitated by the minimal toxic effects exerted by the phospholipid TLR7 agonist, compared·to the unconjugated agonist. The 1V270 therapy inhibited colonization by tumor cells in the lungs in a NK cell dependent manner. Additional experiments revealed that single administration of 1V270 led to tumor-specific CD8⁺ cell-dependent adaptive immune responses that suppressed late stage metastatic tumor growth in the lungs. T cell receptor (TCR) repertoire analyses showed that 1V270 therapy induced oligoclonal tumor-specific T cells in the lungs and regional lymph nodes. Different animals displayed commonly shared TCR clones following 1V270 therapy. Intranasal administration of 1V270 also suppressed lung metastasis and induced tumor-specific adaptive immune responses. These results indicate that systemic 1V270 therapy can induce tumor-specific cytotoxic T cell responses to pulmonary metastatic cancers, and that TCR repertoire analyses can be used to monitor, and to predict, the response to therapy.

In various embodiments, the present disclosure provides a TLR7 ligand of formula (I), or a tautomer, or pharmaceutically acceptable salt or solvate thereof:

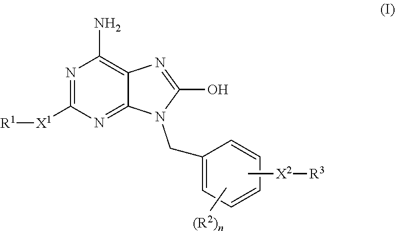

wherein X¹ is —O—, —S—, or —NR^{c}—.
R¹ is hydrogen, (C₁-C₁₀)alkyl, substituted (C₁-C₁₀)alkyl, C₆₋₁₀ aryl, or substituted C₆₋₁₀ aryl, C₅₋₉ heterocyclic, substituted C₅₋₉ heterocyclic.
R^{c} is hydrogen, C₁₋₁₀ alkyl, or substituted C₁₋₁₀ alkyl; or R^{c} and R¹ taken together with the nitrogen to which they are attached form a heterocyclic ring or a substituted heterocyclic ring.

Each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent.

Each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl.

With respect to formula (I), the substituents on any alkyl, aryl or heterocyclic groups include hydroxy, C₁₋₆ alkyl, hydroxyC₁₋₆ alkylene, C₁₋₆ alkoxy, C₃₋₆ cycloalkyl, C₁₋₆ alkoxyC₁₋₆ alkylene, amino, cyano, halo, or aryl.
n is 0, 1, 2, 3 or 4.
X² is a bond or a linking group.
R³ is a phospholipid comprising one or two carboxylic esters.

In one embodiment, the composition of the disclosure comprises nanoparticles comprising a compound of formula (I). As used herein, a nanoparticle has a diameter of about 30 nm to about 600 nm, or a range with any integer between 30 and 600, e.g., about 40 nm to about 250 nm, including about 40 to about 80 or about 100 nm to about 150 nm in diameter. The nanoparticles may be formed by mixing a compound of formula (I), which may spontaneously form nanoparticles, or by mixing a compound of formula (I) with a preparation of lipids, such as phospholipids including but not limited to phosphatidylcholine, phosphatidylserine or cholesterol, thereby forming a nanoliposome. Optionally, a compound of formula (I), a lipid preparation and a glycol such as propylene glycol are combined.

In one embodiment, the present disclosure provides a composition comprising an amount of a compound of Formula (I), or a tautomer thereof, or a pharmaceutically acceptable salt or solvate thereof. Optionally, the composition further comprises an antigen. In one embodiment, the composition having an antigen is administered concurrently, prior to or subsequent to administration of the composition having a compound of formula (I).

In some embodiments, R³ can comprise a group of formula

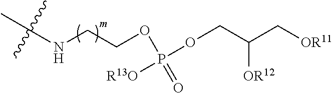

wherein R¹¹ and R¹² are each independently a hydrogen or an acyl group, R¹³ is a negative charge or a hydrogen, and m is 1 to 8, wherein a wavy line indicates a position of bonding, wherein an absolute configuration at the carbon atom bearing OR¹² is R, S, or any mixture thereof.

For example, in an embodiment, m can be 1, providing a glycerophosphatidylethanolamine. More specifically, R¹¹ and R¹² can each be oleoyl groups.

In various embodiments, the phospholipid of R³ can comprise two carboxylic esters and each carboxylic ester includes one, two, three or four sites of unsaturation, epoxidation, hydroxylation, or a combination thereof.

In various embodiments, the phospholipid of R³ can comprise two carboxylic esters and the carboxylic esters of are the same or different. More specifically, each carboxylic ester of the phospholipid can be a C17 carboxylic ester with a site of unsaturation at C8-C9. Alternatively, each carboxylic ester of the phospholipid can be a C18 carboxylic ester with a site of unsaturation at C9-C10.

In various embodiments, X² can be a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. The chain can be interspersed with one or more cycloalkyl, aryl, heterocyclyl, or heteroaryl rings.

In various embodiments, X² can be C(O), or can be any of

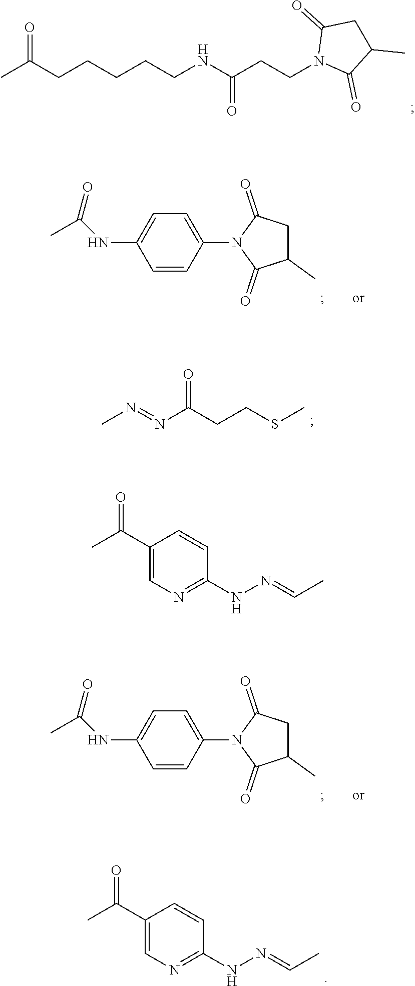

In various embodiments, R³ can be dioleoylphosphatidyl ethanolamine (DOPE).

In various embodiments, R³ can be 1,2-dioleoyl-sn-glycero-3-phospho ethanolamine and X² can be C(O).

In various embodiments, X¹ can be oxygen.

In various embodiments, X¹ can be sulfur, or can be —NR^{c}— where R^{c} is hydrogen, C₁₋₆ alkyl or substituted C₁₋₆ alkyl, where the alkyl substituents are hydroxy, C₃₋₆ cycloalkyl, C₁₋₆ alkoxy, amino, cyano, or aryl. More specifically, X¹ can be —NH—.

In various embodiments, R¹ and R^{c} taken together can form a heterocyclic ring or a substituted heterocyclic ring. More specifically, R¹ and R^{c} taken together can form a substituted or unsubstituted morpholino, piperidino, pyrrolidino, or piperazino ring.

In various embodiments R¹ can be a C₁-C₁₀ alkyl substituted with C₁₋₆ alkoxy.

In various embodiments, R¹ can be hydrogen, C₁₋₄ alkyl, or substituted C₁₋₄ alkyl. More specifically, R¹ can be hydrogen, methyl, ethyl, propyl, butyl, hydroxyC₁₋₄ alkylene, or C₁₋₄ alkoxyC₁₋₄ alkylene. Even more specifically, R¹ can be hydrogen, methyl, ethyl, methoxyethyl, or ethoxyethyl.

In various embodiments, R² can be absent, or R² can be halogen or C₁₋₄ alkyl. More specifically, R² can be chloro, bromo, methyl, or ethyl.

In various embodiments, X¹ can be O, R¹ can be C₁₋₄ alkoxy-ethyl, n can be 1, R² can be hydrogen, X² can be carbonyl, and R³ can be 1,2-dioleoylphosphatidyl ethanolamine (DOPE).

In various embodiments, the compound of Formula (I) can be:

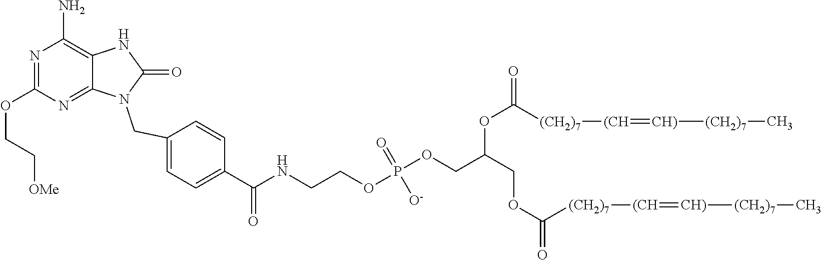

In various embodiments, the compound of formula (I) can be the R-enantiomer of the above structure:

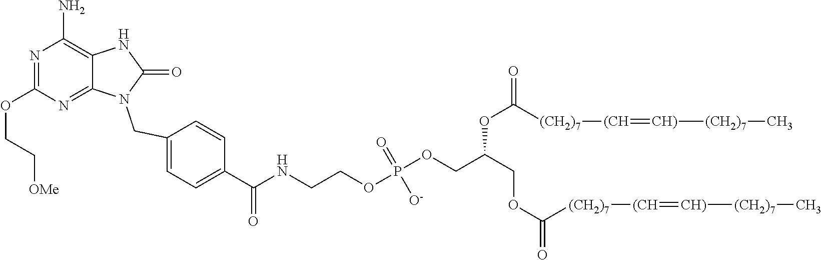

In one embodiment, the TLR7 ligand is:

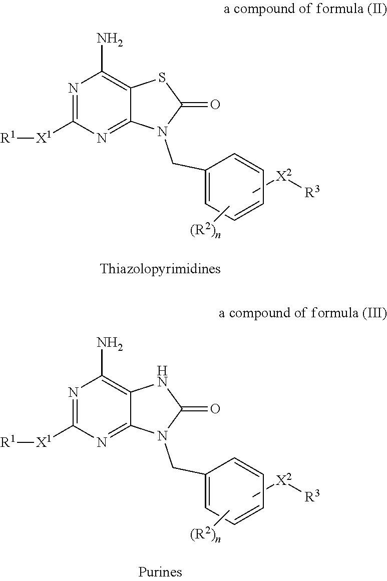

In Formula (II) or (III), X¹ is —O—, —S—, or —NR^{c}—.
R^{c} is hydrogen, C₁₋₁₀ alkyl, or C₁₋₁₀ alkyl substituted by C₃₋₆ cycloalkyl, or R^{c} and R¹ taken together with the nitrogen atom can form a heterocyclic ring or a substituted heterocyclic ring, wherein the substituents are hydroxy, C₁₋₆ alkyl, hydroxy C₁₋₆ alkylene, C₁₋₆ alkoxy, C₁₋₆ alkoxy C₁₋₆ alkylene, or cyano.
R¹ is (C₁-C₁₀)alkyl, substituted (C₁-C₁₀)alkyl, C₆₋₁₀ aryl, or substituted C₆₋₁₀ aryl, C₅₋₉ heterocyclic, substituted C₅₋₉ heterocyc1ic; wherein the substituents on the alkyl, aryl or heterocyclic groups are hydroxy, C₁₋₆ alkyl, hydroxy C₁₋₆ alkylene, C₁₋₆ alkoxy, C₁₋₆ alkoxy C₁₋₆ alkylene, amino, cyano, halogen, or aryl.
R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), —O—C(O)NR^{a}R^{b}, —(C₁-C₆)alkylene-NR^{a}R^{b}, —(C₁-C₆)alkylene-C(O)NR^{a}R^{b}, halo, nitro, or cyano.

Each R^{a} and R^{b} is independently hydrogen, (C₁₋₆)alkyl, (C₃-C₈)cycloalkyl, (C₁₋₆6)alkoxy, halo(C₁₋₆)alkyl, (C₃-C₈)cycloalkyl(C₁₋₆)alkyl, (C₁₋₆) alkanoyl, hydroxy(C₁₋₆)alkyl, aryl, aryl(C₁₋₆)alkyl, aryl, aryl(C₁₋₆)alkyl, Het, Het (C₁₋₆)alkyl, or (C₁₋₆)alkoxycarbony1; wherein X² is a bond or a linking group; wherein R³ is a phospholipid comprising one or two carboxylic esters wherein n is 0, 1, 2, 3, or 4; or a tautomer thereof; or a pharmaceutically acceptable salt thereof.

In formula (I), the alkyl, aryl, heterocyclic groups of R¹ can be optionally substituted with one or more substituents, wherein the substituents are the same or different, and include lower alkyl; cycloalkyl, hydroxyl; hydroxy C₁₋₆ alkylene, such as hydroxymethyl, 2-hydroxyethyl or 3-hydroxypropyl; lower alkoxy; C₁₋₆ alkoxy C₁₋₆ alkyl, such as 2-methoxyethyl, 2-ethoxyethyl or 3-methoxypropyl; amino; alkylamino; dialkyl amino; cyano; nitro; acyl; carboxyl; lower alkoxycarbonyl; halogen; mercapto; C₁₋₆ alkylthio, such as, methylthio, ethylthio, propylthio or butylthio; substituted C₁₋₆ alkylthio, such as methoxyethylthio, methylthioethylthio, hydroxyethylthio or chloroethylthio; aryl; substituted C₆₋₁₀ monocyclic or fused-cyclic aryl, such as 4-hydroxyphenyl, 4-methoxyphenyl, 4-fluorophenyl, 4-chlorophenyl or 3,4-dichlorophenyl; 5-6 membered unsaturated heterocyclic, such as furyl, pyrrolyl, pyrazolyl, imidazolyl, thiazolyl, thienyl, pyridyl or pyrimidinyl; and bicyclic unsaturated heterocyclic, such as indolyl, isoindolyl, quinolyl, benzothiazolyl, chromanyl, benzofuranyl or phthalimido. In certain embodiments, one or more of the above groups can be expressly excluded as a substituent of various other groups of the formulae.

The alkyl, aryl, heterocyclic groups of R² can be optionally substituted with one or more substituents, wherein the substituents are the same or different, and include hydroxyl; C₁₋₆ alkoxy, such as methoxy, ethoxy or propoxy; carboxyl; C₂₋₇ alkoxycarbonyl, such as methoxycarbonyl, ethoxycarbonyl or propoxycarbonyl) and halogen.

The alkyl, aryl, heterocyclic groups of R^{c} can be optionally substituted with one or more substituents, wherein the substituents are the same or different, and include C₃₋₆ cycloalkyl; hydroxyl; C₁₋₆ alkoxy; amino; cyano; aryl; substituted aryl, such as 4-hydroxyphenyl, 4-methoxyphenyl, 4-chlorophenyl or 3,4-dichlorophenyl; nitro and halogen.

The heterocyclic ring formed together with R^{c} and R¹ and the nitrogen atom to which they are attached can be optionally substituted with one or more substituents, wherein the substituents are the same or different, and include C₁₋₆ alkyl; hydroxy C₁₋₆ alkylene; C₁₋₆ alkoxy C₁₋₆ alkylene; hydroxyl; C₁₋₆ alkoxy; and cyano. A specific value for X¹ is a sulfur atom, an oxygen atom or —NR^{c}—.

In other embodiments, the TLR7 ligand has formula (I) wherein R³ is hydrogen, (C₁-C₁₀)alkyl, substituted (C₁-C₁₀)alkyl, C₆₋₁₀ aryl, or substituted C₆₋₁₀ aryl, C₅₋₉ heterocyclic, substituted C₅₋₉ heterocyclic.

In other embodiments, the TLR7 ligand has formula (I), wherein:

R³ is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het(C₁-C₆)alkyl, or (C₁-C₆)alkoxycarbonyl; wherein the substituents on any alkyl, aryl or heterocyclic groups are hydroxy, C₁₋₆alkyl, hydroxyC₁₋₆ alkylene, C₁₋₆ alkoxy, C₃₋₆ cycloalkyl, C₁₋₆ alkoxyC₁₋₆ alkylene, amino, cyano, halo, or aryl.

### TLR4 Ligands

Toll-like receptors (TLRs) are pattern recognition receptors that recognize conserved microbial products, known as pathogen-associated molecular patterns (PAMPs). TLR4 recognizes LPS. TLR4 signaling activates MyD88 and TRIF-dependent pathways. MyD88 pathway activates NF-κB and JNK to induce inflammatory response. TRIF pathway activates IRF3 to induce IFN-α production.

TLR4 is expressed predominately on monocytes, mature macrophages and dendritic cells, mast cells and the intestinal epithelium. TLR modulators (antagonists) for TLR4 include NI-0101 (Hennessy 2010, Id.), 1A6 (Ungaro, R., et al., Am. J. Physiol. Gastrointest. Liver Physiol. 2009, 296:G1167-G1179), AV411 (Ledeboer, A., et al., Neuron Glia Biol. 2006, 2:279-291; Ledeboer, A., et al., Expert Opin. Investig. Drugs 2007, 16:935-950), Eritoran (Mullarkey, M., et al., J. Pharmacol. Exp. Ther. 2003, 305:1093-1102), and TAK-242 (Li, M., et al., Mol. Pharmacol. 2006, 69:1288-1295). TLR modulators (agonists) for TLR4 include Pollinex® Quattro (Baldrick, P., et al., J. Appl. Toxicol. 2007, 27:399-409; DuBuske, L., et al., J. Allergy Clin. Immunol. 2009, 123:S216).

In various embodiments, the present disclosure provides a TLR4 agonist compound having formula (II-A):

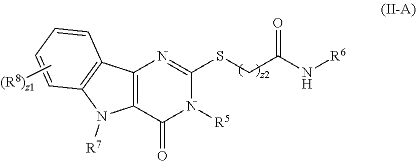

or a pharmaceutically acceptable salt thereof.

In formula (II-A), z1 is an integer from 0 to 4, and z2 is an integer from 0 to 5.
R⁵ is substituted or unsubstituted cycloalkyl, substituted or unsubstituted heterocycloalkyl, substituted or unsubstituted aryl, or substituted or unsubstituted heteroaryl.
R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R⁷ is hydrogen, or substituted or unsubstituted alkyl.
R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R⁵ is R^{5A}-substituted or unsubstituted cycloalkyl, R^{5A} substituted or unsubstituted heterocycloalkyl, R^{5A} substituted or unsubstituted aryl, or R^{5A} substituted or unsubstituted heteroaryl. R^{5A} is independently halogen, —CN, —CF₃, —CCl₃, —OH, —NH₂, —SO₂, —COOH, oxo, nitro, —SH, —CONH₂, —NH—OH, R^{5B}-substituted or unsubstituted alkyl, R^{5B}-substituted or unsubstituted alkynyl, R^{5B}-substituted or unsubstituted heteroalkyl, R^{5B}-substituted or unsubstituted cycloalkyl, R^{5B}-substituted or unsubstituted heterocycloalkyl, R^{5B}-substituted or unsubstituted aryl, or R^{5B}-substituted or unsubstituted heteroaryl. R^{5B} is independently halogen, —CN, —CF₃, —CCl₃, —OH, —NH₂, —SO₂, —COOH, oxo, nitro, —SH, —CONH₂, R^{5C}-substituted or unsubstituted alkyl, R^{5C}-substituted or unsubstituted heteroalkyl, R^{5C}-substituted or unsubstituted cycloalkyl, R^{5C}-substituted or unsubstituted heterocycloalkyl, R^{5C}-substituted or unsubstituted aryl, or R^{5C}-substituted or unsubstituted heteroaryl. R^{5C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

In additional embodiments, R⁶ is R^{6A}-substituted or unsubstituted alkyl, R^{6A} substituted or unsubstituted heteroalkyl, R^{6A} substituted or unsubstituted cycloalkyl, R^{6A} substituted or unsubstituted heterocycloalkyl, R^{6A} substituted or unsubstituted aryl, or R^{6A} substituted or unsubstituted heteroaryl. R^{6A} is independently halogen, —CN, —CF₃, —CCl₃, —OH, —NH₂, —SO₂, —COOH, oxo, nitro, —SH, —CONH₂, R^{6B}-substituted or unsubstituted alkyl, R^{6B}-substituted or unsubstituted heteroalkyl, R^{6B}-substituted or unsubstituted cycloalkyl, R^{6B}-substituted or unsubstituted heterocycloalkyl, R^{6B}-substituted or unsubstituted aryl, or 10 R^{6B}-substituted or unsubstituted heteroaryl. R^{6B} is independently halogen, —CN, —CF₃, —CCl₃, —OH, —NH₂, —SO₂, —COOH, oxo, nitro, —SH, —CONH₂, R^{6C}-substituted or unsubstituted alkyl, R^{6C}-substituted or unsubstituted heteroalkyl, R^{6C}-substituted or unsubstituted cycloalkyl, R^{6C}-substituted or unsubstituted heterocycloalkyl, R^{6C}-substituted or unsubstituted aryl, or R^{6C}-substituted or unsubstituted heteroaryl. R^{6C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

In additional embodiments, R⁷ is hydrogen, or R^{7A}-substituted or unsubstituted alkyl. R^{7A} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

In additional embodiments, R⁸ is independently halogen, —CN, —SH, —OH, —COOH, —NH₂, —CONH₂, nitro, —CF₃, —CCl₃, R^{8A}-substituted or unsubstituted alkyl, R^{8A}-substituted or unsubstituted heteroalkyl, R^{8A} substituted or unsubstituted cycloalkyl, R^{8A}-substituted or unsubstituted heterocycloalkyl, R^{8A} substituted or unsubstituted aryl, or R^{8A}-substituted or unsubstituted heteroaryl. R^{8A} is independently halogen, —CN, —CF₃, —CCl₃, —OH, —NH₂, —SO₂, —COOH, oxo, nitro, —SH, —CONH₂, R^{8B}-substituted or unsubstituted alkyl, R^{8B}-substituted or unsubstituted heteroalkyl, R^{8B}-substituted or unsubstituted cycloalkyl, R^{8B}-substituted or unsubstituted heterocycloalkyl, R^{8B}-substituted or unsubstituted aryl, or R^{8B}-substituted or unsubstituted heteroaryl. R^{8B} is independently halogen, —CN, —CF₃, —CCl₃, —OH, —NH₂, —SO₂, —COOH, oxo, nitro, —SH, —CONH₂, R^{8C}-substituted or unsubstituted alkyl, 8^{4C}-substituted or unsubstituted heteroalkyl, R^{8C}-substituted or unsubstituted cycloalkyl, R^{8C}-substituted or unsubstituted heterocycloalkyl, R^{8C}-substituted or unsubstituted aryl, or R^{8C}-substituted or unsubstituted heteroaryl. R^{8C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl.

In some embodiments, a compound as described herein may include multiple instances of a substituent, e.g., R⁵, R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C}. In such embodiments, each substituent may optional be different at each occurrence and be appropriately labeled to distinguish each group for greater clarity. For example, where each R^{5A} is different, they may be referred to as e.g., R^{5A.1}, R^{5A.2}, R^{5A.3}, R^{5A.4}, R^{5A.5} Similarly, where any of R^{5A}, R^{5B}, R^{5C}, R^{6A}, R^{6B}, R^{6C}, R⁷, R^{7A}, R^{7B}, R^{7C}, R⁸, R^{8A}, R^{8B}, and/or R^{8C} multiply occur, the definition of each occurrence of R^{5A}, R^{5B}, R^{5C}, R^{6A}, R^{6B}, R^{6C}, R′, R^{7A}, R^{7B}, R^{7C}, R⁸, R^{8A}, R^{8B}, and/or R^{8C} assumes the definition of R^{5A}R^{5B}, R^{5C}, R^{6A}, R^{6B}, R^{6C}, R⁷, R^{7A}, R^{7B}, R^{7C}, R⁸, R^{8A}, R^{8B}, and/or R^{8C}, respectively

In another embodiment, there is provided a compound of formula (II-A) as disclosed above, provided, however, that: (i) the compound of formula (II-A) is not

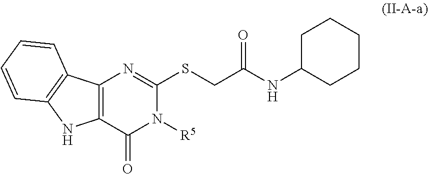

wherein R⁵ is p-fluorophenyl or p-methylphenyl; (ii) the compound is not

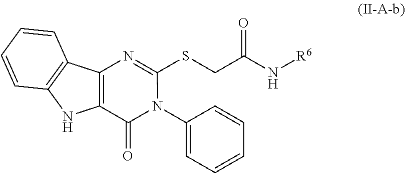

wherein R⁶ is unsubstituted aryl, unsubstituted cyclohexyl, unsubstituted thiazole, or —CH₂-furanyl; or (iii) R⁷ is not hydrogen.

In an additional embodiment, R⁵ is not substituted phenyl. In one embodiment, R⁵ is not p-fluorophenyl or p-methylphenyl.

In one embodiment, the compound does not have the structure of formula (II-A-a) wherein R⁶ is substituted phenyl. In one embodiment, the compound does not have the structure of formula (IIa) wherein R⁶ is p-fluorophenyl or p-methylphenyl.

Further to any embodiment disclosed herein, in one embodiment, R⁶ is not substituted or unsubstituted aryl, unsubstituted cyclohexyl, unsubstituted thiazole, or —CH₂-furanyl. In one embodiment, the compound does not have the structure of formula (IIb) wherein R⁶ is substituted or unsubstituted aryl, substituted or unsubstituted cyclohexyl, substituted or unsubstituted thiazole, or alkyl substituted with a substituted or unsubstituted furanyl. In one embodiment, R⁶ is not unsubstituted aryl, unsubstituted cyclohexyl, unsubstituted thiazole, or —CH₂-furanyl.

Further to any aspect disclosed above, in one embodiment R⁵ is substituted or unsubstituted cycloalkyl or substituted or unsubstituted aryl. In one embodiment, R⁵ is unsubstituted cycloalkyl or unsubstituted aryl.

In one embodiment, R⁵ is substituted or unsubstituted C₆-C₈ cycloalkyl or substituted or unsubstituted phenyl. In one embodiment, R⁵ is substituted or unsubstituted C₆, cycloalkyl or substituted or unsubstituted phenyl.

In one embodiment, R⁵ is R^{5A}-substituted or unsubstituted C6 cycloalkyl or R^{5A}-substituted or unsubstituted phenyl, wherein R^{5A} is a halogen. In one embodiment, R⁵ is R^{5A}-substituted or unsubstituted phenyl, wherein R^{5A} is a halogen. In one embodiment, R⁵ is R^{5A}-substituted or unsubstituted phenyl, wherein R^{5A} is a fluoro. In one embodiment, R⁵ is unsubstituted phenyl.

Further to any embodiment disclosed herein, in one embodiment the compound does not have the structure of Formula (II-A-b) wherein R⁶ is substituted or unsubstituted aryl, substituted or unsubstituted cyclohexyl, substituted or unsubstituted thiazole, or alkyl substituted with a substituted or unsubstituted furanyl.

In one embodiment, R⁶ is substituted or unsubstituted C₄-C₁₂ cycloalkyl, substituted or unsubstituted C₃-C₁₂ alkyl, substituted or unsubstituted aryl, or substituted or unsubstituted heteroaryl. In one embodiment, R⁶ is substituted or unsubstituted C₄-C₁₂ cycloalkyl, substituted or unsubstituted C₄-C₁₂ alkyl, substituted or unsubstituted aryl, or substituted or unsubstituted heteroaryl. In one embodiment, R⁶ is substituted or unsubstituted C₄-C₁₂ cycloalkyl, substituted or unsubstituted C₄-C₁₂ branched alkyl, or substituted or unsubstituted phenyl. In one embodiment, R⁶ is R^{6A}-substituted or unsubstituted C₄-C₁₂ cycloalkyl, R^{6A}-substituted or unsubstituted C₄-C₁₂ branched alkyl, or R^{6A}-substituted or unsubstituted phenyl, wherein R^{6A} is halogen. In one embodiment, R⁶ is R^{6A}-substituted or unsubstituted C₄-C₁₂ cycloalkyl, R^{6A}-substituted or unsubstituted C₄-C₁₂ branched alkyl, or R^{6A}-substituted or unsubstituted phenyl, wherein R^{6A} is fluoro. In one embodiment, R⁶ is unsubstituted C₄-C₁₂ cycloalkyl, unsubstituted C₄-C₁₂ branched alkyl, or R^{6A}-substituted or unsubstituted phenyl, wherein R^{6A} is fluoro. In one embodiment, R⁶ is unsubstituted C₆-C₁₂ cycloalkyl, unsubstituted C₄-C₁₂ branched alkyl, or unsubstituted phenyl. In one embodiment, R⁶ is unsubstituted C₆-C₁₀ cycloalkyl. In one embodiment, R⁶ is unsubstituted C₆-C₈ cycloalkyl. In one embodiment, R⁶ is unsubstituted cyclohexyl.

In one embodiment, R⁷ is hydrogen or substituted or unsubstituted alkyl. In one embodiment, R⁷ is hydrogen or unsubstituted alkyl. In one embodiment, R⁷ is hydrogen or unsubstituted C₁-C₃ alkyl. In one embodiment, R⁷ is hydrogen, methyl or ethyl. In one embodiment, R³ is methyl. In one embodiment, R⁷ is ethyl. In one embodiment, R⁷ is hydrogen.

In one embodiment, zl is 0, 1, 2, 3, or 4. In one embodiment, zl is 0 or 1. In one embodiment, zl is 0. In one embodiment, zl is 1. In one embodiment, z2 is 0, 1, 2, 3, 4, or 5. In one embodiment, z2 is 1.

In one embodiment, R⁸ is independently substituted or unsubstituted alkyl. In one embodiment, R⁸ independently is substituted alkyl. In one embodiment, R⁸ is independently unsubstituted alkyl. In one embodiment, R⁸ is independently substituted or unsubstituted heteroalkyl. In one embodiment, R⁸ is independently substituted heteroalkyl. In one embodiment, R⁸ is independently unsubstituted heteroalkyl. In one embodiment, R⁸ is independently substituted or unsubstituted aryl. In one embodiment, R⁸ is independently substituted or unsubstituted heteroaryl.

In another embodiment, the compound is of formula (II-A-c):

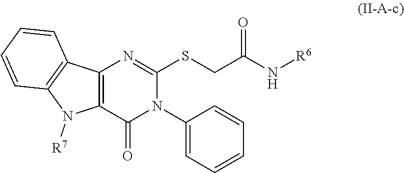

For formula (II-A-c) (above), R⁶ is substituted or unsubstituted alkyl, or substituted or unsubstituted cycloalkyl; and R⁷ is substituted or unsubstituted alkyl. In one embodiment, R⁶ is unsubstituted cycloalkyl, e.g., cyclohexyl, cycloheptyl or cyclooctyl. In one embodiment, R⁶ is unsubstituted alkyl, e.g., 3,3-dimethylbutyl. In one embodiment, R⁷ is unsubstituted alkyl. In one embodiment, R¹⁰ is an alkyl ester.

In another aspect, there is provided a compound having formula (II-A-d):

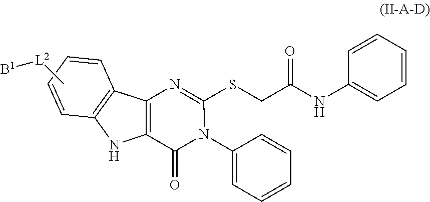

### A-D)

For formula (II-A-d), L² is a linker, and B¹ is a purine base or analog thereof.

In one embodiment, L² is a substituted or unsubstituted alkylene, or a substituted or unsubstituted heteroalkylene. In one embodiment, L² includes a water soluble polymer. A “water soluble polymer” means a polymer which is sufficiently soluble in water under physiologic conditions of e.g., temperature, ionic concentration and the like, as known in the art, to be useful for the methods described herein. An exemplary water soluble polymer is polyethylene glycol.

In one embodiment, the water soluble polymer is —(OCH₂CH₂)ₘ— wherein m is 1 to 100. In one embodiment, L² includes a cleavage element. A “cleavage element” is a chemical functionality which can undergo cleavage (e.g., hydrolysis) to release the compound, optionally including remnants of linker L², and B¹, optionally including remnants of linker L².

Compounds of formula (II-A) as described herein are synthesized, for example, in accordance with the following general scheme:

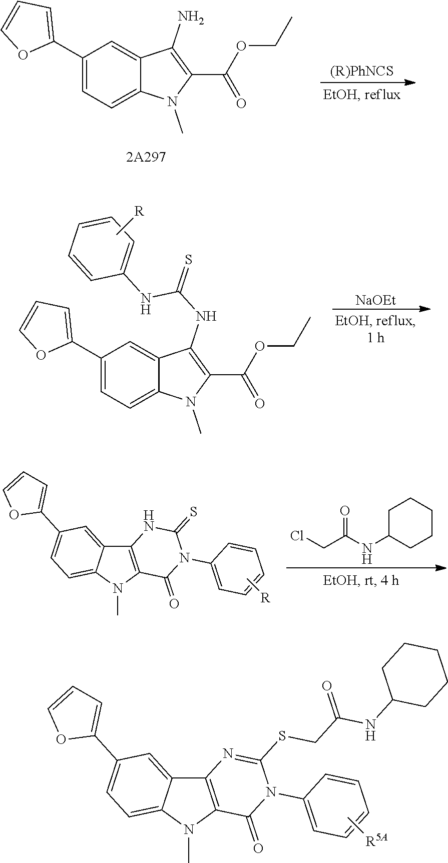

In exemplary embodiments, the present disclosure also provides the following compounds of formula (II-A):

| | |
|---|---|
| 2B182c | 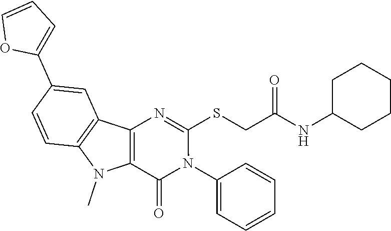 [SMILES: Cn1c2ccc(-c3ccco3)cc2c2nc(SCC(=O)NC3CCCCC3)n(-c3ccccc3)c(=O)c21] |
| 2G023a | 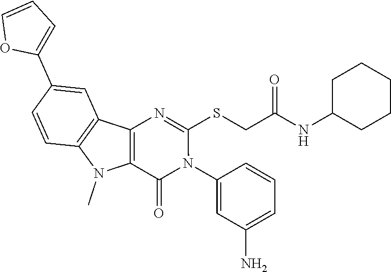 [SMILES: Cn1c2ccc(-c3ccco3)cc2c2nc(SCC(=O)NC3CCCCC3)n(-c3cccc(N)c3)c(=O)c21] |
| 2G053 | 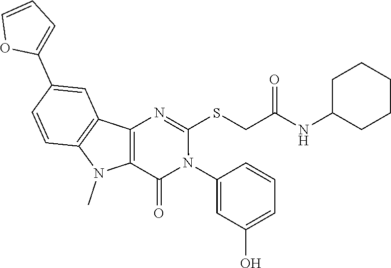 [SMILES: Cn1c2ccc(-c3ccco3)cc2c2nc(SCC(=O)NC3CCCCC3)n(-c3cccc(O)c3)c(=O)c21] |
| 2G112 | 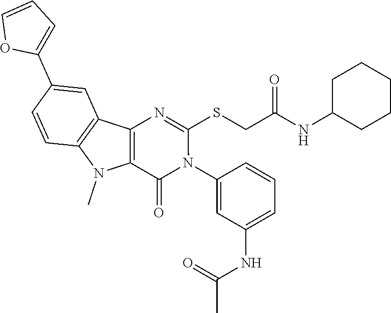 [SMILES: CC(=O)Nc1cccc(-n2c(SCC(=O)NC3CCCCC3)nc3c4cc(-c5ccco5)ccc4n(C)c3c2=O)c1] |
| 2G113 | 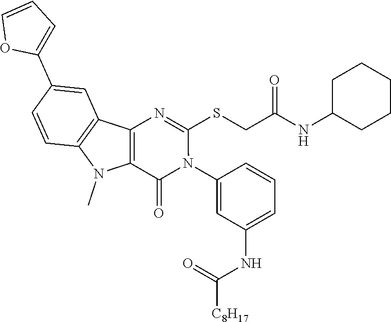 |
| 2G107 | 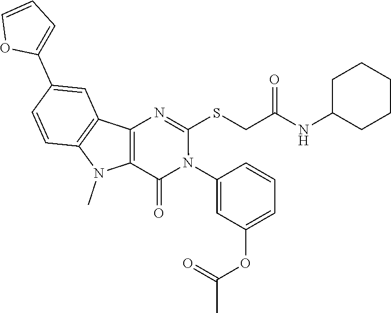 [SMILES: CC(=O)Oc1cccc(-n2c(SCC(=O)NC3CCCCC3)nc3c4cc(-c5ccco5)ccc4n(C)c3c2=O)c1] |
| 2G108 | 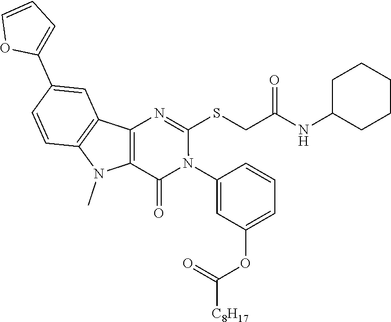 |
| 2G157 | 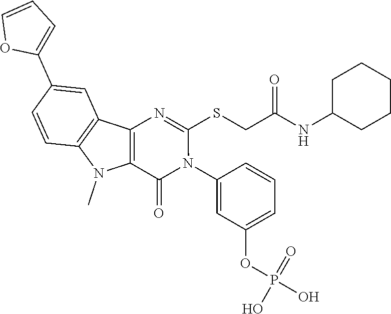 [SMILES: Cn1c2ccc(-c3ccco3)cc2c2nc(SCC(=O)NC3CCCCC3)n(-c3cccc(OP(=O)(O)O)c3)c(=O)c21] |
| 2G154 | 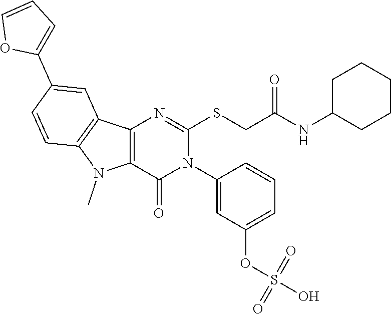 [SMILES: Cn1c2ccc(-c3ccco3)cc2c2nc(SCC(=O)NC3CCCCC3)n(-c3cccc(OS(=O)(=O)O)c3)c(=O)c21] |
| 2G202 | 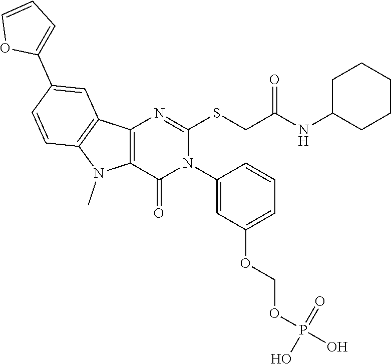 [SMILES: Cn1c2ccc(-c3ccco3)cc2c2nc(SCC(=O)NC3CCCCC3)n(-c3cccc(OCOP(=O)(O)O)c3)c(=O)c21] |
| 2E255 | 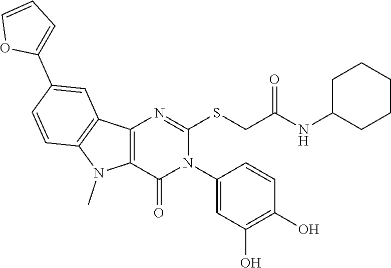 [SMILES: Cn1c2ccc(-c3ccco3)cc2c2nc(SCC(=O)NC3CCCCC3)n(-c3ccc(O)c(O)c3)c(=O)c21] |
| 2G193 | 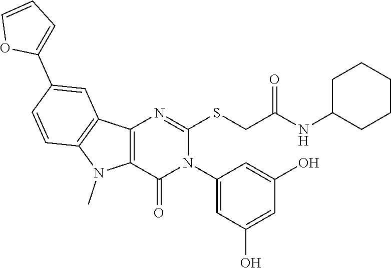 [SMILES: Cn1c2ccc(-c3ccco3)cc2c2nc(SCC(=O)NC3CCCCC3)n(-c3cc(O)cc(O)c3)c(=O)c21] |
| 2G177 | 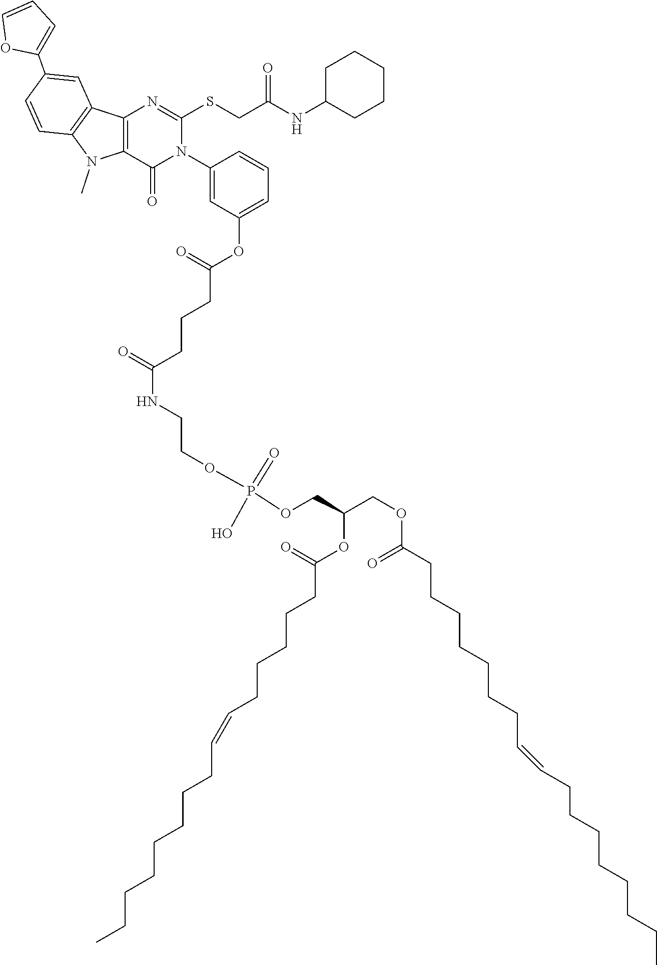 [SMILES: CCCCCCCC/C=C\CCCCCCCC(=O)OC[C@H](COP(=O)(O)OCCNC(=O)CCCC(=O)Oc1cccc(-n2c(SCC(=O)NC3CCCCC3)nc3c4cc(-c5ccco5)ccc4n(C)c3c2=O)c1)OC(=O)CCCCC/C=C\CCCCCCCC] |
| 2G22 | 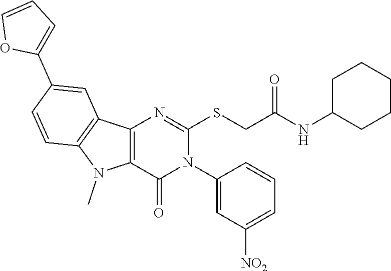 [SMILES: Cn1c2ccc(-c3ccco3)cc2c2nc(SCC(=O)NC3CCCCC3)n(-c3cccc([N+](=O)[O-])c3)c(=O)c21] |
| 2G004 | 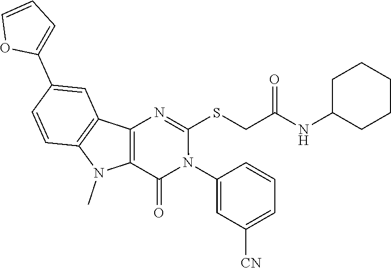 [SMILES: Cn1c2ccc(-c3ccco3)cc2c2nc(SCC(=O)NC3CCCCC3)n(-c3cccc(C#N)c3)c(=O)c21] |
| 2G013 | 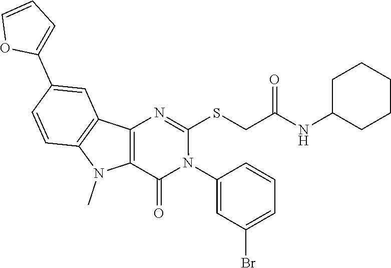 [SMILES: Cn1c2ccc(-c3ccco3)cc2c2nc(SCC(=O)NC3CCCCC3)n(-c3cccc(Br)c3)c(=O)c21] |
| 2G023b | 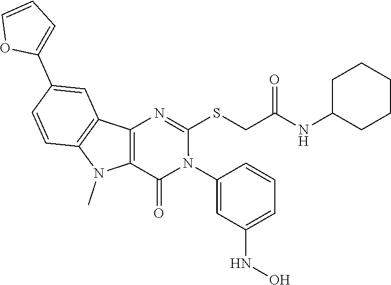 [SMILES: Cn1c2ccc(-c3ccco3)cc2c2nc(SCC(=O)NC3CCCCC3)n(-c3cccc(NO)c3)c(=O)c21] |
| 2G016 | 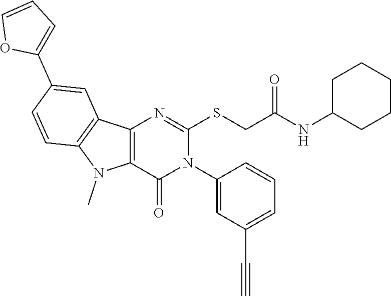 [SMILES: C#Cc1cccc(-n2c(SCC(=O)NC3CCCCC3)nc3c4cc(-c5ccco5)ccc4n(C)c3c2=O)c1] |
| 2G010 | 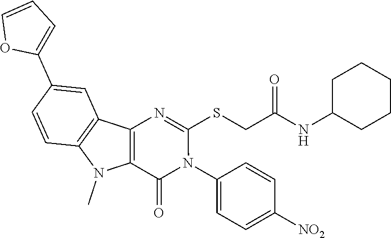 [SMILES: Cn1c2ccc(-c3ccco3)cc2c2nc(SCC(=O)NC3CCCCC3)n(-c3ccc([N+](=O)[O-])cc3)c(=O)c21] |
| 2G002 | 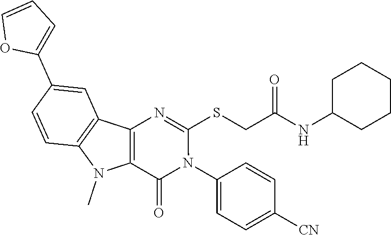 [SMILES: Cn1c2ccc(-c3ccco3)cc2c2nc(SCC(=O)NC3CCCCC3)n(-c3ccc(C#N)cc3)c(=O)c21] |
| 2G019 | 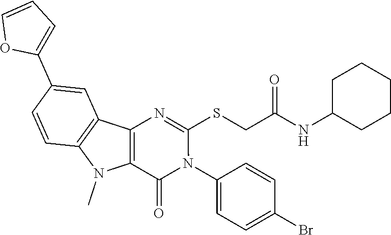 [SMILES: Cn1c2ccc(-c3ccco3)cc2c2nc(SCC(=O)NC3CCCCC3)n(-c3ccc(Br)cc3)c(=O)c21] |
| 2G036a | 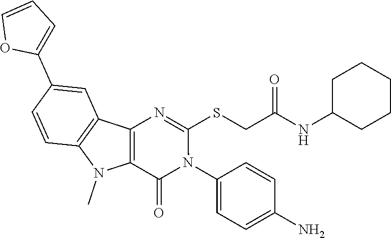 [SMILES: Cn1c2ccc(-c3ccco3)cc2c2nc(SCC(=O)NC3CCCCC3)n(-c3ccc(N)cc3)c(=O)c21] |

### Routes and Formulations

Administration of compositions having one or more adjuvants and optionally another active agent, e.g., one or more antigens, or administration of a composition having one or more antigens and a composition having one or more adjuvants, can be via any of suitable route of administration, particularly parenterally, for example, intravenously, intra-arterially, intraperitoneally, intrathecally, intraventricularly, intraurethrally, intrasternally, intracranially, intranasally, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compounds (a conjugate or other active agent) may be formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

The compositions alone or in combination with other active agents can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

Thus, the compositions alone or in combination with another active agent, e.g., an antigen, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the composition optionally in combination with an active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of conjugate and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the phospholipid conjugate optionally in combination with another active compound may be incorporated into sustained-release preparations and devices.

The composition optionally in combination with another active compound may also be administered intravenously or intraperitoneally by infusion or injection. Solutions of the antigen(s), and adjuvant(s) optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating compound(s) in the required amount in the appropriate solvent with various of the other ingredients enumerated above, as required, followed by filter sterilization. In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the antigen(s) and adjuvant(s) optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to enhance the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the disclosure provides various dosage formulations of the antigen(s) and adjuvant(s) optionally in combination with another active compound for inhalation delivery. For example, formulations may be designed for aerosol use in devices such as metered-dose inhalers, dry powder inhalers and nebulizers.

Examples of useful dermatological compositions which can be used to deliver compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages can be determined by comparing their in vitro activity, and in vivo activity in animal models. Methods for the extrapolation of effective dosages in mice, and other animals, to humans are known to the art; for example, see U.S. Pat. No. 4,938,949. The ability of an adjuvant to act as a TLR agonist may be determined using pharmacological models which are well known to the art, including the procedures disclosed by Lee et al., Proc. Natl. Acad. Sci. USA, 100: 6646 (2003).

Generally, the concentration of the phospholipid optionally in combination with another active compound in a liquid composition, such as a lotion, will be from about 0.1-25 wt-%, e.g., from about 0.5-10 wt-%. The concentration in a semi-solid or solid composition such as a gel or a powder will be about 0.1-5 wt-%, e.g., about 0.5-2.5 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the antigen(s) and adjuvant(s) optionally in combination with another active compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day.

The antigen(s) and adjuvant(s) optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This can achieve plasma levels of about 500-750 uM, which can be effective to kill cancer cells. In managing the patient, the therapy should be initiated at a lower dose and incd depending on the patient's global response.

A specific antigen includes an amino acid, a carbohydrate, a peptide, a protein, a nucleic acid, a lipid, a body substance, or a cell such as a microbe.

A specific peptide has from 2 to about 20 amino acid residues.

Another specific peptide has from 10 to about 20 amino acid residues.

A specific antigen includes a carbohydrate.

A specific antigen is a microbe. A specific microbe is a virus, bacteria, or fungi.

Specific bacteria are Bacillus anthracis, Listeria monocytogenes, Francisella tularensis, Salmonella, or Staphylococcus. Specific Salmonella are S. typhimurium or S. enteritidis. Specific Staphylococcus include S. aureus.

Specific viruses are RNA viruses, including RSV and influenza virus, a product of the RNA virus, or a DNA virus, including herpes virus. A specific DNA virus is hepatitis B virus.

A specific antigen includes one or more cancer antigens such as one or more tumor-associated antigens (TAAs) or tumor specific antigens (TSAs)

The invention will be further described by the following non-limiting examples.

### Example 1

Three high throughput screens (HTSs) using the Maybridge compound library (Thermo Fisher Scientific) were employed to identify, characterize and optimize compounds that induce the release of immunostimulatory extracellular vesicles (EVs) by antigen-presenting cells and enhance the protective efficacy of vaccine adjuvants. Three high throughput screens (HTSs) were conducted using the Maybridge compound library (Thermo Fisher Scientific).

For immune screening, a THP-1 human monocytic cell line equipped with the NF-κB- and interferon sensitive response elements (ISRE)-reporter construct was used. For screening extracellular vesicle release, THP-1 cells with CD63 Turboluciferase (Tluc) CD9 Emerald Green Fluorescent Protein (EmGFP) were used. UC San Diego Moores Cancer Center Biostatistics and informatics Shared Resources processed the HTS data and identified hit candidates. Eighty compounds were selected by in vitro cytokine production and by medicinal chemistry inspection and subjected to in vivo adjuvant activity evaluation. 8 compounds showing 4 distinct adjuvant activity profiles were selected.

### Hit Candidate Selection Process

80 compounds were selected for in vivo adjuvant evaluation following the three HTS using extracellular vesicles (EV) release CD63 THP-1 Tluc, NF-κB THP-1, and ISRE THP-1 CellSensor lines. C57BL/6 mice were immunized with ovalbumin as a model antigen mixed with each compound on days 0 and 21. Antigen specific immunoglobulin levels were determined by ELISA one week after the immunization boost (day 28). As in vitro adjuvant activity assay, the expression of co-stimulatory molecules and MHC-class II on murine bone marrow derived dendritic cells (mBMDC) was determined The above data were plotted on a heatmap together with cytokine inductions and HTS data from which 8 compounds were selected belonging to 4 distinct adjuvant activity profiles.

### Calcium Influx Inducing Compounds

### #634 Induces Intracellular Ca²⁺ Levels

EVs play an important role in intercellular communication and regulation of cells, especially in the immune system, where EVs can participate in antigen presentation and may have adjuvant effects. To identify small molecule compounds that can increase EV release and enhance vaccines' immunogenicity, three independent HTS on the same large 27,895 compound library were screened using release EV-associated tetraspanin (CD63), NF-κB, and ISRE-THP-1 reporter cell lines. In addition, further evaluation for induction of IL-12 and cell viability in murine BMDC and assessment from a medicinal chemistry perspective resulted in the selection of 84 hits.

Calcium signaling is a key factor for both EV release and adjuvant activity. Intracellular Ca²⁺ increase leads to plasma membrane EV biogenesis (Taylor et al., 2020; Messenger et al., 2018). In addition, calcium signaling plays multiple roles in the activation, migration, and maturation of dendritic cells (DCs) (Rao & Hogan, 2009; Vig & Kinet, 2009). Another report indicated that calcium ionophore enhances EV release (Messenger et al., 2018; Kramer-Albers et al., 2007) and induced maturation and activation of DCs (Czerniecki et al., 1997). In addition, it was demonstrated that small molecule Ca²⁺ channel activators enhance the known adjuvant activities as co-adjuvant in our previous work (Saito et al., 2022; Saito et al., 2021). Based on the above knowledge, it was hypothesized that small molecule compounds which can induce intracellular Ca²⁺ levels could induce EV biogenesis release and enhance innate immune responses. Ca²⁺ increase was examined after adding the selected 84 hit candidate compounds by a ratiometric Ca²⁺ indicator, Fura-2, assay (FIG. 1A). Two compounds, #634 and #504 induced a higher intracellular Ca²⁺ increase than vehicle control (DMSO) (FIG. 1A). #634 rapidly increased intracellular Ca²⁺ which was sustained for at least 25 min similar to the calcium ionophore, lonomycin by Fura-8 assay (a ratiometric Ca²⁺ indicator), (FIG. 1B). In the Ca²⁺ add-back assay, #634 did not increase intracellular Ca²⁺ levels without extracellular Ca²⁺, while intracellular Ca²⁺ influx was restored by adding the Ca²⁺ in the medium (1.8 mM) (FIG. 1C).

### #634 Shows Vaccine Adjuvant Activity In Vivo

Eighty-four hit candidate compounds were also screened for in vivo adjuvant activities using C57BL/6 mice and model antigen ovalbumin (OVA). Mice (n=5 for each group) were immunized with OVA (20 μg/animal) mixed with each compound (200 nmol/injection) on days 0 and 21. Monophosphorylated Lipid A (MPLA, 1 μg/animal/injection) was used as a positive control, and 10% DMSO was used as vehicle control (Veh). Sera were collected on day 28, and OVA-specific immunoglobulins IgG1 and IgG2c were determined by ELISA (FIG. 1D). Anti-OVA IgG1 and IgG2c levels in the sera of the animals that received OVA adjuvanted with #634 appeared to be higher than the levels from animals immunized without adjuvant, although there were no significant differences.

### #634 Induces Genes Related to Ca2+ Signaling

As an initial assessment of the mechanism of action of #634, the global gene expression induced by #634 in mBMDCs was examined using RNA-seq analysis. #634 modulated expression of 103 genes compared to Veh (FDR s; 0.05, and fold change >2) (86 genes: up-regulated, 17 genes: down-regulated). These findings are visualized in FIG. 2A using a volcano plot. In the Kyoto Encyclopedia of Genes and Genomes (KEGG) pathway database, Gene Set Enrichment Analysis (GSEA) of genes whose expression was affected by #634 revealed the top positively and negatively enriched pathways (FIG. 2B). To examine the effects of #634 on the expression of genes related to calcium signaling, each of the 103 genes was examined for their relationship to calcium signaling pathways. FIG. 2C shows seven calcium-related genes (2 genes: down-regulated, 5 genes: up-regulated) in the 103 genes. These results imply that #634 acts via calcium signaling pathways.

### #634 Enhances EV Release

Compound #634 was selected by three HTS, including CD63 reporter THP-1 cells. To validate the enhancement of EV release by #634, EV particle numbers were assessed in the culture supernatants of mBMDCs cultured with #634 (10 μM) for 46-48 h using microfluidic resistive pulse sensing (MRPS) with an nCS1 instrument. The EVs in the culture supernatants were isolated following the multistep differential ultracentrifugation protocol (Shpigelman et al., 2021). #634 increased the number of EV particles released in the culture supernatant compared to the Veh control (FIG. 3A). MRPS showed that the release of particles of diameter less than 150 nm was significantly enhanced by #634 (FIG. 3B). Immunoblots of isolated EVs confirmed enrichment for the tetraspanins CD81 and Tsg101 and attenuation of the endoplasmic reticulum-associated protein, Calnexin (FIG. 3C). The amounts of total RNA from EV released by #634-treated mBMDC (EV₅₃₄) and EVs released by Veh-treated mBMDC (EVᵥₑₕ) were comparable as measured by the RNeasy Mini Kit using Nanodrop (FIG. 3D).

EVs Derived from BMDCs Treated with #634 Stimulate DO 11.10 T Cell Proliferation

To assess if the EVs from #634 treated BMDCs (EV₅₃₄) would be effective in T cell priming, carboxyfluorescein succinimidyl ester (CFSE)-labeled CD4⁺ T cells expressing OVA-specific T cell receptors (from DO11.10 mice) were co-cultured with EV₅₃₄ in the presence of OVA MHC class II peptides (OVA₃₂₃₋₃₃₉). MPLA (1 μg/ml) was used as a positive control, as Qazi et al., demonstrated that EVs released from TLR4 ligand-matured mBMDC directly loaded with peptide elicited more robust T cell activation (Qazi et al., 2009). Ionomycin (1 μM) was used as a positive control of intracellular Ca²⁺ inducer. T cell proliferation was monitored by CFSE dilution using flow cytometric assay (FIG. 4). EV₅₃₄ induced significantly higher T cell proliferation as well as EVs from MPLA or lonomycin treated BMDCs than EVᵥₑₕ (FIGS. 3A, and B).

Enhancement of DO11.10 T cell proliferation by EV₅₃₄ was dose-dependent (FIG. 3C). To examine the possibility that EV₅₃₄ directly activates T cells to enhance their proliferation without TCR engagement, wild-type BALB/c CD4⁺ T cells were incubated with EV₅₃₄ in the absence of the OVA₃₂₃₋₃₃₉ peptide. EV₅₃₄ did not induce proliferation in the absence of antigen (FIG. 5D). These data indicate that EV₅₃₄ enhances the proliferation of DO.11.10 CD4⁺ T cells mediated by antigen engagement with TCR.

### SAR Study of #634 Analogs

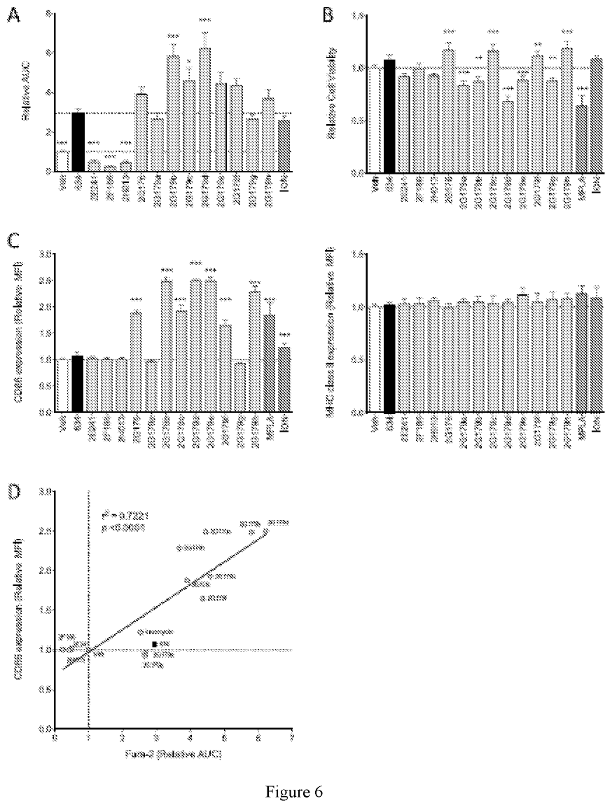

The Ca²⁺ inducer #634 could increase EV release and enhanced cell proliferation in the presence of TCR engagement. To evaluate Ca²⁺ influx associated with EV functions, Ca influx inhibitors EGTA, BAPTA-AM, or BTP2 were tested. However, these inhibitors were cytotoxic which was inadequate for a 48 hours culture, and increased EV release by mBMDC by the inhibitors alone. Alternatively, a structure-activity relationship (SAR) study of #634 was conducted and it was assessed whether the potencies of Ca²⁺ inducers correlated to the number of EVs released and function. In an SAR study of #634, the elements of the #634 scaffold are necessary for the induction of intracellular Ca²⁺ (Table 1) were assessed. The intracellular Ca²⁺ levels and cell viabilities of mBMDC were measured by a Fura-2 and MTT assay following incubation with 12 analogs, respectively (FIGS. 6A-B). 2E241, 2F186, and 2H013 did not induce Ca²⁺ influx. Three compounds, 2G179b, 2G179c, 2G179d, increased intracellular Ca²⁺ levels more than that of #634. Intracellular Ca²⁺ increase is important for DC maturation (Shumilina et al., 2011), and the calcium ionophore ionomycin up-regulates CD86 in human DC (Ramadan et al., 2001). Hence, the expression of co-stimulatory molecules, including CD86 and MHC class II, was assessed after 24 hours incubation of mBMDC with the analogs. The flow cytometric analysis showed that some analogs increased the expression of CD86 in BMDCs (FIG. 6C). A scatter plot displays an overall linear relationship between intracellular Ca²⁺ induction and CD86 expression in BMDCs (FIG. 6D). Three non-Ca²⁺ inducers did not increase CD86 expression (FIG. 6D).

TABLE 1
Chemical structure of a small exploratory series of #634 analogs

| Compounds | Structures |
|---|---|
| 2E213 | 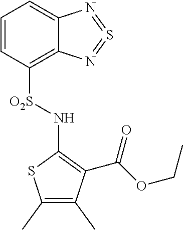 [SMILES: CCOC(=O)c1c(NS(=O)(=O)c2cccc3c2N=S=N3)sc(C)c1C] #634 C [symbol] H [symbol] N₃O₄S₃ |
| 2H013 | 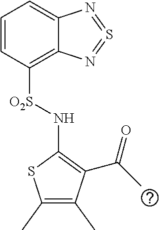 [SMILES: CC(=O)c1c(NS(=O)(=O)c2cccc3c2N=S=N3)sc(C)c1C] carboxylic acid analog of 634 C₁₃H₁₁N₃O₄S₃ |
| 2F186 | 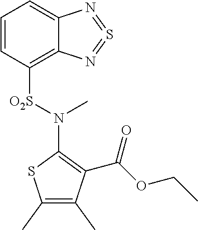 [SMILES: CCOC(=O)c1c(N(C)S(=O)(=O)c2cccc3c2N=S=N3)sc(C)c1C] N-methyl analog C₁₆H₁₇N₃O₄S₃ |
| 2E241 | 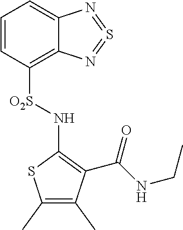 [SMILES: CCNC(=O)c1c(NS(=O)(=O)c2cccc3c2N=S=N3)sc(C)c1C] ethylamide analog C₁₅H₁₆N₄O₃S₃ |
| 2G176 | 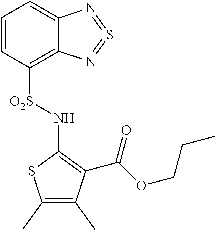 [SMILES: CCCOC(=O)c1c(NS(=O)(=O)c2cccc3c2N=S=N3)sc(C)c1C] propyl ester C₁₆H₁₇N₃O₄S₃ |
| 2G179a | 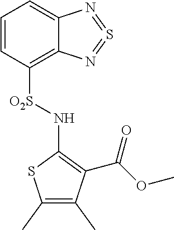 [SMILES: COC(=O)c1c(NS(=O)(=O)c2cccc3c2N=S=N3)sc(C)c1C] methyl ester C₁₄H₁₃N₃O₄S₃ |
| 2G179b | 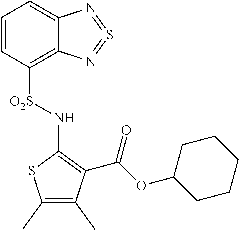 [SMILES: Cc1sc(NS(=O)(=O)c2cccc3c2N=S=N3)c(C(=O)OC2CCCCC2)c1C] hexyl ester C₁₉H₂₃N₃O₄S₃ |
| 2G179c | 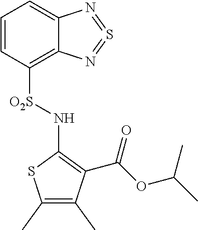 [SMILES: Cc1sc(NS(=O)(=O)c2cccc3c2N=S=N3)c(C(=O)OC(C)C)c1C] isopropyl ester C₁₆H₁₇N₃O₄S₃ |
| 2G179d | 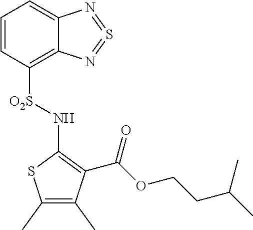 [SMILES: Cc1sc(NS(=O)(=O)c2cccc3c2N=S=N3)c(C(=O)OCCC(C)C)c1C] isoamyl ester C₁₈H₂₁N₃O₄S₃ |
| 2G179e | 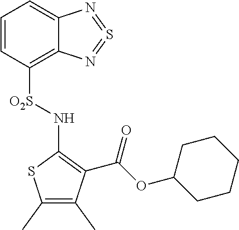 [SMILES: Cc1sc(NS(=O)(=O)c2cccc3c2N=S=N3)c(C(=O)OC2CCCCC2)c1C] cyclohexyl ester C₁₉H₂₁N₃O₄S₃ |
| 2G179f | 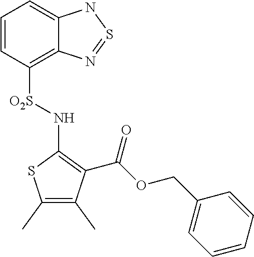 benzyl ester C₂₀H₁₇N₃O₄S₃ |

[symbol]

indicates data missing or illegible when filed

### Selection of Analogs of #504

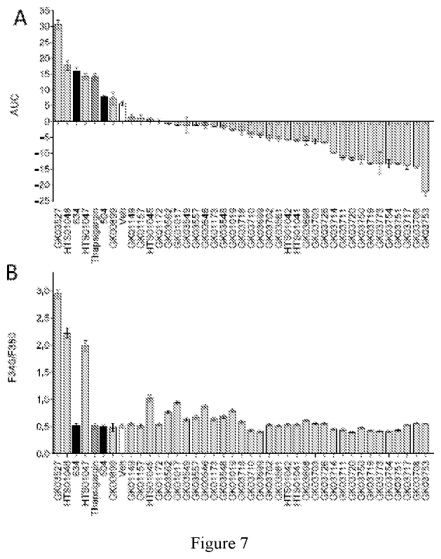

Compound #504 was identified in the Intracellular Ca²¹ influx screen by Fura-2 assay in addition to #634 (FIG. 7A). To determine the correlation between Ca²⁺ inducing abilities and EV function, an SAR study of #504 was performed using repurchased compounds (Table 2). Intracellular Ca²⁺ increase was determined after adding the thirty-seven #504 analogs by a Fura-2 assay (FIG. 7A). In parallel, compound autofluorescence was assessed in the same condition of the Fura-2 assay to eliminate the false positives (FIG. 71B). A Ca²⁺ inducer from the endoplasmic reticulum, Thapsigargin, was used as a positive control. No compounds in 37 analogs were identified as intracellular Ca inducers because three compounds by Fura-2 assay, GK03527, HTS01048, HTP01047, were autofluorescence positives.

TABLE 2
Chemical structure of a small exploratory series of #504 analogs

| | MSAR | |
|---|---|---|
| Structure | Modification | Compounds |
| 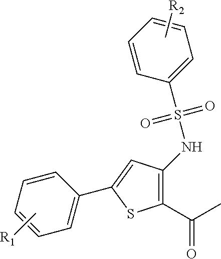 | Ketone | GK03710 GK03718 GK03719 GK03714 GK03720 GK03708 GK03750 GK03754 GK03711 GK03753 GK03751 GK03773 GK03548 GK03717 GK03549 |
| 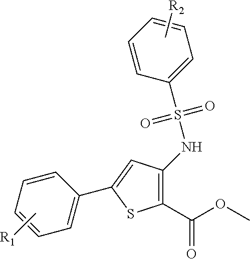 | Ester | GK00546 GK03527 HTS01048 HTS01047 |
| 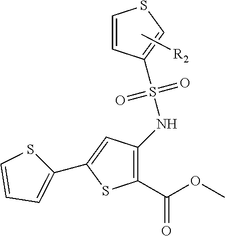 | Ester | 504 GK00899 HTS01042 |
| 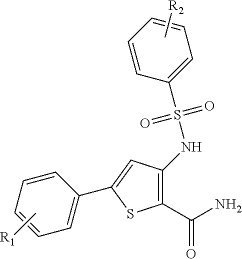 | Amide | GK01017 GK01173 GK01019 GK01149 GK01172 GK01157 GK03726 GK03561 GK03557 GK03562 |
| 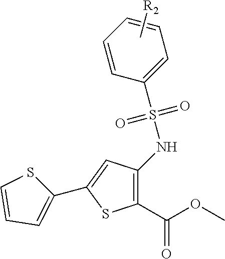 | Ester | GK03698 GK03699 GK03702 GK03703 |
| 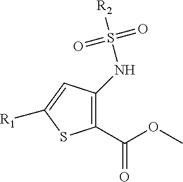 | Ester | HTS01045 HTS01041 |

### Structure-Activity Relationship, Study of Hit Candidate Compound #645

A few chemotypes were selected as hit candidates for initial structure-activity relationship (SAR) studies based on the in vivo and in vitro adjuvanticity experiments.

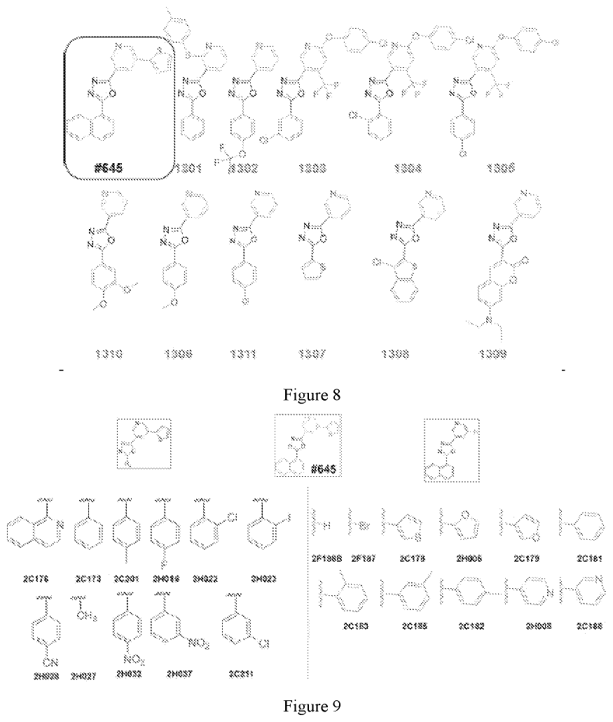

Based on the HTS, the hit candidate compound #645 that belongs to 3-pyridyl-oxadiazole chemotype was found to be a good adjuvant (in vivo) that enhanced the upregulation of CD63 and other co-stimulatory molecules including CD80, 83, 86 and MHC class II (in vitro). Thus, SAR studies for this chemotype were pursued. Available compounds bearing the central 3-pyridyl-oxadiazole core ring system from the Maybridge library as shown in FIG. 8. These compounds were then analyzed for induction of IL-12 compared to hit compound #645. While none of these compounds were more potent than #645, the 4-chlorophenyl (#1311) and 3,4-dimethoxyphenyl (#1310) substituted compounds were found to be as potent as #645.

Since most of these compounds had multiple modifications at once compared to compounds #645, one ring system at a time was modified to systematically approach SAR studies. The first set of compounds modified at the naphthyl moiety included isoquinoline (20176), phenyl (20173), substituted phenyls including 4-methyl (20201), 4-fluoro (21H019), 4-cyano (21H028), 4-nitro (2H032), 3-nitro (2H037), 3-chloro (2C211), 2-chloro (2H022), 2-iodo (2H023) and desphenyl analog 2H027. The second set consisted of 3-pyridyl unsubstituted compound 2F188B and several 3-substituted analogs including 3-bromo (2F187), 3-(3-thienyl) (2C178), 3-(2-furanyl) analog 2H005, 3-(2-furanyl) (2C179), 3-phenyl (2C181), 3-o-tolyl (2C183), 3-m-tolyl (2C185), 3-p-tolyl (2C182), 3-(4-pyridyl) (2H008) and 3-(3-pyridyl) (2C188). The structures of all these synthesized analogs are shown in FIG. 9.

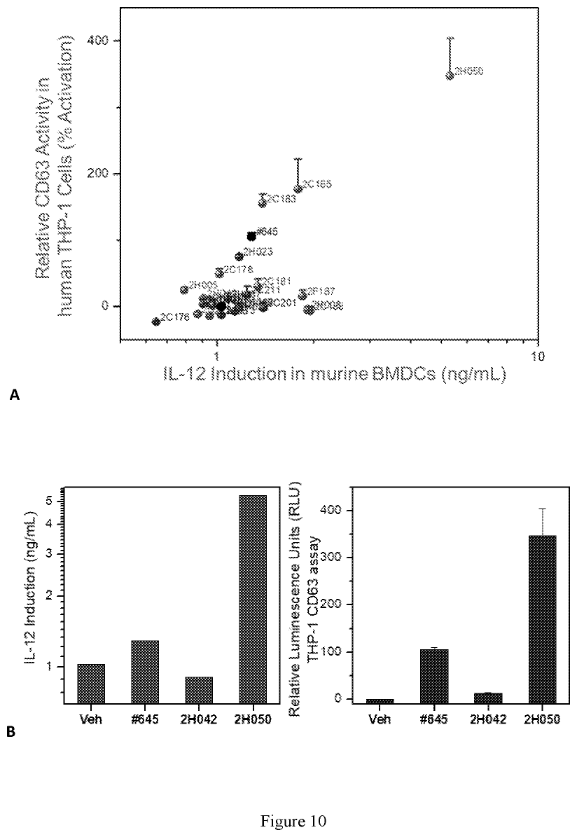

All these analogs were evaluated for induction of IL-12 cytokine at 10 μM, followed by dose response IL-12 induction for selected analogs. These analogs were also evaluated for exosome release by quantitating EV release in the supernatant post 48 hours incubation with CD63-Tluc-CD9-EmGFP THP-1 reporter cells. The scatter plot with % activation in the EV release on X-axis and IL-12 induction (by AUC) on Y-axis is shown in FIG. 10.

Some 2-oxadiazole substituted analogs (in blue) showed potency in IL-12 induction but did not enhance exosome release. However, 5-(m-tolyl) substituted pyridyl analog 2C185 was found to enhance exosome release and IL-12 induction.

Initial SAR studies on the 3-pyridyl-oxadiazole chemotype (original lead compound—#645) suggested importance of the naphthalene ring at the C2-position of the oxadiazole, while the C5-substitution on the pyridine ring showed structure based variation in potency. Especially, the 5-pyridine substituted o- and m-tolyl analogs, 2C183 and 2C185 respectively; induced IL-12 in BMDCs and enhanced exosome release in CD63 reporter THP-1 cells. Moving forward, we undertook SAR studies by varying the central oxadiazole core. Synthesis of the oxadiazole ring in the center requires separate naphthalene and pyridyl bearing synthons. Thus, modification of this ring system to obtain different analogs may need different synthons bearing these substituents. These analogs and the individual synthetic schemes to synthesize these analogs are shown below. This included many heteroaromatic 5-membered ring systems including the triazole ring analogs, tetrazole analogs, and pyrazole analogs. The syntheses of these analogs were prioritized based on availability of reagents and synthetic feasibility.

1,2,3-triazole analog 2H050: This analog was synthesized as shown in Scheme 1. Briefly, Suzuki coupling chemistry was utilized to obtain 3-bromo-5-thiophenyl pyridine synthon 2H043 from commercially available reagents. The 3-azido synthon was then attempted to obtain 2H043 by copper mediated bromo-displacement reaction, however we obtained the 3-amino analog 2H048, which was then converted to the 3-azido analog 2H049 by azide displacement of diazodized activated amine. Finally, the copper mediated “Click Chemistry” reaction between 2H049 and commercially available 1-ethynylnaphthalene yielded the triazole analog 2H050 (Scheme 1).

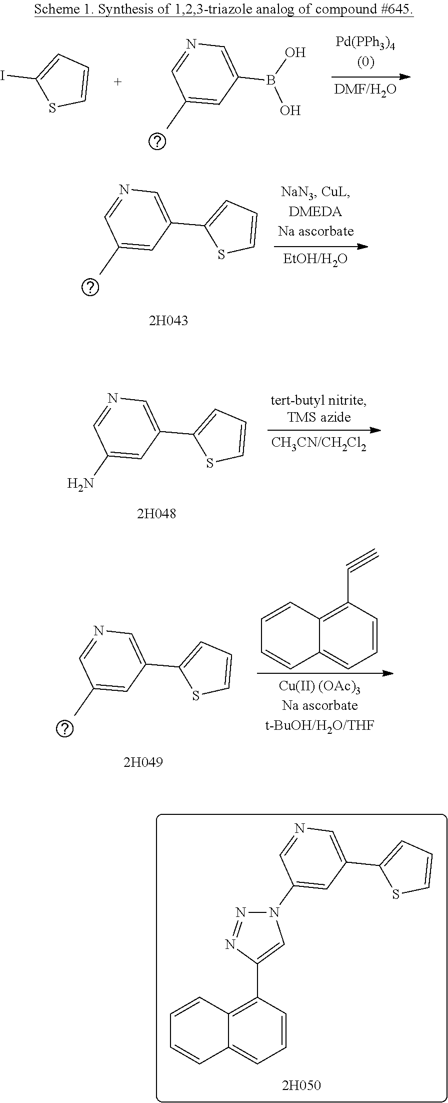

2,3,4-triazole analog 2H042: This analog was synthesized as shown in Scheme 2. Briefly, the advanced intermediate in Scheme 1, 2H043 was reacted by Sonogashira coupling chemistry to obtain 3-TMS-acetylene-pyridine synthon 2H039. The TMS protecting group was then removed by tetrabutylammonium fluoride (TBAF) solution to obtain 3-acetylene pyridine synthon. In parallel, 1-Amino naphthalene was converted to 1-azidonaphthalene by siazotisation and displacement using sodium nitrite and sodium azide. Finally, the copper mediated “Click Chemistry” reaction between 2H040 and 2E260 yielded the triazole analog 2H042 (Scheme 2).

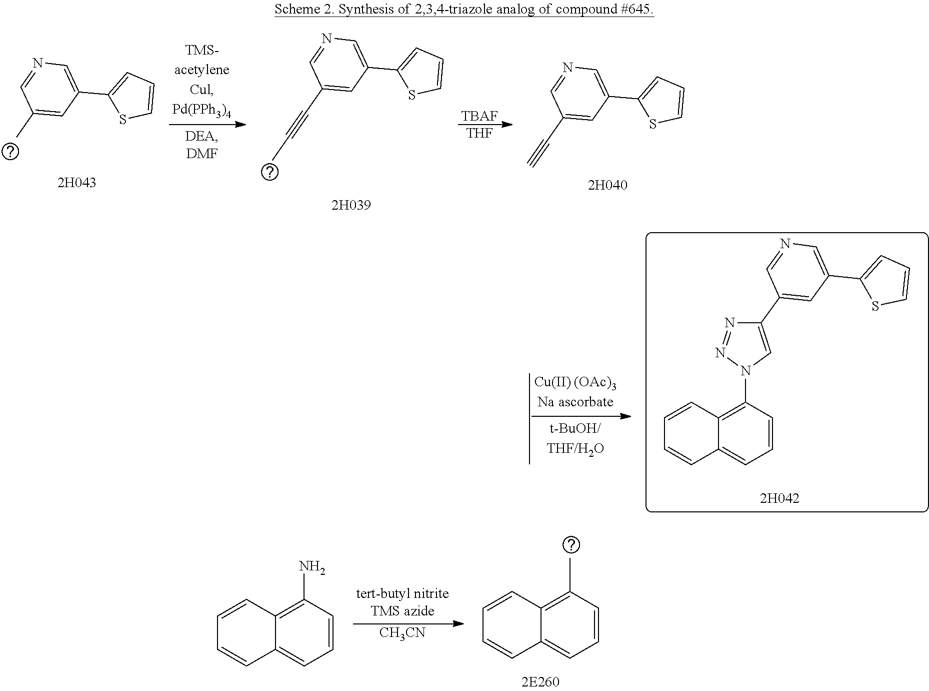

1,2,3,5-tetrazole analog 2E279: This analog was synthesized as shown in Scheme 3. Briefly, the 1-naphthaldehyde was treated with benzenesulfonohydrazide to obtain bis-sulfonohydrazide intermediate 2E278. Separately, 3-amino bearing pyridine synthon 2H048 was treated with sodium nitrite in HCl to generate the diazo intermediate which was combined with 2E278 to obtain the tetrazole analog 2E279 (Scheme 3).

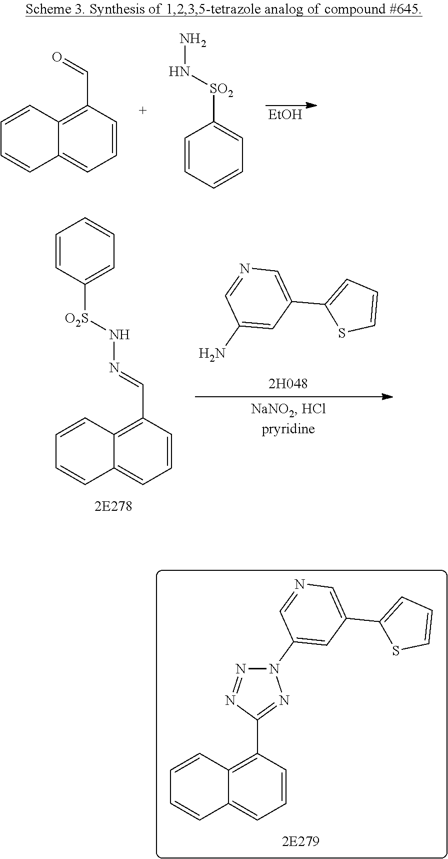

2,3,4,5-tetrazole analog 2E277: This analog was synthesized as shown in Scheme 4. First, the C3-carbaldehyde pyridine synthon 2H051 was obtained by Suzuki chemistry reaction starting with 5-bromonicotinaldehyde. The further synthesis involved similar chemistry as for 2E279 including formation of bis-sulfonohydrazide intermediate 2H052 and fusing it with diazonium salt derived from 1-naphthylamine to obtain the tetrazole analog 2E277 (Scheme 4).

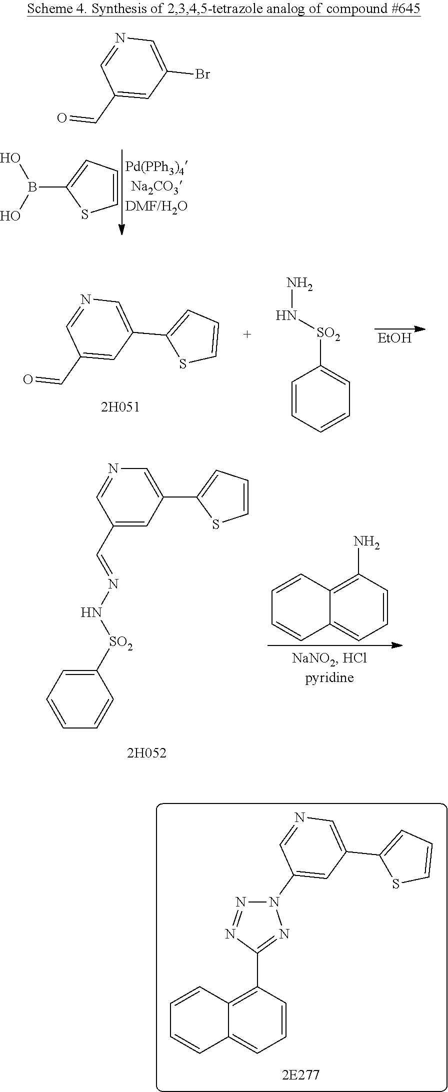

1,5-pyrazole analog 2C214: This analog was synthesized as shown in Scheme 5 in one synthetic step by arylation of 3-(naphthalen-1-yl)-1H-pyrazole with 2H043 using CuI as catalyst and potassium carbonate as base (Scheme 5).

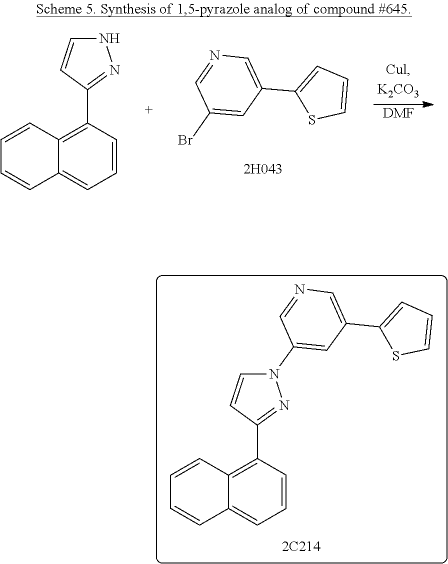

2,3-pyrazole analog 2E280: This analog was synthesized as shown in Scheme 6 using synthons used in synthesis of other analogs. The bis-sulfonohydrazide 2E278 was fused with 3-ethylnylpyridine synthon 2H040 to obtain 2,3-pyrazole analog 2E280 (Scheme 6).

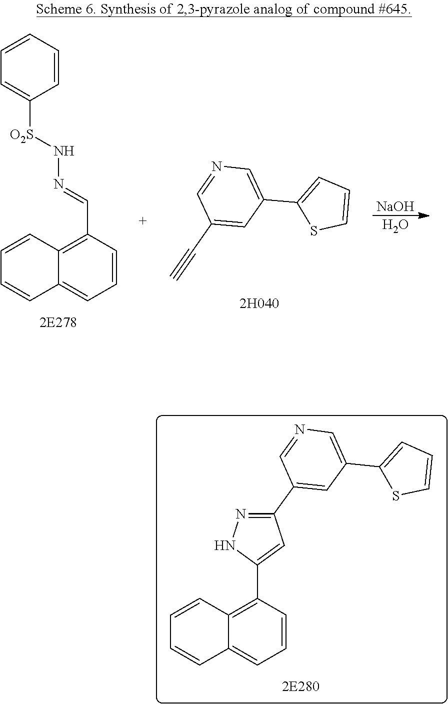

Of all these synthesized compounds, the two triazoles 2H042 and 2H050 were evaluated in induction of IL-12 and for enhancement of exosome release in THP-1 CD63 reporter assay. Compound 2H042 was not potent while 2H050 significantly enhanced cytokine IL-12 induction (FIG. 10B).

FIG. 10A shows a summary of activities for all the #645 analogs for induction of cytokine IL-12 in murine BMDCs and CD63 activity in human THP-1 reporter cells. Compounds data points are colored by the site of modification.

### Example 2

Extracellular vesicles (EVs) play an important role in intercellular communication and regulation of cells, especially in the immune system where EVs can participate in antigen presentation and may have adjuvant effects. We aimed to identify small molecule compounds that can increase EV release and thereby enhance the immunogenicity of vaccines. A THP-1 reporter cell line engineered to release EV-associated tetraspanin (CD63)-Turbo-luciferase was employed to quantitatively measure EVs released in culture supernatants as a readout of a high throughput screen (HTS) of 27,895 compounds. In parallel, the cytotoxicity of the compounds was evaluated by PrestoBlue dye assay. For screening immunostimulatory potency, two additional independent HTS were performed on the same compound library using NF-κB and interferon-stimulated response element THP-1 reporter cell lines. Hit compounds were then identified in each of the 3 HTS's, using a “Top X” and a Gaussian Mixture Model approach to rule out false positive compounds and to increase the sensitivity of the hit selection. Thus, 644 compounds were selected as hits which were further evaluated for induction of IL-12 in murine bone-marrow derived dendritic cells (mBMDCs) and for effects of cell viability. The resulting 130 hits were then assessed from a medicinal chemistry perspective to remove compounds with functional group liabilities. Finally, 80 compounds were evaluated as vaccine adjuvants in vivo using ovalbumin as a model antigen. 18 compounds with adjuvant activity were analyzed for their ability to induce the expression of co-stimulatory molecules on mBMDCs. The full complement of data was then used to cluster the compounds into 4 distinct biological activity profiles. These compounds were also evaluated for quantitation of EV release and spider plot overlays were generated to compare the activity profiles of compounds within each cluster. This tiered screening process identified two compounds that belong to the 4-thieno-2-thiopyrimidinescaffold with identical screening profiles supporting data reproducibility and validating the overall screening process. Correlation patterns in the adjuvanticity data suggested a role for CD63 and NF-κB pathways in potentiating antigen-specific antibody production. Thus, the three independent cell-based HTS campaigns led to identification of immunostimulatory compounds that release EVs and have adjuvant activity.

### INTRODUCTION

Although vaccination against common pathogens is gaining broader acceptance, there remains an unmet need for widely effective adjuvants that can elicit sustained immune responses to targeted antigens (Reed et al., 2013). Vaccine adjuvants act as immunopotentiators that are co-administered with subunit, inactivated or attenuated antigens (Tregoning et al., 2018). In the past decade, there have been advances with adjuvants that have improved the response to varicella, influenza and hepatitis B vaccines in populations with reduced immune responses (Tregoning et al., 2018). Although the adjuvants boost protective efficacy of the vaccines, they often elicit local inflammation at the site of injection in some cases accompanied by flu-like symptoms, reduce patient acceptance especially for vaccines that require annual or booster injections (Petrovsky, 2015; Nanishi et al., 2020). Adjuvants that utilize intracellular communication pathways to enhance antigen presentation and the needed cognate cellular interactions could potentially activate the immune system in a manner that is not as abruptly inflammatory.

Extracellular vesicles (EVs) act as a carrier of cell-type-specific molecules including those involved in innate immune responses, such as cytokines, chemokines, adhesion molecules, other proteins, lipids, peptides, coding and non-coding RNAs (including microRNAs), and DNA fragments (Valadi et al., 2007; Skog et al., 2008; Cossetti et al., 2014; Yanez-Mo et al., 2015). Adhesion molecules integrated into the EV outer surface membrane direct binding to potential target cells while other molecules act as ligands to cellular receptors. EVs can also encapsulate additional proteins or nucleic acids that can convey specific intercellular communications. These properties enable EVs to play modulating roles in mediating immune responses to pathogens and tumors (Campos et al., 2015; Wang et al., 2017). Hence, it was hypothesized that small molecule compounds which can simultaneously enhance innate immune responses and EV biogenesis and release could add immunomodulation modalities, and potentially increase antigen delivery to distal lymphoid organs, leading to improved vaccine efficacy and reduced toxicity.

Recently, a human monocytic leukemia THP-1 reporter cell line engineered with a fusion construct for the expression of EV-associated tetraspanins (CD63 and CD9) linked to Turbo-luciferase (Tluc) and Emerald Green Fluorescent Protein (EmGFP) (CD63 Tluc-CD9-EmGFP THP-1 cells) was prepared to quantitatively measure release of EVs in culture supernatants (Shpigelman et al., 2021). Using this reporter cell line, Tluc activity levels were found to be correlated with concentrations of released EVs in the culture supernatant as measured by nanoparticle tracking (Shpigelman et al., 2021). Here, this cell line is utilized for a high throughput screening (HTS) of a library of 27,895 compounds. Additionally, the same library was screened with two additional THP-1 reporter cell lines for NF-κB and interferon-stimulated response element (ISRE) activation, respectively. Based on “Top X” and “Gaussian mixture model” (GMM) hit detection methods, 644 compounds were identified as hits. Further studies probing into the immunological properties as well as selection based on chemical structural features narrowed the selection to 80 compounds that were assessed in vivo for adjuvant activity. All these studies led to the identification of distinct chemotypes that display immunostimulatory effects and enhance the production of EVs.

### Results

### High Throughput Screenings

Previously, compounds were identified by HTS that induce NF-κB activation or prolong the activation of NF-κB and/or ISRE, using a library from a small molecule diversity collection (SMDC, UCSF) consisting of about 170,000 compounds (Pu et al., 2012; Chan et al., 2017a; Shukla et al., 2018). However, EV release assay was much more complex and required the use of expensive exosome-depleted media under precise incubation conditions. Thus, performing CD63 HTS in this large compound library, for which the NF-κB induction data (Pu et al., 2012) was difficult to achieve. It was of interest to obtain smaller compound libraries with extensive chemical space diversity. Thus, commercial libraries developed by Maybridge (Leeds, United Kingdom) consisting of two subset libraries which are representative of the diversity of the two very different compound collections were selected: 1) the Maybridge HitFinder library of 14,400 compounds representative of the entire Maybridge Screening Collection of over 53,000 members, and 2) the Maybridge HitCreator library of 14,000 compounds representative of the diversity of a collection of 550,000 compounds. Another benefit of the Maybridge library is a lack of CAS registry numbers for a large portion of compounds allowing for freedom of intellectual property. Compound purchase, transfer, acquisition, export and quality control led to elimination of 505 compounds (<2%) thus obtaining a final list of 27,895 compounds for the HTS.

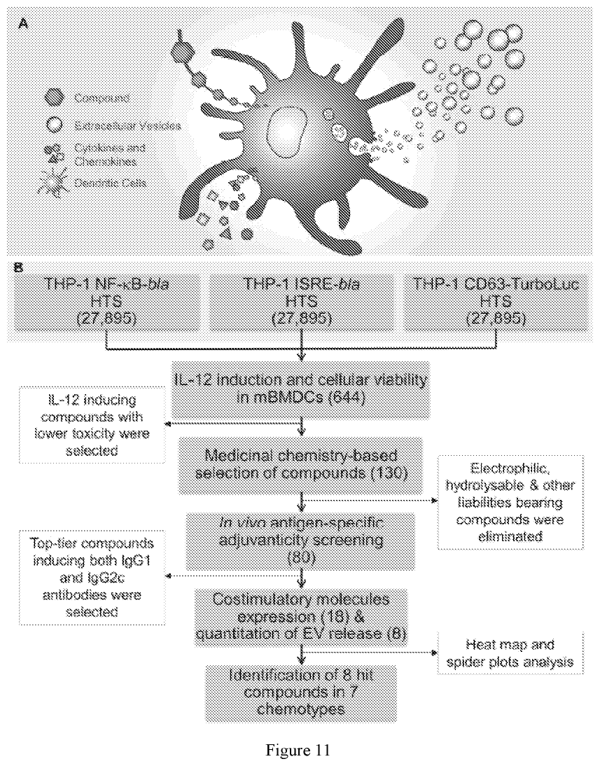

The overall HTS workflow strategy is shown in FIG. 11B. To identify compounds that induce both immune responses and EV release, three sets of screens were performed using the following reporter cell lines: NF-κB-bla, ISRE-bla, and CD63-Tluc-CD9-EmGFP THP-1 cells. In order to verify the feasibility of these assays, about 2,211 test compounds (8% of all library compounds) were randomly selected and assessed in a pilot screen for CD63 (Shpigelman et al., 2021), NF-κB and ISRE. Each screen was done in duplicate as two independent experiments (experiment 1 and 2) run on different days. The duplicate screening format allowed us to better understand the reproducibility of the activity response in the NF-κB and ISRE assays as it was found that these FRET based assay readouts have been historically less reproducible (Pu et al., 2012). Also, in the case of CD63 HTS, the pilot screen helped validate the assay (Shpigelman et al., 2021) and formed the basis for evaluating methods to be utilized for hit selection.

### Hit Selection Methods

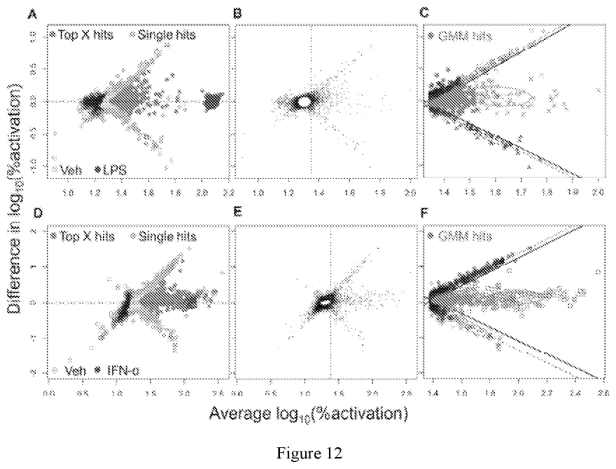

One of the most common methods utilized in selection of hits from HTS is Top X (McFayden et al., 2005). Thus, this method was initially employed for hit selection. Because each compound was evaluated in 2 independent experiments, many of the false positives which usually dominate FRET-based screens could be eliminated. To this end, an MA plot (log fold change in activation vs. average activation) was obtained by plotting the difference in the log 10% activation data for the two different experiments on the Y-axis, against the average of these data on X-axis, for each compound. These plots for NF-κB and ISRE HTS (FIGS. 12A,D, respectively) show the cluster of compounds (yellow circles) which were identified as hits only in one experiment and were thus considered “false positives” while the compounds that were identified as hits in both the experiments (red circles) were marked as “Top X hits.” Since this was a very diverse HTS library within a relatively small set of compounds, to increase the sensitivity of hits detection a Gaussian mixture modeling approach was used, applied separately to the NF-κB and ISRE screens. In this approach, the % activation values from the two independent experiments for each test compound were first used to construct a MA plot, and based on the plot we built a bivariate Gaussian mixture model (GMM), and this was used to cluster compounds into hit or non-hit categories. Since this method was heavily influenced by the large proportion of the non-hits, a null cluster in which the majority of compounds had activity levels similar to those of vehicle (Veh, 0.5% DMSO), was first identified (FIGS. 12B,E) using an initial GMM. The compounds with average % activation values lower than the maximum value of this null cluster (red dotted line, FIGS. 12B,E) were removed from subsequent analysis. GMM were then fitted to the remaining data, where the Bayesian Information Criterion (BIC) was used to determine the optimal number, shape and orientation of clusters. Apparent false positive clusters (grey/black colored compounds clustered along the black lines) were identified to construct linear boundaries (black lines). Compounds in the remaining clusters within these boundaries were considered to be hits (FIGS. 12C,F).

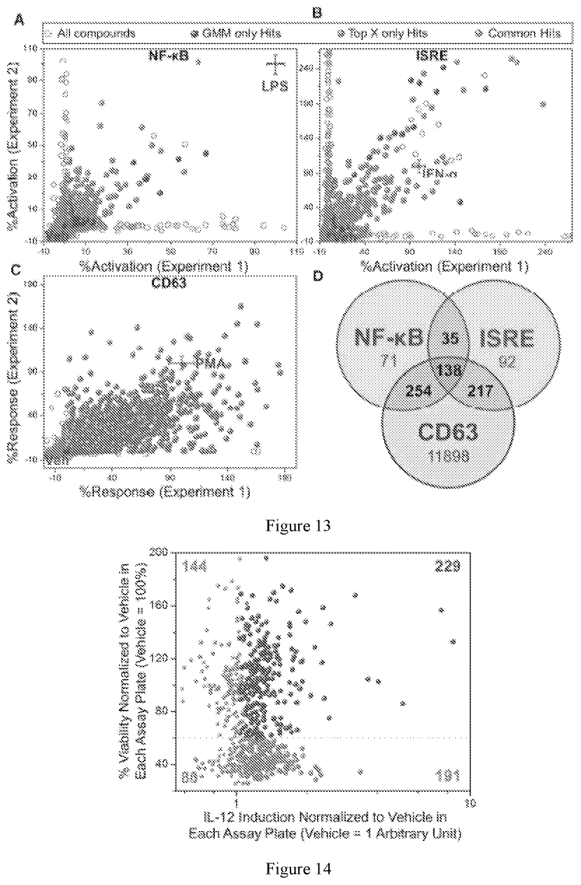

Using these two methods for identification of hits, 398 Top X hits and 497 GMM hits were identified, of which 319 hits were common between both methods, as depicted by different colored spheres in FIG. 13A. Thus, a total of 576 hits were identified from the NF-κB HTS. Similarly, for the ISRE screen, we identified 481 Top X hits and 444 GMM hits, of which 383 hits were common between both (FIG. 13B) leading to a total of 542 ISRE hits. FIGS. 13A,B show the variability of % activation data between experiments 1 and 2 in both NF-κB and ISRE HTS assays, respectively.

In contrast, the CD63 HTS showed a very good correlation between the two independent experiments. Thus, only Top X hits were used, using an average of the two independent screens. To cover even weak CD63 inducers with good NF-κB or ISRE activity, we chose to use the mean of Veh wells within an assay plate as the threshold to identify Top X hits, which led to 12,954 compounds. To further narrow down the number of hits to compounds that have activity in a minimum of 2 out of these 3 HTS, compounds were segregated into 161 triple hits (compounds identified as a hit in CD63, NF-κB and ISRE HTS), and 3 sets of dual hits including 296 CD63 and NF-κB hits, 231 CD63 and ISRE hits and 37 NF-κB and ISRE hits. Then, based on the cell viability data obtained from the PrestoBlue assay, the number of hits were distributed with the number of hits decreasing with increasing cell viability cut-off. However, because cytotoxicity of a compound can often be circumvented by subsequent structure-activity relationship (SAR) studies, the cut-off for hit selection was kept as compounds having more than 40% cell viability. A Venn diagram in FIG. 13D shows the number of hits selected by each screen based on this 40% viability cut-off in PrestoBlue assay. Thus, a total of 644 compounds which were identified as a hit in at least 2 experiments and had viability of more than 40% by PrestoBlue were selected for further analysis.

An efficiency of compound screening methods and hit identification techniques is determined by hit confirmation rates. Thus, since all the compounds in the pilot screen were part of the HTS, we used the pilot screen as an independent confirmation screen to estimate confirmation rates. A Venn diagram was first generated for both NF-κB and ISRE screens using the number of hits from the following three sets 1. Pilot screen hits, 2. Hits identified by Top X method in HTS, and 3. Hits identified by GMM method in HTS. Based on these numbers, hit confirmation rates were calculated for all three HTS. Comparing the combination of Top X and clustering based hit identification methods utilized earlier for NF-κB HTS to the combination of Top X and GMM utilized here, increased confirmation rates were found, from 31.5% in a prior similar HTS (Pu et al., 2012) to 67.6% in the current HTS for Top X method and 79.2% for the GMM method. The high confirmation rates were likely due to increased information available from the evaluation of compounds in duplicate. By using similar methodology, the hit confirmation rates for ISRE HTS were calculated as 57.7% for the Top X method and 60.9% for the GMM method. For CD63 HTS, the hit conformation rate was 52.6% by the Top X method only.

### Immunostimulatory Cytokine Induction

The selected 644 compounds that consisted of 138 triple hits, 254 CD63 and NF-κB hits, 217 CD63 and ISRE hits, and 35 NF-κB and ISRE hits were cherry picked from the original HTS source plates to evaluate their immune stimulating activities in primary mBMDCs. These mBMDCs were incubated with compounds (10 μM, in triplicates) overnight and NF-κB downstream cytokine IL-12 release in the culture supernatant was measured by ELISA while the remaining cells in the plates were measured for viability by 3-(4,5-dimethylthiazol-2-yl)-2,5-diphenyltetrazolium bromide (MIT) assay. DMSO (0.5%) was used as a Veh control to determine the baseline IL-12 induction, and the IL-12 levels for all the compounds were normalized to Veh (IL-12 induced by Veh=1). Similarly, the cell viabilities measured by MIT were normalized to Veh as 100%. The scatter plot in FIG. 16 demonstrates the relative viability on the Y-axis and the normalized IL-12 inducing activity on the X-axis. Compounds were identified that induced IL-12 more than 3 standard deviations above the mean of Veh in each plate and categorized them by cell viability into 2 groups. 229 compounds having viabilities above 60% (blue spheres) and 191 compounds having viabilities below 60% (red spheres), were identified as shown in FIG. 16. These 229 hits were then rescreened for IL-12 induction at 5 μM compound concentration to further confirm IL-12 inducing potency which led to identification of 130 compounds that induce IL-12 more than the mean+SD above Veh in each plate.

### Medicinal Chemistry Based Elimination of Hits

In an effort to narrow down the selection of hits for further in vivo adjuvanticity screening, each compound structure was evaluated for electrophilic characteristics, and presence of reactive and/or unstable functionalities, including Michael acceptors, hydrolyzable esters, reactive thioureas, and other indicators of pan-assay interference compounds (PAINS) (Baell and Holloway, 2010; Baell and Walters, 2014). Of the 130 compounds identified earlier, some of the compounds bearing such functionalities were removed. Thus, the selected 80 compounds were sourced from the vendor and purchased in sufficient quantities (5-10 mg) to perform further bioactivity evaluation in an in vivo adjuvanticity screen.

In vivo Adjuvanticity Screening

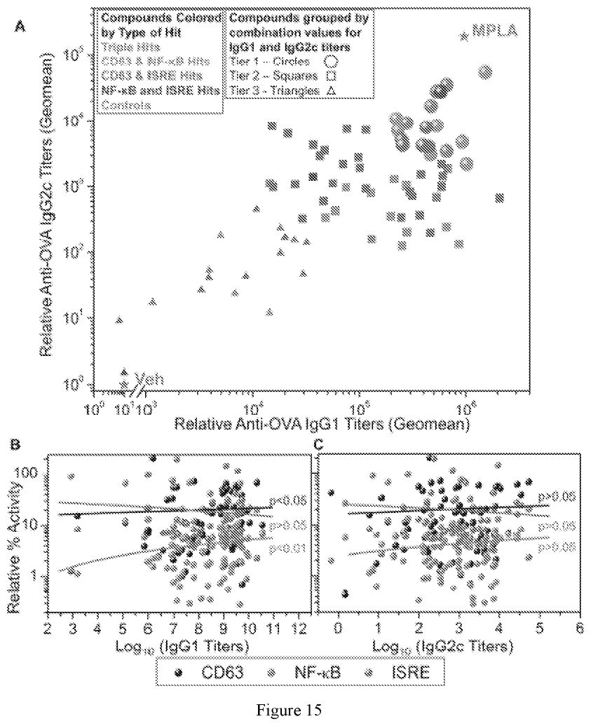

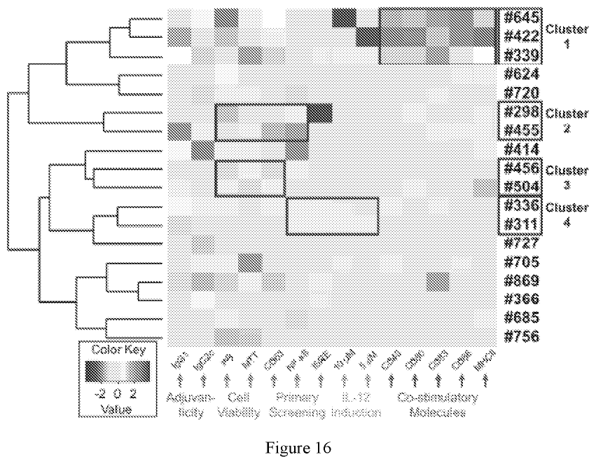

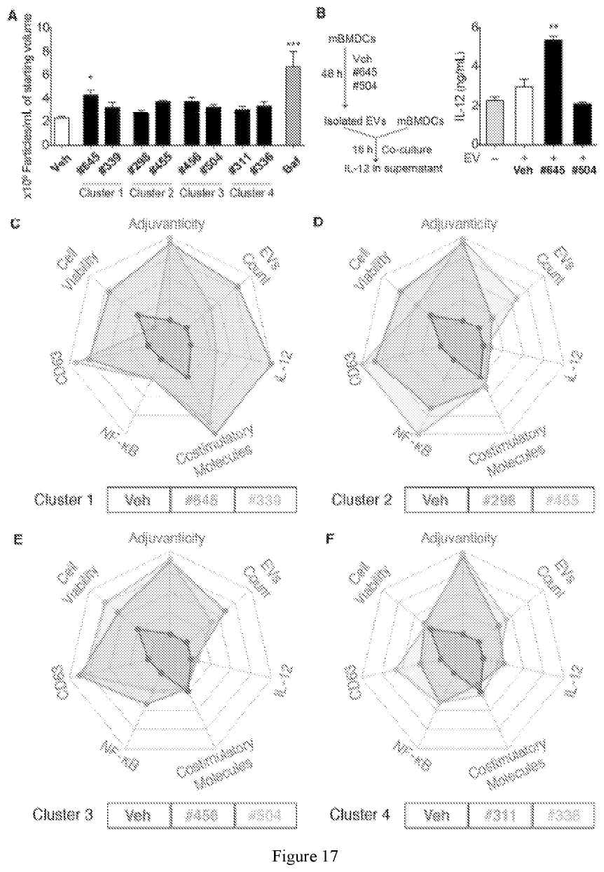

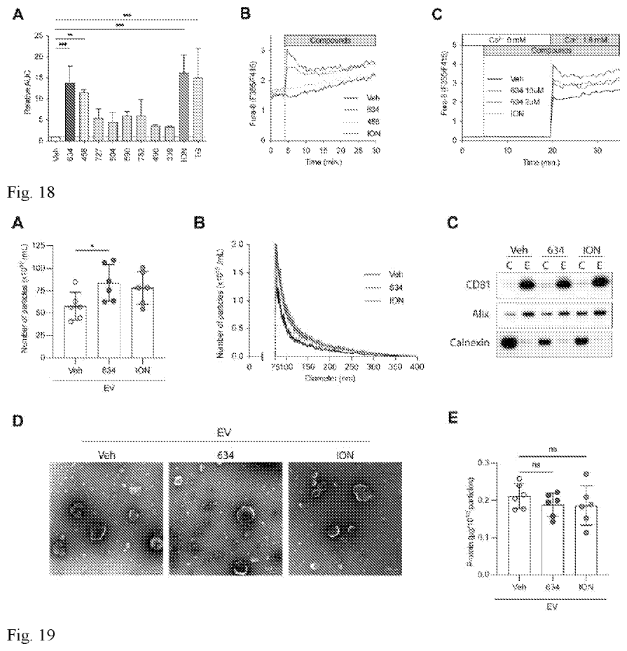

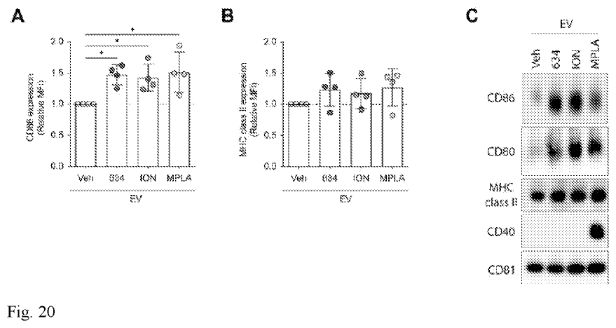

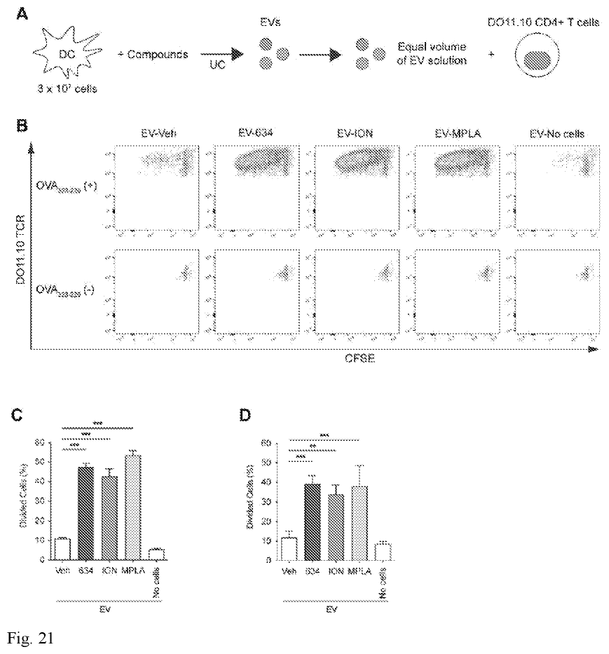

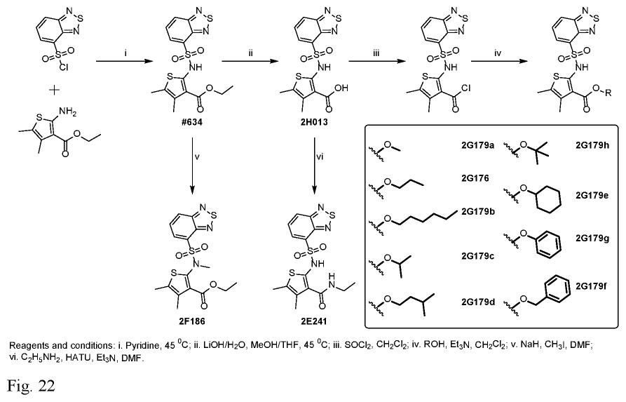

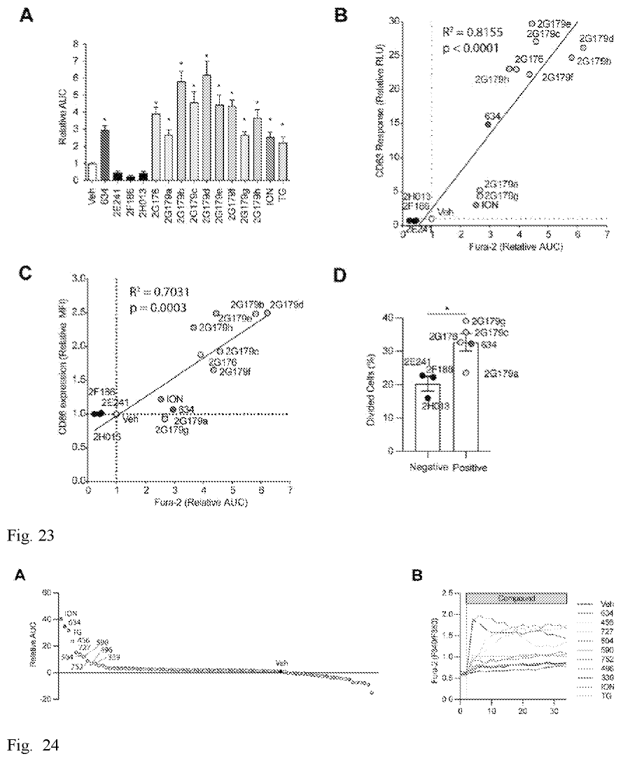

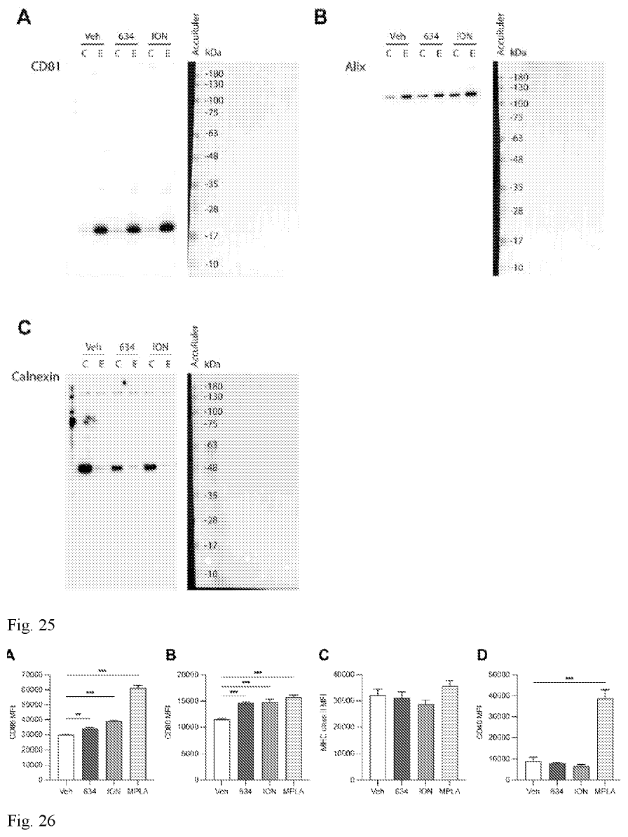

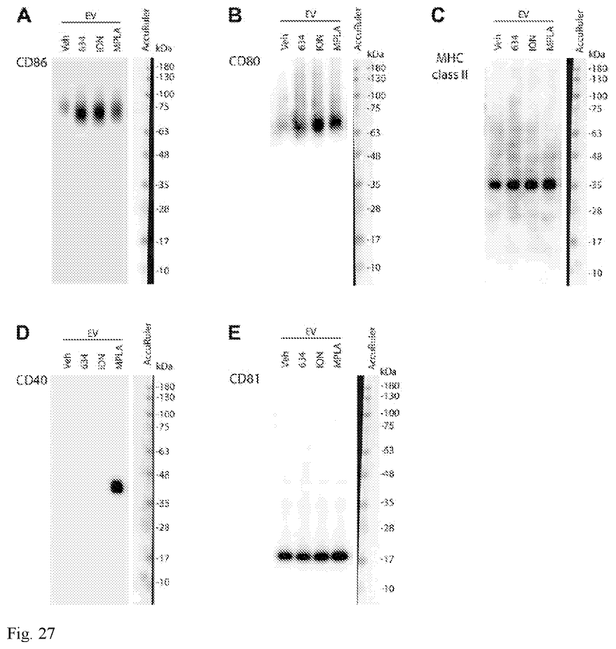

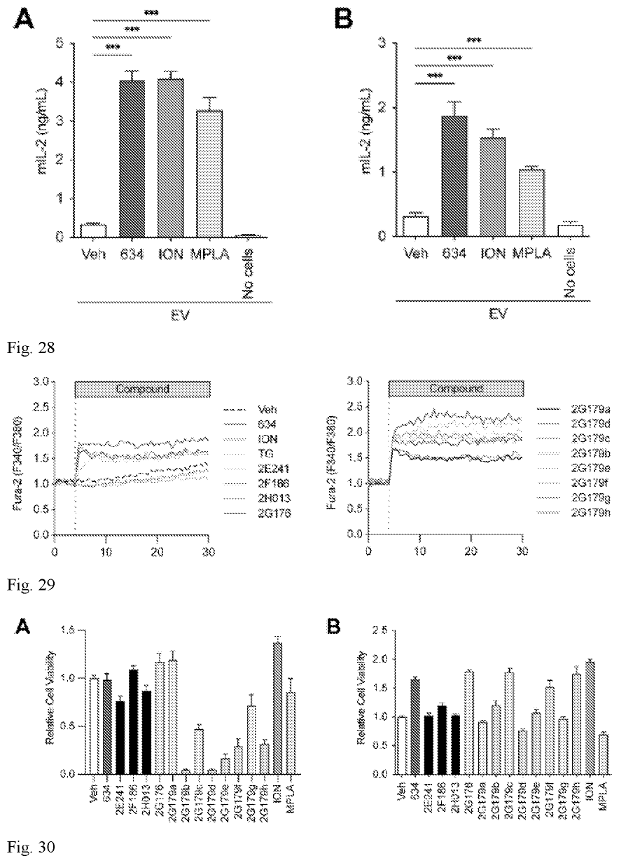

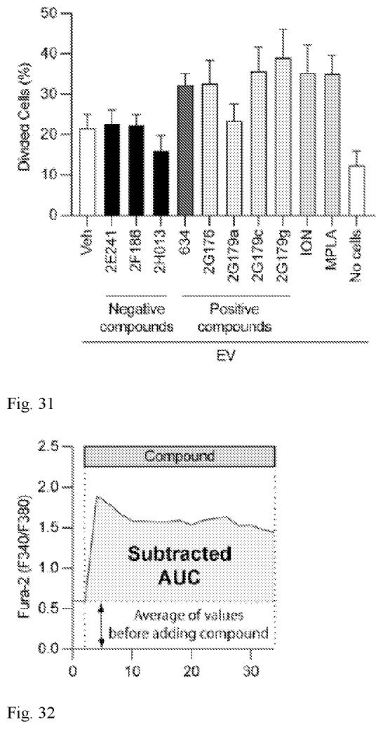

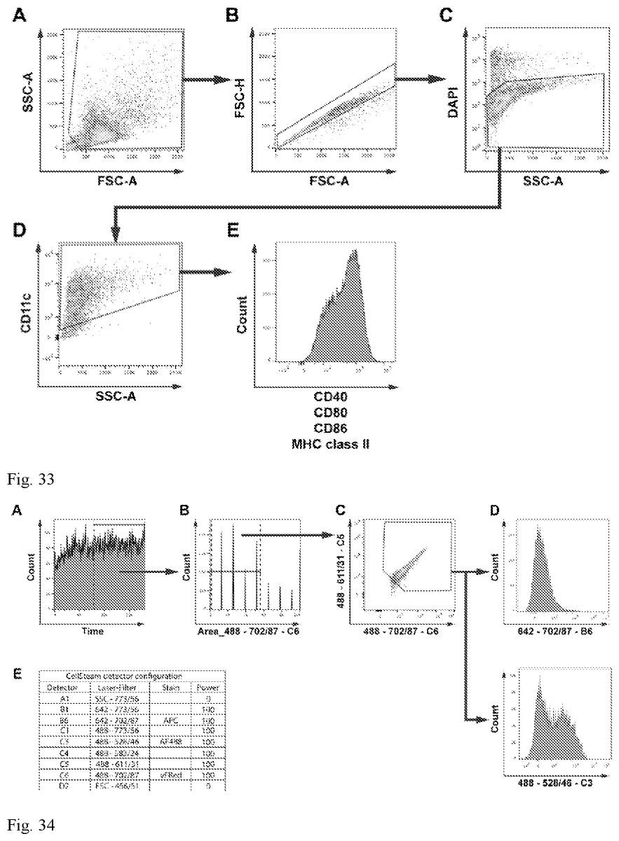

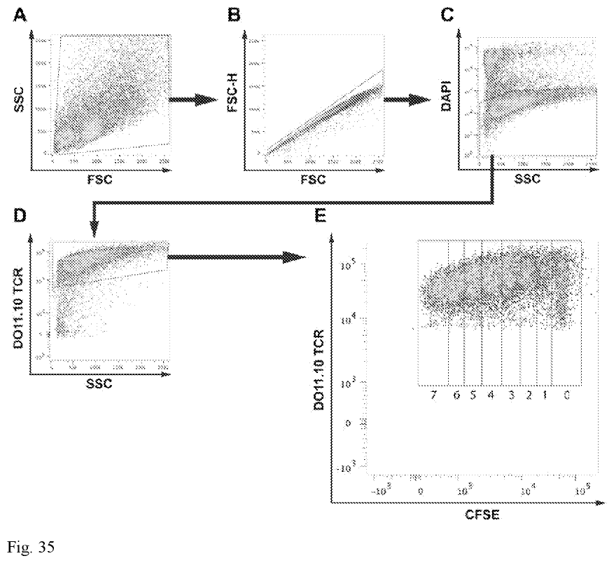

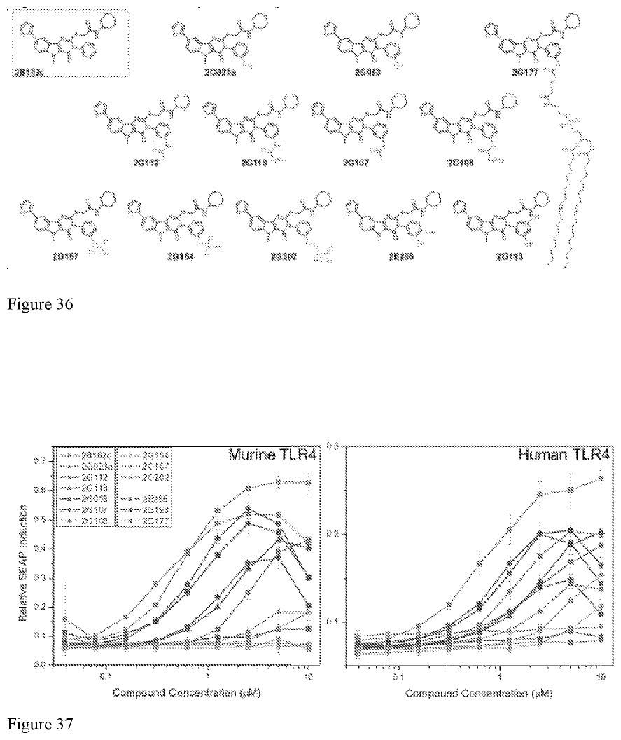

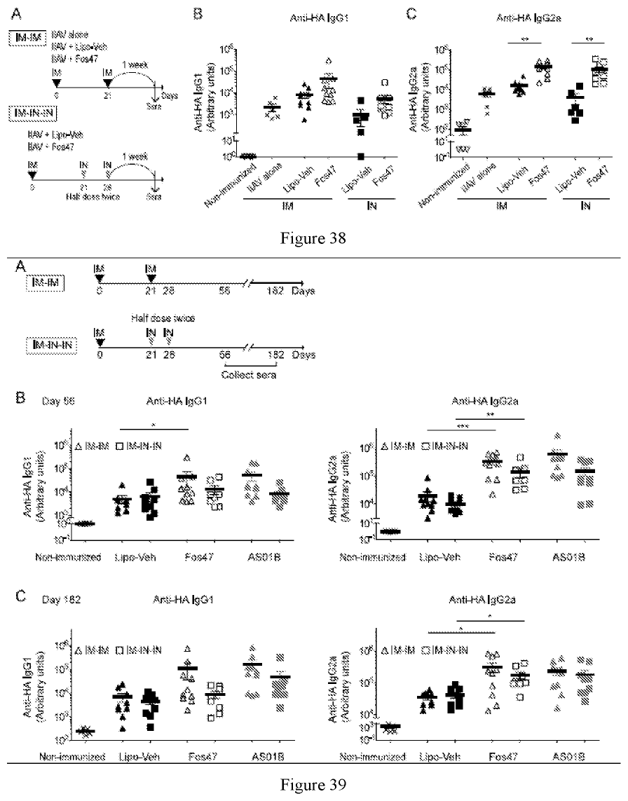

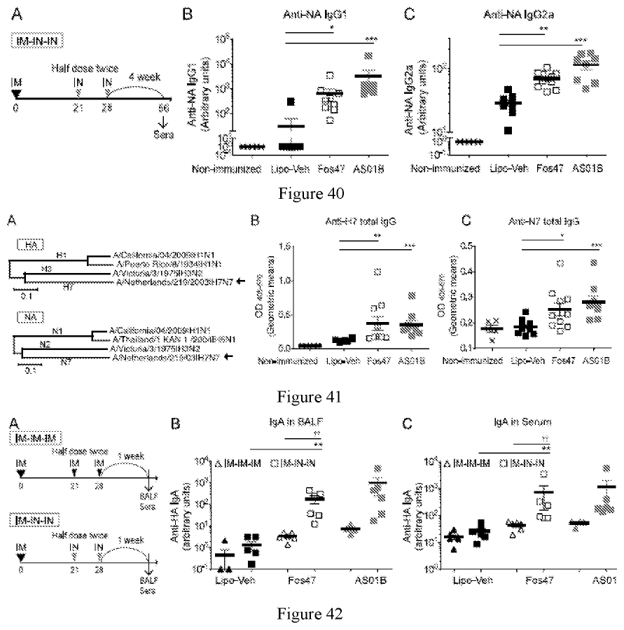

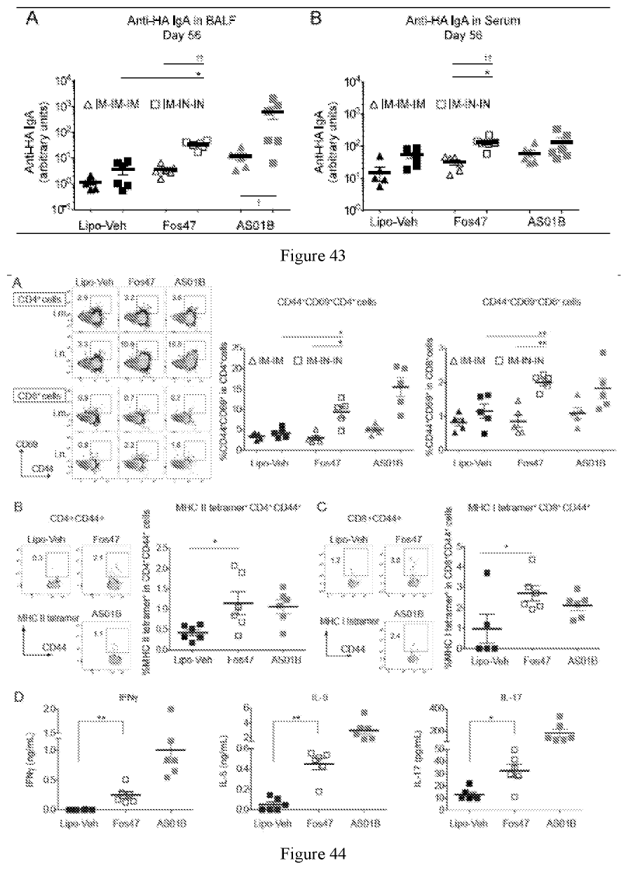

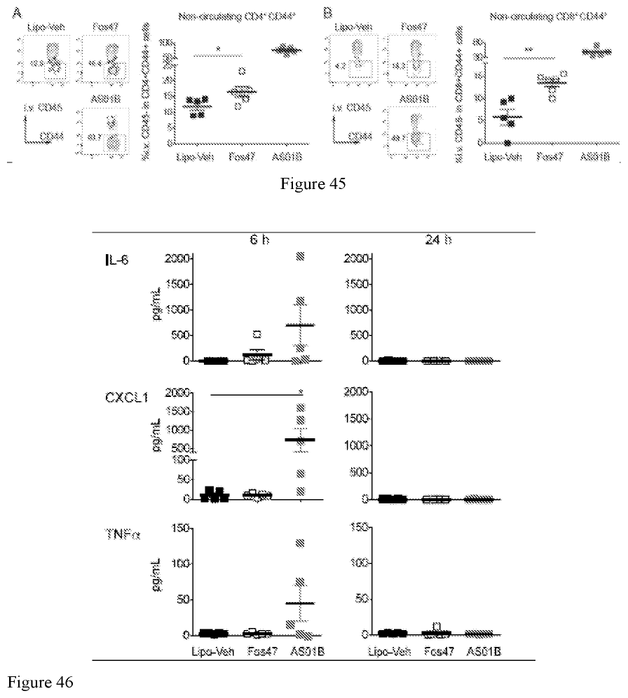

The selected 80 compounds were first evaluated for purity and identity by HPLC-MS and compounds which were less than 90% pure were purified by Prep-HPLC. These were then evaluated for vaccine adjuvant activity in mice using a model antigen, ovalbumin (OVA). C57BL/6 mice (n=3 for each compound) were immunized with OVA (20 μg/animal) mixed with 200 nmol/injection compound on days 0 and 21. Monophosphorylated Lipid A (MPLA, 1 μg/animal/injection) was used as a positive control and 10% DMSO was used as vehicle control (Veh). Sera were collected on day 28 and OVA-specific immunoglobulins IgGI (Th2 type) and IgG2c (ThI type) were determined by ELISA (Sato-Kaneko et al., 2020). These data are presented as a scatter plot in FIG. 15A that shows the distribution of compounds by their adjuvanticity profiles in inducing ThI and Th2 responses. The data points are colored based on the type of hit (triple hits or three different dual hits) and the shape of the data point represents the adjuvanticity tier (FIG. 15A, Tier 1: circles, Tier 2: squares and Tier 3: triangles). These tiers were obtained by first calculating log₁₀ transformed values of the IgG1 and IgG2c titers and normalizing these values in each set for compounds between 0 and 10 (10 for MPLA and o for Veh). This was followed by averaging these values for IgG1 and IgG2c to obtain a combination value for each compound, where Tier 1 compounds had values >8, Tier 2 compounds had values between 6 and 8 while Tier 3 compounds had this combination value less than 6. The dominant presence of CD63 and NF-κB hits in the Tier 1 compounds suggests the involvement of NF-κB and CD63 activation pathways for adjuvant activities. Thus, we probed the correlation between these primary screening data and the adjuvanticity to understand if these pathways involve any particular common mechanism to induce immunoglobulins. FIGS. 15B,C shows correlation data of primary screening data with IgG1 and IgG2c antibody titers, respectively. The analysis revealed a correlation of IgGI titers with NF-κB and CD63 assays, but less so with IgG2c titers. Since we aimed to discover compounds that induce good adjuvanticity, we selected Tier 1 compounds (18 compounds, circles in FIG. 15A) to probe into mechanisms related to immune stimulation.

### Co-Stimulatory Molecules Expression

Antigen presenting cells (APCs) such as dendritic cells play important roles in innate immune responses to transduce signals for subsequent humoral immunity (Liechtenstein et al., 2012). Costimulatory molecules, including CD80/86, CD40, and MHC class II molecules, are expressed on APCs and bind to their corresponding receptors on naive or memory T cells, signalling T cell proliferation or maturation (Liechtenstein et al., 2012). While we revealed that the selected compounds had cytokine inducing effects and in vivo adjuvanticity when used with an antigen, the effect of these compounds on APC function was unknown. Hence, to examine whether these 18 compounds enhance maturation of immature dendritic cells, facilitating antigen-presenting cell function, mBMDCs were treated with 10 μM compound or Veh overnight and the expression of costimulatory molecules (CD40, CD80, CD83, CD86 and MHC class II) on CD11c⁺ cells was examined by flow cytometry (FIG. 16). Of all these compounds, #645, #422, and #339 notably enhanced the expression of costimulatory molecules (FIG. 16, cluster 1).

### Heat Map Presentation of Compound Activity Profiles

Starting the HTS with 27,895 compounds, 18 hits were selected. It was queried if there was any coherence in biological activity that would have driven the selection. Thus, the compounds were clustered by their activity profiles' including adjuvanticity (IgGI ang IgG2c), cell viability (PrestoBlue and MTT assays), primary HTS (CD63, NF-κB, and ISRE), IL-12 induction (10 and 5 μM compound concentration), and co-stimulatory molecules expression (CD40, CD80, CD83, CD86, and MHC class II). All these data were normalized using negative and positive controls to remove batch effects and each variable was standardized before heat maps were generated (FIG. 18). Based on hierarchical clustering (shown on the left in FIG. 16), we were able to group compounds into four clusters. Cluster 1 consisted of 3 compounds #645, #422, and #339. This group of compounds showed similar effects on the induction of co-stimulatory molecules in mBMDCs. Next, cluster 2 (#298 and #455) and cluster 3 (#456 and #504) consisted of compounds with similarity in their cell viability profiles as well as in the primary screenings. The last cluster 4 (#336 and #311) also showed similar bioactivity in primary screenings as well as IL-12 induction.

### EV Characterization and Spider Plots

To quantitate EV release from mBMDCs, these clustered 8 compounds were selected (compound #422 had very poor aqueous solubility and was not evaluated for EV particle count) and EV particle numbers assessed in the culture supernatants of mBMDCs cultured with 10 μM compound for 48 hours using microfluidic resistive pulse sensing (MRPS) with a nCS1 Instrument (Spectradyne, Signal Hill, CA). The EVs in the culture supernatants were isolated following the multistep differential ultracentrifugation protocol as previously described (Shpigelman et al., 2021). Only compound #645 increased the number of EV particles released in the culture supernatant compared to Yeh control (FIG. 19A). Immunoblots of isolated EV pellets confirmed enrichment for the tetraspanins CD81 and Tsgl0l. It was also examined if the immunogenicity was from direct compound stimulation of cells or could be transferred by EVs from stimulated cells (FIG. 17B). Two compounds were selected as relatively high and low inducers of EV release (#645 and #504, respectively). The EVs from #645-treated cells stimulated higher levels of IL-12 release than those from Yeh-treated cells indicating that the EVs from the cells are capable of innate immune stimulation.

To compare activity of the clustered compounds from the heat map above, their multiple bioactivities, including adjuvanticity and co-stimulatory molecules expression, were used to generate spider plot overlays along with the EV particle count data. For cluster 1, compounds #645 and #339 had equipotent activity in CD63 and NF-κB assays and co-stimulatory molecules expression, although they varied in IL-12 induction and cell viability, which translated as well to quantities of EVs (FIG. 17C). Thus, #645 that belongs to a 3-pyridyl-oxadiazole scaffold was revealed as a promising lead chemotype. For cluster 2, compounds #298 and #455, both had moderate effects on induction of cytokine IL-12 and expression of co-stimulatory molecules, but had similar CD63 and NF-κB activities in the primary HTS and both showed high values (indicating possible cell proliferation) in the MTT assays (FIG. 17D). The EV particle counts inversely varied with MTT assay data suggesting distinct properties from cluster 1 compounds and pointed towards mechanisms that may involve increased formation of EVs due to decreased cell numbers (cells breaking down to release EVs). Cluster 3, consisting of compounds #456 and #504, had similar activity profiles as that of cluster 2 compounds in terms of higher MTT values inversely relating to EV particle count size but had much lower NF-κB inducing activity (FIG. 17E). Cluster 4, consisting of compounds #311 and #336, showed very similar activity profiles, Through a rigorous tiered screening process, we discovered that these 2 compounds share structural similarity and belonged to the same 4-thieno-2-thiopyrimidine chemotype (FIG. 17F).

### DISCUSSION

In immune responses, EVs have been reported to play immune-stimulatory and -suppressive roles. As a screening tool we utilized a human cell line from a lineage that has antigen presentation functions. Dendritic cells (DCs) are the most effective cell type for antigen presentation to immune cells and EVs released from DC can stimulate pro-inflammatory responses (Zitvogel et al., 1998; Escudier et al., 2005; Morse et al., 2005; Besse et al., 2016). Moreover, EVs are known to play a role in acquired immunity, as EVs released from macrophages and DCs display major histocompatibility (MHC) class I and II molecules, co-stimulatory molecules (CD80 and CD86), and the adhesion protein ICAM-1 (CD54) on their surface (Admyre et al., 2006; Schorey et al., 2015; Wen et al., 2017; Lindenbergh and Stoorvogel, 2018). Antigen presentation to T cells can occur directly by MHC molecules loaded with the antigenic peptide on the surface of EVs or the EVs can be taken up by DCs or macrophages and the antigen processed to be presented by their MHC surface molecules. A HTS was developed to specifically identify chemicals that would increase biogenesis and release of EVs to enhance antigen specific immune responses in an adjuvant role. #645 was identified, exhibiting not only intrinsic immunostimulatory activity but also induce release of immunostimulatory EVs (FIGS. 17A,B). As #645 enhanced the expression of MHC class II and costimulatory molecules on mBMDCs, the EVs isolated from #645-treated mBMDCs may also have these surface proteins, contributing to antigen presentation and augmenting T cell responses. Furthermore, the in vitro co-culture study with EVs and mBMDCs showed that the EV released from #645-treated mBMDCs induced IL-12 release (FIG. 17F), suggesting that these EVs may also induce innate immune responses from other surface interactions like PRRs.

The use of EVs as a vaccine platform is under intensive investigation. EVs can be harvested from the supernatants of cells engineered to produce antigens and/or have specific cargos like mRNA (Kanuma et al., 2017; Anticoli et al., 2018; Jafari et al., 2020). In the first case the released EVs can preserve the native conformation of the antigenic proteins for delivery to the lymphoid system. In the second system the vaccine recipient would express the proteins at the site of delivery. The distribution of EVs administered in vivo is dependent on the cell source of the EVs and the route of administration (Wiklander et al., 2015). EVs administered intravenously in mice are rapidly cleared from the circulation, with a half-life of 2-4 minutes with complete clearance after 4 hours (Takahashi et al., 2013). These EVs preferentially accumulate in the liver and spleen and are largely taken up by macrophages, which participate in the clearance of EVs (Imai et al., 2015). After half an hour EVs start to be eliminated by hepatic and renal clearance mechanisms which is completed within roughly 6 hours (Takahashi et al., 2013; Lai et al., 2014). EVs that are administered subcutaneously have less hepatic uptake and a slower clearance. The long term aim of our strategy is to use a chemically controlled release of EVs at the site of antigen administration, that would enable the recipients' cells to produce EVs continuously over time. This release is likely to be slower than a bolus of EV administration, but the continuous production would potentially overcome the limitation of rapid clearance.

The CD63 HTS demonstrated that there are many different chemical scaffolds that induce the release of EVs, further reinforcing that the release of EVs is an important intercellular communication mechanism potentially inducing the stimulation of multiple intracellular pathways. To reduce our pool of hit candidates, we performed parallel screens on two other reporter lines, NF-κB and ISRE-bla reporter cells utilizing the same parent THP-1 cells. Compounds from each of the intersecting groups were able to stimulate an in vivo immune response to a test antigen above that of the antigen without any adjuvant. This indicates that the use of a triple screen still allowed for a broad ability to capture multiple potential leads without skewing to a single mechanism of action. Choosing Maybridge compound library for HTS enabled the identification of relatively unexplored compounds. The tiered screening process selected two compounds that belong to 4-thieno-2-thiopyrimidine scaffold having identical screening profiles for cytokine stimulation and cell surface marker induction thus supporting an internal reproducibility and validating our overall screening.

Although three parallel screens to identify compounds were used, the initial in vivo testing indicated that compounds with all three activities were effective adjuvants. Among the three HTS, the compounds that had the ability to stimulate EV release and NF-κB activity with or without ISRE activity were found to be the most potent adjuvants. The compounds that were the most effective that had ISRE activity also had NF-κB activity and high EV release (triple hits). In contrast, others have identified molecules that stimulate type I interferon release and ISRE activity that resulted in potent activity as adjuvants (Martinez-Gil et al., 2013). The present findings may be relative only to the compounds that were included in the library and may not be generalizable to other systems. In addition, compounds were screened for in vitro toxicity with both PrestoBlue and MTT assays. Thus, it is not likely that NF-κB and CD63 inducing activity was due to toxicity. Although this study did not formally evaluate the safety profiles of the active compounds in vivo, externally isolated and administered EVs have been reported as having an acceptable safety profile including after multiple rounds of administration. (Maji et al., 2017; Zhu et al., 2017; Mendt et al., 2018; Saleh et al., 2019).

Thus, the identification of novel chemical scaffolds that are effective in vivo adjuvants is described. Screening for the release of EVs in addition to the activation of known immunologic signaling pathways added an additional dimension that identified robust scaffolds for further SAR studies of vaccine adjuvant activity. Chemical induction of EV release may prolong the delivery of antigen to the lymphoid system.

### Materials and Methods

### Cell Lines

CellSensor® NFκB-bla human monocytic leukemic THP-1 cell line was purchased from Thermo Fisher Scientific (Waltham, MA). The ISRE-bla THP-1 cell line was developed as described earlier (Shukla et al., 2018). These cell lines contain NF-κB and ISRE reporter constructs that uses a β-lactamase reporter gene which on activation results in beta-lactamase production and shifts the fluorescence emission of the beta-lactamase substrate [LiveBLAzer™-FRET B/G (CCF4-AM), Thermo Fisher Scientific] to favor coumarin (460 nm emission) over fluorescein (530 nm emission).

CD63-Tluc-CD9EmGFP THP-1 reporter cells were prepared by Thermo Fisher Scientific as described previously (Shpigelman et al., 2021). Briefly, a construct with dual reporters consisting of two tetraspanins, CD63 and CD9 reporter constructs; CD63-Tluc and CD9-EmGFP, was transduced into THP-1 cells. The Tluc activities of EVs shed from CD63-Tluc-CD9EmGFP reporter cells in the culture supernatant were quantitatively measured for EV release.

All THP-1 reporter cells were maintained in 4-(2-hydroxyethyl)-1-piperazineethanesulfonic acid (HEPES) buffered RPMI 1640 medium (#72400, Thermo Fisher Scientific) supplemented with 10% dialyzed FBS (dFBS, #26400044, Thermo Fisher Scientific), 100 U/ml penicillin, 100 μg/ml streptomycin, 1 mM sodium pyruvate, 1×MEM non-essential amino acids (NEAA), and 5 μg/ml blasticidin at 37° C. in 5% CO₂. All the HTS assay validations were carried out in assay medium OptiMEM®I Reduced Serum Medium (#31985-070, Thermo Fisher Scientific) in 384-well plates (#3712, Corning).

### Reagents

The Maybridge library series, including the Maybridge HitFinder library (14,303 compounds) and the Maybridge HitCreator library (13,592 compounds) were purchased from Thermo Fisher Scientific (Leeds, United Kingdom) (Supplementary Table 3).

LPS used as a positive control for the NF-κB HTS was obtained from Sigma-Aldrich (St. Louis, MO). MPLA was purchased from Invivogen (San Diego, CA). Human IFN-α (#11101-1, PBLAssay Science) was used as a positive control for ISRE-bla HTS. Phorbol 12-myristate 13-acetate (PMA, BP685-1, Thermo Fisher Scientific) was used as a positive control for HTS using CD63-Tluc-CD9EmGFP reporter cells. MTT was purchased from Thermo Fisher Scientific. Ovalbumin (OVA) was obtained from Worthington Biochemical Co. (Lakewood, NJ). PBS (#14190, Thermo Fisher Scientific) filtered through a 0.02 μm inorganic membrane filter (#6809-2002, Millipore, Burlington, MA) was used to wash and dilute EVs.

### High Throughput Screens and Statistical Analysis

The robotic HTS using the three reporter cells were performed using 384-well plates by the SelectScreen™ service, Thermo Fisher Scientific (Madison, WI) (Pu et al., 2012; Chan et al., 2017b; Shukla et al., 2018). For HTS using NF-κB-bla and ISRE-bla THP-1 cells, LPS (100 ng/ml) and human IFN-α (50 nM) were used as the positive controls, respectively, while 0.5% DMSO was used as vehicle (Veh). The cells were incubated with compounds (10 μM) for 5 hours, and LiveBLAzer™ FRET BIG substrate (CCF4-AM) mixture was added. Fluorescence was measured at an excitation wavelength of 405 nm, and emission wavelengths of 465 and 535 nm. The background values were subtracted from the raw values (cell-free wells at the same fluorescence wavelength). Emission ratios were calculated by dividing background-subtracted values from em1ss1on wavelength of 465 nm by those from emission wavelength of 535 nm. The response ratio (RR) was calculated as follows (emission ratio of a test well)/(average emission ratio of wells with Veh). Further, for comparison of activity, “% activation” for each compound was computed within the plate as 100×(compound RR−average Veh RR)/(average LPS RR−average Veh RR).

In the HTS using CD63-Tluc-CD9EmGFP reporter cells (CD63 HTS), PMA (10 ng/ml) was used as a positive control (Shpigelman et al., 2021) and 0.5% DMSO was used as negative control (vehicle, Veh). Briefly, the harvested cells were resuspended in assay media containing 10% exosome-depleted PBS (#A2720801, Thermo Fisher Scientific) and plated at 2×10⁵ cells/mL (50 μL/well of 384-well plates) (#3674, Corning). Test compounds were added at a final concentration of 10 μM to cells and incubated for 48 hours at 37° C. Subsequently, the plate was centrifuged, supernatant (25 μL) was transferred, and chemiluminescent was measured as recombinant luciferase activity (RLU) after 10 minutes incubation with TurboLuc assay reagent (TurboLuc™ One-Step Glow Assay kit, Thermo Fisher Scientific). The response that measures activation and subsequent release of EVs was calculated using the following formula; % response=100×(compound RLU−average Veh RLU)/(average PMA RLU−average Veh RLU). The viability of cells was assessed using PrestoBlue reagent (Thermo Fisher Scientific). Briefly, PrestoBlue reagent (2.5 μl) was added to the remaining cells and incubated for 30 minutes at room temperature, followed by fluorescence readout at (Ex 560 nm/Em 590 nm) which was normalized to fluorescence data of the Veh wells within the plate to obtain “% viability” calculated as 100×(compound fluorescence/average Veh fluorescence).

### Data and Statistical Analysis for Selection of Hits

In each of the 3 screens, compounds were assayed twice in two replicate HTS experiments (experiments 1 and 2 performed on different days). The HTS readout was evaluated as % activation as mentioned above). For the NF-κB and ISRE HTS, hit compounds were identified using two statistical methods 1) “Top X” threshold approach and 2) “Gaussian mixture model (GMM)” approach. A compound identified by either of these methods was considered a hit. However, for the CD63 HTS, only the Top X method was used to identify hits.

Top X method: In the Top X method, all compounds with % activation values above a given threshold were selected. The threshold was computed for each plate, using the Veh wells (cells treated with 0.5% DMSO), as the mean+3SD of % activation from these wells. Any selected compound was considered to be a false-positive if both coumarin and fluorescein values were extreme outliers according to the manufacturer's instruction (Thermo Fisher Scientific). When a test compound was selected as a hit in both of the 2 independent HTS experiments, it is reported it as a Top X hit (FIGS. 12A,D). For the CD63 HTS, we used the mean % response of the Veh wells as the per-plate threshold value.

GMM method: Since all compounds were assayed twice in two independent experiments, we could identify hits using a GMM. In this approach % activation data from the two independent experiments were used to construct bivariate GMM (Hastie and Tibshirani, 1996) implemented in the R-mclust package (Scrucca et al., 2016). These models were used to cluster compounds into hit or non-hit categories Briefly, the arbitrary number of 20 units was added to all % activation values to ensure all values were greater than O and the data were log 10 transformed. The two independent experiments were visualized using MA (log ratio vs. average) plots, in which the X-axis was the average of log₁₀ (% activation +20) for two replicate values for each test compound and the Y-axis was the difference in these values. A GMM was then fit to the plotted data, where the Bayesian Information Criterion (BIC) was used to determine the optimal number, shape and orientation of the gaussian clusters. Since this method was heavily influenced by a large number of the compounds with low % activation values, a two-step approach was employed: at the initial step, a null cluster was identified in which the majority of compounds had % activation levels similar to those from Veh-treated wells (FIGS. 12B,E). This null cluster was removed, as were all compounds with average activity values lower than the maximum value of this null cluster (red dotted line, FIGS. 12B,E). At the second step, a second GMM was fitted, using the remaining data. Apparent false-positive clusters were identified and used to construct linear boundaries, and finally compounds from the remaining clusters within these boundaries were considered GMM hits (FIGS. 12C,F). The GMM method ensured both a larger number of hits and also higher confirmation rate when data from an initial independent pilot screen was used to estimate the hit confirmation rate.

R statistical software (R version 3.6.1, www.r-project.org) was used for selection of hits. For the data other than HTS, Prism 6 (GraphPad Software, San Diego, CA) statistical software was used to obtain p-values for comparison between groups (p<0.05 was considered significant) and for Spearman's rank correlation to test for a non-zero correlation between antigen-specific antibodies and HTS data.

### Animals

Wild type C57BL/6 mice were purchased from the Jackson Laboratories. All animal experiments received prior approval by the Institutional Animal Care and Use Committee (IACUC) for UC San Diego.

Generation of mBMDCs

mBMDCs were prepared from bone marrow cells harvested from femurs of C57BL/6 mice as previously described (Lutz et al., 1999; Datta et al., 2003). Briefly, murine bone marrow cells were harvested from C57BL/6 mice. The cells were cultured with murine granulocyte-macrophage colony-stimulating factor (GM-CSF, 20 ng/ml) for 7-8 days. Non-adherent cells were harvested and used for experiments.

### Cell Viability Assay

mBMDCs (10⁵ cells/200 μL/well) were treated with 10 and 5 μM of a test compound in 96-well plates overnight. After 18 hours of drug treatment, MTT (0.5 mg/ml) was added to each well. The cells were lysed after 6-8 hours incubation, and absorbance values at 570 and 650 nm were measured. PrestoBlue reagent (#A13261, Thermo Fisher Scientific) was used for cell viability assay in CD63 HTS as described earlier.

### Assessment of Cytokine Levels Using Primary Cells

mBMDCs (10⁵ cells/200 μL/well) were plated in wells of 96-well plates and treated with test compound (5 μM or 10 μM) or vehicle (0.5% DMSO) overnight. IL-12 levels in the culture supernatants were assessed by ELISA as previously described (Sato-Kaneko et al., 2021).

In vivo Assessment of Adjuvanticity of Compounds

C57BL/6 mice were intramuscularly injected with OVA (20 μg/mouse) mixed with a test compound (200 nmols/mouse) or MPLA (1 μg/mouse) or Veh (10% DMSO) in 50 μL total volume on days 0 and 21 and bled on day 28. OVA-specific IgG1 and IgG2c levels in sera were evaluated by ELISA as described previously (Chan et al., 2009).

### Flow Cytometric Analysis for Costimulatory Molecules

mBMDCs (10⁵ cells/200 μL/well) were treated with a test compound (10 μM) or Veh (0.5% DMSO) overnight and then the costimulatory molecule expression on mBMDCs was evaluated using flow cytometry. The cells were stained with antibodies for CD11c, CD80, CD83, CD86, CD40, and MHC class II. Dead cells (DAPI high) were excluded from the analysis. Percent positive population of CD80, CD83, CD86, CD40, or MHC class II in the gated CD11c population were analyzed.

### Heat Maps

Variables used to make a heat map were normalized and scaled by subtracting the mean and dividing by the standard deviation. Hierarchical clustering was performed, where the distance measure was the Spearman rank correlation. The R-gplots package was used to make heat maps.

### EV Isolation by Differential Ultracentrifugation

EVs were isolated following the protocol described in the previous study with minor modifications (Shpigelman et al., 2021). Conditioned culture media (40 ml) was spun at 300 g for 10 minutes to remove debris. Supernatants were subsequently spun at 2,000 g for another 10 minutes followed by the 10,000 g step for 30 minutes. Next, 30 ml of supernatants were transferred to 31.5 ml open-top polypropylene UC tubes (358,126, Beckman Coulter Life Sciences, CA) and spun at 100,000 g_{avg} for 3 hours in an SW28 rotor (K-Factor: 2,554) by Beckman Optima XL-90 Ultracentrifuge (Beckman Coulter Life Sciences). The supernatants were then gently aspirated (leaving about 50 μL), and pellets resuspended in 30 ml cold filtered PBS. Re-suspended pellets were then spun under the same conditions as the prior spin, followed by another round of gentle aspiration and resuspension to a final volume of 50 μL in cold filtered PBS. All centrifugation steps were performed at 4° C., and resultant samples were stored at −80° C. until use. All relevant data of our experiments have been submitted to the EV-TRACK knowledgebase (EV-TRACK ID: EV220165, https://evtrack.org/index.php)(Van Deun et al., 2017).

Evaluation of EV Concentrations Released by mBMDCs

mBMDCs (7.5×10⁵/ml, total 40 ml) were incubated with 10 μM test compound or vehicle (0.01% DMSO) in RPMI 1640 (#11875, Thermo Fisher Scientific) supplemented with exosome depleted PBS (#A27208, Thermo Fisher Scientific) in a T182 flask (#25-211, Genesee Scientific, San Diego, CA) for 46-48 hours and EVs were isolated from culture supernatant by differential centrifugation as described above. EV particle concentrations and particle size/distribution were determined by MRPS technology with nCS1 particle analyzer utilizing C-400 cartridges (Spectradyne, Signal Hill, CA). EV samples were diluted 100-fold in 1% Tween 20-PBS and run on the nCD1 instrument. All results were analyzed using the nCS1 Data Analyzer (Spectradyne). To exclude false particle events, we applied the following peak filters: Transit time (μs) from 0 to 80, symmetry from 0.2 to 4.0, diameter (nm) from 75 to 400, signal to noise ratio (S/N) at least 10.

### Spider Plots

In a spider plot, each axis represents one of the variables to be displayed. To reduce the number of axes and make for a more interpretable visualization, selected assay readouts within the same category were combined by averaging the scaled individual variables. The final derived variables each underwent min-max normalization, i. e, x_new=(x−min)/(max−min), where min and max are minimum and maximum values of variable x, with the min (max) taken over the set of candidate compounds. The innermost net of a spider plot marks the minimum value over all the compounds, whereas the outer most net marks the maximum. R-fmsb package was used to make spider plots.

### Immunoblotting

mBMDCs were lysed with radioimmune precipitation assay buffer (RIPA) supplemented with protease inhibitor cocktail (Roche, Manheim, Germany) and a phosphatase inhibitor (Millipore). The total protein in the samples was quantitated by Pierce micro BCA Protein Assay Kit. Two μg of cell lysate or EVs were mixed with 4×NuPAGE sample buffer (Thermo Fisher Scientific) under reducing condition with dithiothreitol (DTT, Sigma) for Tsg101 or nonreducing condition (without DTT) for CD81. When DTT, a reducing agent, was used, samples were also denatured at 95° C. for 5 minutes prior to loading. After fractionation on NuPAGE 4-12% Bis-Tris Gels (Thermo Fisher Scientific), samples were blotted onto Immobilon-P PVDF membranes (#IPVH00010, Sigma) and blocked for 1 hour in 5% BSA-TBS-Tat RT. The blots were then incubated with primary antibodies (Ab): anti-CD81 Ab (1:1,000 dilution), anti-Tsg101 Ab (1:500 dilution) overnight at 4° C. with gentle agitation. After washing, the membranes were incubated with corresponding secondary antibody for 30 minutes at RT with gentle agitation. Blots were developed with ProSignal Dura ECL Reagent (Prometheus Protein Biology Products, Genesee Scientific, San Diego, CA) and visualized using a ChemiDoc Imaging System (Bio-Rad Laboratories, Hercules, CA). AccuRuler Prestained Protein Ladder (Lamda Biotech, St. Louis, MO) was used for the molecular weight markers.

Co-Culture Study with mBMDCs and Isolated EVs

mBMDCs (7.5×10⁵/ml, total 40 ml in TI82 flask) were treated with 10 μM #645, #504 or Veh (0.01% DMSO) in RPMI 1640 (Thermo Fisher Scientific) supplemented with exosome depleted PBS (10%, Thermo Fisher Scientific) for 46-48 hours. The EVs were isolated from the conditioned media and resuspended in 50 μL filtered PBS at the final step as described above. Freshly prepared mBMDCs (10⁵ cells/100 μL) were mixed with 7 μL of the EVs in a well of 96-well plate and incubated for 18 h. IL-12 levels in the culture supernatants were assessed by ELISA.

### Example 3

Extracellular vesicles (EVs) transfer antigens and immunomodulatory molecules in immunologic synapses as a part of intracellular communication, and EVs equipped with immunostimulatory functions have been utilized for vaccine formulation. Hence, small molecule compounds that increase immunostimulatory EVs released by antigen-presenting dendritic cells (DCs) were investigated for enhancement of vaccine immunogenicity. Previously a high-throughput screening was conducted on a 28K compound library using three THP-1 reporter cell lines with CD63 Turbo-Luciferase, NF-κB, and interferon-sensitive response element (ISRE) reporter constructs, respectively. Because intracellular Ca²⁺ elevation enhances EV release, 80 hit compounds were screened and compound 634 was identified as a Ca²⁺ influx inducer. 634 enhanced EV release in murine bone marrow-derived dendritic cells (mBMDCs). In addition, 634 increased costimulatory molecule expression on the surface of EVs and the parent cells. EVs isolated from 634-treated mBMDCs induced T-cell proliferation in the presence of antigenic peptides. To assess the roles of intracellular Ca²⁺ elevation on immunostimulatory EV release, structure-activity relationship (SAR) studies of 634 were performed. The analogs that retained the ability to induce Ca²⁺ influx induced more EVs with immunostimulatory properties from mBMDCs than did those that lacked the ability to induce Ca²⁺ influx. The levels of Ca²⁺ induction of synthesized analogs correlated with the numbers of EVs released and with costimulatory molecule expression on the parent cells. Collectively, the study presents that a small molecule, 634, enhances the release of EVs with immunostimulatory potency via induction of Ca²⁺ influx. This agent is a novel tool for EV-based immune studies and for vaccine development.

Compound #634 which not only enhanced exosome release as assayed by THP-1 CD63 reporter cells, but also induced calcium uptake into cells as its unique mechanism of action. Thus, it was of interest to pursue SAR studies to identify more potent compounds in this series. The structure of compound #634 is shown in FIG. 18A. It is a sulfonamide analog formed by coupling of benzothiadiazole ring and substituted thiazole ring. Thus, further SAR studies in this scaffold were pursued. The different sites of modification are shown in different colors in FIG. 18B.

Modification of the alkyl esters group: In our previous focused SAR studies, #634 was scale-up synthesized and used as starting material to synthesize several ester analogs. The ester functional group on compound #634 was hydrolyzed to obtain carboxylic acid analog 2H013. The acid was then treated with thionyl chloride to obtain acid chloride intermediate which was reacted with several different alkyl and aryl alcohols to obtain different ester analogs. This includes methyl ester 2G179a, n-propyl ester 2G176, n-hexyl ester 2G179b, isopropyl ester 2G179c, iso-pentyl ester 2G179d, tert-butyl ester 2G179 h, cyclohexyl ester 2G179e, phenyl ester 2G179g and benzyl ester 2G179f. Modification of ester was well tolerated and that potency to induce calcium influx as measured by Fura-2 assay was either retained or slightly enhanced by alkyl esters, whereas aromatic ester analog (2G179g) lost potency. The syntheses of all these analogs are shown in Scheme 1. Based on the potency of the aliphatic esters, we also synthesized N-Boc protected amino handle bearing compound 2G198 which was deprotected to obtain compound 2G201. This compound was used a common intermediate to obtain compounds labeled with biotin (2G281a), rhodamine (2G281b), and fluorescein (2G281c) (Scheme 1B). In parallel, reaction of 2H013 with ethylamine and HATU led to an amide analog 2E241 and de-esterification of compound 2H013 led to compound 2G182. However, both these analogs led to loss of potency suggesting the necessity of retaining ester functional group at C3-position of thiophene. Other analogs include compound bearing a cyano group (2G275) and oxadiazole replacing the ester functionality as in compound 2G280 (Scheme 1C).

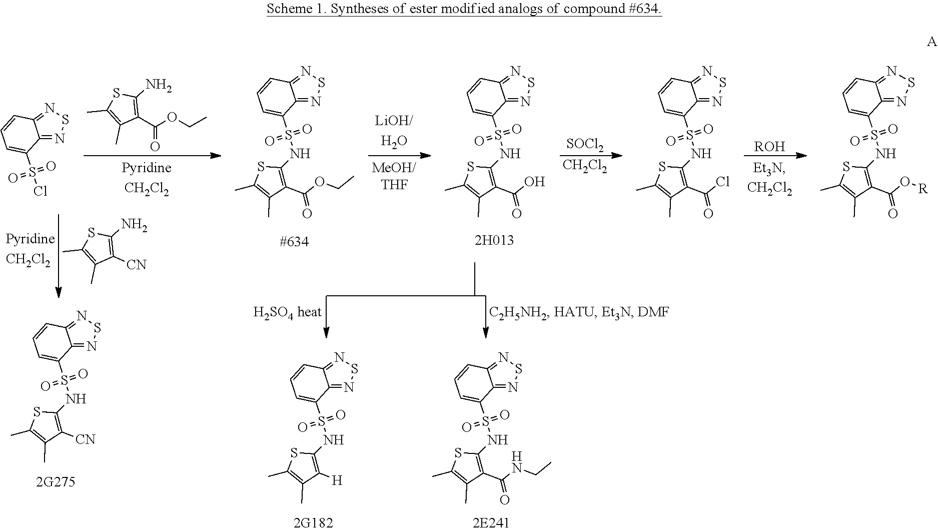

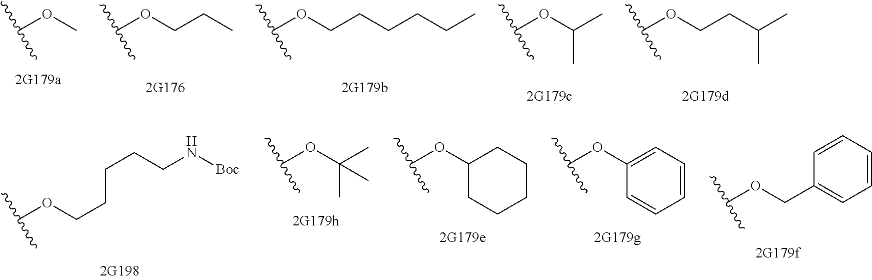

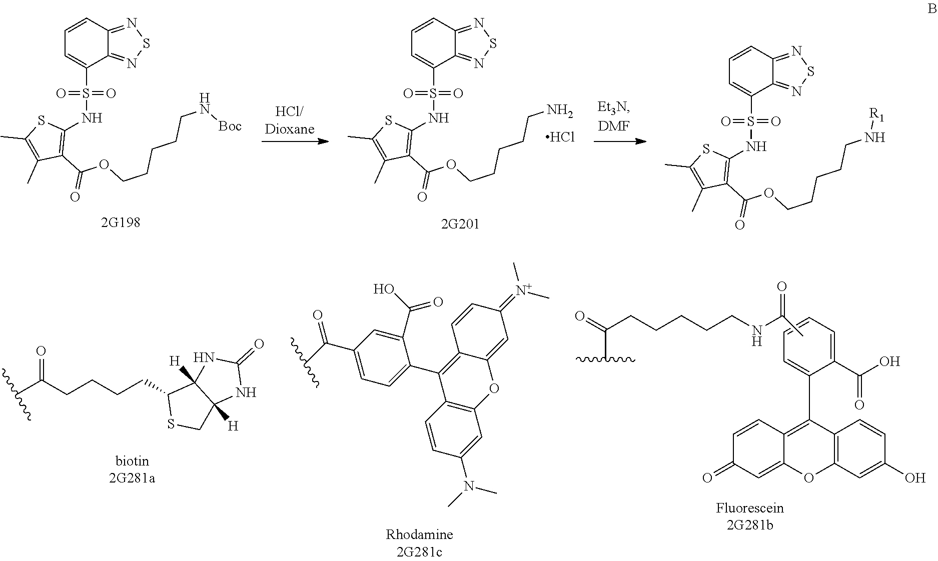

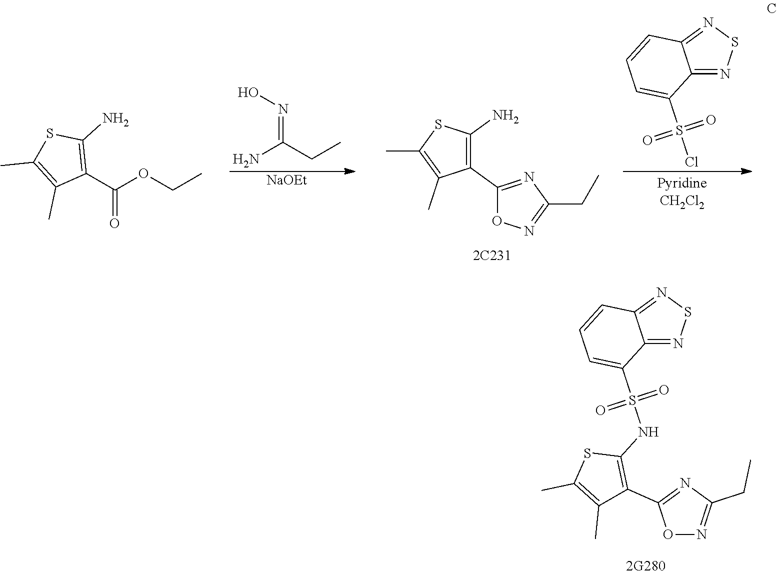

Modification of the benzothiazole ring system: Sulfur group on the structure with valence electrons is a liability from chemistry perspective and thus wanted to see if we can replace it with other functional groups. The sulfur atom was removed by reaction of compound #634 with iron powder in acetic acid to obtain the diamino analog 2G211 (Scheme 2A). Next, the sulfur atom was replaced with carbon to obtain imidazole analog 2G225 and to account for the size of the sulfur, an extra carbon was added to obtain C2-methyl imidazole analog 2G224. Both these analogs were obtained from 2G211 by reaction with formic acid and acetic acid, respectively (Scheme 1A). In parallel, 2-hydroxy imidazole analog 2C217 was obtained by reaction of 2G211 with triphosgene (Scheme 2A). A sulfur bioisostere namely selenium compound was obtained by reaction with selenium oxide to obtain 2G272 and a benzotriazole analog 2G274 was obtained by reaction diazotization of the amine of 2G211.

The next of compounds were obtained by reaction of different sulfonyl chlorides with the ethyl 2-amino-4,5-dimethylthiophene-3-carboxylate synthon as shown in Scheme 2B We eliminated the thiadiazole ring to obtain phenyl analog 2E281, replaced the thiadiazole ring with phenyl ring to obtain naphthyl analog 2G222. The next oxadiazole analog 2G223 was obtained where with oxygen atom replaced the sulfur atom. A bioisostere of sulfur atom as alkyne yielded quinoxaline analog 2H064 and the removal of nitrogen atom at position 5 on this analog led to quinoline analog 2H069, while removal of nitrogen at position 8 led to compound 2C230. A positional isomer of compound #634 (2E287) was synthesized, where the sulfonamide group was attached on the position C3 of the benzothiadiazole ring and a C4-bromo analog 2G270 was obtained to further probe the derivatization of this ring system.

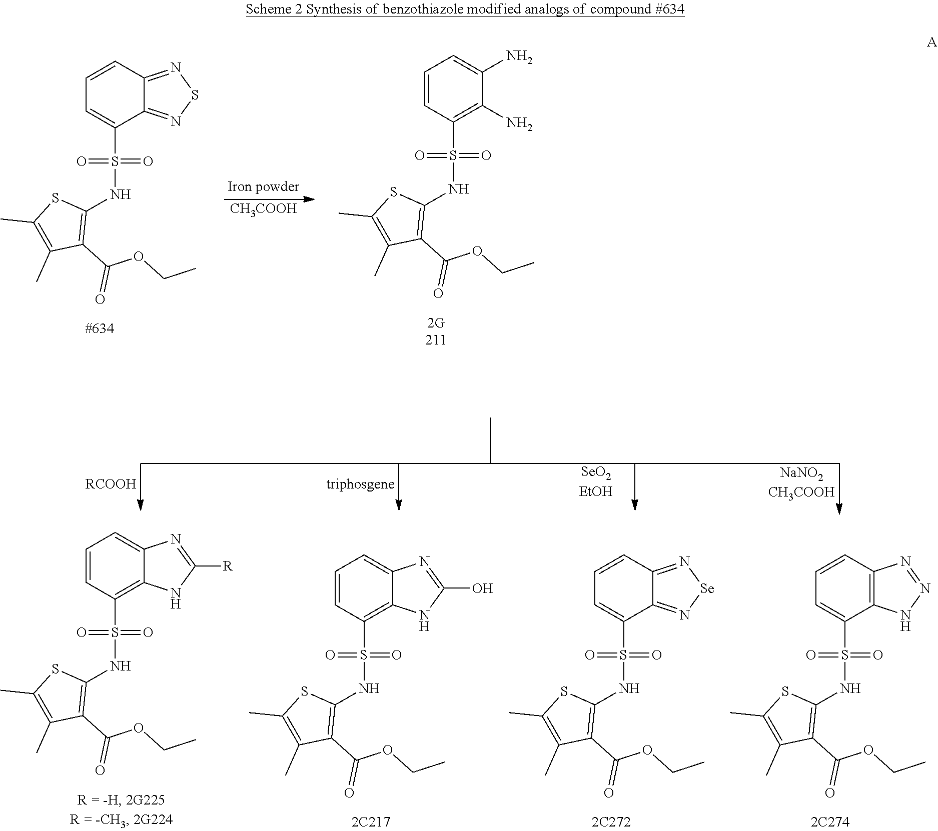

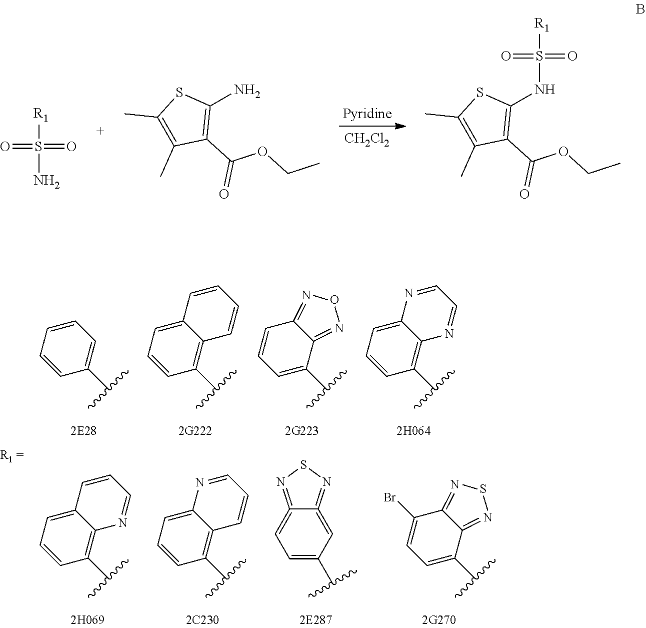

Modification of the sulfonamide group: Sulfonamide group of compound #634 was either alkylated to obtain N-methyl analog 2F186 (Scheme 3A) or was replaced with carboxamide to obtain analog 2F238 which was synthesized by HATU coupling reaction (Scheme 3B).

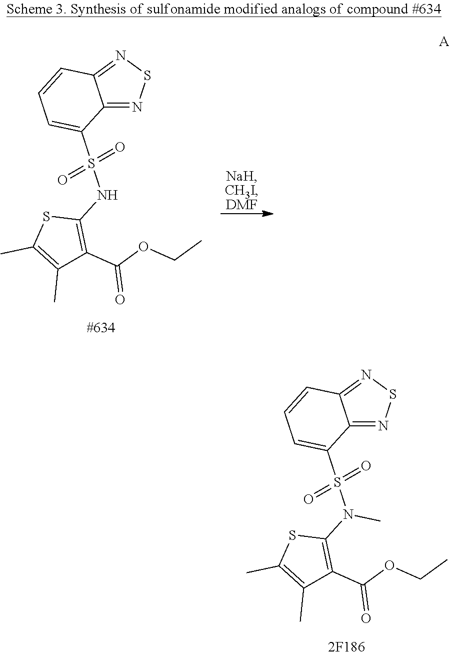

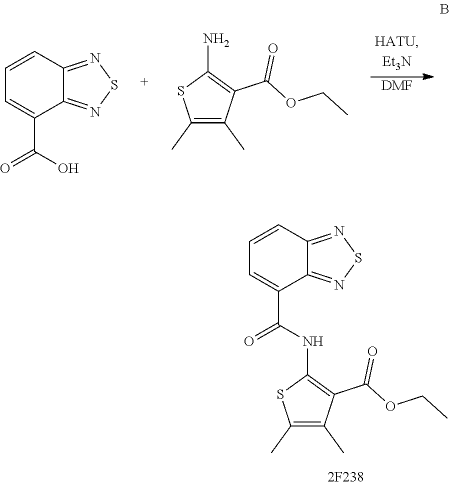

Modification of the thiophene ring: Next, the SAR studies focused on the thiophene ring. All the compounds were synthesized using the chemistry similar to one used for synthesis of compound #634. Briefly, benzo[c][1,2,5]thiadiazole-4-sulfonyl chloride was reacted with different aminothiophene synthons in presence of pyridine as base to obtain thiophene modified analogs of compound #634 (Scheme 5). Probed into elimination were 4,5-dimethyls (2C228), 4-methyl (2G244), and 5-methyl (2G245). The orientation of the thiophene ring was changed to obtain isomer 2G246b. The 4,5-dimethyls were expanded in the form of benzothiophene (2H076), cyclohexyl thiophene (2C229), cyclopentylthiophene (2H079), N-Boc protected tetrahydrothienopyridine (2G249) and its N-Boc deprotected free amine analog 2G255. Other analogs include thiazole (2G252) and 2-methyl thiazole (2G254) ring systems. Another analog designed for further derivatization include 2-bromo substituted analog 2G258. Additionally, to explore the feasibility o fusing the amino group of 2G255 as handle, derivatives were made including N-acetyl analog 2G276, N-ethyl analog 2G279 and N-isopropyl analog 2G287 as shown in Scheme 4B.

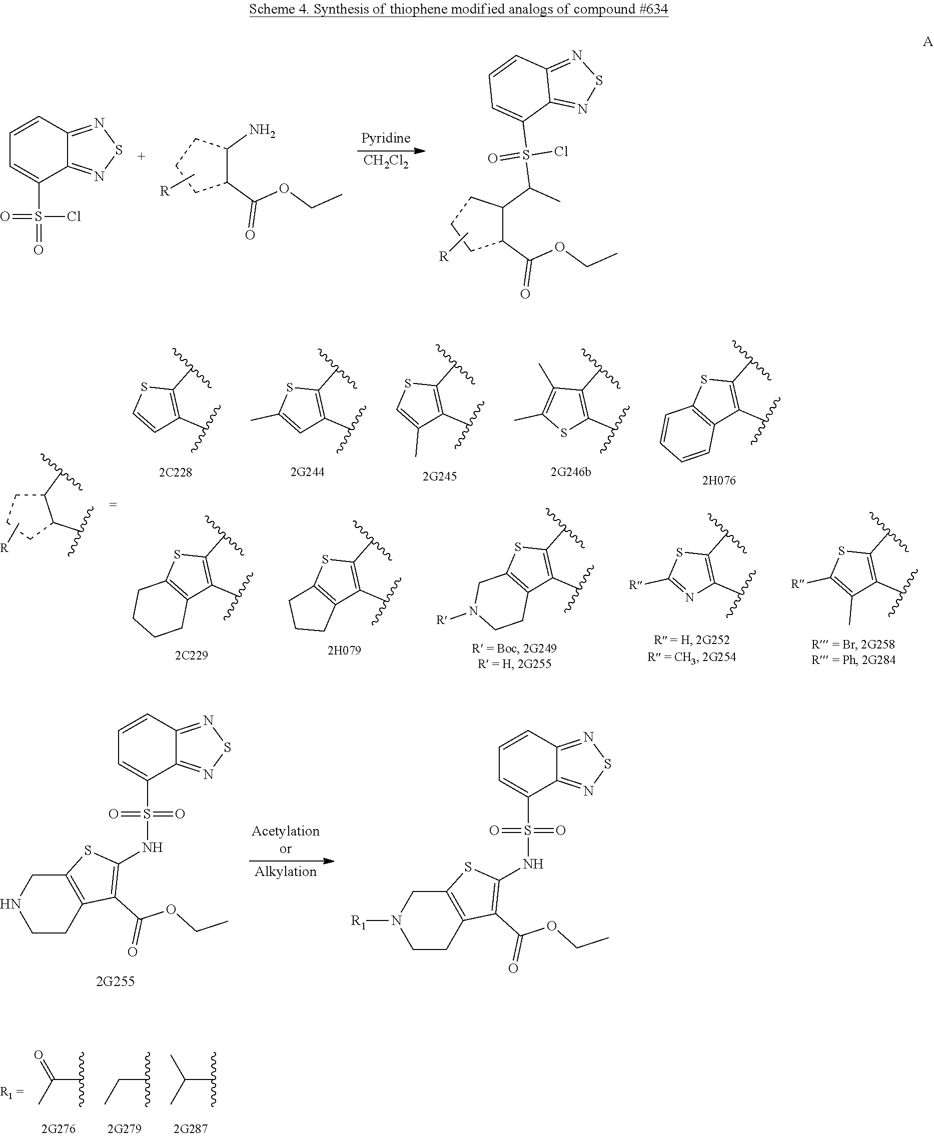

Syntheses of hybrid analogs with another hit compound #504: Based on the HTS, we identified another compound #504 bearing a sulfonamide functional group and potent in calcium Fura-2 assay (structure is shown in Scheme 5). Thus, two hybrid analogs were synthesized by combining the substituents of compounds #634 with #504 as shown in Scheme 5. Compound 2H063 and 2H080 were synthesized using the same chemistry for #634 with commercially available reagents.

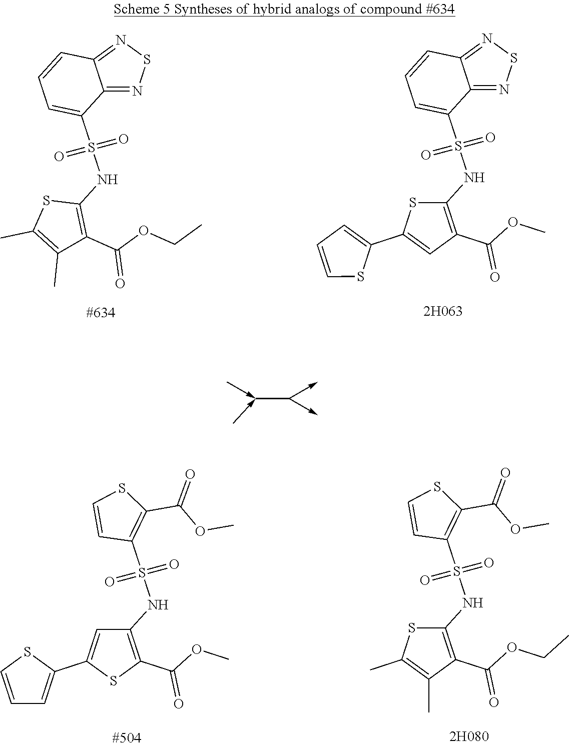

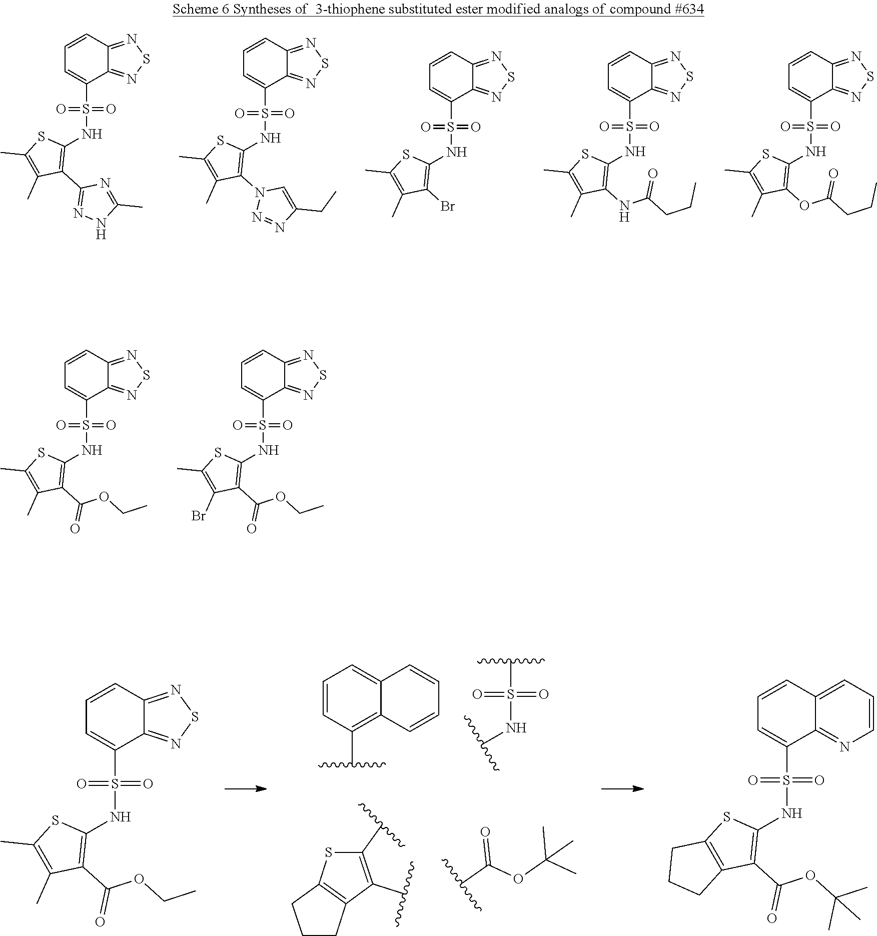

### Results

### Compound 634 Induces Ca²⁺ Influx

Small molecule Ca²⁺ channel activators used as a coadjuvant enhance vaccine adjuvant activity (Saito et al., 2022; Saito et al., 2021). Triggering Ca²⁺ influx rapidly increases intracellular Ca²⁺ concentration, which enhances plasma membrane EV biogenesis. Thus, it was postulated that small molecule compounds that increase intracellular Ca²⁺ in APCs would enhance the release of immune stimulatory EVs. Eighty hit compounds selected by the triple HTS were further analyzed by a ratiometric Ca²⁺ indicator assay in a human monocytic cell line (THP-1 cells) (FIG. 24A). lonomycin (ION) and thapsigargin (TG), calcium ionophore and store-operated calcium entry (SOCE) inducer, respectively, were used as positive controls. In a validation assay, compounds 634 and 456 significantly increased intracellular Ca²⁺ compared to vehicle control (Veh, 0.5% DMSO) (FIGS. 18A and FIGS. 24A and B). The top two hits, 634 and 456, were further assayed for Ca²⁺ influx in mouse primary bone marrow-derived dendritic cells (mBMDCs) using a ratiometric Ca²⁺ indicator. Compound 634 induced Ca²⁺ influx in mBMDCs, while 456 failed to elevate intracellular Ca²⁺ levels (FIG. 28B). Compound 634 was non-toxic to mBMDC at 10 μM, which triggered Ca²⁺ influx. Thus, compound 634 was selected for further characterization.

The increase of intracellular Ca²⁺ induced by 634 was comparable to ionomycin (1 μM) (FIG. 18B). In the Ca²⁺ add-back assay in mBMDC, 634 did not change the intracellular Ca²⁺ concentration in the absence of extracellular Ca²⁺, while intracellular Ca²⁺ levels were restored by adding Ca²⁺ in the medium (1.8 mM) (FIG. 18C). Differential expression analysis at the gene level (R-limma, www.r-project.org) comparing 634 against vehicle control in mBMDC showed that among 103 genes whose expression was modulated, seven genes related to calcium-signaling were affected by 634 (5 genes up-regulated and 2 down-regulated) at Benjamini-Hochberg FDR≤0.05, and fold-change>2 (86 genes up-regulated, 17 genes down-regulated). These results imply that 634 acts via Ca²⁺ signaling pathways.

### 634 Increases EV Release

To examine whether 634 enhances EV release, the numbers and size distributions of EVs from 634 (10 μM) treated mBMDCs were assessed using microfluidic resistive pulse sensing (MRPS) with a Spectradyne nCS1™ instrument. ION (1 μM) was used as a positive control (Messenger et al., 2018; Dircin et al., 2017). The EVs were isolated using a multistep differential ultracentrifugation protocol (Shukla et al., 2022). 634 significantly increased the number of EVs released in the culture supernatant compared to the Veh control by 45% (p<0.05, FIG. 19A). The size distribution was similar among EVs induced by Veh, 634, and ION (EVᵥₑₕ, EV₆₃₄, and EV_{ION}, respectively) (FIG. 19B). EVs were further confirmed by enrichment of tetraspanin CD81 and Alix and reduced calnexin by immunoblots (FIGS. 19C and 25). EVᵥₑₕ, EV₆₃₄, and EV_{ION} were morphologically similar, as confirmed by transmission electron microscopy (TEM) (FIG. 19D). The protein contents of EVᵥₑₕ, EV₆₃₄, and EV_{ION} were comparable, as measured by Micro BCA Assay Kit (FIG. 19E). Collectively, these data indicated that 634 increased the number of EVs released in the culture supernatant by mBMDCs, whereas it did not influence the protein contents or size distribution of EVs.

### EV₆₃₄ Displays Higher Expression of Costimulatory Molecules on the Surface

T cell activation requires antigen displayed on APC interacting with costimulatory and MHC molecules. Calcium signaling regulates APC function (Rao & Hogan, 2009; Vig & Kinet, 2009). In the context of EVs, costimulatory molecules such as CD86 and MHC class II are expressed on EVs from parent DCs (Lindenbergh & Stoorvogel, 2018). Thus, it was hypothesized that 634 increases the expression of costimulatory molecules and MHC class II on mBMDCs that are subsequently transferred to the surface of released EVs. To test this notion, mBMDCs were treated with Veh (0.5% DMSO), 634 (10 μM), ION (1 μM), or MPLA (1 μg/mL) overnight, and the expression levels of CD86, CD80, MHC class II, and CD40 on mBMDCs were analyzed by flow cytometry (FIG. 26). The TLR4 ligand, MPLA, was used as a positive control (Qazi et al., 2009). 634 and ION, as well as MPLA, increased CD86 and CD80 expression on mBMDCs (FIGS. 26A and 26B). Next the presence of costimulatory molecules on EVs was evaluated using high-resolution flow cytometry, with an Amnis® CellStream® (Crooks et al., 2021) (FIGS. 31A and 31B), and by immunoblots (FIGS. 20C and 27). Higher levels of CD86 expression were detected on EV₆₃₄ and EV_{ION}, similar to those on EV_{MPLA} in comparison to EVᵥₑₕ, (p<0.05). This upregulation was also confirmed by immunoblots of isolated EVs (FIGS. 20C and 27A). The immunoblots showed that CD80 expression was higher on EV₆₃₄ than on EVᵥₑₕ (FIGS. 20C and 27C). These results for EV₆₃₄ are consistent with those for parent mBMDCs treated with 634.

EVs Derived from mBMDCs Treated with 634 Stimulate DO11.10 T Cell Proliferation

The above data demonstrates that EV₆₃₄ carried the costimulatory molecules CD86 and CD80 that are needed to prime naïve T cells (Lindenbergh & Stoorvogel, 2018). To evaluate whether EV₆₃₄ induces T cell proliferation, carboxyfluorescein succinimidyl ester (CFSE)-labeled CD4⁺ T cells expressing ovalbumin (OVA)-specific T cell receptors (TCR, DO11.10 mice) were co-cultured with EV₆₃₄ in the presence of OVA MHC class II peptides (OVA₃₂₃₋₃₃₉) (Hsu et al., 2003; Naslund et al., 2013) (FIG. 21A). MPLA (1 μg/mL) was used as a positive control (Qazi et al., 2009). EVs isolated from the medium without mBMDCs (EV_{No cells}) served as negative controls. The amounts of EVs added to the T cell culture were normalized by the volumes of the culture supernatants. T cell proliferation was monitored by CFSE dilution using flow cytometry and quantified as percentages of divided T cells, as well as by IL-2 release into the culture supernatants (Roederer, 2011) (FIGS. 21B, 21C, and 28A). In the presence of OVA₃₂₃₋₃₃₉, EV₆₃₄ induced significantly higher T cell proliferation and IL-2 release, equivalent to EV_{MPLA} or EV_{ION} (p<0.001, FIGS. 20B, 20C). In contrast, no T cell proliferation was detected by particles isolated from the medium (EV_{No cells}) or in the absence of OVA₃₂₃₋₃₃₉ (FIG. 21B). These data indicate that EV₆₃₄ stimulates T cell proliferation in a TCR-dependent manner.

In the above studies, the dosage of EVs was normalized by the volumes of culture supernatants (Qazi et al., 2009). By this approach, it could not be distinguish whether the induction of T cell proliferation was attributable to the increased EV number or to the immunostimulatory qualities of EVs. To address this question, an equal number of EVs (10⁹/mL) were cultured with CFSE-labeled DO11.10 CD4⁺ T cells. EV₆₃₄ maintained higher levels of T cell proliferation and IL-2 release compared to EVᵥₑₕ (p<0.001) when equal numbers of EVs were used (FIGS. 21D and 28B). These data indicate that 634 induced not only a higher number of EVs, but also induced EVs with TCR-dependent T cell activation capacity.

### Structure-Activity Relationship (SAR) Studies of 634

It was demonstrated that EV₆₃₄ could prime naïve T cells in a TCR-dependent manner. To confirm that Ca²⁺ influx is associated with immunostimulatory EV functions, we added to the mBMDC culture chelators for extracellular or intracellular calcium (EGTA, and BAPTA-AM, respectively). However, these chelators were toxic and alone enhanced particle release from dead and dying cells. Next, 634 analogs were synthesized that had lost the ability to induce Ca²⁺ influx in focused SAR studies by modification at the sulfonamide, and at the carboxyl ester on the thiophene ring of the scaffold.

Since compound 634 was not commercially available for bulk purchase, its synthesis was undertaken. Starting with ethyl 2-amino-4,5-dimethylthiophene-3-carboxylate, benzo[c][1,2,5]thiadiazole-4-sulfonyl chloride and pyridine as base, compound 634 was obtained in good yields (44.5%). Next, using compound 634 as a common synthon, we first de-esterified the ethyl ester to obtain free carboxylic acid analog 2H013. Then the acid was converted to acid chloride with thionyl chloride to obtain an advanced reactive intermediate which was reacted with several different alcohol reagents to obtain ester modified analogs. These included analogs with alkyl esters of varying chain lengths, such as 2G179a (methyl ester), 2G176 (propyl ester), 2G179b (hexyl ester) as well as branched alkyl analogs including 2G179c (isopropyl ester), 2G179d (isopentyl ester), 2G179 h (tert-butyl ester), and 2G179e (cyclohexyl ester). Additionally, analogs were prepared bearing phenolic (2G179g) and benzylic (2G179f) ester functionalities. An ethylamide analog of 634 (2E241) was synthesized to probe the necessity of the ester functional group for Ca²⁺ mobilization. The sulfonamide group was also altered by N-methylation to obtain compound 2F186 (FIG. 22).

Ca²⁺ Influx Inducing 634 Analogs Release EVs that Promote T Cell Proliferation

To examine the properties of the 634 analogs, mBMDCs were incubated with 12 of the compounds and followed intracellular Ca²⁺ levels (FIGS. 23A and 29) by Fura-2 assay. Three analogs, 2E241 (amide analog), 2F186 (N-methyl sulfonamide analog), and 2H013 (carboxylic analog), did not induce Ca²⁺ influx in mBMDCs according to the Fura-2 assay. The other nine analogs each statistically increased intracellular Ca²⁺ levels, compared to Veh (FIGS. 23A and 29). EV release and costimulatory molecule expression by the 634 analogs were screened by CD63 Tluc reporter cells (Shpigelman et al., 2021) and by FACS in mBMDCs, respectively. Three analogs that lost the ability of Ca²⁺ influx, 2E241, 2F186 and 2H013, did neither increase luciferase activities in the culture supernatant of CD63 Tluc reporter cells, nor co-stimulatory expression on mBMDC. Furthermore, the levels of intracellular Ca²⁺ increase induced by the 634 analogs positively correlated with CD63 Tluc reporter cell responses and costimulatory molecule expression (p<0.001, FIGS. 23B and 23C).

To confirm that Ca²⁺ influx is associated with immunostimulatory EV functions, DO 11.10 CD4⁺ T cell proliferation assays were performed using EVs released by 634 analogs-treated mBMDC. For EV function assay, five agents (2G179b, 2G179d, 2G179e, 2G179f, and 2G179 h) highlighted in gray in FIGS. 23A, B, and C were excluded due to the higher toxicity in exosome-depleted FBS medium (FIGS. 40A and B) (Eitan et al., 2015). EVs were isolated from 48 hours culture supernatants of mBMDCs treated with Ca²⁺ influx positive (634, 2G176, 2G179a, 2G179c, 2G179g) and negative (2E241, 2F186, and 2H013) compounds. When T cell proliferation was expressed as relative values of divided cells (EVᵥₑₕ=1), EVs from mBMDCs treated with Ca²⁺ influx positive compounds elicited significantly higher T cell proliferation than did Ca²⁺ influx negative compounds (FIGS. 23D and 31). These results imply that the analogs that retain the ability to increase intracellular Ca²⁺ level induced higher immunostimulatory EV release than analogs that lost this ability, and ester and sulfonamide functional groups are necessary for immunostimulatory function of EVs.

### CONCLUSIONS

Utilizing three parallel HTS, eighty hit compounds were selected that enhance EV release with NF-κB activity and ISRE activity (Shpigelman et al., 2021). In this study, the eighty hit compounds were screened for Ca²⁺ influx, and compound 634 was identified as a Ca²⁺ influx inducer. Compound 634 did not only enhance EV release in mBMDCs but also increased costimulatory molecule expression on the surface of EVs and parental dendritic cells. EVs released from 634-treated mBMDCs induced T-cell proliferation in a TCR-dependent manner. The SAR studies suggested that 634 analogs bearing the ester functional group retained the ability to induce Ca²⁺ influx and induced immunostimulatory EV release from mBMDCs, as compared to the amide, carboxylic acid, and N-methyl sulfonamide analogs, all of which lost the ability to induce Ca²⁺ influx. Collectively, compound 634 enhances immunostimulatory EV release via induction of Ca²⁺ influx. This agent and its analogs should be useful tools for the development of effective EV-based vaccines.

### Materials and Methods

Compounds synthesis. Compound 634 and 12 analogs were synthesized in our laboratory as described in FIG. 22. These compounds were dissolved in DMSO (#D2438, MilliporeSigma, Temecula, CA) to obtain stock solutions (2 mM).

Animals. Wild-type BALB/c mice and DO11.10 mice were purchased from the Jackson Laboratory. All animal experiments were approved by the Institutional Animal Care and Use Committee for UC San Diego.

Generation of mBMDCs. mBMDCs were prepared from bone marrow cells harvested from femurs of BALB/c mice as previously described (Lutz et al., 1999; Datta et al., 2003). mBMDCs were washed with RPMI 1640 medium and incubated with compound or vehicle (0.5% DMSO) in RPMI 1640 supplemented with exosome depleted FBS in a T182 flask (7.5×10⁵ cells/mL, total 40 ml) for 46-48 hours. The culture supernatants were used for EV isolation.

Cell lines. THP-1 cells were cultured in RPMI 1640 medium supplemented with 10% dialyzed FBS supplemented with 100 U/mL penicillin, 100 μg/mL streptomycin, and 50 μM 2-mercaptoethanol. CD63 Tluc reporter cell line (Shpigelman et al., 2021) were cultured in RPMI 1640 medium supplemented with 10% dialyzed FBS, 100 U/mL penicillin, 100 μg/mL streptomycin, 1 mM sodium pyruvate, 1×MEM non-essential amino acids, and 5 μg blastidin. Both cell types were maintained in humidified conditions with 5% CO₂ at 37° C.

Calcium Influx assay. Ca²⁺ influx was measured using Fura-2 or Fura-8 reagents.

EV isolation by differential ultracentrifugation. EVs were isolated as described previously with minor modifications (Shpigelman et al., 2021). The detailed information is described in the Supporting information. All relevant data was submitted to the EV-TRACK knowledgebase (EV TRACK ID: EV220366, https://evtrack.org/index.php) (Consortium et al., 2017).

Measurement of EV concentrations. EV particle numbers and particle size distribution were determined by MRPS technology with an nCS1™ particle analyzer utilizing C-400 cartridges (Spectradyne, Signal Hill, CA). EV samples were diluted 200-fold in 1% Tween20-filtered PBS. All results were analyzed using the nCS1™ Data Analyzer (Spectradyne). To exclude false particle events, we applied background subtraction and the following peak filters: Transit time (μs) from 0 to 100, symmetry from 0.2 to 4.0, diameter (nm) from 75 to 400, and signal to noise ratio (S/N) at least 10.

Immunoblotting. The immunoblotting was performed using anti-CD81, anti-Alix, anti-Calnexin, anti-CD86, anti-CD80, anti-MHC class II, and anti-CD40 antibodies as primary antibodies as previously described by us (Shpigelman et al., 2021).

Transmission electron microscopy. For the morphological characterization of EVs, negative stain transmission electron microscopy was performed as previously described (Shpigelman et al., 2021).

Costimulatory Molecule Expression Analysis. Costimulatory molecule expression on mBMDC was measured by flow cytometry assay as described previously (Shukla et al., 2022). The detailed information is shown in FIG. 33.

High-resolution single EV analysis by imaging flow cytometry. EVs were characterized using a commercially available reagent, vFC™ assay kit (#CBS, Cellarcus Biosciences, La Jolla, CA), using the Amnis® CellStream® Flow Cytometer equipped with 488 and 642 nm lasers (Luminex, Austin, TX) according to manufacturer's instruction (Crooks et al., 2021) with some modification. Detailed information for reagents used in flow cytometry analysis is shown in FIG. 34.

Antigen-Specific T cell proliferation assay and ELISA. Transgenic OVA₃₂₃₋₃₂₉ specific CD4⁺ cells were isolated from DO11.10 mice splenocytes using EasySep™ Mouse CD4⁺ T cell isolation kit (negative selection). CFSE (4 μM)-labeled DO.11.10 CD4⁺ T cells were co-cultured with an equal volume or equal number (3.13×10⁹ or 3.99×10⁹ EV particles) of EVs in the presence of OVA₃₂₃₋₃₃₉ peptide (Hsu et al., 2003). IL-2 in the supernatant was tested by Mouse IL-2 DuoSet ELISA kit. Cells were stained with anti-mouse DO11.10 clonotypic TCR antibody, and antigen-specific CD4⁺ T cell proliferation was evaluated by CFSE dilution using MACSQuant flow cytometer (Meltyni Biotec, San Diego, CA). Cell proliferation was quantitated by percentages of divided cells relative to the original population (Roederer, 2011) (Figure S12). Data were analyzed using FlowJo (version 10.8.1, FlowJo, Ashland, OR).

CD63 Tluc-CD9 EmGFP THP-1 reporter cell assay. The reporter cell assay was carried out as described previously (Shukla et al., 2022).

Cell viability assay. The cell viability was measured by MTT assay as described previously (Shukla et al., 2022).

Statistical analysis. To compare multiple groups, one-way ANOVA with Dunnett's post hoc test was applied. To compare two groups, the two-tailed Mann-Whitney test was used (Immune cell populations). Prism 9 software (GraphPad Software, San Diego, CA) was used. P values smaller than 0.05 were considered statistically significant.

### Supporting Information

Additional data of cytokine levels, expression of costimulatory molecules, and cell viability, original data of Ca²⁺ influx assay and immunoblot, compound data of 1H, 13C NMR, HRMS, and LC-MS, and the information of biological experiments are detailed in material and methods.

TABLE A
RNA sequencing analysis of mBMDCs treated with compound 634

| | Gene | | | |
|---|---|---|---|---|
| | Symbol | Description | FC | adj. P. Val |
| Up-regulated | Oit3 | oncoprotein induced | 2.02 | 2.70E−04 |
| | | transcript 3 | | |
| | Sla2 | Src-like-adaptor 2 | 2.04 | 9.69E−03 |
| | Htr7 | 5-hydroxytryptamine | 2.38 | 6.28E−09 |
| | | (serotonin) receptor 7 | | |
| | Cdh1 | cadherin 1 | 2.61 | 4.05E−09 |
| | Ppm1e | protein phosphatase 1E | 2.77 | 3.35E−02 |
| | | (PP2C domain containing) | | |
| Down- | Cdhr1 | cadherin-related family | 0.40 | 4.42E−02 |
| regulated | | member 1 | | |
| | Hrh1 | histamine receptor H1 | 0.46 | 2.60E−05 |

mBMDCs were treated with compound 634 (5 μM) or Veh (0.1% DMSO) in triplicate for 5 hours. RNA was isolated, and RNA-seq analysis was performed. 634 modulated expression of 103 genes compared to Veh (FDR≤0.05, and fold change >2) (86 genes: up-regulated, 17 genes: down-regulated). Five up-regulated and two down-regulated genes related to Ca²⁺ signaling were identified by Gene Summaries from NCBI RNA reference sequence collection (RefSeq) Database, Gene Ontology (GO) Biological Process, GO Molecular Function, or KEGG pathway. Data were analyzed by UC San Diego Moore Cancer Center Biostatistics and Bioinformatics Shared Resources.

TABLE B
Antibodies

| | | | |
|---|---|---|---|
| | Dilution | | |
| Antibodies (clone) | Factor | Source | Catalog # |

Immunoblotting

| | | | |
|---|---|---|---|
| Anti-CD81 (D5O2Q) | 1000 | Cell Signaling | 10037 |
| Anti-Alix (3A9) | 1000 | Cell Signaling | 2171 |
| Anti-Calnexin (EPR3633(2)) | 1000 | Abcam | ab133615 |
| Anti-CD86 (E5W6H) | 1000 | Cell Signaling | 19589S |
| Anti-CD80 (E6J6N) | 1000 | Cell Signaling | 54521 |
| Anti-MHC class II (M5/114) | 1000 | Abcam | ab139365 |
| Anti-CD40 (E227J) | 1000 | Cell Signaling | 86165 |
| Anti-Rabbit IgG, HRP | 5000 | Cell Signaling | 7074 |
| Anti-mouse IgG, HRP | 5000 | Cell Signaling | 7076 |
| Anti-rat IgG, HRP | 5000 | Cell Signaling | 7077 |

Flow Cytometry

| | | | |
|---|---|---|---|
| Anti-DO-11.10 clonotype TCR | 300 | BD | 56524 |
| | | Biosciences | |
| Anti-CD11c, APC (N418) | 400 | eBioscience | 17-0114-82 |
| Anti-CD86, PE (GL1) | 500 | eBioscience | 12-0862-82 |
| Anti-CD80, PE (16-10A1) | 150 | eBioscience | 12-0801-82 |
| Anti-MHC class II [I-A], FITC | 400 | BD | 553547 |
| (AMS-32.1) | | Biosciences | |
| Anti-CD40, PE (1C10) | 200 | eBioscience | 12-0401-83 |
| Anti-CD16/32 (2.4G2) | 300 | BD | 553142 |
| | | Biosciences | |

Vesicle Flow Cytometry

| | | | |
|---|---|---|---|
| vFRed | 100 | Cellarcus | CBS4 |
| | | Biosciences | |
| Anti-MHC class II [I-A/I-E], | 10 | BD | 562352 |
| Alexa Fluor 488 (M5/114) | | Biosciences | |
| Anti-CD86, APC (GL1) | 10 | BD | 558703 |
| | | Biosciences | |

TABLE C
Reagents used in each experiment

| Reagents | Source | Catalog # |
|---|---|---|
| MPLA | Invivogen (San Diego, | tlrl-mpls |
| | CA) | |
| PMA | Invivogen | tlrl-pma |
| Ionomycin | Tocris (UK) | 1704 |
| Thapsigargin | Tocris | 1138 |
| OVA₃₂₃₋₃₃₉ | AnaSpec (Fremount, | AS-27024k |
| (ISQAVHAAHAEINEAGR) | CA) | |
| 1 × PBS | Thermo Fisher | 14190 |
| | Scientific (Waltham, | |
| | MA) | |
| GM-CSF | BioLegend (San | 576308 |
| | Diego, CA) | |

Cell culture

| | | |
|---|---|---|
| RPMI 1640 | Thermo Fisher | 11875 |
| | Scientific | |
| Dialyzed FBS | Atlanta Biologicals | S12850H |
| | (Atlanta, GA) | |
| Penicillin, Streptomycin | Thermo Fisher | 15140122 |
| | Scientific | |
| 2-mercaptoethanol | | |
| Sodium pyruvate | Thermo Fisher | 11360-070 |
| | Scientific | |
| MEM non-essential amino | Thermo Fisher | 11140-050 |
| acids | Scientific | |
| Blastidin | Invivogen | Ant-bl |

EV isolation

| | | |
|---|---|---|
| Exosome depleted FBS | Thermo Fisher | A27208 |
| | Scientific | |
| T182 flask | Thermo Fisher | 25-211 |
| | Scientific | |
| 31.5 mL open-top polypropylene | Beckman Coulter Life | 358126 |
| UC tube | Sciences (Brea, CA) | |
| 0.02 μm inorganic membrane | MilliporeSigma | 6809-2002 |
| filter | (Temecula, CA) | |

T cell proliferation assay and ELISA

| | | |
|---|---|---|
| EasySep Mouse CD4+ T cell | STEMCELL | 19852 |
| isolation kit | Technologies | |
| | (Vancouver, BC, | |
| | Canada) | |
| CFSE | Thermo Fisher | C34554 |
| | Scientific | |
| Mouse IL-2 DuoSet ELISA kit | R&D systems | DY402 |
| | (Minneapolis, MN) | |
| anti-mouse DO11.10 clonotypic | BD Biosciences (San | 562524 |
| TCR antibody | Diego, CA) | |
| Stain Buffer | BD Biosciences | 554657 |

Calcium Influx assay

| | | |
|---|---|---|
| Fura-2-AM | Abcam (Boston, MA) | Ab120873 |
| Fura-8-AM | ATT Bioquest | 21055 |
| | (Sunnyvale, CA) | |
| 1 × HBSS | Corning (Union City, | 14175 |
| | CA) | |
| Pluronic F127 | Thermo Fisher | P3000MP |
| | Scientific | |
| Immunoblotting | | |
| PhosphoSafe extraction reagent | MilliporeSigma | 71296 |
| Protease inhibitor mixture | Roche (Manheim, | 11697498001 |
| | Germany) | |
| Micro BCA Assay Kit | Thermo Fisher | 23235 |
| | Scientific | |
| NuPAGE sample buffer | Thermo Fisher | NP007 |
| | Scientific | |
| NuPAGE 4-12% Bis-Tris Gels | Thermo Fisher | NP0329 |
| | Scientific | |
| Immobilon-P PVDF membranes | MilliporeSigma | IPVH00010 |
| ProSignal Dura ECL | Thermo Fisher | 34076 |
| | Scientific | |
| AccuRuler Prestained Protein | Lamda Biotech (St. | 26616 |
| Ladder | Louis, MO) | |

CD63 Tluc-CD9 EmGFP THP-1 reporter cell assay

| | | |
|---|---|---|
| TurboLuc ™ Luciferase One- | Thermo Fisher | 88264 |
| Step Glow Assay Kit | Scientific | |

### Materials and Methods: Chemical Synthesis

Chemical reagents were purchased as at least reagent grade from commercial vendors unless otherwise specified and used without further purification. Solvents were purchased from Fischer Scientific (Pittsburgh, PA) and were either used as purchased or redistilled with an appropriate drying agent.

Instrumentation. Analytical TLC was performed using precoated TLC silica gel 60 F₂₅₄ aluminum sheets purchased from EMD (Gibbstown, NJ) and visualized using UV light. Flash chromatography was carried out using a Biotage Isolera One (Charlotte, NC) system for normal phase column chromatography or Teledyne ISCO ACCQPrep HP150 for C18-reverse phase column chromatography using the specified solvent. Reaction monitoring and purity analysis were done using an Agilent 1260 LC/6420 Triple Quad mass spectrometer (Santa Clara, CA) with Onyx Monolithic C18 (Phenomenex, Torrance, CA) column. Purity of all final compounds was above 95% (also see LC-MS spectra in Supporting Information for all final compounds). All final compounds were analyzed by high resolution MS (HRMS) using an Agilent 6230 ESI-TOFMS (Santa Clara, CA). 1H and ¹³C NMR spectra were obtained on a Varian 500 with XSens probe (Varian, Inc., Palo Alto, CA). The chemical shifts are expressed in parts per million (ppm) using suitable deuterated NMR solvents.

### EXPERIMENTAL SECTION

Ethyl 2-(benzo[c][1,2,5]thiadiazole-4-sulfonamido)-4,5-dimethylthiophene-3-carboxylate (634). In a round bottom, benzo[c][1,2,5]thiadiazole-4-sulfonyl chloride (400 mg, 1.71 mmol) and ethyl 2-amino-4,5-dimethylthiophene-3-carboxylate (340 mg, 5.17 mmol) were dissolved in anhydrous CH₂Cl₂ (1 mL), followed by the addition of pyridine (413 μL, 1.29 mmol) to the reaction mixture and stirred overnight. Solvent was then removed, and the residue was recrystallized in isopropanol to yield 302 mg of 634 (44.5% yield). ¹H NMR (500 MHz, CHLOROFORM-d) δ 11.10 (s, 1H), 8.34 (d, J=7.09 Hz, 1H), 8.24 (d, J=8.56 Hz, 1H), 7.71 (t, J=7.80 Hz, 1H), 4.28 (q, J=7.09 Hz, 2H), 2.16 (s, 3H), 2.08 (s, 3H), 1.35 (t, J=7.21 Hz, 3H). ¹³C NMR (126 MHz, CHLOROFORM-d) δ 165.1, 155.2, 148.8, 145.5, 132.2, 130.3, 130.0, 127.9, 127.1, 123.0, 114.5, 60.8, 29.7, 14.2, 12.4. HRMS for C₁₅H₁₅N₃O₄S₃ [M+Na⁺] calculated 420.0117, found 420.0118.

2-(benzo[c][1,2,5]thiadiazole-4-sulfonamido)-4,5-dimethylthiophene-3-carboxylic acid (2H013). In a reaction vial, compound 634 (300 mg, 0.756 mmol) was dissolved in a 1:1 ratio of methanol (1 mL) and tetrahydrofuran (1 mL). In a separate vial, lithium hydroxide (47.4 mg, 1.129 mmol) was dissolved in water (0.4 mL, 20% of the organic volume) and added to the reaction mixture. The reaction was heated to 45° C. and allowed to stir overnight. Solvent was then removed and extracted with ethyl acetate, water, and hydrochloric acid. The organic layer was collected, and the residue was purified by HPLC prep system to yield 218 mg of compound 2H013 (78.4% yield). ¹H NMR (500 MHz, CHLOROFORM-d) δ 11.00 (s, 1H), 8.29 (d, J=6.85 Hz, 1H), 8.20 (d, J=8.56 Hz, 1H), 7.66 (t, J=7.80 Hz, 1H), 2.14 (s, 6H). ¹³C NMR (126 MHz, CHLOROFORM-d) δ 169.9, 155.2, 148.7, 148.3, 132.1, 130.8, 129.8, 127.9, 127.4, 123.3, 113.1, 14.4, 12.5. HRMS for C₁₃H₁₁N₃O₄S₃Na [M+Na]⁺ calculated 391.9804, found 391.9802.

2-(benzo[c][1,2,5]thiadiazole-4-sulfonamido)-4,5-dimethylthiophene-3-carbonyl chloride (2F230). Compound 2H013 (15 mg, mmol) was dissolved in anhydrous CH₂Cl₂ (0.5 mL). Thionyl chloride (0.5 mL) was then added and stirred at room temperature for 30 minutes. A 1:1 volume ratio of CH₂Cl₂ and thionyl chloride is used. Solvent was then removed and washed with CH₂Cl₂ multiple times. The residue was not purified and used an intermediate. 100% yield is assumed to yield 15.7 mg of 2F230.

Propyl 2-(benzo[c][1,2,5]thiadiazole-4-sulfonamido)-4,5-dimethylthiophene-3-carboxylate (2G176). In a reaction vial N-propanol (0.3 mL) and triethylamine (TEA) (6 μL, 0.043 mmol) were mixed and added to compound 2F230 (8.4 mg, 0.022 mmol). The reaction mixture was stirred at room temperature until completion and purified by HPLC prep to yield 2.4 mg of compound 2G176 (28.6% yield). ¹H NMR (500 MHz, CHLOROFORM-d) δ 11.15 (s, 1H), 8.34 (d, J=7.09 Hz, 1H), 8.16-8.28 (m, 1H), 7.70 (dd, J=7.09, 8.80 Hz, 1H), 4.18 (t, J=6.60 Hz, 2H), 2.16 (s, 3H), 2.08 (s, 3H), 1.69-1.79 (m, 2H), 0.98 (t, J=7.46 Hz, 3H). ¹³C NMR (126 MHz, CHLOROFORM-d) δ 165.3, 155.2, 148.9, 145.6, 132.2, 130.3, 130.0, 127.9, 127.1, 122.9, 114.5, 66.5, 21.9, 14.3, 12.4, 10.7. HRMS for C₁₆H₁₇N₃O₄S₃Na [M+Na]⁺ calculated 434.0273, found 434.0271.

Methyl 2-(benzo[c][1,2,5]thiadiazole-4-sulfonamido)-4,5-dimethylthiophene-3-carboxylate (2G179a). In a reaction vial, anhydrous methanol in excess (0.5 mL) and TEA (11.3 μL. 0.081 mmol) were mixed and added to compound 2F230 (15.7 mg, 0.041 mmol). The reaction mixture was stirred at 40° C. until completion. Solvent was then removed, and the residue was purified by HPLC prep system to yield 11.5 mg of 2G179a (74.2% yield). ¹H NMR (500 MHz, CHLOROFORM-d) δ 10.99 (s, 1H), 8.34 (d, J=7.00 Hz, 1H), 8.24 (d, J=8.56 Hz, 1H), 7.71 (t, J=8.80 Hz, 1H), 3.82 (s, 3H), 2.16 (s, 3H), 2.07 (s, 3H). ¹³C NMR (126 MHz, CHLOROFORM-d) δ 165.4, 155.2, 148.8, 145.5, 132.2, 130.3, 130.0, 127.9, 127.1, 123.1, 114.4, 51.7, 14.2, 12.4. HRMS for C₁₄H₁₃N₃O₄S₃Na [M+Na]⁺ calculated 405.9960, found 405.9961.

Hexyl 2-(benzo[c][1,2,5]thiadiazole-4-sulfonamido)-4,5-dimethylthiophene-3-carboxylate (2G179b). Compound 2F230 (15.7 mg, 0.041 mmol), TEA (11.3 μL, 0.081 mmol), and N-hexanol in excess (0.5 mL) were reacted according to the general procedure of compound 2G179a to yield 5.9 mg of compound 2G179b (32.1% yield). ¹H NMR (500 MHz, CHLOROFORM-d) δ 11.16 (s, 1H), 8.35 (d, J=7.09 Hz, 1H), 8.24 (d, J=8.80 Hz, 1H), 7.71 (t, J=7.80 Hz, 1H), 4.22 (t, J=6.72 Hz, 2H), 2.16 (s, 3H), 2.08 (s, 3H), 1.70 (quin, J=7.09 Hz, 2H), 1.36-1.42 (m, 2H), 1.30-1.35 (m, 4H), 0.90 (t, J=6.72 Hz, 3H). ¹³C NMR (126 MHz, CHLOROFORM-d) δ 165.3, 155.2, 148.9, 145.6, 132.2, 130.3, 130.0, 127.9, 127.1, 122.9, 114.5, 65.0, 31.4, 28.5, 25.7, 22.5, 14.3, 14.0, 12.4. HRMS for C₁₉H₂₃N₃O₄S₃Na [M+Na]⁺ calculated 476.0743, found 476.0741.

Isopropyl 2-(benzo[c][1,2,5]thiadiazole-4-sulfonamido)-4,5-dimethylthiophene-3-carboxylate (2G179c). Compound 2F230 (15.7 mg, 0.041 mmol), TEA (11.3 μL, 0.081 mmol), and 2-propanol in excess (0.5 mL) were reacted according to the general procedure of compound 2G179a to yield 7.6 mg of compound 2G179c (45.5% yield). ¹H NMR (500 MHz, CHLOROFORM-d) δ 11.20 (s, 1H), 8.29-8.39 (m, 1H), 8.18-8.28 (m, 1H), 7.70 (dd, J=7.09, 8.80 Hz, 1H), 5.10-5.21 (m, 1H), 2.15 (s, 3H), 2.06 (s, 3H), 1.30 (d, J=6.36 Hz, 6H). ¹³C NMR (126 MHz, CHLOROFORM-d) δ 164.8, 155.2, 148.9, 145.4, 132.2, 130.3, 130.0, 127.9, 127.1, 122.9, 114.7, 68.6, 21.9, 14.3, 12.4. HRMS for C₁₆H₁₇N₃O₄S₃Na [M+Na]⁺ calculated 434.0273, found 434.0269.

Isopentyl 2-(benzo[c][1,2,5]thiadiazole-4-sulfonamido)-4,5-dimethylthiophene-3-carboxylate (2G179d). Compound 2F230 (15.7 mg, 0.041 mmol), TEA (11.3 μL, 0.081 mmol), and iso amyl alcohol in excess (0.5 mL) were reacted according to the general procedure of compound 2G179a to yield 4.9 mg of compound 2G179d (27.5% yield). ¹H NMR (500 MHz, CHLOROFORM-d) δ 11.14 (s, 1H), 8.34 (d, J=7.09 Hz, 1H), 8.20-8.29 (m, 1H), 7.70 (dd, J=7.09, 8.80 Hz, 1H), 4.25 (t, J=6.85 Hz, 2H), 2.15 (s, 3H), 2.07 (s, 3H), 1.68-1.76 (m, 1H), 1.59 (q, J=6.93 Hz, 2H), 0.95 (s, 3H), 0.93 (s, 3H). ¹³C NMR (126 MHz, CHLOROFORM-d) δ 165.3, 155.2, 152.8, 148.9, 145.6, 132.2, 130.3, 127.9, 127.1, 122.9, 114.4, 63.4, 37.2, 25.1, 22.4, 14.3, 12.4. HRMS for C₁₈H₂₁N₃O₄S₃Na [M+Na]⁺ calculated 462.0586, found 462.0582.

Cyclohexyl 2-(benzo[c][1,2,5]thiadiazole-4-sulfonamido)-4,5-dimethylthiophene-3-carboxylate (2G179e). Compound 2F230 (15.7 mg, 0.041 mmol), TEA (11.3 μL, 0.081 mmol), and cyclohexanol in excess (0.5 mL) were reacted according to the general procedure of compound 2G179a to yield 5.6 mg of compound 2G179e (30.6% yield). ¹H NMR (500 MHz, CHLOROFORM-d) δ 11.22 (s, 1H), 8.30-8.40 (m, 1H), 8.18-8.28 (m, 1H), 7.71 (dd, J=7.09, 8.80 Hz, 1H), 4.96 (quin, J=4.20 Hz, 1H), 2.16 (s, 3H), 2.09 (s, 3H), 1.84-1.93 (m, 2H), 1.71-1.77 (m, 2H), 1.37-1.60 (m, 6H). ¹³C NMR (126 MHz, CHLOROFORM-d) δ 164.7, 155.2, 148.9, 145.5, 132.2, 130.3, 130.0, 127.9, 127.1, 122.8, 114.8, 73.3, 31.5, 29.7, 25.3, 23.5, 14.5, 12.4. HRMS for C₁₉H₂₁N₃O₄S₃Na [M+Na]⁺ calculated 474.0586, found 474.0582.

Benzyl 2-(benzo[c][1,2,5]thiadiazole-4-sulfonamido)-4,5-dimethylthiophene-3-carboxylate (2G179f). Compound 2F230 (15.7 mg, 0.041 mmol), TEA (11.3 μL, 0.081 mmol), and benzyl alcohol in excess (0.5 mL) were reacted according to the general procedure of compound 2G179a to yield 3.2 mg of compound 2G179f (17.2% yield). ¹H NMR (500 MHz, CHLOROFORM-d) δ 11.13 (s, 1H), 8.28-8.37 (m, 1H), 8.21 (d, J=8.80 Hz, 1H), 7.69 (dd, J=7.09, 8.56 Hz, 1H), 7.33-7.42 (m, 5H), 5.27 (s, 2H), 2.14 (s, 3H), 2.06 (s, 3H). ¹³C NMR (126 MHz, CHLOROFORM-d) δ 164.9, 155.2, 148.7, 146.1, 135.5, 132.2, 130.3, 129.9, 128.7, 128.4, 128.4, 127.9, 127.1, 123.0, 114.1, 66.5, 14.4, 12.4. HRMS for C₂₀H₁₇N₃O₄S₃Na [M+Na]⁺ calculated 482.0273, found 482.0267.

Phenyl 2-(benzo[c][1,2,5]thiadiazole-4-sulfonamido)-4,5-dimethylthiophene-3-carboxylate (2G179g). In a reaction vial, phenol (36.5 mg, 0.88 mmol) and TEA (6 μL, 0.043 mmol) were dissolved in CH₂Cl₂ (0.5 mL) and was added to compound 2F230 (15.7 mg, 0.022 mmol). The reaction mixture was stirred at 40° C. until completion. Solvent was removed and the residue was purified by HPLC prep system to yield 5.2 mg of 2G179g (28.8% yield). ¹H NMR (500 MHz, CHLOROFORM-d) δ 10.92 (s, 1H), 8.36 (d, J=6.36 Hz, 1H), 8.20-8.30 (m, 1H), 7.72 (dd, J=7.09, 8.80 Hz, 1H), 7.45 (t, J=7.83 Hz, 2H), 7.31 (t, J=7.40 Hz, 1H), 7.06 (d, J=7.58 Hz, 2H), 2.21 (s, 6H). ¹³C NMR (126 MHz, CHLOROFORM-d) δ 163.6, 155.2, 149.8, 148.8, 147.3, 132.2, 130.3, 129.9, 129.6, 127.9, 127.2, 126.2, 123.3, 121.6, 113.5, 14.4, 12.5. HRMS for C₁₉H₁₅N₃O₄S₃Na [M+Na]⁺ calculated 468.0117, found 468.0114.

Tert-butyl 2-(benzo[c][1,2,5]thiadiazole-4-sulfonamido)-4,5-dimethylthiophene-3-carboxylate (2G179 h). Compound 2F230 (15.7 mg, 0.041 mmol), TEA (11.3 μL, 0.081 mmol), and tert butanol in excess (0.5 mL) were reacted according to the general procedure of compound 2G179a to yield 5.4 mg of compound 2G179 h (31.2% yield). ¹H NMR (500 MHz, CHLOROFORM-d) δ 11.28 (s, 1H), 8.28-8.38 (m, 1H), 8.18-8.28 (m, 1H), 7.70 (dd, J=7.09, 8.80 Hz, 1H), 2.14 (s, 3H), 2.04 (s, 3H), 1.52 (s, 9H). ¹³C NMR (126 MHz, CHLOROFORM-d) δ 164.5, 155.2, 148.9, 144.9, 132.3, 130.3, 130.0, 128.0, 127.0, 122.8, 115.8, 82.2, 28.2, 14.4, 12.4. HRMS for C₁₇H₁₉N₃O₄S₃Na [M+Na]⁺ calculated 448.0430, found 448.0434.

Ethyl 4,5-dimethyl-2-(N-methylbenzo[c][1,2,5]thiadiazole-4-sulfonamido)thiophene-3-carboxylate (2F186). Compound 2E213 (10 mg, 0.025 mmol) and potassium carbonate (6.7 mg, 0.050 mmol) was dissolved in anhydrous dimethylformamide. Iodomethane (1.71 μL, 0.028 mmol) was added to the reaction mixture and stirred at 45° C. until completion. The reaction mixture was extracted with ethyl acetate, water and hydrochloric acid. Solvent was removed, and the residue was purified by HPLC prep to yield 5.7 mg of 2F186 (54.8% yield). ¹H NMR (500 MHz, CHLOROFORM-d) δ 8.25 (d, J=8.80 Hz, 1H), 8.13 (d, J=7.00 Hz, 1H), 7.66 (t, J=8.60 Hz, 1H), 3.97 (q, J=7.09 Hz, 2H), 3.64 (s, 3H), 2.21 (s, 3H), 2.16 (s, 3H), 1.26 (t, J=7.21 Hz, 3H). ¹³C NMR (126 MHz, CHLOROFORM-d) δ 163.1, 155.5, 149.7, 140.8, 132.4, 132.4, 131.7, 131.3, 128.2, 126.5, 60.7, 41.9, 14.0, 13.3, 13.3. HRMS for C₁₆H₁₈N₃O₄S₃ [M+H]⁺ calculated 412.0454, found 412.0457.

2-(benzo[c][1,2,5]thiadiazole-4-sulfonamido)-N-ethyl-4,5-dimethylthiophene-3-carboxamide (2E241). Compound 2H013 (10 mg, 0.027 mmol) was dissolved in anhydrous CH₂Cl₂ and ethylamine in THE (2M) (40 μL, 0.081 mmol) was added to the reaction mixture. Solvent was removed, and the residue was purified by HPLC prep to yield 3 mg of 2E241 (28.0% yield). ¹H NMR (500 MHz, CHLOROFORM-d) δ 10.01 (br. s., 1H), 8.26 (d, J=4.16 Hz, 1H), 8.25 (d, J=2.20 Hz, 1H), 7.70 (t, J=7.80 Hz, 1H), 6.22 (br. s., 1H), 3.39 (quin, J=6.80 Hz, 2H), 2.12 (s, 3H), 2.08 (s, 3H), 1.20 (t, J=7.21 Hz, 3H). ¹³C NMR (126 MHz, CHLOROFORM-d) δ 164.8, 155.2, 149.0, 136.7, 132.0, 130.0, 128.3, 128.1, 127.0, 126.2, 34.7, 14.7, 13.5, 12.8. HRMS for C₁₅H₁₇N₄O₃S₃ calculated [M+H]⁺ calculated 397.0457, found 397.0457.

### Materials and Methods: Biological Experiments

Calcium Influx assay. mBMDCs or THP-1 cells were loaded with ratiometric Ca²⁺ indicator, Fura-2-AM (4 μM) or Fura-8-AM (4 μM) in HBSS assay buffer [1×HBSS, 10 mM HEPES (pH 7.4), 1.8 mM CaCl₂, 0.8 mM MgCl₂, and 0.1% BSA] containing 0.04% Pluronic F127 at 37° C. for 40 minutes and at RT for additional 20 minutes. OD_{340/380} (emission) and OD₅₁₀ nm (excitation) were read for Fura-2 by a fluorescence plate reader (Tecan2000, #30016056, TECAN, San Jose, CA). For Fura-8, OD 355/415 nm (excitation) and OD540 (emission) were read. Data were presented as OD ratios for 340/380 or 355/415 as representative of changes in the intracellular Ca²⁺ level. The baseline-subtracted AUC of 340/380 ratios was calculated using GraphPad Prism (version 9, GraphPad Software, San Diego, CA).

EV isolation by differential ultracentrifugation. Conditioned culture medium (40 mL) was spun at 300 g for 10 minutes, at 2,000 g for another 10 minutes, followed by 10,000 g for 30 minutes. Next, 30 mL of supernatant was transferred to 31.5 polypropylene UC tubes and spun at 100,000 g_{avg} for 3 hours in an SW28 rotor (K-Factor: 2,554) by Beckman Optima XL-90 Ultracentrifuge (Beckman Coulter Life Sciences). The supernatant was aspirated (leaving about 50 μL), and the pellet was resuspended in 30 mL cold-filtered PBS. The resuspended pellet was spun under the same conditions as the previous spin, followed by another round of gentle aspiration and resuspension to a final volume of 50 μL in cold-0.02 micron filtered PBS. All centrifugation steps were performed at 4° C., and resultant samples were stored at −80° C. until use.

Immunoblotting. mBMDCs were lysed with PhosphoSafe extraction reagent supplemented with protease inhibitors. The total protein in the samples was quantitated by micro BCA Assay Kit. Two μg protein of cell or EVs lysates were mixed with 4′ NuPAGE sample buffer under reducing condition with dithiothreitol (DTT) for Alix, Calnexin, CD86, CD80, MHC class II, and CD40 or nonreducing condition (without DTT) for CD81. Samples were also denatured at 95° C. for 5 minutes prior to loading. After fractionation on NuPAGE 4-12% Bis-Tris Gels, proteins were blotted onto Immobilon-P PVDF membranes and blocked for 1 hour in 5% BSA-TBS-T at RT. The blots were then incubated with primary antibodies (Abs): anti-CD81, anti-Alix, anti-Calnexin, anti-CD86, anti-CD80, anti-MHC class II, and anti-CD40 Abs (1:1,000 dilution) overnight at 4° C. with gentle agitation. After washing, the membranes were incubated with the corresponding secondary antibody for 30 minutes at RT with gentle agitation. Blots were developed with ProSignal Dura ECL and visualized using a ChemiDoc Imaging System. AccuRuler Prestained Protein Ladder was used for the molecular weight markers.

Transmission electron microscopy. Formvar-carbon-coated copper grids (400 mesh, Ted Pella, Redding, CA, US) were placed on 10 μL drops of each sample solution displayed on a parafilm sheet. After allowing the material to adhere to the grids for 5 minutes, grids were washed three times by rinsing through more than 200 μL drops of milli-Q water before being left for 1 minute on 2% (wt/vol) uranyl acetate in water. Excess solution was removed with 11 μm Whatman filter paper, and grids were left to dry for 20 minutes before viewing. Grides were examined using an FEI Tecnai Spirit G2 BioTWIN transmission electroscope equipped with a bottom mount Eagle 4k (16 megapixels) camera (FEI, Hillsboro, OR, US).

Costimulatory Molecule Expression Analysis. mBMDCs (10⁶ cells/mL) were incubated with 10 μM compound, 1 μM lonomycin and 1 μg/mL MPLA for 20 to 24 hours. 0.5% DMSO was used as vehicle. Cells were incubated with anti-mouse CD16/32 antibody for blocking FcR and stained with anti-CD11c, anti-CD40, anti-CD80, anti-CD83, anti-CD80, or anti-MHC class II antibodies for 30 minutes at 4° C. Cells were stained with 4′,6-diamino-2-phenylindole (DAPI) for 10 minutes at RT. Data were acquired using MACSQuant Analyzer 10 (Miltenyi Biotec, Germany) and analyzed with FlowJo (version 10.8.1, Becton Dickinson, Ashland, OR)). The gating strategy is shown in FIG. 33.

High-resolution single EV analysis by imaging flow cytometry. In brief, samples were diluted 1:64 in Vesicle Staining Buffer (Cellarcus Biosciences) and stained with an antibody cocktail of vFRed, anti-CD86, and anti-MHC class II Abs for 1 hour at room temperature. Samples were diluted 1:200 in Vesicle Staining Buffer before acquisition. Data were acquired using the Cellstream instrument with FSC and SSC turned off and all other lasers set to 100% of the maximum power. Each sample was run for 20 seconds at sample volumetric flow rate of 3.66 μL/minutes. Data were analyzed using FlowJo. Details for antibodies and Flow cytometer configuration are shown in FIG. 34.

Cell viability assay. mBMDCs (2×10⁶ cells/200 μL/well in RPMI1640 supplemented with dialyzed 10% FBS, or 1.5×10⁶ cells/200 μL/well in RPMI1640 supplemented with 10% exosome depleted FBS) were treated with 10 μM of each test compound in 96-well plates. After 46-48 hours of compound treatment, MTT (0.5 mg/mL) was added to each well. The cells were lysed after overnight incubation, and absorbance values at 570 and 650 nm were measured.

CD63 Tluc-CD9 EmGFP THP-1 reporter cell assay. CD63 Tluc CD9 EmGFP THP-1 reporter cell (designated as CD63 Tluc reporter cells) were incubated with 10 μM test compounds, ION (1 μM), or 0.5% DMSO (negative control) in the RPMI 1640 supplemented with exosome-depleted FBS at 5×10⁴ cells in 200 μL/well for a 96-well plate for 48 hours at 37° C. Subsequently, the plate was centrifuged, the supernatant was transferred, and chemiluminescence was measured by TurboLuc™ Luciferase One-Step Glow Assay Kit. The response that measures activation and subsequent release of EVs was calculated using the following formula; % response=100×(compound RLU−average Veh RLU)/(average PMA RLU−average Veh RLU).

RNA-seq and Data Analysis. mBMDCs (1.5×10⁶ cells, 10⁶ cells/mL×1.5 mL) were treated with Veh (0.1% DMSO), 634 (5 μM) for 5 hours, and then the total RNA was isolated using Quick-RNA™ Miniprep Kit (Zymo Research). Each group has triplicates. RNA-seq was performed by the sequencing core at the UC San Diego institute for Genomic Medicine Genomics Center. Briefly, paired-ended sequencing was performed on the Illumina NovaSeq 6000. Reads were aligned to the mouse reference genome (mm10) using STAR (ver. 2.5.1), and mRNA expression levels were calculated per gene using RSEM (ver. 1.3.0). Genes were filtered if more than 24 out of 27 of the samples (including compound 634, there were a total of 8 testing compounds and DMSO in triplicated samples) had counts <10. Raw counts were then quantile normalized and used as expression values in the subsequent analysis. Also, if mouse gene IDs corresponded with multiple human genes, the genes with highest variance in expression values were kept (Miller et al., 2011). A total of 11759 genes remained. Limma (liner models for microarray, using Rlimma package) trend tests were used for differential analysis based on log 2 expression values. The Benjamini-Hochberg procedure was applied to control the false discovery rate (FDR). A gene was considered significantly changed if FDR<0.05 and fold change>2. If log 2 fold change in expression for a test compound vs control was greater than 0, it was said to be up-regulated; otherwise, it was down-regulated. RNA-seq data have been deposited in ArrayExpress, https://www.ebi.ac.uk/arrayexpress/(accession no. E-MTAB-12377) (Hayashi, 2022).

### Example 4

In one embodiment, vaccines for veterinary use are envisioned. Thus, along with exploration of TLR4 and TLR7 combination adjuvant in liposomal formulation, other formulations, e.g., formulating TLR agonists with alum, may be employed, thus allowing for: 1. Additional adjuvant effects of alum, 2. A vehicle for drug delivery, and 3. A viable option to develop the vaccine for veterinary use compared to liposomal formulations. Thus, SAR studies were focused on introducing hydrophilic functional groups that can offer binding to aluminum hydroxide and aluminum phosphate in alum.

Earlier SAR studies (18 compounds) led to identification of compounds 2G023a and 2G053 bearing a N³-(3-aminophenyl) and N³-(3-hydroxyphenyl) substituted pyrimidoindoles; respectively, which were found to be equipotent to compound 28182c. Both these amine and hydroxy handle bearing compounds, 2G023a and 2G053, were derivatized to obtain acetylated analogs (2G107 and 2G112) and octanoylated analogs (2G108 and 2G113) respectively (FIG. 36).

The TLR4 agonism activities in both murine and human HEK-TLR4 reporter cells are shown in FIG. 38. The ester linked derivatives of 2G053 including 2G107 and 2G108 are both potent likely due to labile ester bond cleavage releasing the potent parent compound 2G053 suggesting that the derivatization of 3-hydroxy substituent can be utilized to generate prodrug-like compounds, however amide linked derivatives of 2G023a including 2G112 and 2G113 did not retain equal potency. Based on these data, 2G053 was employed as lead compound and the O-sulphate (2G154), 0-phosphate (2G154), and O-methylenephosphate (2G202) analogs as well as other bidentate 3-hydroxy containing analogs including 3,4-dihydroxy phenyl compound 2E255 and 3,5-dihydroxy phenyl analog 2G197 were synthesized.

These analogs could efficiently bind to alum and thus provide an alternate vehicle for formulation in addition to providing adjuvant effects of alum. The TLR4 agonistic activity showed that O-methylenephosphate (2G202) was weakly active in TLR4 agonism assay while the direct aryl 0-phosphate and 0-sulphate analogs did not retain potency suggesting the efficiency of the alkyl phosphate analog as a prodrug (FIG. 37). These prodrugs may have slow release of the potent 2G053.

Amongst the di-hydroxy analogs, the 3,4-dihydroxy analog 2E255 was completely inactive while the 3,5-dihydroxy analog 2G197 retained partial potency which is consistent from the previous SAR results where 4-substituted analogs were found to be inactive (FIG. 37). Next, this hydrolytic capability of the ester was utilized by connecting 3-hydroxy of 2G053 to obtain a lipid conjugate (2G177) with 1,2-dioleoyl-sn-glycero-3-phosphoethanolamine (DOPE) as shown in FIG. 11 with an aim to obtain compounds with better liposomal formulation ability compared to 28182c as lipid chain in this compound would allow for increased incorporation in to the liposomal bilayers. The bioactivity analysis showed that even though the DOPE conjugate was slightly less potent than 2G053 the potency at the highest concentration was better (FIG. 37). This difference could be attributable to slow release and less toxicity.

### Example 5

### Vaccine Adjuvant Efficacy of Fos47 by Intramuscular and Intranasal Administration

Intranasal/pulmonary vaccine administration has significant advantages over other routes of administration, e.g., convenient, easy and non-invasive administration, and less systemic adverse effects, that improve the compliance of vaccine use. Long-term observation following intramuscular (IM) and intranasal/pulmonary (IN) immunization studies showed that mice immunized with the Fos47 adjuvanted vaccine had higher antigen-specific antibody levels for over 10 months compared to those of the control group. The levels of antibody titers were comparable to that of AS01B, a licensed vaccine adjuvant and TLR4 agonist. Vaccination protocol with IM following IN boosting induced higher IgA in bronchial alveolar lavages (BAL) as well as serum, compared to the IM boosting regimen. Induction of antigen specific IgG by IN boosting was equivalent to the IM boosting protocol. Furthermore, IN boosting protocol induced antigen-specific pulmonary resident memory CD4+ and cos+ T cells. IN administration of Fos47 induced minimal systemic and local inflammatory cytokines and chemokines, lower than IN-administered AS01B. These data implied that IM-followed by IN boosting protocol is a safe and effective vaccine administration regimen.

A combination of intramuscular and intranasal/pulmonary vaccines adjuvanted with Fos47 induces local and systemic antiviral immune responses against the influenza virus

### INTRODUCTION

The intramuscular (IM) prime-boost immunization regimen (IM-IM) using inactivated influenza A virus (IIAV) plus Fos47 and showed that the IM-IM vaccine with IIAV plus Fos47 induced antigen-specific antibody production (PMID: 32636840). While most licensed vaccines for influenza are intramuscularly administered and enhance robust antiviral serum IgG production, those vaccines have less impact on mucosa (immunity at the infection site. On the other hand, vaccines administered via mucosal/pulmonary route are reported to promote mucosal immunity and prevent pathogen entry. (PMID: 34437109, PMID: 34647956). Hence, it hypothesized that vaccination by IM priming and intranasal/pulmonary (IN) boosting could further enhance systemic and local antiviral immunity.

Effect of IN Boosting with IIAV Plus Fos47 on Serum IgG is Comparable to IM Boosting

The effects of the IM-IM regimen and an IM prime IN boost regimen on IgG responses was compared. BALB/c mice were IM primed with IIAV [A/California/04/2009 (H1N1)pdm09] adjuvanted with Fos47 (1 nmol 1V270 plus 200 nmol 2B182C in 50 μL in a liposomal formulation) or vehicle (Lipo-Veh, blank liposomes) on day 0. The mice were either IM boosted with a full dose (50 μL) on day 21 (IM-IM) or IN administered with a half dose of the same agents on days 21 and 28 (IM-IN-IN) (FIG. 38A). For the latter, a half dose of the vaccine (25 μL) was used due to the limitation of IN dosing volume. Sera were collected on day 7 after the last boost, and antibody responses against hemagglutinin (HA) of A/California/04/2009 (H1N1) were assessed by ELISA. Anti-HA IgG1 production was moderately increased by IN boosting with Fos47 (IN-Fos47) compared to IN boosting with Lipo-Veh (IN-Lipo-Veh), while anti-HA IgG2a levels in the IN-Fos47 group were significantly higher than those in the IN-Lipo-Veh group (FIGS. 38B and 38C). The IgG2a induction levels by IN-Fos47 were comparable to IgG2a levels by IM boosting with Fos47.

Antigen-Specific IgG2a Levels Induced by IN Boosting with IIAV/Fos47 Lasted for 6 Months

The durability of antigen-specific IgG induced by the IM-IM and the IM-IN-IN vaccination regimens was examined. Antigen-specific serum IgG levels on days 56 and 182 were evaluated by ELISA (FIG. 39A). Lipo-Veh (blank liposomes) was used as vehicle control (negative control). AS01B, a licensed liposomal combination adjuvant with MPLA and saponin served as a positive control. The effect of IM-IN vaccination with Fos47 or AS01B on antigen-specific IgG1 production was as low as Lipo-Veh on days 56 and 182, while the IgG2a levels induced by IN-Fos47 were significantly higher than those of IN-Lipo-Veh and lasted for 6 months (FIGS. 39B and 39C).

IM-IN-IN Combination Regimen with Fos47 Enhanced Antibody Responses to Neuraminidase and Phylogenetically Distinct Virus Strains

It was previously shown that antibodies induced by the IM-IM vaccination with IIAV plus Fos47 could bind not only to HA but also to neuraminidase (NA) (PMID: 32636840). In addition, the antibodies were cross-reactive to induce these broad antibody responses. Mice were vaccinated following the IM-IN-IN regimen and 4 weeks later, sera were collected to test serum IgG levels (FIG. 40A). ELISA for IgG against NA of homologous influenza virus (A/California/04/2009 (H1N1) showed that the IM-IN-IN regimen with Fos47 induced significantly higher levels of anti-NA IgG1 and anti-NA IgG2a than those of the IM-IN-IN group with Lipo-Veh (FIGS. 40B and 40C). Next, the cross-reactivity of antibodies to HA and NA proteins from antigenically distinct virus strains was evaluated. Total IgG levels against H7 and N7 from A/Netherland/219/2003 (H7N7) were evaluated by ELISA (FIG. 41A). IM-IN-IN regimen with Fos47 enhanced anti-H7 and N₇ total IgG production compared to IgG levels in IM-IN-IN group with Lipo-Veh (FIGS. 41B and 41C), suggesting that the antibody induced by IM-IN-IN vaccination with Fos47 could provide cross-reactive antibody responses.

Antigen-Specific IgA Levels in the Lungs were Significantly Enhanced by Intranasal Boosting with Fos47

In respiratory infectious diseases such as influenza, secretory IgA in the mucosal area plays essential roles in preventing virus entry by blocking attachment and entry of pathogens (PMID: 34437109, PMID: 34647956, PMID: 27168245). Next it was tested whether the IM-IN-IN regimen with Fos47 enhanced antigen-specific IgA responses. Mice were IM primed with IIAV plus Fos47 on day 0, and then boosted on days 21 and 28 via IM or IN route (IM-IM-IM or IM-IN-IN, FIG. 41A). Sera and bronchoalveolar lavage fluids (BALF) were collected to test IgA secretion levels on day 35. The IgA levels in BALFs were significantly induced by IM-IN-IN regimen with Fos47 compared to IgA levels induced by IM-IN-IN regimen with Lipo-Veh, whereas Fos47 by IM route did not induce significant levels (FIG. 42B). Furthermore, IgA secretion levels in sera were increased by IN-Fos47 (FIG. 41C). IgA levels induced by IM primed and IN boosting regimen with Fos47 lasted through day 56 (FIGS. 44A and 44B).

The IM-IN-IN Regimen with Fos47 Enhanced Local and Systemic Antigen-Specific T Cell Responses.

To block respiratory viral infections, both humoral immunity and cellular immunity are critical (PMID: 21216425, PMID: 34211186, PMID: 33353987). Recent studies showed that IN immunization with live attenuated influenza virus or influenza virus enhanced antiviral T cell responses in the lung (PMID: 27468427, PMID: 34970279). To examine whether T cell responses in the lung are enhanced by IN vaccination adjuvanted with Fos47, vaccinated mice were sacrificed on day 56. After lung perfusion to remove circulating blood cells and immune cells, immune cells in the lung were isolated. CD4⁺ CD44⁺CD69⁺ and CD8⁺CD44⁺CD69⁺ activated T cells in the lung were examined by flow cytometry. Flow cytometric analysis revealed that the percentages of CD44⁺CD69⁺ activated T cells in CD4⁺ and CD8⁺ T cells were significantly increased by IN boosting with Fos47 (FIG. 42A). The IM-IM vaccination with Fos47 had minimal effects on T cell responses in the lungs and this was significantly improved by IN-boosting with Fos47 (FIG. 42A). To further investigate whether these enhanced T cell responses induced by IN-boosting Fos47 are antigen-specific, lung immune cells isolated from IN boosted mice were analyzed by flow cytometry using MHC I tetramer and MHC II tetramer reagents, which can detect TCRs on T cells specific to IYSTVASSL and SFERFEIFPKE, respectively. The proportions of MHC II tetramer+ in CD4⁺CD44⁺ memory T cells and MHC I tetramer⁺ in CD8⁺CD44⁺ memory T cells in the lungs were increased by IN-Fos47 (FIGS. 42B and 42C). To examine the tissue residency of those T cell subsets in the lung, we employed an in vivo antibody labeling technique to identify resident T cells and exclude circulating immune cells (PMID: 24385150, PMID: 27468427). Mice were intravenously (i.v.) injected with anti-CD45-PE/Cy7 antibody 3 minutes prior to euthanasia. The lungs were then perfused, and lung cells were isolated. With this approach, circulating cells are labeled with the antibody, whereas cells retained within tissues are protected from antibody labeling (i.v. CD45⁻). i.v. co45-cells are considered non-circulating (or tissue-resident) cells. Flow cytometric analysis showed that the percentage of i.v. CD45⁻ non-circulating cells in CD4⁺CD44⁺ and CD8⁺CD44⁺ cells were increased in mice vaccinated by IM-IN-IN regimen with Fos47 compared to those in mice vaccinated with Lipo-Veh (FIG. 44).

Next recall T cell responses were examined after IM-IN-IN vaccination regimen. Splenocytes isolated from IN boosted mice were re-stimulated with HA ex vivo. IFNγ, IL-5 and IL-17 levels, Th1, Th2 and ThI 7-mediated cytokines, respectively (PMID: 20383174), were evaluated by ELISA. The levels of IFNγ, IL-5 and IL-17 in the culture supernatants were enhanced in the IN-Fos47 group compared to the IN-Lipo-Veh group (FIG. 44). These results suggest that the IM-IN-IN vaccination with Fos47 enhanced local and systemic T cell responses.

In Administration with Fos47 Did not Induce Local and Systemic Reactogenicity.

To assess whether IN administration of Fos47 may cause adverse effects, pro-inflammatory cytokine and chemokine secretion levels were measured in BALF at 6 and 24 hours by Multiplex Cytokine/chemokine assay. IL-6, TNFα and KC levels in BALFs induced by IN-administered Fos47 were as low as those by IN-administered Lipo-Veh (FIG. 44A). The same pattern was observed in serum IL-6, TNFα and KC levels (FIG. 46). Because chronic and robust type I interferon expression often cause immunosuppression, it was tested whether Fos47 induces prolonged expression of type I IFN downstream genes. Cells were collected from BALFs (BAL cells) at 2 to 48 hours post IN delivery of Lipo-Veh, Fos47 or AS01B and evaluated gene expression of interferon stimulated genes (ISG15 and ISG56) by Quantigene. Time course gene expression analysis showed that IN-Fos47 did not enhance ISG15 or ISG56 expression, whereas AS01B significantly increased expression of both genes (FIG. 45B). Mobility is another measure for assessing vaccine adverse effects. Therefore, it was observed mice behavior after IN-administration of Lipo-Veh, Fos47 and AS01B and there was no significant change in any treatment groups. As IN-administered TLRs cause anorexic behavior (PMID: 18480244). Body weight changes following IN-administration were noted. Mice in the IN Fos47 group maintained 99.6% of their initial body weights on day 2, whereas AS01B-administered mice decreased to 97.6% of their initial body weights on day 2 and recovered thereafter (FIG. 44C).

### REFERENCES

Admyre et al., Eur. J. Immunol., 36:1772 (2006).
Ambattu et al., Commun. Biol., 3:553 (2020).
Anand, Commun. Integr. Biol., 3:405 (2014).
Anticoli et al., Biotechnol. J., 13:e1700443 (2018).
Baell & Holloway, J. Med. Chem., 53:2719 (2010)
Baell & Walters, Nature, 513:481 (2014).
Bansal et al., J. Immunol., 207:2405 (2021).
Barry et al., Nature, 493:106 (2013).
Besse et al., Oncoimmunology, 5:e1071008 (2016).
Brahmer et al., N. Engl. J. Med., 373:123 (2015).
Camargo et al., Curr. Biol., 17:2054 (2007).
Campos et al., J. Immunol. Res., 2015:832057 (2015).
Cerundolo et al., J. Immunol. Ref., 175:6123 (2016).
Chan et al., ACS Comb. Sci., 19:533 (2017a).
Chan et al., Bioconjug. Chem., 20:1194 (2009).
Chan et al., J. Med. Chem., 60:9142 (2017b).
Chatterjee et al., Cancer Res., 74:5008 (2014).
Cherfils-Vicini et al., J. Clin. Invest., 120:1285 (2010).
Chiappinelli et al., Cell, 162:974 (2015).
Cho et al., J. Immunol., 168:4907 (2002).
Consortium et al., Nat. Methods, 14:228 (2017).
Cossetti et al., Mol. Cell., 56:193 (2014).
Cottini et al., Nat. Med., 20:599 (2014).
Crooks et al., Plos Pathog., 17:e1009807 (2021).
Czerniecki et al., J. Immunol., 159:3823 (1997).
Dajon et al., Oncoimmunology, 4:c991 (2015)
Datta et al., J. Immunol., 170:4102 (2003).
Dewey et al., Curr. Biol., 25:2751 (2015).
Dong et al., Cell, 130:1120 (2007).
Dranoff, Nat. Rev. Immunol., 12:61 (2011).
Durcin et al., J. Extracell. Vesicles, 6:1305677 (2017).
Eitan et al., J. Extracell. Vesicles, 4:26373 (2015).
Engel et al., Expert Rev. Clin. Pharmacol., 4:275 (2011).
Escudier et al., J. Transl. Med., 3:10 (2005).
Fernandez-Poma et al., Cancer Res., 77:3672 (2017).
Fuertes et al., Trends Immunol., 34:67 (2013).
Gajewski et al., Nat. Immunol., 14:1014 (2013).
Geissc et al., J. Am. Acad. Dermatol., 50:722 (2004).
Godebu et al., Transl. Med., 12:27.1 (2014).
Goff et al., J. Virol., 89:3221 (2015).
Hagemann et al., J. Immunol., 175:1197 (2005).
Hanahan and Weinberg, Cell, 144:646 (2011).
Hansen et al., Trends Cell Biol., 25:499 (2015).
Hart et al., J. Immunol., 175:1636 (2005).
Harvey et al., Nat. Rev. Cancer, 13:246 (2013).
Hastie & Tibshirani, J. R. Stat. Soc. Ser. B (Methodological), 58:155. (1996).
Hayashi et al., Melanoma Reseach., 21:66 (2011).
Hayashi, ArrayExpress. https://www.ebi.ac.uk/arrayexpress/. Deposited 30 Oct. 2022.
Hsu et al., J. Immunother., 26:440 (2003).
Ikeda et al., Oncol. Rep., 37:2603 (2017).
Imai et al., J. Extracell. Vesicles, 4:26238 (2015).
Jafari et al., Biodrugs, 34:567 (2020).
Jesus et al., Eur. J. Pharm. Biopharm., 133:1 (2018).
Johnson and Halder, Nat. Rev. Drug Discov., 13:63 (2014).
Junt and Barchet, Nat. Rev. Immunol., 15:529 (2015).
Kalluri & LeBleu, Science, 367: (2020).
Kanuma et al., J. Immunol., 198:4707 (2017).
Kasai et al., Eur. J. Immunol., 10:175 (1980).
Keder et al., Curr. Biol., 25:2739 (2015).
Kim et al., Surgery, 136:295 (2004).
Kitamura et al., Nat. Rev. Immunol., 15:73 (2015).
Koga-Yamakawa et al., Int. J. Cancer, 132:580 (2013).
Komurov et al., BMC Genomics, 13:282 (2012).
Krsmer-Albers et al., Proteom. Clin. Appl., 1:1446 (2007).
Lai et al., ACS Nano, 8:483 (2014).
Lambert et al., Cell, 168:670 (2017).
Lei et al., Onco Targets Ther., 9:545 (2016).
Liechtenstein et al., Immunol. Endocr. Metab. Agents Med. Chem., 12:224 (2012).
Lindenbergh & Stoorvogel, Annu. Rev. Immunol., 36:435 (2018).
Liu et al., Blood, 109:4336 (2007).
Liu et al., Cell, 164:406 (2016).
Lutz et al., J. Immunol. Methods, 223:77 (1999).
Maji et al., J. Appl. Toxicol., 37:310 (2017).
Martinez-Gil et al., J. Virol., 87:1290 (2013).
McFayden et al., Cheminformatics in Drug Disc., 141-7 (2005).
Mendt et al., JCI Insight, 3:e99263 (2018).
Meng et al., Genes Dev., 30:1 (2016).
Meng et al., Nat. Commun., 6:8357 (2015).
Messenger et al., J. Cell. Biol., 217:2877 (2018).
Mi et al., Nat. Protoc., 8:1551 (2013).
Miller et al., Bmc Bioinformatics, 12:322 (2011).
Miyama et al., Sci. Rep., 7:3663 (2017).
Mizuno et al., BMC Med. Genomics, 2:18 (2009).
Mohammed et al., J. Thorac. Imaging, 1:26 (2011).
Montecalvo et al., J. Immunol., 180:3081 (2008).
Mori et al., Cell, 156:893 (2014).
Moroishi et al., Cell, 167:1525 (2016).
Moroishi et al., Genes Dev., 29:1271 (2015b).
Moroishi et al., Nat. Rev. Cancer, 15:73 (2015a).
Morse et al. J. Transl Med., 3:9 (2005).
Moya and Halder, Cell Res., 24:137 (2014).
Nakayama, Front. Immunol., 5:672 (2015).
Nanishi et al., Curr. Opin. Pediatr., 32:125 (2020).
Näslund et al., J. Immunol., 190:2712 (2013).
Nowell et al., Nat. Cell Biol., 18:168 (2016).
Ochi and Graffeo, J. Clin. Invest., 122:4118 (2012).
Ostraml-Rosenberg and Fenselau, J. Immunol., 200:422 (2018).
Petrovsky, Drug Saf., 38:1059N (2015).
Pico de Coana et al., Trends Mol. Med., 21:482 (2015).
Pu et al., Stat. Med., 31:4175 (2012).
Pulaski et al., Curr. Protoc. Immunol., 20:20.2 (2001).
Qazi et al., Blood, 113:2673 (2009).
Quail and Joyce, Oncologist, 13:859 (2008).
Ramadan et al., Clin. Exp. Immunol., 125:237 (2001).
Ran et al., Nat. Protoc., 8:2281 (2013).
Rao & Hogan, Immunol. Rev., 231:5 (2009).
Reed et al., Nat. Med., 19:1597 (2013).
Robbins and Morelli, Nat. Rev. Immunol., 14:195 (2014).
Roederer, Cytom. Part A, 79A:95 (2011).
Roederer, Interpretation of cellular proliferation data: Avoid the panglossian. Cytom Part A, 79A:95 (2011).
Sabanovic et al., Biology, 10:94 (2021).
Saito et al., Acs. Chem. Biol., 17:217 (2022).
Saito et al., Front. Immunol., 12:701445 (2021).
Saleh et al., Nanoscale, 11:6990 (2019).
Sanmarco et al., Biochim Biophys. Acta—Mn Basis Dis., 1863:R57 (2017).
Santos & Almeida, Front. Immunol., 12:711565 (2021).
Sato-Kaneko et al., Front. Immunol., 11:1207 (2020).
Sato-Kaneko et al., Insight, 2 (2017).
Sato-Kaneko et al., Proc. Natl. Acad. Sci. USA, 118:e2025718118 (2021).
Savage et al., Br. T. Cancer, 74:1482 (1996).
Savina et al., J. Biol. Chem., 278:20083 (2003).
Schorey et al., Embo. Rep., 16:24 (2015).
Schwanhausser et al., Nature, 473:337 (2011).
Scrucca et al., R. J., 8:289 (2016).
Senovilla et al., Science, 337:1678 (2012).
Sharma and Allison, Cell, 161:205 (2015).
Shpigelman et al., Front. Pharmacol., 12:668609 (2021).
Shuilina et al., Am. J. Physiol. Cell. Physiol., 300:C1205 (2011).
Shukla et al., Front. Pharmacol., 13:869649 (2022).
Shukla et al., SLAS Discov., 23:960 (2018).
Sistigu et al., Nat. Med., 20:1301 (2014).
Skog et al., Nat. Cell. Biol., 10:1470 (2008).
Smith & Hall, Brain, 117:1351 (1994).
Spitzer et al., Cell, 168:487 (2017).
Steeg, Nat. Rev. Cancer, 16:201 (2016).
Straten et al., Proc. Natl. Acad. Sci. U.S.A., 95:8785 (1998).
Subudhi et al., Proc. Natl. Acad. Sci., 2:11919.
Takahashi et al., J. Biotechnol., 165:77 (2013).
Taniguchi et al., Nature, 519:57 (2015).
Taylor et al., J. Extracell. Vesicles, 9:1734326 (2020).
Topalian et al., Nat. Rev. Cancer, 16:275 (2016).
Tregoning et al., Vaccin. Immunother., 14:550 (2018).
Valadi et al., Nat. Cell. Biol., 9:654 (2007).
Valastyan and Weinberg, Cell, 147:275 (2011).
Van Deun et al., Nat. Methods, 14:228 (2017).
Venturi et al., Nat. Rev. Immunol., 8:231 (2008).
Vig & Kinet, Nat. Immunol., 10:21 (2009).
Wang et al., Cancer Metastasis Rev., 33:173 (2014).
Wang et al., Front Pharmacol., 8:300 (2017).
Wang et al., Mol. Cancer Ther., 9:1788 (2010).
Wen et al., J. Extracell. Vesicles, 6:1400370 (2017).
Wiklander et al., J. Extracell Vesicles, 4:26316 (2015).
Wolchok et al., N. Engl. J. Med., 369:122 (2013).
Wolchok et al., N. Engl. J. Med., 377:1345 (2017).
Wu et al., Innate Immun., 6:315 (2014).
Yáñez-Mó et al., J. Extracell. Vesicles, 4:27066 (2015).
Yoshida et al., Immunogenetics, 52:35 (2000).
Yu et al., Cell, 163:811 (2015).
Zhang et al., BMC Bioinformatics., 18:129 (2017).
Zhao et al., Genes Dev., 21:2747 (2007).
Zhao et al., Genes Dev., 22:1962 (2008).
Zhou et al., Cancer Cell, 16:425 (2009).
Zhu et al., J. Extracell Vesicles, 6:1324730 (2017).
Zitvogel et al., Nat. Med., 4:594 (1998).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

We claim:

1. A method to enhance an immune response in a mammal, comprising administering to a mammal in need thereof a composition comprising an effective amount of a compound that alters calcium mobilization and/or increases immunoenhancing extracellular vesicle (EVs) production.

2. The method according to claim 1, wherein the compound is of formula (IV) or a pharmaceutically acceptable salt thereof: 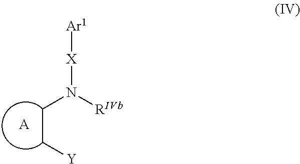
wherein
Ar¹ is selected from the group consisting of monocyclic or bicyclic C6-C10-aryl and bicyclic 9- to 10-membered heteroaryl (wherein 1-4 heteroaryl members are independently selected from N, O, S, and Se), optionally substituted with 1 to 3 R^{IVc};
Ring A is a monocyclic or bicyclic 5- to 10-membered fully or partially saturated heterocycloalkyl (wherein 1 to 4 ring members are independently selected from N, O, and S) or 5- to 10-membered heteroaryl (wherein 1-4 heteroaryl members are independently selected from N, O, and S), optionally substituted with 1 to 3 R^{IVc};
X is —SO₂—, —(HN)S(O)—, or —C(O)—;
Y is a moiety selected from the group consisting of 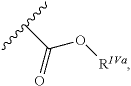
(a) wherein R is selected from the group consisting of H, C₁-C₆-alkyl, C₃-C₁₀-cycloalkyl, C₆-C₁₀-aryl, —C₁-C₆-alkyl(C₆-C₁₀-aryl), —C₁-C₆-alkyl-NRR′ (wherein R and R′ are independently selected from H, C₁-C₆-alkyl, —C(O)OC₁-C₆-alkyl, —C(O)C₀-C₆-alkyl(fluorophore or biotin)); or
(b) H, halo, CN, C(O)NRR′, N(R)C(O)(C₁-C₆-alkyl), 5- to 10-membered heteroaryl (wherein 1-4 heteroaryl members are independently selected from N, O, and S) that is optionally substituted with 1 to 3 R^{IVc}, and —C(O)(C₁-C₆)(biotin);
R^{IVb} is H or C₁-C₆-alkyl; and
R^{IVc} in each instance is independently selected from the group consisting of C₁-C₆-alkyl, OH, NH₂, halo, —C(O)C₁-C₆-alkyl, —C(O)OC₁-C₆-alkyl, C₆-C₁₀-aryl, and 5- to 10-membered heteroaryl (wherein 1-4 heteroaryl members are independently selected from N, O, and S) optionally substituted with 1 to 3 C₁-C₆-alkyl.

3. The method according to claim 2, wherein X is —SO₂—.

4. The method according to claim 2 or 3, wherein Y is (a)

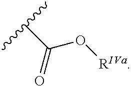

5. The method according to claim 2 or 4, wherein Ar¹ is a bicyclic 9- to 10-membered heteroaryl (wherein 1-4 heteroaryl members are independently selected from N, O, S, and Se), optionally substituted with 1 to 3 R^{IVc}.

6. The method according to any of claims 2 to 5, wherein Ar¹ is selected from the group consisting of optionally substituted:

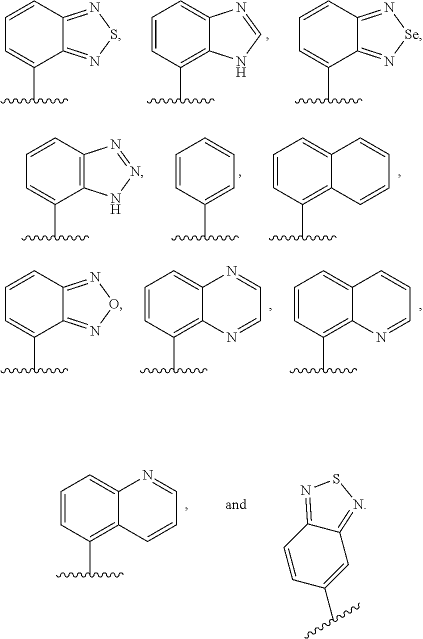

7. The method according to any of claims 2 to 6, wherein Ring A is a 5- to 10-membered heteroaryl (wherein 1-4 heteroaryl members are independently selected from N, O, and S), optionally substituted with 1 to 3 R^{IVc}.

8. The method according to any of claims 2 to 7, wherein Ring A is a 5-membered heteroaryl (wherein 1-2 heteroaryl members are independently selected from N and S), optionally substituted with 1 to 3 R^{IVc}.

9. The method according to any of claims 2 to 6, wherein Ring A is selected from the group consisting of optionally substituted:

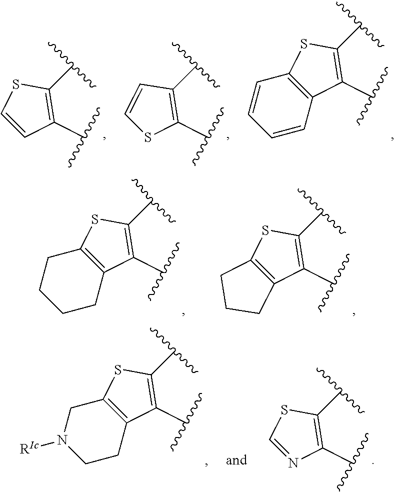

10. The method according to claim 2 or 4, wherein R^{IVa} is selected from the PGP-160 C₁ group consisting of H, C₁-C₆-alkyl, C₃-C₁₀-cycloalkyl, C₆-C₁₀-aryl, —C₁-C₆-alkyl(C₆-C₁₀-aryl), —C₁-C₆-alkyl-NRR′ (wherein R and R′ are independently selected from H, C₁-C₆-alkyl, and —C(O)OC₁-C₆-alkyl).

11. The method according to claim 2, wherein the compound is of formula (IVA): 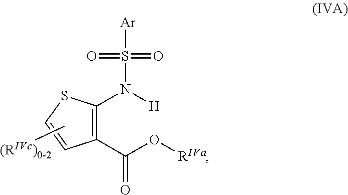
wherein
Ar¹ is a bicyclic 9- to 10-membered heteroaryl (wherein 1-4 heteroaryl members are independently selected from N, O, S, and Se), optionally substituted with 1 to 3 R^{IVc}.

12. The method according to claim 2, wherein the compound is one selected from the following table:

| | |
|---|---|
| 634 | 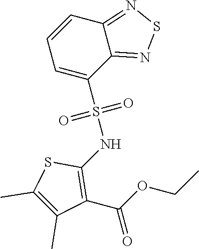 [SMILES: CCOC(=O)c1c(NS(=O)(=O)c2cccc3nsnc23)sc(C)c1C] |
| 2H013 | 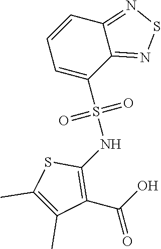 [SMILES: Cc1sc(NS(=O)(=O)c2cccc3nsnc23)c(C(=O)O)c1C] |
| 2G275 | 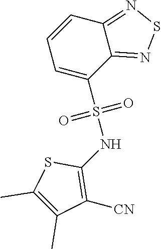 [SMILES: Cc1sc(NS(=O)(=O)c2cccc3nsnc23)c(C#N)c1C] |
| 2G182 | 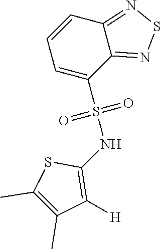 [SMILES: Cc1cc(NS(=O)(=O)c2cccc3nsnc23)sc1C] |
| 2E241 | 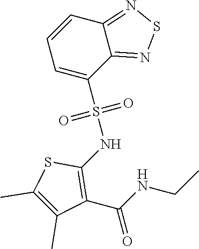 [SMILES: CCNC(=O)c1c(NS(=O)(=O)c2cccc3nsnc23)sc(C)c1C] |
| 2G179a | 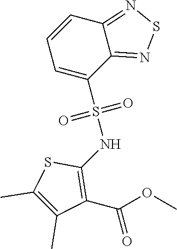 |
| 2G176 | 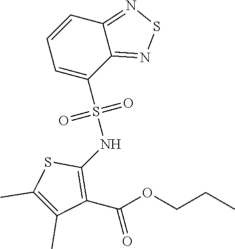 [SMILES: CCCOC(=O)c1c(NS(=O)(=O)c2cccc3nsnc23)sc(C)c1C] |
| 2G179b | 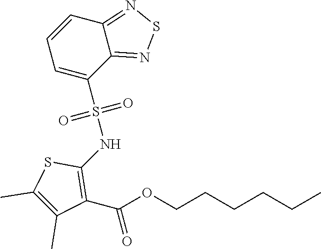 [SMILES: CCCCCCOC(=O)c1c(NS(=O)(=O)c2cccc3nsnc23)sc(C)c1C] |
| 2G179c | 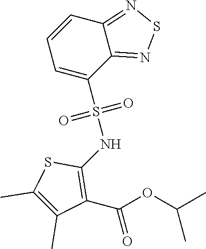 [SMILES: Cc1sc(NS(=O)(=O)c2cccc3nsnc23)c(C(=O)OC(C)C)c1C] |
| 2G179d | 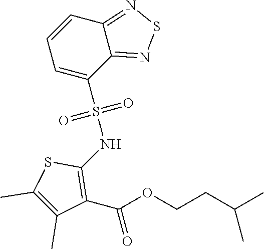 [SMILES: Cc1sc(NS(=O)(=O)c2cccc3nsnc23)c(C(=O)OCCC(C)C)c1C] |
| 2G198 | 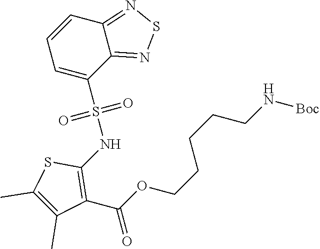 [SMILES: Cc1sc(NS(=O)(=O)c2cccc3nsnc23)c(C(=O)OCCCCCNC(=O)OC(C)(C)C)c1C] |
| 2G179h | 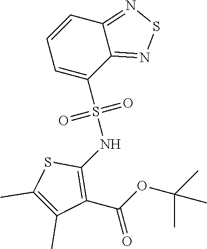 [SMILES: Cc1sc(NS(=O)(=O)c2cccc3nsnc23)c(C(=O)OC(C)(C)C)c1C] |
| 2G179e | 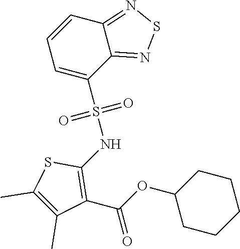 [SMILES: Cc1sc(NS(=O)(=O)c2cccc3nsnc23)c(C(=O)OC2CCCCC2)c1C] |
| 2G179g | 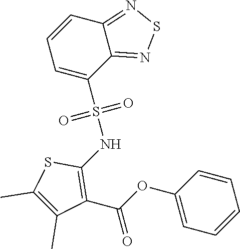 [SMILES: Cc1sc(NS(=O)(=O)c2cccc3nsnc23)c(C(=O)Oc2ccccc2)c1C] |
| 2G179f | 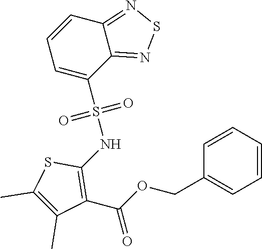 [SMILES: Cc1sc(NS(=O)(=O)c2cccc3nsnc23)c(C(=O)OCc2ccccc2)c1C] |
| 2G201 | 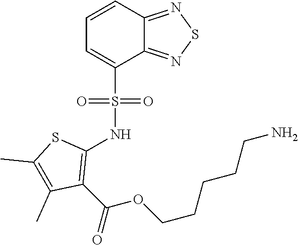 [SMILES: Cc1sc(NS(=O)(=O)c2cccc3nsnc23)c(C(=O)OCCCCCN)c1C] |
| 2G281a | 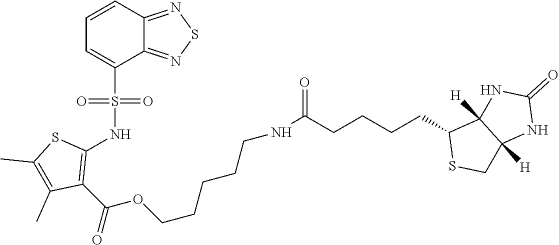 [SMILES: Cc1sc(NS(=O)(=O)c2cccc3nsnc23)c(C(=O)OCCCCCNC(=O)CCCC[C@H]2SC[C@H]3NC(=O)N[C@H]32)c1C] |
| 2G281c | 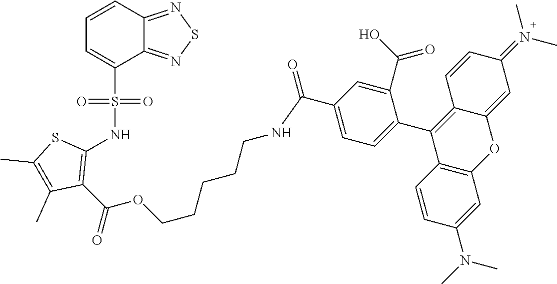 |
| 2G281b | 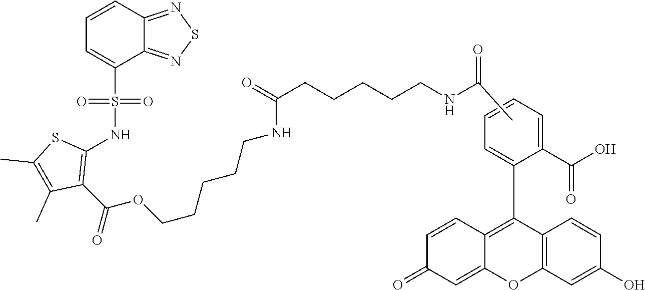 |
| 2G280 | 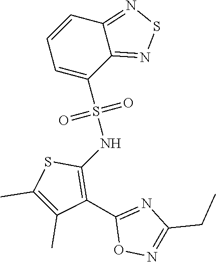 [SMILES: CCc1noc(-c2c(NS(=O)(=O)c3cccc4nsnc34)sc(C)c2C)n1] |
| 2G211 | 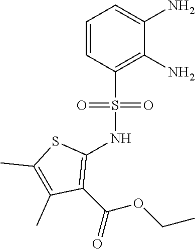 [SMILES: CCOC(=O)c1c(NS(=O)(=O)c2cccc(N)c2N)sc(C)c1C] |
| 2G225 | 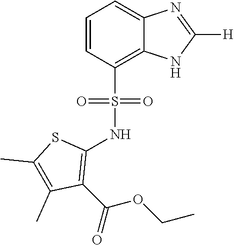 [SMILES: CCOC(=O)c1c(NS(=O)(=O)c2cccc3nc[nH]c23)sc(C)c1C] |
| 2G224 | 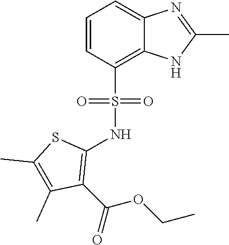 |
| 2C217 | 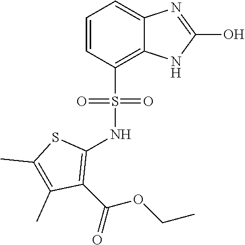 [SMILES: CCOC(=O)c1c(NS(=O)(=O)c2cccc3nc(O)[nH]c23)sc(C)c1C] |
| 2G272 | 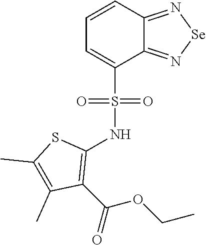 [SMILES: CCOC(=O)c1c(NS(=O)(=O)c2cccc3n[se]nc23)sc(C)c1C] |
| 2G274 | 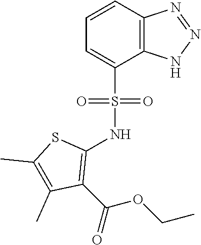 [SMILES: CCOC(=O)c1c(NS(=O)(=O)c2cccc3nn[nH]c23)sc(C)c1C] |
| 2G281 | 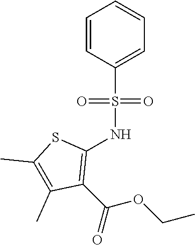 [SMILES: CCOC(=O)c1c(NS(=O)(=O)c2ccccc2)sc(C)c1C] |
| 2G222 | 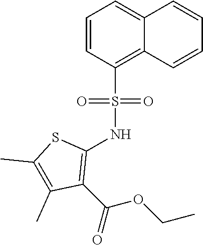 [SMILES: CCOC(=O)c1c(NS(=O)(=O)c2cccc3ccccc23)sc(C)c1C] |
| 2G223 | 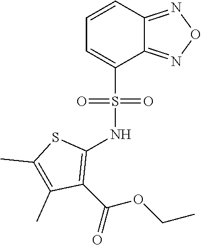 [SMILES: CCOC(=O)c1c(NS(=O)(=O)c2cccc3nonc23)sc(C)c1C] |
| 2H064 | 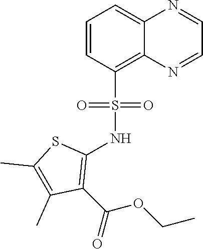 [SMILES: CCOC(=O)c1c(NS(=O)(=O)c2cccc3nccnc23)sc(C)c1C] |
| 2H069 | 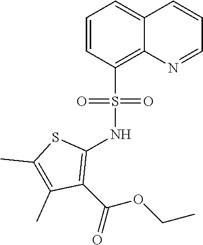 [SMILES: CCOC(=O)c1c(NS(=O)(=O)c2cccc3cccnc23)sc(C)c1C] |
| 2C230 | 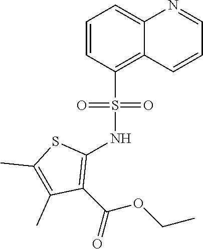 [SMILES: CCOC(=O)c1c(NS(=O)(=O)c2cccc3ncccc23)sc(C)c1C] |
| 2E287 | 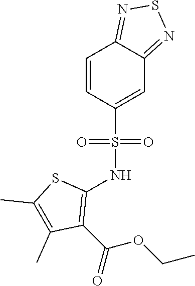 [SMILES: CCOC(=O)c1c(NS(=O)(=O)c2ccc3nsnc3c2)sc(C)c1C] |
| 2G270 | 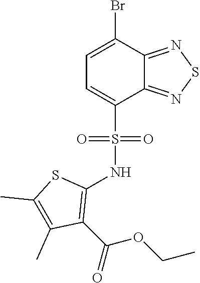 [SMILES: CCOC(=O)c1c(NS(=O)(=O)c2ccc(Br)c3nsnc23)sc(C)c1C] |
| 2F186 | 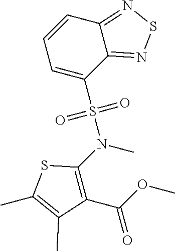 |
| 2F238 | 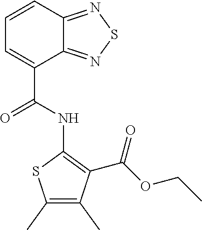 [SMILES: CCOC(=O)c1c(NC(=O)c2cccc3nsnc23)sc(C)c1C] |
| 2C228 | 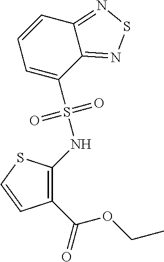 [SMILES: CCOC(=O)c1ccsc1NS(=O)(=O)c1cccc2nsnc12] |
| 2G244 | 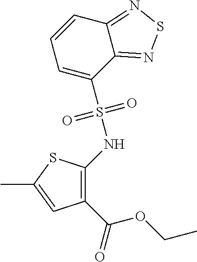 [SMILES: CCOC(=O)c1cc(C)sc1NS(=O)(=O)c1cccc2nsnc12] |
| 2G245 | 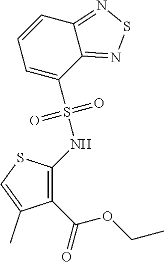 [SMILES: CCOC(=O)c1c(C)csc1NS(=O)(=O)c1cccc2nsnc12] |
| 2G246b | 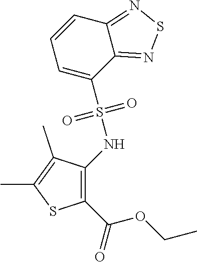 [SMILES: CCOC(=O)c1sc(C)c(C)c1NS(=O)(=O)c1cccc2nsnc12] |
| 2H076 | 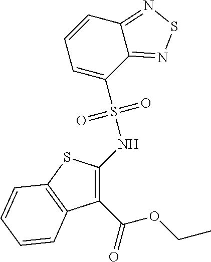 [SMILES: CCOC(=O)c1c(NS(=O)(=O)c2cccc3nsnc23)sc2ccccc12] |
| 2C229 | 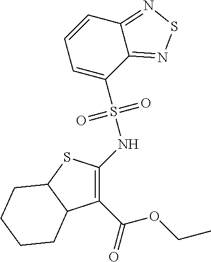 [SMILES: CCOC(=O)C1=C(NS(=O)(=O)c2cccc3nsnc23)SC2CCCCC12] |
| 2H079 | 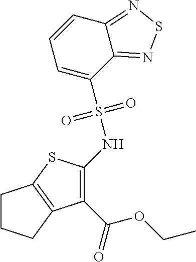 [SMILES: CCOC(=O)c1c(NS(=O)(=O)c2cccc3nsnc23)sc2c1CCC2] |
| 2G255 | 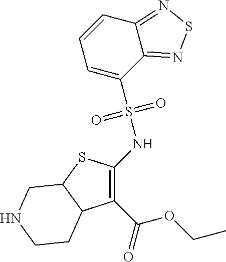 [SMILES: CCOC(=O)C1=C(NS(=O)(=O)c2cccc3nsnc23)SC2CNCCC12] |
| 2G249 | 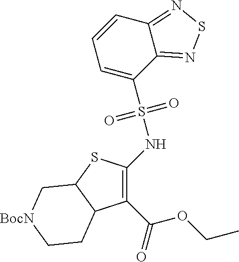 [SMILES: CCOC(=O)C1=C(NS(=O)(=O)c2cccc3nsnc23)SC2CN(C(=O)OC(C)(C)C)CCC12] |
| 2G252 | 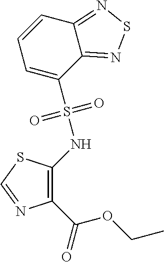 [SMILES: CCOC(=O)c1ncsc1NS(=O)(=O)c1cccc2nsnc12] |
| 2G254 | 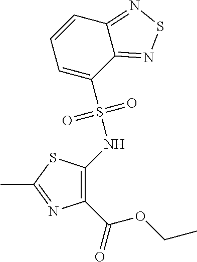 [SMILES: CCOC(=O)c1nc(C)sc1NS(=O)(=O)c1cccc2nsnc12] |
| 2G258 | 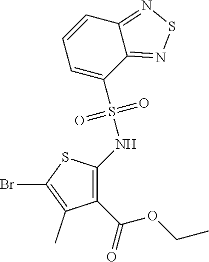 [SMILES: CCOC(=O)c1c(NS(=O)(=O)c2cccc3nsnc23)sc(Br)c1C] |
| 2G284 | 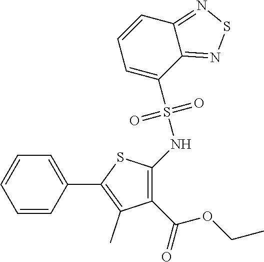 [SMILES: CCOC(=O)c1c(NS(=O)(=O)c2cccc3nsnc23)sc(-c2ccccc2)c1C] |
| 2G276 | 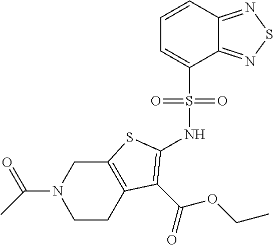 [SMILES: CCOC(=O)c1c(NS(=O)(=O)c2cccc3nsnc23)sc2c1CCN(C(C)=O)C2] |
| 2G279 | 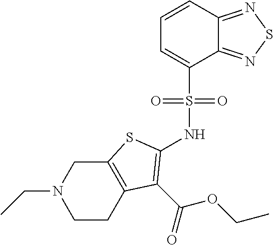 [SMILES: CCOC(=O)c1c(NS(=O)(=O)c2cccc3nsnc23)sc2c1CCN(CC)C2] |
| 2G287 | 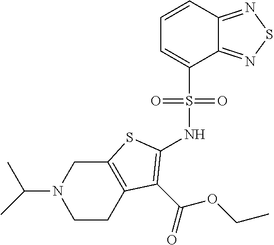 [SMILES: CCOC(=O)c1c(NS(=O)(=O)c2cccc3nsnc23)sc2c1CCN(C(C)C)C2] |
| 2H063 | 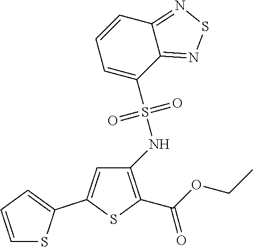 [SMILES: CCOC(=O)c1sc(-c2cccs2)cc1NS(=O)(=O)c1cccc2nsnc12] |
| 2H080 | 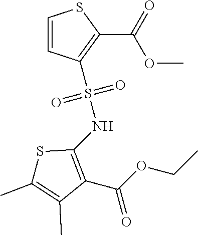 [SMILES: CCOC(=O)c1c(NS(=O)(=O)c2ccsc2C(=O)OC)sc(C)c1C] |
| | 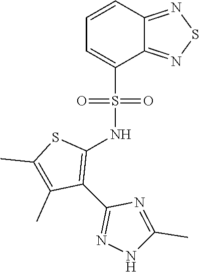 [SMILES: Cc1nc(-c2c(NS(=O)(=O)c3cccc4nsnc34)sc(C)c2C)n[nH]1] |
| | 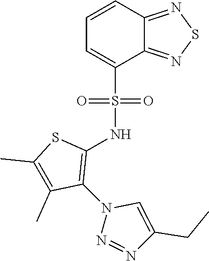 [SMILES: CCc1cn(-c2c(NS(=O)(=O)c3cccc4nsnc34)sc(C)c2C)nn1] |
| | 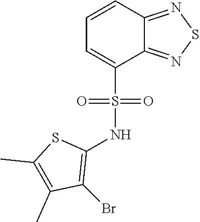 [SMILES: Cc1sc(NS(=O)(=O)c2cccc3nsnc23)c(Br)c1C] |
| | 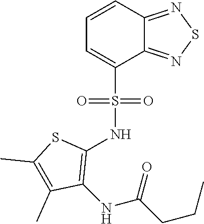 [SMILES: CCCC(=O)Nc1c(NS(=O)(=O)c2cccc3nsnc23)sc(C)c1C] |
| | 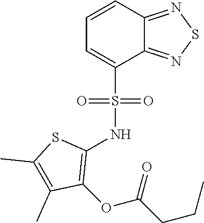 [SMILES: CCCC(=O)Oc1c(NS(=O)(=O)c2cccc3nsnc23)sc(C)c1C] |
| | 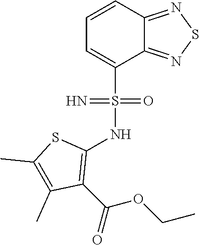 [SMILES: CCOC(=O)c1c(NS(=N)(=O)c2cccc3nsnc23)sc(C)c1C] |
| | 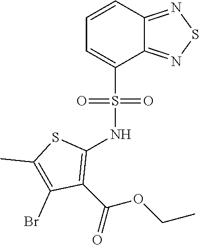 [SMILES: CCOC(=O)c1c(NS(=O)(=O)c2cccc3nsnc23)sc(C)c1Br] |
| | 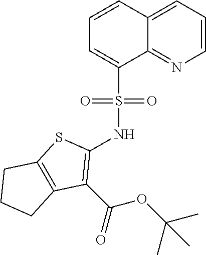 [SMILES: CC(C)(C)OC(=O)c1c(NS(=O)(=O)c2cccc3cccnc23)sc2c1CCC2] |

13. The method according to claim 1, wherein the compound is of formula (V) or a pharmaceutically acceptable salt thereof: 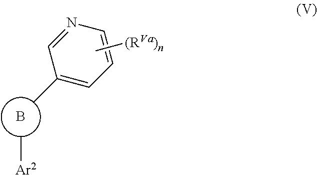
wherein
Ring B is 5-membered heteroaryl (wherein 1-4 heteroaryl members are independently selected from N, O, and S);
Ar² is selected from the group consisting of C₁-C₆-alkyl, monocyclic or bicyclic C₆-C₁₀-aryl, monocyclic or bicyclic 5- to 10-membered heteroaryl (wherein 1-4 heteroaryl members are independently selected from N, O, and S), and monocyclic or bicyclic 5- to 10-membered fully or partially saturated heterocycloalkyl (wherein 1 to 4 ring members are independently selected from N, O, and S), wherein Ar² is optionally substituted with 1 to 3 R^{IIIb};
R^{Va} is selected from the group consisting of C₁-C₆-alkyl, C₁-C₆-haloalkyl, halo, C₆-C₁₀-aryl, —O(C₆-C₁₀-aryl), —S(C₆-C₁₀-aryl), and 5- to 10-membered heteroaryl (wherein 1-4 heteroaryl members are independently selected from N, O, and S), wherein aryl or heteroaryl is optionally substituted with 1 to 3 substituents selected from C₁-C₆-alkyl and halo;
n is 0, 1, or 2; and
R^{Vb} is selected from the group consisting of C₁-C₆-alkyl, —OC₁-C₆-alkyl, C₁-C₆-haloalkyl, —OC₁-C₆-haloalkyl, halo, oxo, NO₂, CN, NRR′ (wherein R and R′ are independently selected from H and C₁-C₆-alkyl).

14. The method according to claim 13, wherein Ring B is selected from the group consisting of:

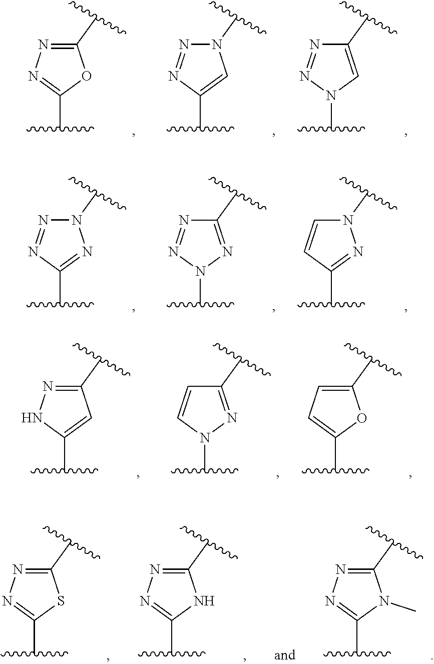

15. The method according to claim 13 or 14, wherein Ar² is optionally substituted monocyclic or bicyclic C₆-C₁₀-aryl.

16. The method according to any of claims 13 to 15, where Ar² is optionally substituted phenyl.

17. The method according to any of claims 13 to 15, where Ar² is optionally substituted naphthyl.

18. The method according to claim 13 or 14, wherein Ar² is optionally substituted monocyclic or bicyclic 5- to 10-membered heteroaryl (wherein 1-4 heteroaryl members are independently selected from N, O, and S).

19. The method according to any of claims 13, 14, and 18, wherein Ar² is optionally substituted monocyclic 5- to 6-membered heteroaryl (wherein 1-4 heteroaryl members are independently selected from N, O, and S).

20. The method according to any of claims 13 to 19, wherein n is 1 and R^{IIIa} is optionally substituted thiophenyl.

21. The method according to claim 13, wherein the compound is one selected from the following table:

| | |
|---|---|
| 645 | 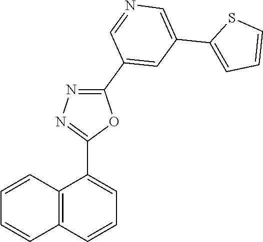 [SMILES: c1csc(-c2cncc(-c3nnc(-c4cccc5ccccc45)o3)c2)c1] |
| 1301 | 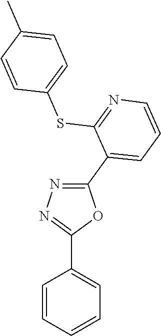 [SMILES: Cc1ccc(Sc2ncccc2-c2nnc(-c3ccccc3)o2)cc1] |
| 1302 | 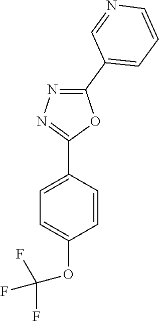 [SMILES: FC(F)(F)Oc1ccc(-c2nnc(-c3cccnc3)o2)cc1] |
| 1303 | 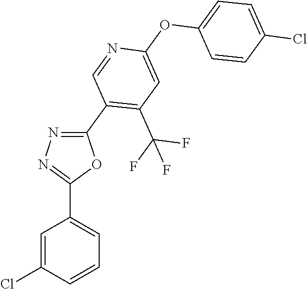 [SMILES: FC(F)(F)c1cc(Oc2ccc(Cl)cc2)ncc1-c1nnc(-c2cccc(Cl)c2)o1] |
| 1304 | 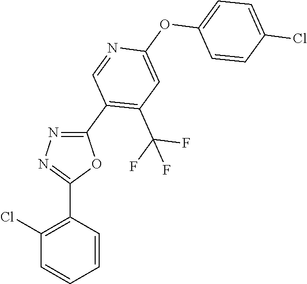 [SMILES: FC(F)(F)c1cc(Oc2ccc(Cl)cc2)ncc1-c1nnc(-c2ccccc2Cl)o1] |
| 1305 | 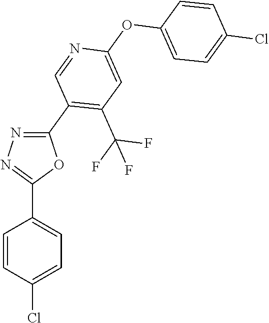 [SMILES: FC(F)(F)c1cc(Oc2ccc(Cl)cc2)ncc1-c1nnc(-c2ccc(Cl)cc2)o1] |
| 1310 | 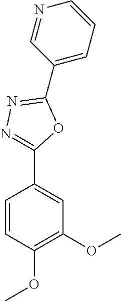 [SMILES: COc1ccc(-c2nnc(-c3cccnc3)o2)cc1OC] |
| 1306 | 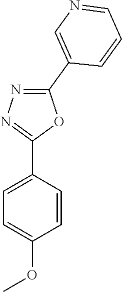 [SMILES: COc1ccc(-c2nnc(-c3cccnc3)o2)cc1] |
| 1311 | 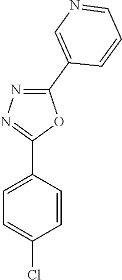 [SMILES: Clc1ccc(-c2nnc(-c3cccnc3)o2)cc1] |
| 1307 | 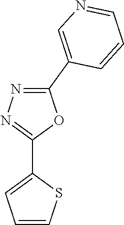 [SMILES: c1cncc(-c2nnc(-c3cccs3)o2)c1] |
| 1308 | 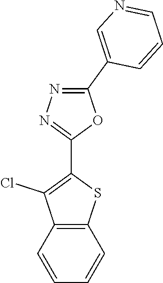 [SMILES: Clc1c(-c2nnc(-c3cccnc3)o2)sc2ccccc12] |
| 1309 | 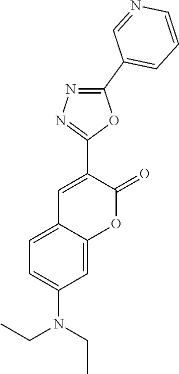 [SMILES: CCN(CC)c1ccc2cc(-c3nnc(-c4cccnc4)o3)c(=O)oc2c1] |
| 2C176 | 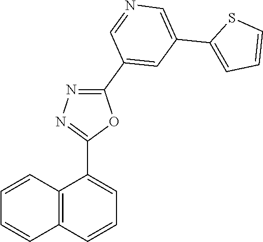 [SMILES: c1csc(-c2cncc(-c3nnc(-c4cccc5ccccc45)o3)c2)c1] |
| 2C173 | 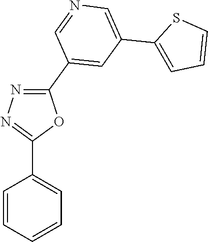 [SMILES: c1ccc(-c2nnc(-c3cncc(-c4cccs4)c3)o2)cc1] |
| 2C201 | 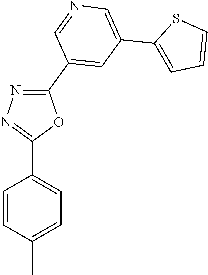 [SMILES: Cc1ccc(-c2nnc(-c3cncc(-c4cccs4)c3)o2)cc1] |
| 2H019 | 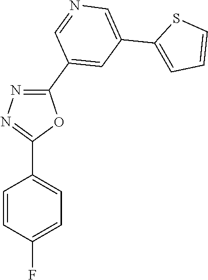 [SMILES: Fc1ccc(-c2nnc(-c3cncc(-c4cccs4)c3)o2)cc1] |
| 2H022 | 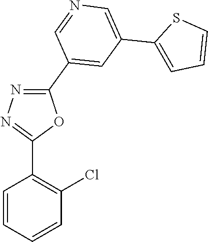 [SMILES: Clc1ccccc1-c1nnc(-c2cncc(-c3cccs3)c2)o1] |
| 2H023 | 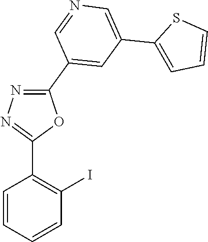 [SMILES: Ic1ccccc1-c1nnc(-c2cncc(-c3cccs3)c2)o1] |
| 2H028 | 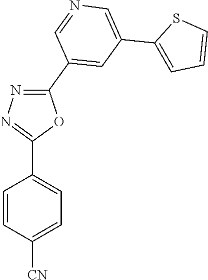 [SMILES: N#Cc1ccc(-c2nnc(-c3cncc(-c4cccs4)c3)o2)cc1] |
| 2H027 | 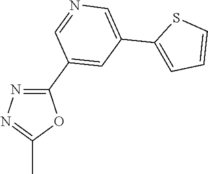 [SMILES: Cc1nnc(-c2cncc(-c3cccs3)c2)o1] |
| 2H032 | 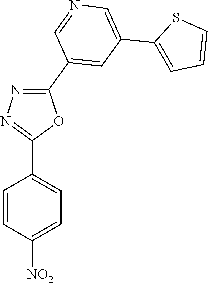 [SMILES: O=[N+]([O-])c1ccc(-c2nnc(-c3cncc(-c4cccs4)c3)o2)cc1] |
| 2H037 | 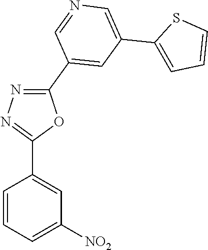 [SMILES: O=[N+]([O-])c1cccc(-c2nnc(-c3cncc(-c4cccs4)c3)o2)c1] |
| 2C211 | 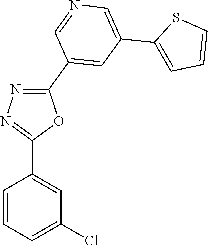 [SMILES: Clc1cccc(-c2nnc(-c3cncc(-c4cccs4)c3)o2)c1] |
| 2F188B | 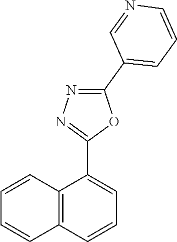 [SMILES: c1cncc(-c2nnc(-c3cccc4ccccc34)o2)c1] |
| 2F187 | 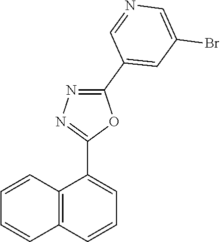 [SMILES: Brc1cncc(-c2nnc(-c3cccc4ccccc34)o2)c1] |
| 2C178 | 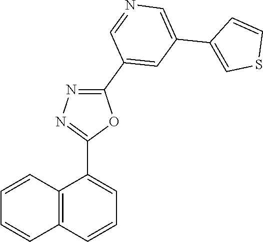 [SMILES: c1ccc2c(-c3nnc(-c4cncc(-c5ccsc5)c4)o3)cccc2c1] |
| 2H005 | 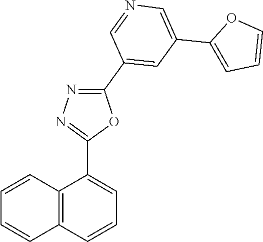 [SMILES: c1coc(-c2cncc(-c3nnc(-c4cccc5ccccc45)o3)c2)c1] |
| 2C179 | 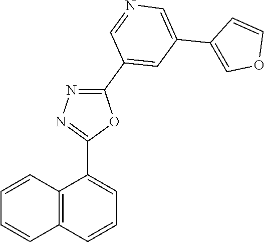 [SMILES: c1ccc2c(-c3nnc(-c4cncc(-c5ccoc5)c4)o3)cccc2c1] |
| 2C181 | 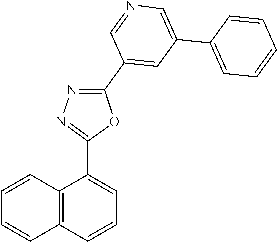 [SMILES: c1ccc(-c2cncc(-c3nnc(-c4cccc5ccccc45)o3)c2)cc1] |
| 2C183 | 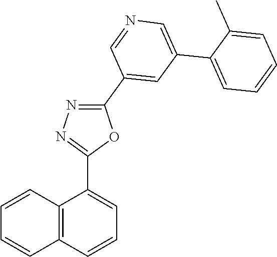 [SMILES: Cc1ccccc1-c1cncc(-c2nnc(-c3cccc4ccccc34)o2)c1] |
| 2C185 | 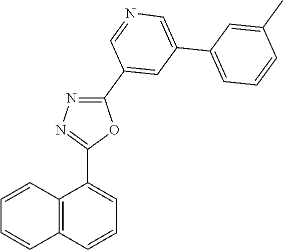 [SMILES: Cc1cccc(-c2cncc(-c3nnc(-c4cccc5ccccc45)o3)c2)c1] |
| 2C182 | 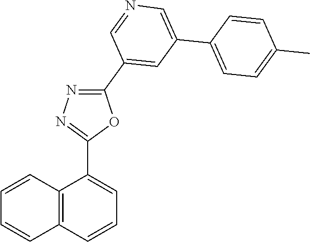 [SMILES: Cc1ccc(-c2cncc(-c3nnc(-c4cccc5ccccc45)o3)c2)cc1] |
| 2H008 | 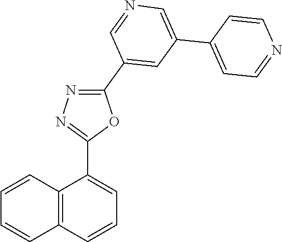 [SMILES: c1ccc2c(-c3nnc(-c4cncc(-c5ccncc5)c4)o3)cccc2c1] |
| 2C188 | 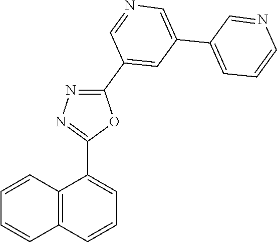 [SMILES: c1cncc(-c2cncc(-c3nnc(-c4cccc5ccccc45)o3)c2)c1] |
| 2H050 | 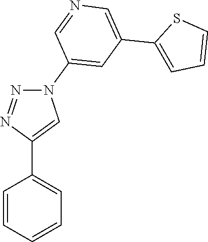 [SMILES: c1ccc(-c2cn(-c3cncc(-c4cccs4)c3)nn2)cc1] |
| 2H042 | 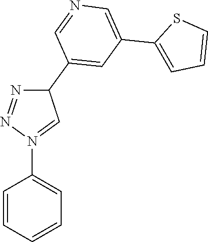 |
| 2E279 | 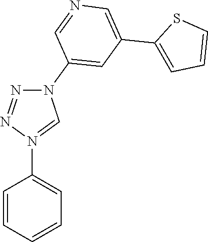 |
| 2E277 | 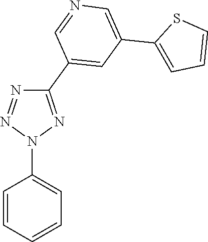 [SMILES: c1ccc(-n2nnc(-c3cncc(-c4cccs4)c3)n2)cc1] |
| 2C214 | 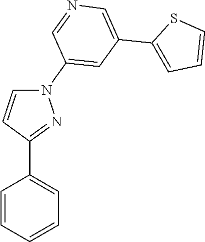 [SMILES: c1ccc(-c2ccn(-c3cncc(-c4cccs4)c3)n2)cc1] |
| 2E280 | 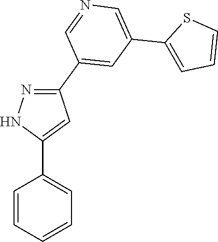 [SMILES: c1ccc(-c2cc(-c3cncc(-c4cccs4)c3)n[nH]2)cc1] |
| | 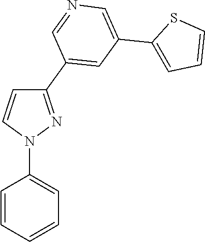 [SMILES: c1ccc(-n2ccc(-c3cncc(-c4cccs4)c3)n2)cc1] |
| | 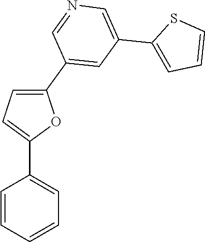 [SMILES: c1ccc(-c2ccc(-c3cncc(-c4cccs4)c3)o2)cc1] |
| | 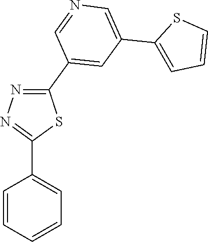 [SMILES: c1ccc(-c2nnc(-c3cncc(-c4cccs4)c3)s2)cc1] |
| | 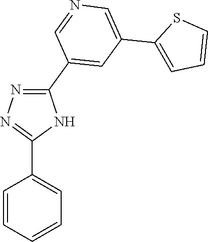 [SMILES: c1ccc(-c2nnc(-c3cncc(-c4cccs4)c3)[nH]2)cc1] |
| | 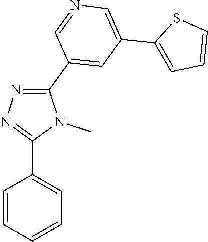 [SMILES: Cn1c(-c2ccccc2)nnc1-c1cncc(-c2cccs2)c1] |

22. The method of any one of claims 1 to 21 wherein the compound is an adjuvant.

23. The method of any one of claims 1 to 22 further comprising administering one or more antigens.

24. The method of claim 23 wherein the compound and the one or more antigens are in a composition.

25. The method of claim 23 or 24 wherein the antigen is from a microbe.

26. The method of claim 23 or 24 wherein the antigen is a cancer antigen.

27. A method to enhance the immune response of a mammal, comprising:
administering to the mammal an effective amount of a composition comprising a compound of formula (II-A) or a pharmaceutically acceptable salt thereof, formula (I) or a pharmaceutically acceptable salt thereof, or a combination thereof, wherein formula (II-A) comprises: 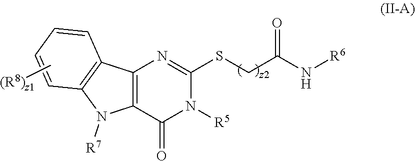
wherein
z1 is an integer from 0 to 4, and z2 is an integer from 0 to 5;
R⁵ is R^{5A}-substituted or unsubstituted cycloalkyl, R^{5A}-substituted or unsubstituted heterocycloalkyl, R^{5A}-substituted or unsubstituted aryl, or R^{5A}-substituted or unsubstituted heteroaryl.
R^{5A} is independently halogen, —CN, —CF₃, —CCl₃, —OH, —NH₂, —SO₂, —COOH, oxo, nitro, —SH, —CONH₂, —NH—OH, R^{5B}-substituted or unsubstituted alkyl, R^{5B}-substituted or unsubstituted alkynyl, R^{5B}-substituted or unsubstituted heteroalkyl, R^{5B}-substituted or unsubstituted cycloalkyl, R^{5B}-substituted or unsubstituted heterocycloalkyl, R^{5B}-substituted or unsubstituted aryl, or R^{5B}-substituted or unsubstituted heteroaryl;
R^{5B} is independently halogen, —CN, —CF₃, —CCl₃, —OH, —NH₂, —SO₂, —COOH, oxo, nitro, —SH, —CONH₂, R^{5C}-substituted or unsubstituted alkyl, R^{5C}-substituted or unsubstituted heteroalkyl, R^{5C}-substituted or unsubstituted cycloalkyl, R^{5C}-substituted or unsubstituted heterocycloalkyl, R^{5C}-substituted or unsubstituted aryl, or R^{5C}-substituted or unsubstituted heteroaryl;
R^{5C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl;
R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R⁷ is hydrogen, or substituted or unsubstituted alkyl; and
R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein formula (I) comprises: 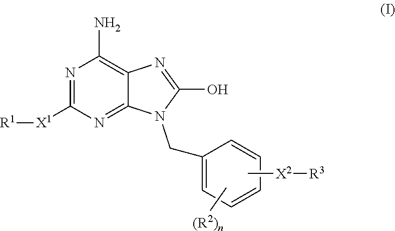
wherein X¹ is —O—, —S—, or —NR^{c}—;
R¹ is hydrogen, (C₁-C₁₀)alkyl, substituted (C₁-C₁₀)alkyl, C₆₋₁₀ aryl, or substituted C₆₋₁₀ aryl, C₅₋₉ heterocyclic, substituted C₅₋₉ heterocyclic;
R^{c} is hydrogen, C₁₋₁₀ alkyl, or substituted C₁₋₁₀ alkyl; or R^{c} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b}(carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are selected from hydroxy, C₁₋₆alkyl, hydroxyC₁₋₆alkylene, C₁₋₆ alkoxy, C₃₋₆-cycloalkyl, C₁₋₆ alkoxyC₁₋₆alkylene, amino, cyano, halo, and aryl;
n is 0, 1, 2, 3 or 4;
X² is a bond or a linking group;
R³ is a phospholipid comprising one or two carboxylic esters;
or both.

28. The method of claim 28 wherein the animal is a bovine, caprine, swine, ovine, equine, canine or feline.

29. The method of claim 28 wherein the mammal is a human

30. The method of claim 28 or 29 further comprising administering one or more antigens.

31. The method of claim 30 wherein the compound and the one or more antigens are in a composition.

32. The method of claim 30 or 31 wherein the antigen is from a microbe.

33. The method of claim 30 or 31 wherein the antigen is a cancer antigen.

34. The method of any one of claims 28 to 33 wherein the composition comprises liposomes comprising the compound of formula (I), formula (IIA), or both.

35. The method of any one of claims 28 to 33 wherein the composition is intranasally administered.

36. The method of any one of claims 28 to 33 wherein the composition is intramuscularly administered.

37. A compound of formula (IV) or a pharmaceutically acceptable salt thereof: 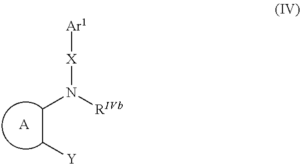
wherein
Ar¹ is selected from the group consisting of monocyclic or bicyclic C₆-C₁₀-aryl and bicyclic 9- to 10-membered heteroaryl (wherein 1-4 heteroaryl members are independently selected from N, O, S, and Se), optionally substituted with 1 to 3 R^{IVc};
Ring A is a monocyclic or bicyclic 5- to 10-membered fully or partially saturated heterocycloalkyl (wherein 1 to 4 ring members are independently selected from N, O, and S) or 5- to 10-membered heteroaryl (wherein 1-4 heteroaryl members are independently selected from N, O, and S), optionally substituted with 1 to 3 R^{IVc};
X is —SO₂—, —(HN)S(O)—, or —C(O)—;
Y is a moiety selected from the group consisting of 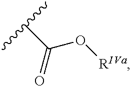
(a) wherein R is selected from the group consisting of H, C₁-C₆-alkyl, C₃-C₁₀-cycloalkyl, C₆-C₁₀-aryl, —C₁-C₆-alkyl(C₆-C₁₀-aryl), —C₁-C₆-alkyl-NRR′ (wherein R and R′ are independently selected from H, C₁-C₆-alkyl, —C(O)OC₁-C₆-alkyl, —C(O)C₀-C₆-alkyl(fluorophore or biotin)); or
(b) H, halo, CN, C(O)NRR′, N(R)C(O)(C₁-C₆-alkyl), 5- to 10-membered heteroaryl (wherein 1-4 heteroaryl members are independently selected from N, O, and S) that is optionally substituted with 1 to 3 R^{IVc}, and —C(O)(C₁-C₆)(biotin);
R^{IVb} is H or C₁-C₆-alkyl; and
R^{IVc} in each instance is independently selected from the group consisting of C₁-C₆-alkyl, OH, NH₂, halo, —C(O)C₁-C₆-alkyl, —C(O)OC₁-C₆-alkyl, C₆-C₁₀-aryl, and 5- to 10-membered heteroaryl (wherein 1-4 heteroaryl members are independently selected from N, O, and S) optionally substituted with 1 to 3 C₁-C₆-alkyl;
with the proviso that the compound is not:

| | |
|---|---|
| 634 | 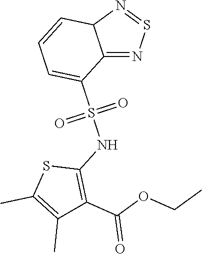 |

38. The compound according to claim 37, wherein X is —SO₂—.

39. The compound according to claim 37 or 38, wherein Y is (a)

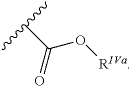

40. The compound according to claim 37 or 39, wherein Ar¹ is a bicyclic 9- to 10-membered heteroaryl (wherein 1-4 heteroaryl members are independently selected from N, O, S, and Se), optionally substituted with 1 to 3 R^{IVc}.

41. The compound according to any of claims 37 to 40, wherein Ar¹ is selected from the group consisting of optionally substituted:

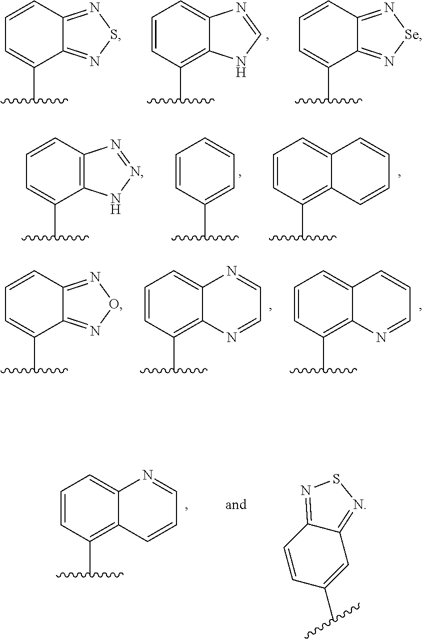

42. The compound according to any of claims 37 to 41, wherein Ring A is a 5- to 10-membered heteroaryl (wherein 1-4 heteroaryl members are independently selected from N, O, and S), optionally substituted with 1 to 3 R^{IVc}.

43. The compound according to any of claims 37 to 42, wherein Ring A is a 5-membered heteroaryl (wherein 1-2 heteroaryl members are independently selected from N and S), optionally substituted with 1 to 3 R^{IVc}.

44. The compound according to any of claims 37 to 41, wherein Ring A is selected from the group consisting of optionally substituted:

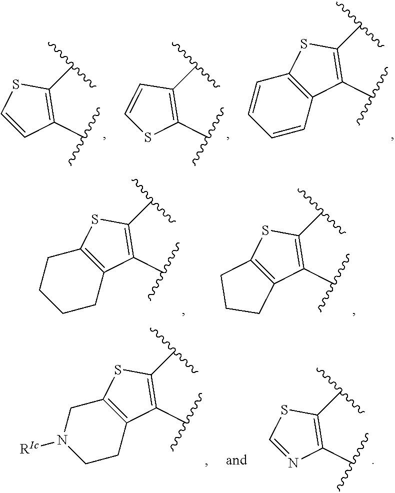

45. The compound according to claim 37 or 39, wherein R^{IVa} is selected from the group consisting of H, C₁-C₆-alkyl, C₃-C₁₀-cycloalkyl, C₆-C₁₀-aryl, —C₁-C₆-alkyl(C₆-C₁₀-aryl), —C₁-C₆-alkyl-NRR′ (wherein R and R′ are independently selected from H, C₁-C₆-alkyl, and —C(O)OC₁-C₆-alkyl).

46. The compound according to claim 37, wherein the compound is of formula (IVA): 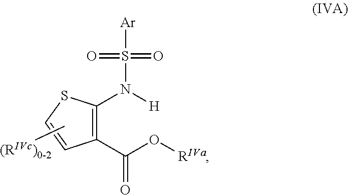
wherein
Ar¹ is a bicyclic 9- to 10-membered heteroaryl (wherein 1-4 heteroaryl members are independently selected from N, O, S, and Se), optionally substituted with 1 to 3 R^{IVc}.

47. The compound according to claim 37, wherein the compound is one selected from the following table:

| | |
|---|---|
| 2H013 | 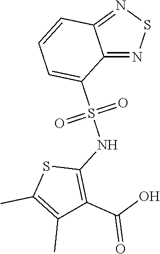 [SMILES: Cc1sc(NS(=O)(=O)c2cccc3nsnc23)c(C(=O)O)c1C] |
| 2G275 | 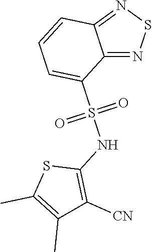 [SMILES: Cc1sc(NS(=O)(=O)c2cccc3nsnc23)c(C#N)c1C] |
| 2G182 | 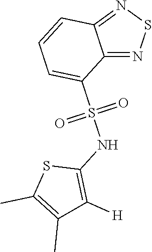 [SMILES: Cc1cc(NS(=O)(=O)c2cccc3nsnc23)sc1C] |
| 2E241 | 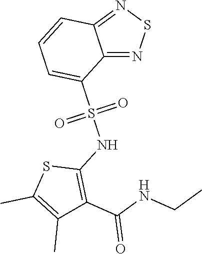 [SMILES: CCNC(=O)c1c(NS(=O)(=O)c2cccc3nsnc23)sc(C)c1C] |
| 2G179a | 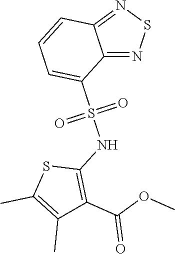 |
| 2G176 | 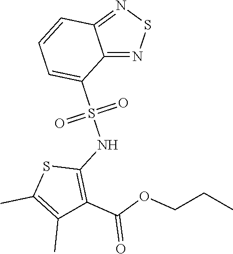 [SMILES: CCCOC(=O)c1c(NS(=O)(=O)c2cccc3nsnc23)sc(C)c1C] |
| 2G179b | 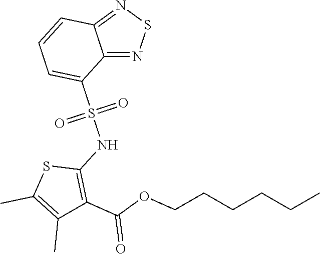 [SMILES: CCCCCCOC(=O)c1c(NS(=O)(=O)c2cccc3nsnc23)sc(C)c1C] |
| 2G179c | 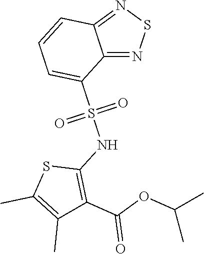 [SMILES: Cc1sc(NS(=O)(=O)c2cccc3nsnc23)c(C(=O)OC(C)C)c1C] |
| 2G179d | 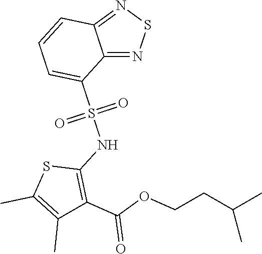 [SMILES: Cc1sc(NS(=O)(=O)c2cccc3nsnc23)c(C(=O)OCCC(C)C)c1C] |
| 2G198 | 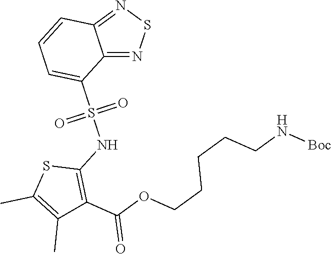 [SMILES: Cc1sc(NS(=O)(=O)c2cccc3nsnc23)c(C(=O)OCCCCCNC(=O)OC(C)(C)C)c1C] |
| 2G179h | 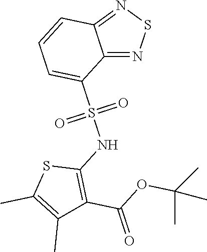 [SMILES: Cc1sc(NS(=O)(=O)c2cccc3nsnc23)c(C(=O)OC(C)(C)C)c1C] |
| 2G179e | 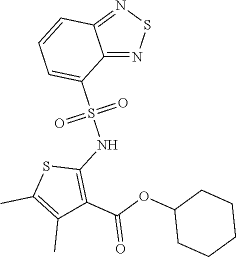 [SMILES: Cc1sc(NS(=O)(=O)c2cccc3nsnc23)c(C(=O)OC2CCCCC2)c1C] |
| 2G179g | 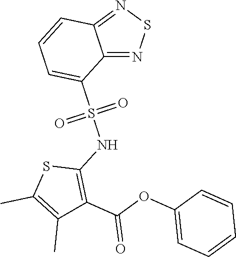 [SMILES: Cc1sc(NS(=O)(=O)c2cccc3nsnc23)c(C(=O)Oc2ccccc2)c1C] |
| 2G179f | 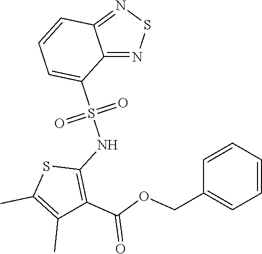 [SMILES: Cc1sc(NS(=O)(=O)c2cccc3nsnc23)c(C(=O)OCc2ccccc2)c1C] |
| 2G201 | 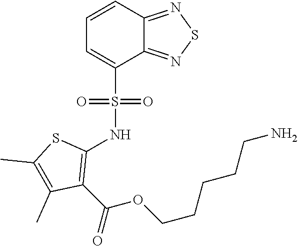 [SMILES: Cc1sc(NS(=O)(=O)c2cccc3nsnc23)c(C(=O)OCCCCCN)c1C] |
| 2G281a | 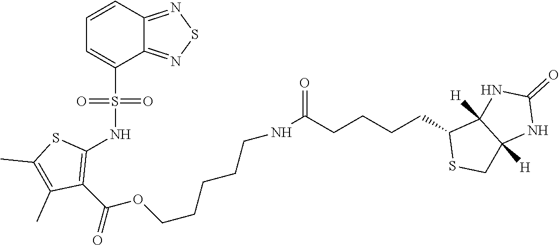 [SMILES: Cc1sc(NS(=O)(=O)c2cccc3nsnc23)c(C(=O)OCCCCCNC(=O)CCCC[C@H]2SC[C@H]3NC(=O)N[C@H]32)c1C] |
| 2G281c | 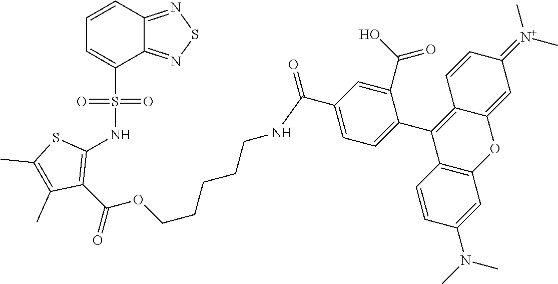 |
| 2G281b | 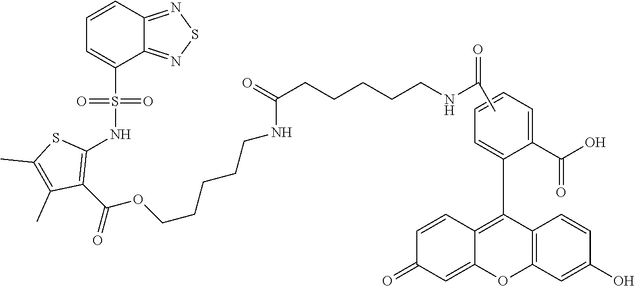 |
| 2G280 | 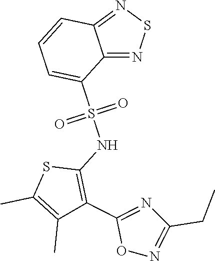 [SMILES: CCc1noc(-c2c(NS(=O)(=O)c3cccc4nsnc34)sc(C)c2C)n1] |
| 2G211 | 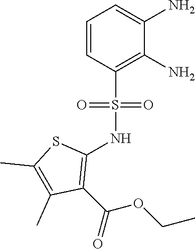 [SMILES: CCOC(=O)c1c(NS(=O)(=O)c2cccc(N)c2N)sc(C)c1C] |
| 2G225 | 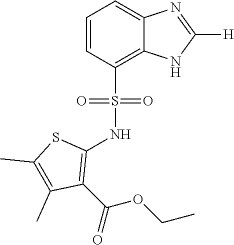 [SMILES: CCOC(=O)c1c(NS(=O)(=O)c2cccc3nc[nH]c23)sc(C)c1C] |
| 2G224 | 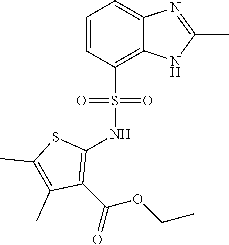 |
| 2C217 | 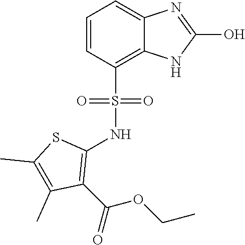 [SMILES: CCOC(=O)c1c(NS(=O)(=O)c2cccc3nc(O)[nH]c23)sc(C)c1C] |
| 2G272 | 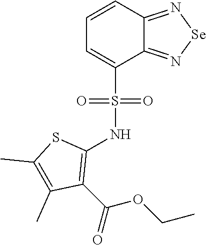 [SMILES: CCOC(=O)c1c(NS(=O)(=O)c2cccc3n[se]nc23)sc(C)c1C] |
| 2G274 | 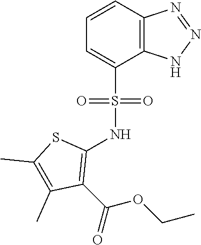 [SMILES: CCOC(=O)c1c(NS(=O)(=O)c2cccc3nn[nH]c23)sc(C)c1C] |
| 2G281 | 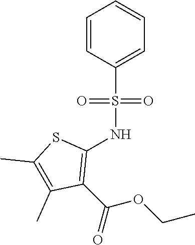 [SMILES: CCOC(=O)c1c(NS(=O)(=O)c2ccccc2)sc(C)c1C] |
| 2G222 | 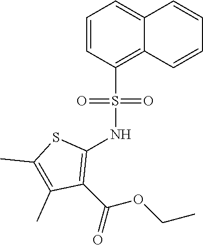 [SMILES: CCOC(=O)c1c(NS(=O)(=O)c2cccc3ccccc23)sc(C)c1C] |
| 2G223 | 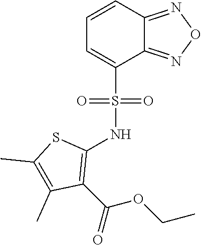 [SMILES: CCOC(=O)c1c(NS(=O)(=O)c2cccc3nonc23)sc(C)c1C] |
| 2H064 | 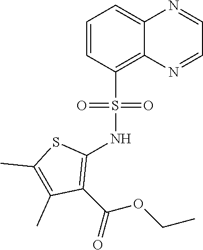 [SMILES: CCOC(=O)c1c(NS(=O)(=O)c2cccc3nccnc23)sc(C)c1C] |
| 2H069 | 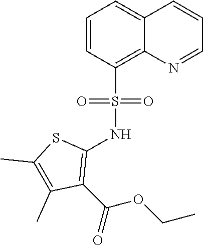 [SMILES: CCOC(=O)c1c(NS(=O)(=O)c2cccc3cccnc23)sc(C)c1C] |
| 2C230 | 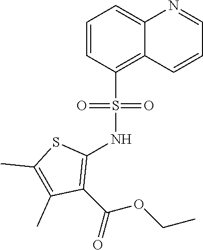 [SMILES: CCOC(=O)c1c(NS(=O)(=O)c2cccc3ncccc23)sc(C)c1C] |
| 2E287 | 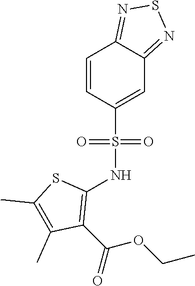 [SMILES: CCOC(=O)c1c(NS(=O)(=O)c2ccc3nsnc3c2)sc(C)c1C] |
| 2G270 | 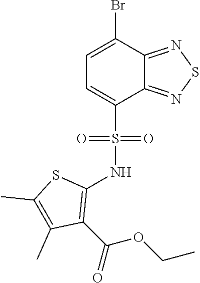 [SMILES: CCOC(=O)c1c(NS(=O)(=O)c2ccc(Br)c3nsnc23)sc(C)c1C] |
| 2F186 | 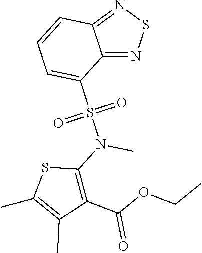 [SMILES: CCOC(=O)c1c(N(C)S(=O)(=O)c2cccc3nsnc23)sc(C)c1C] |
| 2F238 | 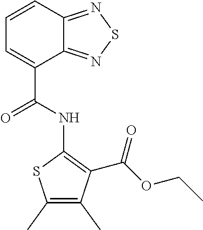 [SMILES: CCOC(=O)c1c(NC(=O)c2cccc3nsnc23)sc(C)c1C] |
| 2C228 | 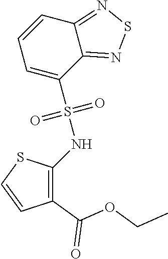 [SMILES: CCOC(=O)c1ccsc1NS(=O)(=O)c1cccc2nsnc12] |
| 2G244 | 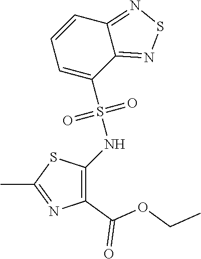 [SMILES: CCOC(=O)c1nc(C)sc1NS(=O)(=O)c1cccc2nsnc12] |
| 2G245 | 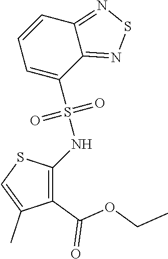 [SMILES: CCOC(=O)c1c(C)csc1NS(=O)(=O)c1cccc2nsnc12] |
| 2G246b | 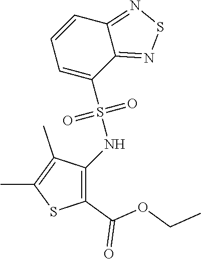 [SMILES: CCOC(=O)c1sc(C)c(C)c1NS(=O)(=O)c1cccc2nsnc12] |
| 2H076 | 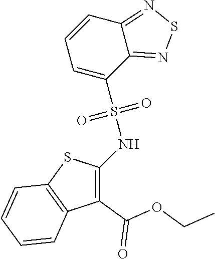 [SMILES: CCOC(=O)c1c(NS(=O)(=O)c2cccc3nsnc23)sc2ccccc12] |
| 2C229 | 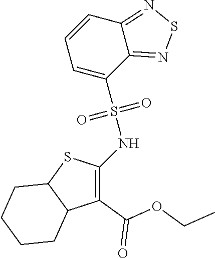 [SMILES: CCOC(=O)C1=C(NS(=O)(=O)c2cccc3nsnc23)SC2CCCCC12] |
| 2H079 | 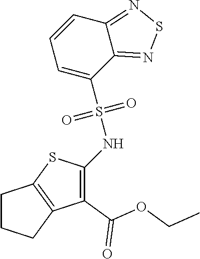 [SMILES: CCOC(=O)c1c(NS(=O)(=O)c2cccc3nsnc23)sc2c1CCC2] |
| 2G255 | 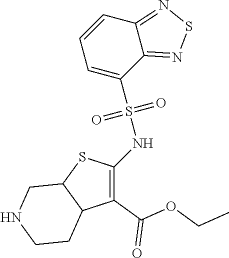 [SMILES: CCOC(=O)C1=C(NS(=O)(=O)c2cccc3nsnc23)SC2CNCCC12] |
| 2G249 | 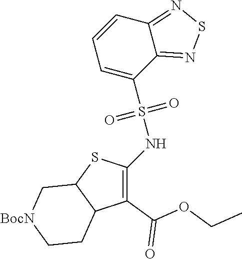 [SMILES: CCOC(=O)C1=C(NS(=O)(=O)c2cccc3nsnc23)SC2CN(C(=O)OC(C)(C)C)CCC12] |
| 2G252 | 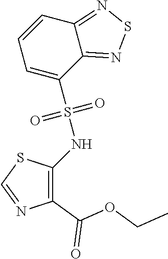 [SMILES: CCOC(=O)c1ncsc1NS(=O)(=O)c1cccc2nsnc12] |
| 2G254 | 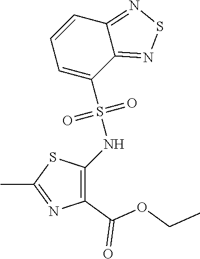 [SMILES: CCOC(=O)c1nc(C)sc1NS(=O)(=O)c1cccc2nsnc12] |
| 2G258 | 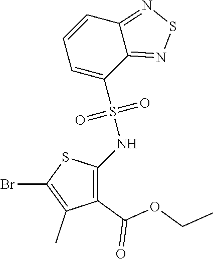 [SMILES: CCOC(=O)c1c(NS(=O)(=O)c2cccc3nsnc23)sc(Br)c1C] |
| 2G284 | 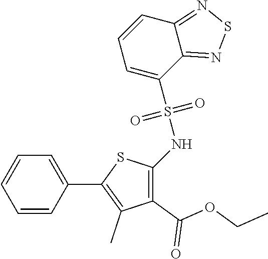 [SMILES: CCOC(=O)c1c(NS(=O)(=O)c2cccc3nsnc23)sc(-c2ccccc2)c1C] |
| 2G276 | 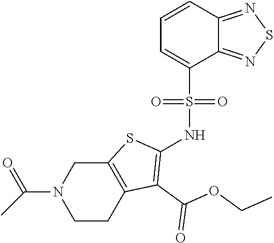 [SMILES: CCOC(=O)c1c(NS(=O)(=O)c2cccc3nsnc23)sc2c1CCN(C(C)=O)C2] |
| 2G279 | 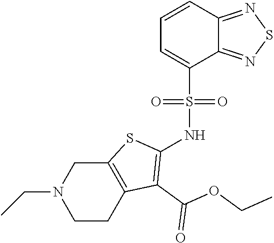 [SMILES: CCOC(=O)c1c(NS(=O)(=O)c2cccc3nsnc23)sc2c1CCN(CC)C2] |
| 2G287 | 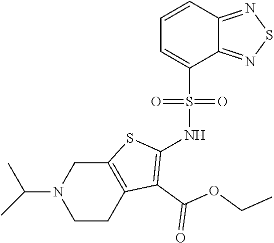 [SMILES: CCOC(=O)c1c(NS(=O)(=O)c2cccc3nsnc23)sc2c1CCN(C(C)C)C2] |
| 2H063 | 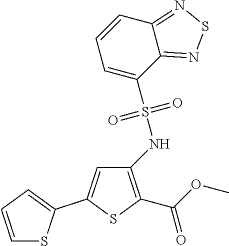 [SMILES: COC(=O)c1sc(-c2cccs2)cc1NS(=O)(=O)c1cccc2nsnc12] |
| 2H080 | 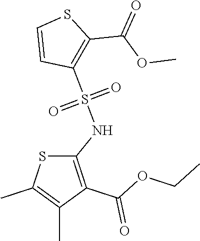 [SMILES: CCOC(=O)c1c(NS(=O)(=O)c2ccsc2C(=O)OC)sc(C)c1C] |
| | 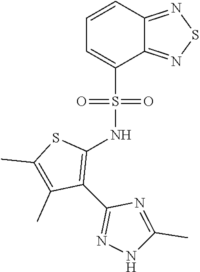 [SMILES: Cc1nc(-c2c(NS(=O)(=O)c3cccc4nsnc34)sc(C)c2C)n[nH]1] |
| | 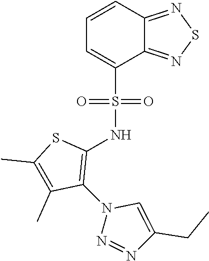 [SMILES: CCc1cn(-c2c(NS(=O)(=O)c3cccc4nsnc34)sc(C)c2C)nn1] |
| | 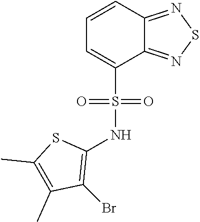 [SMILES: Cc1sc(NS(=O)(=O)c2cccc3nsnc23)c(Br)c1C] |
| | 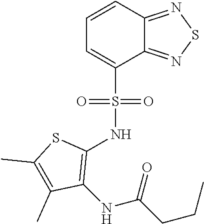 [SMILES: CCCC(=O)Nc1c(NS(=O)(=O)c2cccc3nsnc23)sc(C)c1C] |
| | 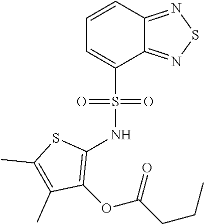 [SMILES: CCCC(=O)Oc1c(NS(=O)(=O)c2cccc3nsnc23)sc(C)c1C] |
| | 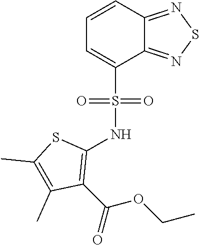 [SMILES: CCOC(=O)c1c(NS(=O)(=O)c2cccc3nsnc23)sc(C)c1C] |
| | 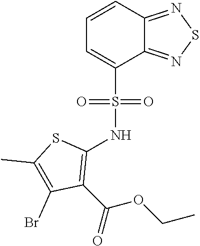 [SMILES: CCOC(=O)c1c(NS(=O)(=O)c2cccc3nsnc23)sc(C)c1Br] |
| | 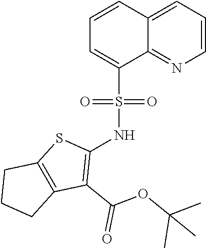 [SMILES: CC(C)(C)OC(=O)c1c(NS(=O)(=O)c2cccc3cccnc23)sc2c1CCC2] |

48. A compound of formula (V) or a pharmaceutically acceptable salt thereof: 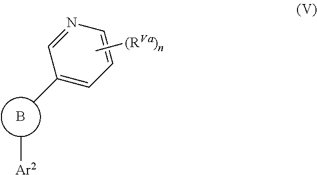
wherein
Ring B is 5-membered heteroaryl (wherein 1-4 heteroaryl members are independently selected from N, O, and S);
Ar² is selected from the group consisting of C₁-C₆-alkyl, monocyclic or bicyclic C₆-C₁₀-aryl, monocyclic or bicyclic 5- to 10-membered heteroaryl (wherein 1-4 heteroaryl members are independently selected from N, O, and S), and monocyclic or bicyclic 5- to 10-membered fully or partially saturated heterocycloalkyl (wherein 1 to 4 ring members are independently selected from N, O, and S), wherein Ar² is optionally substituted with 1 to 3 R^{IIIb};
R^{Va} is selected from the group consisting of C₁-C₆-alkyl, C₁-C₆-haloalkyl, halo, C₆-C₁₀-aryl, —O(C₆-C₁₀-aryl), —S(C₆-C₁₀-aryl), and 5- to 10-membered heteroaryl (wherein 1-4 heteroaryl members are independently selected from N, O, and S), wherein aryl or heteroaryl is optionally substituted with 1 to 3 substituents selected from C₁-C₆-alkyl and halo;
n is 0, 1, or 2; and
R^{Vb} is selected from the group consisting of C₁-C₆-alkyl, —OC₁-C₆-alkyl, C₁-C₆-haloalkyl, —OC₁-C₆-haloalkyl, halo, oxo, NO₂, CN, NRR′ (wherein R and R′ are independently selected from H and C₁-C₆-alkyl);
with the proviso that the compound is not any of the following:

| | |
|---|---|
| 645 | 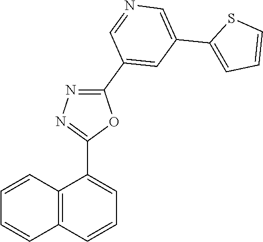 [SMILES: c1csc(-c2cncc(-c3nnc(-c4cccc5ccccc45)o3)c2)c1] |
| 1301 | 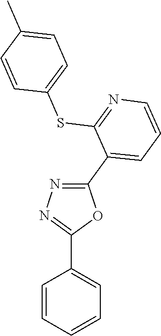 [SMILES: Cc1ccc(Sc2ncccc2-c2nnc(-c3ccccc3)o2)cc1] |
| 1302 | 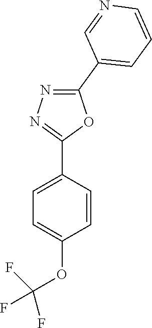 [SMILES: FC(F)(F)Oc1ccc(-c2nnc(-c3cccnc3)o2)cc1] |
| 1303 | 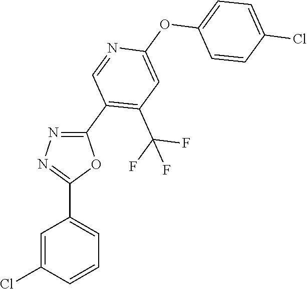 [SMILES: FC(F)(F)c1cc(Oc2ccc(Cl)cc2)ncc1-c1nnc(-c2cccc(Cl)c2)o1] |
| 1304 | 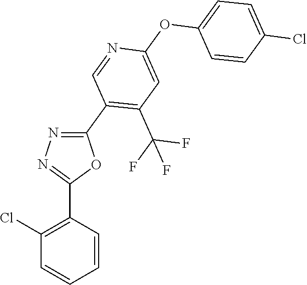 [SMILES: FC(F)(F)c1cc(Oc2ccc(Cl)cc2)ncc1-c1nnc(-c2ccccc2Cl)o1] |
| 1305 | 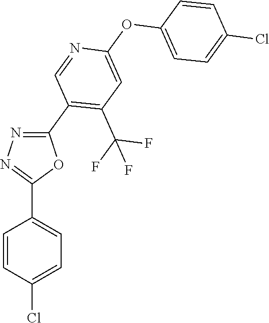 [SMILES: FC(F)(F)c1cc(Oc2ccc(Cl)cc2)ncc1-c1nnc(-c2ccc(Cl)cc2)o1] |
| 1310 | 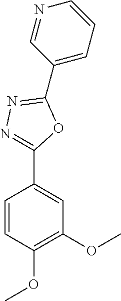 [SMILES: COc1ccc(-c2nnc(-c3cccnc3)o2)cc1OC] |
| 1306 | 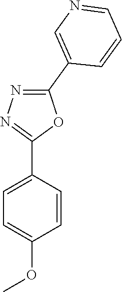 [SMILES: COc1ccc(-c2nnc(-c3cccnc3)o2)cc1] |
| 1311 | 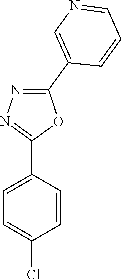 [SMILES: Clc1ccc(-c2nnc(-c3cccnc3)o2)cc1] |
| 1307 | 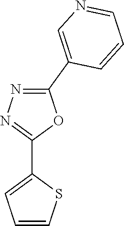 [SMILES: c1cncc(-c2nnc(-c3cccs3)o2)c1] |
| 1308 | 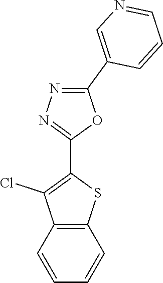 [SMILES: Clc1c(-c2nnc(-c3cccnc3)o2)sc2ccccc12] |
| 1309 | 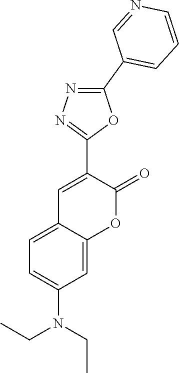 [SMILES: CCN(CC)c1ccc2cc(-c3nnc(-c4cccnc4)o3)c(=O)oc2c1] |

49. The compound according to claim 48, wherein Ring B is selected from the group consisting of:

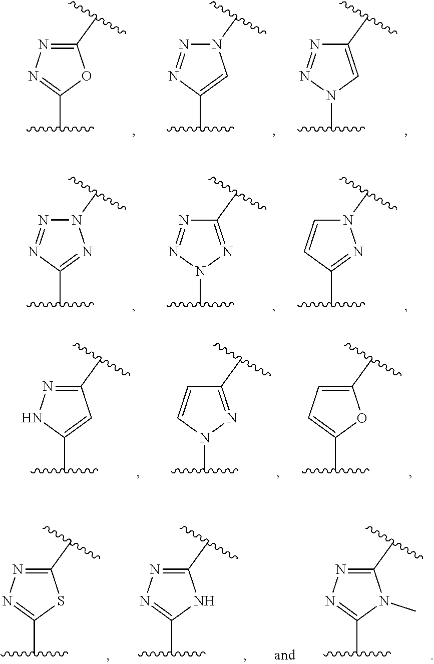

50. The compound according to claim 48 or 49, wherein Ar² is optionally substituted monocyclic or bicyclic C₆-C₁₀-aryl.

51. The compound according to any of claims 48 to 50, where Ar² is optionally substituted phenyl.

52. The compound according to any of claims 48 to 50, where Ar² is optionally substituted naphthyl.

53. The compound according to claim 48 or 49, wherein Ar² is optionally substituted monocyclic or bicyclic 5- to 10-membered heteroaryl (wherein 1-4 heteroaryl members are independently selected from N, O, and S).

54. The compound according to any of claims 48, 49, and 53, wherein Ar² is optionally substituted monocyclic 5- to 6-membered heteroaryl (wherein 1-4 heteroaryl members are independently selected from N, O, and S).

55. The compound according to any of claims 48 to 54, wherein n is 1 and R^{IIIa} is optionally substituted thiophenyl.

56. The compound according to claim 48, wherein the compound is one selected from the following table:

| | |
|---|---|
| 2C176 | 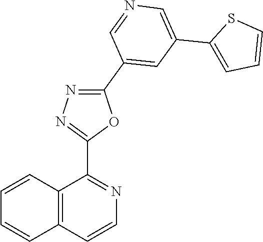 [SMILES: c1csc(-c2cncc(-c3nnc(-c4nccc5ccccc45)o3)c2)c1] |
| 2C173 | 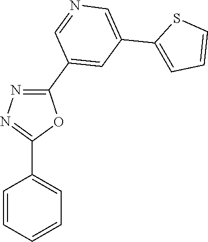 [SMILES: c1ccc(-c2nnc(-c3cncc(-c4cccs4)c3)o2)cc1] |
| 2C201 | 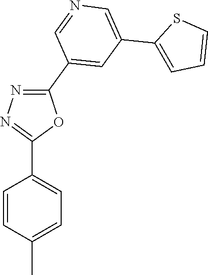 [SMILES: Cc1ccc(-c2nnc(-c3cncc(-c4cccs4)c3)o2)cc1] |
| 2H019 | 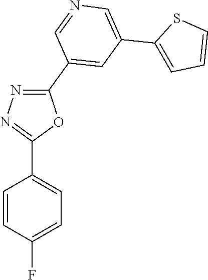 [SMILES: Fc1ccc(-c2nnc(-c3cncc(-c4cccs4)c3)o2)cc1] |
| 2H022 | 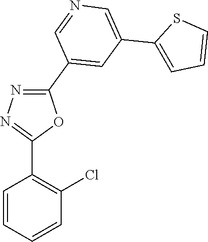 [SMILES: Clc1ccccc1-c1nnc(-c2cncc(-c3cccs3)c2)o1] |
| 2H023 | 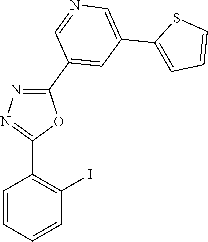 [SMILES: Ic1ccccc1-c1nnc(-c2cncc(-c3cccs3)c2)o1] |
| 2H028 | 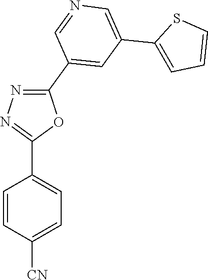 [SMILES: N#Cc1ccc(-c2nnc(-c3cncc(-c4cccs4)c3)o2)cc1] |
| 2H027 | 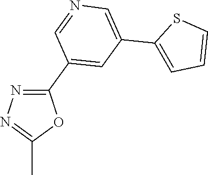 [SMILES: Cc1nnc(-c2cncc(-c3cccs3)c2)o1] |
| 2H032 | 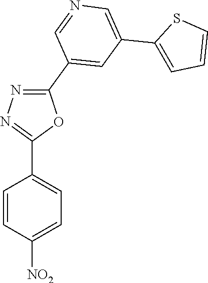 [SMILES: O=[N+]([O-])c1ccc(-c2nnc(-c3cncc(-c4cccs4)c3)o2)cc1] |
| 2H037 | 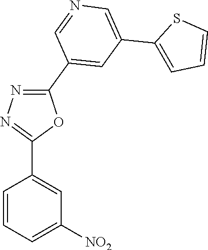 [SMILES: O=[N+]([O-])c1cccc(-c2nnc(-c3cncc(-c4cccs4)c3)o2)c1] |
| 2C211 | 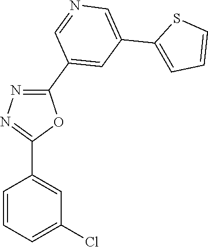 [SMILES: Clc1cccc(-c2nnc(-c3cncc(-c4cccs4)c3)o2)c1] |
| 2F188B | 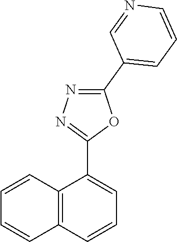 [SMILES: c1cncc(-c2nnc(-c3cccc4ccccc34)o2)c1] |
| 2F187 | 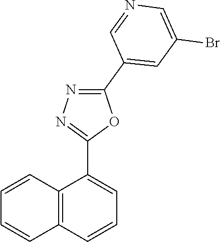 [SMILES: Brc1cncc(-c2nnc(-c3cccc4ccccc34)o2)c1] |
| 2C178 | 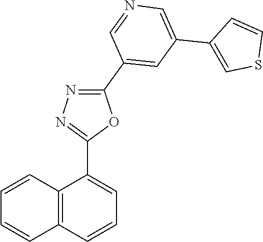 [SMILES: c1ccc2c(-c3nnc(-c4cncc(-c5ccsc5)c4)o3)cccc2c1] |
| 2H005 | 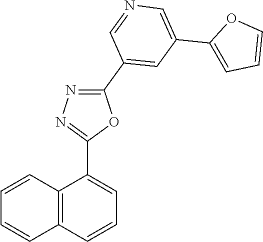 [SMILES: c1coc(-c2cncc(-c3nnc(-c4cccc5ccccc45)o3)c2)c1] |
| 2C179 | 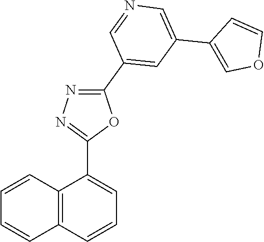 [SMILES: c1ccc2c(-c3nnc(-c4cncc(-c5ccoc5)c4)o3)cccc2c1] |
| 2C181 | 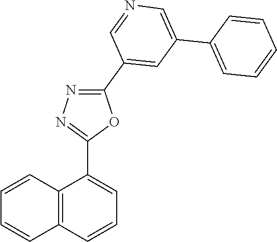 [SMILES: c1ccc(-c2cncc(-c3nnc(-c4cccc5ccccc45)o3)c2)cc1] |
| 2C183 | 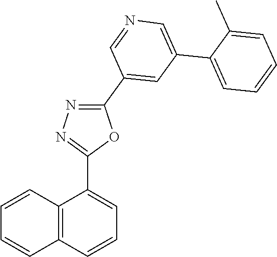 [SMILES: Cc1ccccc1-c1cncc(-c2nnc(-c3cccc4ccccc34)o2)c1] |
| 2C185 | 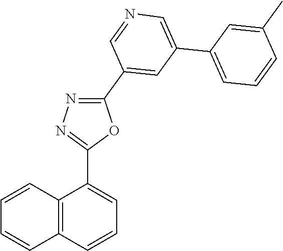 [SMILES: Cc1cccc(-c2cncc(-c3nnc(-c4cccc5ccccc45)o3)c2)c1] |
| 2C182 | 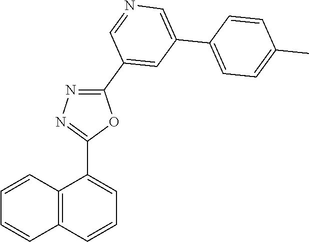 [SMILES: Cc1ccc(-c2cncc(-c3nnc(-c4cccc5ccccc45)o3)c2)cc1] |
| 2H008 | 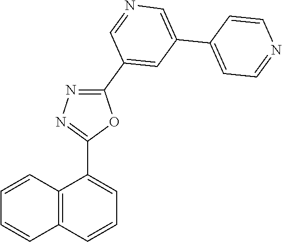 [SMILES: c1ccc2c(-c3nnc(-c4cncc(-c5ccncc5)c4)o3)cccc2c1] |
| 2C188 | 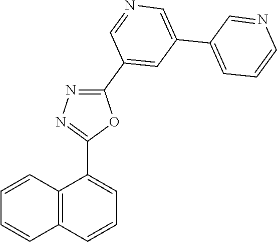 [SMILES: c1cncc(-c2cncc(-c3nnc(-c4cccc5ccccc45)o3)c2)c1] |
| 2H050 | 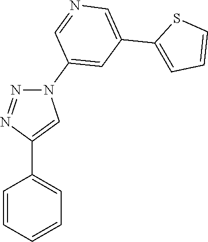 [SMILES: c1ccc(-c2cn(-c3cncc(-c4cccs4)c3)nn2)cc1] |
| 2H042 | 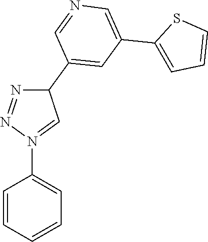 |
| 2E279 | 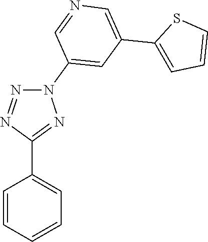 [SMILES: c1ccc(-c2nnn(-c3cncc(-c4cccs4)c3)n2)cc1] |
| 2E277 | 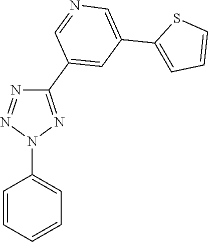 [SMILES: c1ccc(-n2nnc(-c3cncc(-c4cccs4)c3)n2)cc1] |
| 2C214 | 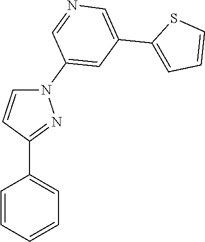 [SMILES: c1ccc(-c2ccn(-c3cncc(-c4cccs4)c3)n2)cc1] |
| 2E280 | 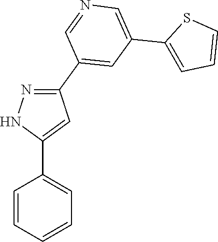 [SMILES: c1ccc(-c2cc(-c3cncc(-c4cccs4)c3)n[nH]2)cc1] |
| | 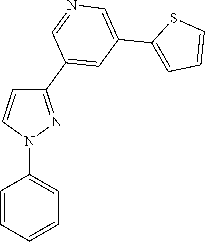 [SMILES: c1ccc(-n2ccc(-c3cncc(-c4cccs4)c3)n2)cc1] |
| | 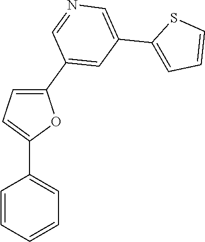 [SMILES: c1ccc(-c2ccc(-c3cncc(-c4cccs4)c3)o2)cc1] |
| | 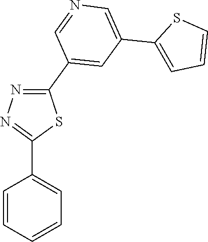 [SMILES: c1ccc(-c2nnc(-c3cncc(-c4cccs4)c3)s2)cc1] |
| | 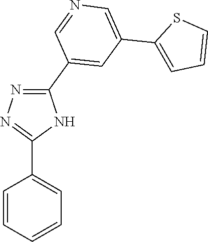 [SMILES: c1ccc(-c2nnc(-c3cncc(-c4cccs4)c3)[nH]2)cc1] |
| | 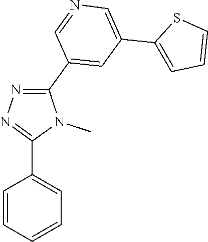 [SMILES: Cn1c(-c2ccccc2)nnc1-c1cncc(-c2cccs2)c1] |

57. A composition comprising a compound of formula (IV) or formula (V) and one or more antigens.

58. A compound of formula (II-A) or a pharmaceutically acceptable salt thereof, formula (I) or a pharmaceutically acceptable salt thereof, or a combination thereof: 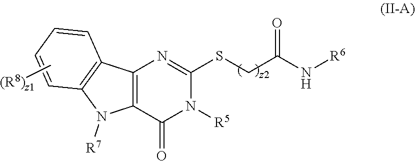
wherein
z1 is an integer from 0 to 4, and z2 is an integer from 0 to 5;
R⁵ is R^{5A}-substituted or unsubstituted cycloalkyl, R^{5A}-substituted or unsubstituted heterocycloalkyl, R^{5A}-substituted or unsubstituted aryl, or R^{5A}-substituted or unsubstituted heteroaryl.
R^{5A} is independently halogen, —CN, —CF₃, —CCl₃, —OH, —NH₂, —SO₂, —COOH, oxo, nitro, —SH, —CONH₂, —NH—OH, R^{5B}-substituted or unsubstituted alkyl, R^{5B}-substituted or unsubstituted alkynyl, R^{5B}-substituted or unsubstituted heteroalkyl, R^{5B}-substituted or unsubstituted cycloalkyl, R^{5B}-substituted or unsubstituted heterocycloalkyl, R^{5B}-substituted or unsubstituted aryl, or R^{5B}-substituted or unsubstituted heteroaryl;
R^{5B} is independently halogen, —CN, —CF₃, —CCl₃, —OH, —NH₂, —SO₂, —COOH, oxo, nitro, —SH, —CONH₂, R^{5C}-substituted or unsubstituted alkyl, R^{5C}-substituted or unsubstituted heteroalkyl, R^{5C}-substituted or unsubstituted cycloalkyl, R^{5C}-substituted or unsubstituted heterocycloalkyl, R^{5C}-substituted or unsubstituted aryl, or R^{5C}-substituted or unsubstituted heteroaryl;
R^{5C} is independently halogen, —CN, —CF₃, —CCl₃, —OH, —NH₂, —SO₂, —COOH, oxo, nitro, —SH, —CONH₂, unsubstituted alkyl, unsubstituted heteroalkyl, unsubstituted cycloalkyl, unsubstituted heterocycloalkyl, unsubstituted aryl, or unsubstituted heteroaryl;
R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
R⁷ is hydrogen, or substituted or unsubstituted alkyl; and
R⁸ is independently halogen, —CN, —SH, —OH, —COOH, —NH₂, —CONH₂, nitro, —CF₃, —CCl₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

59. The compound according to claim 58, wherein the compound is selected from the following table:

| | |
|---|---|
| 2B182c | 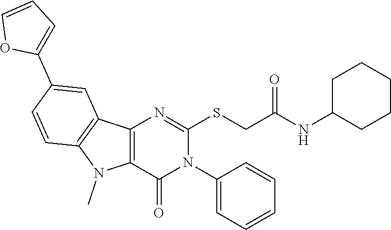 [SMILES: Cn1c2ccc(-c3ccco3)cc2c2nc(SCC(=O)NC3CCCCC3)n(-c3ccccc3)c(=O)c21] |
| 2G023a | 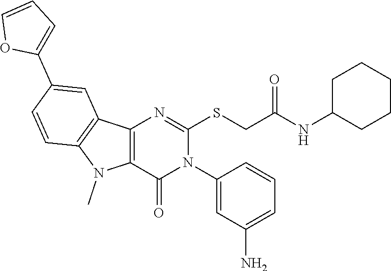 [SMILES: Cn1c2ccc(-c3ccco3)cc2c2nc(SCC(=O)NC3CCCCC3)n(-c3cccc(N)c3)c(=O)c21] |
| 2G053 | 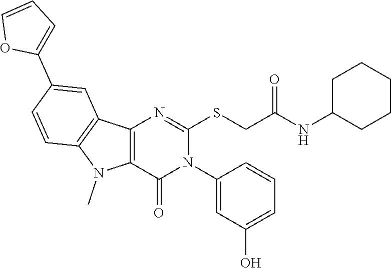 [SMILES: Cn1c2ccc(-c3ccco3)cc2c2nc(SCC(=O)NC3CCCCC3)n(-c3cccc(O)c3)c(=O)c21] |
| 2G112 | 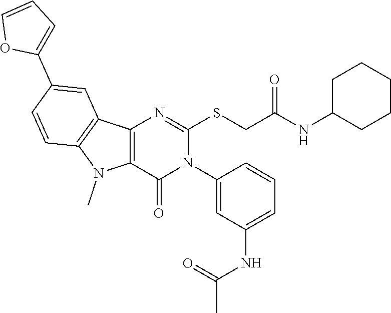 [SMILES: CC(=O)Nc1cccc(-n2c(SCC(=O)NC3CCCCC3)nc3c4cc(-c5ccco5)ccc4n(C)c3c2=O)c1] |
| 2G113 | 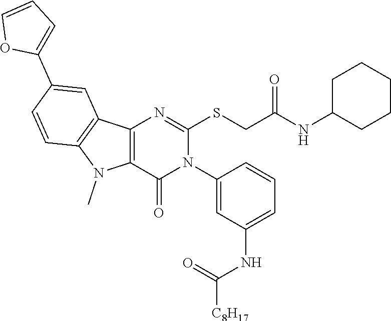 |
| 2G107 | 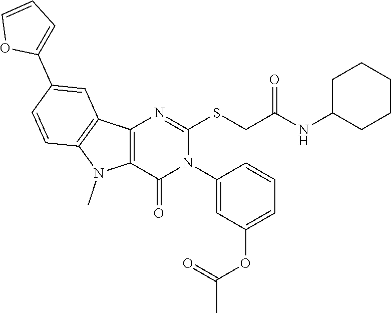 [SMILES: CC(=O)Oc1cccc(-n2c(SCC(=O)NC3CCCCC3)nc3c4cc(-c5ccco5)ccc4n(C)c3c2=O)c1] |
| 2G108 | 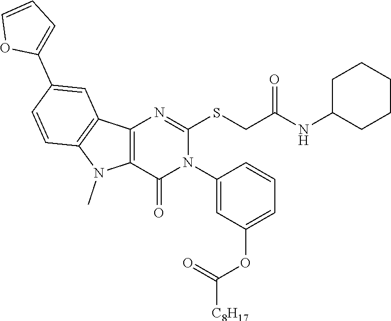 |
| 2G157 | 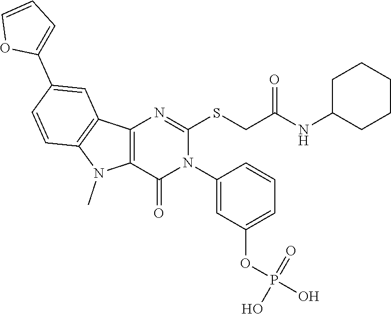 [SMILES: Cn1c2ccc(-c3ccco3)cc2c2nc(SCC(=O)NC3CCCCC3)n(-c3cccc(OP(=O)(O)O)c3)c(=O)c21] |
| 2G154 | 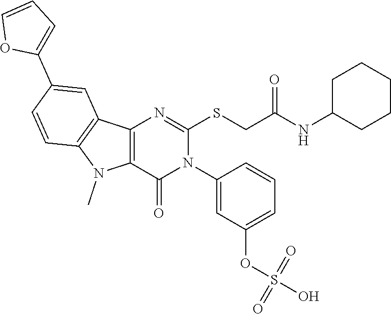 [SMILES: Cn1c2ccc(-c3ccco3)cc2c2nc(SCC(=O)NC3CCCCC3)n(-c3cccc(OS(=O)(=O)O)c3)c(=O)c21] |
| 2G202 | 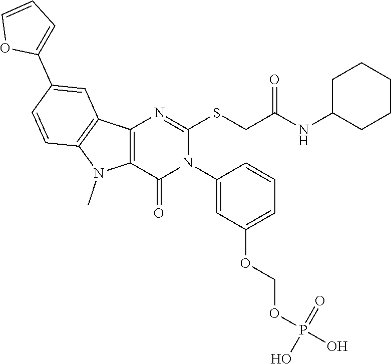 [SMILES: Cn1c2ccc(-c3ccco3)cc2c2nc(SCC(=O)NC3CCCCC3)n(-c3cccc(OCOP(=O)(O)O)c3)c(=O)c21] |
| 2E255 | 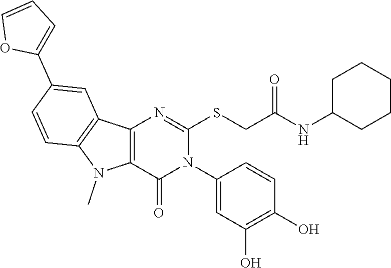 [SMILES: Cn1c2ccc(-c3ccco3)cc2c2nc(SCC(=O)NC3CCCCC3)n(-c3ccc(O)c(O)c3)c(=O)c21] |
| 2G193 | 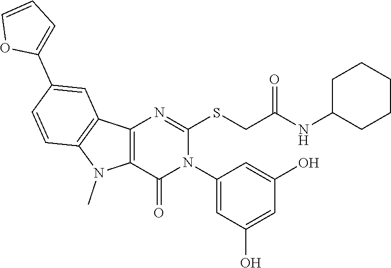 [SMILES: Cn1c2ccc(-c3ccco3)cc2c2nc(SCC(=O)NC3CCCCC3)n(-c3cc(O)cc(O)c3)c(=O)c21] |
| 2G177 | 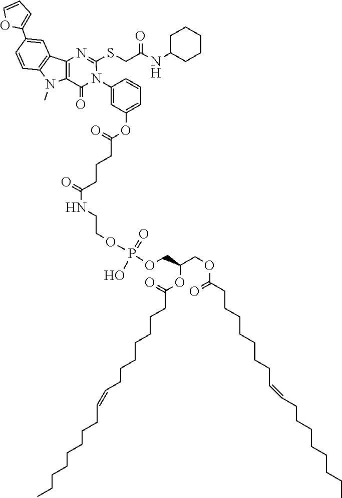 [SMILES: CCCCCCCC/C=C\CCCCCCCC(=O)OC[C@H](COP(=O)(O)OCCNC(=O)CCCC(=O)Oc1cccc(-n2c(SCC(=O)NC3CCCCC3)nc3c4cc(-c5ccco5)ccc4n(C)c3c2=O)c1)OC(=O)CCCCCCC/C=C\CCCCCCCC] |
| 2G22 | 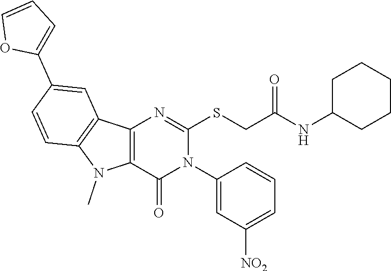 [SMILES: Cn1c2ccc(-c3ccco3)cc2c2nc(SCC(=O)NC3CCCCC3)n(-c3cccc([N+](=O)[O-])c3)c(=O)c21] |
| 2G004 | 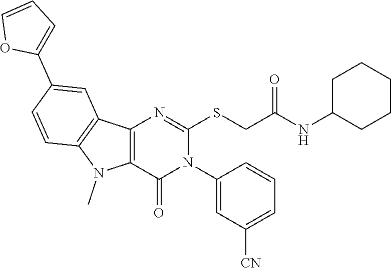 [SMILES: Cn1c2ccc(-c3ccco3)cc2c2nc(SCC(=O)NC3CCCCC3)n(-c3cccc(C#N)c3)c(=O)c21] |
| 2G013 | 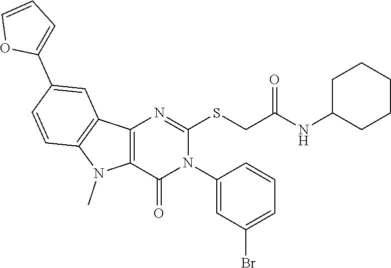 [SMILES: Cn1c2ccc(-c3ccco3)cc2c2nc(SCC(=O)NC3CCCCC3)n(-c3cccc(Br)c3)c(=O)c21] |
| 2G023b | 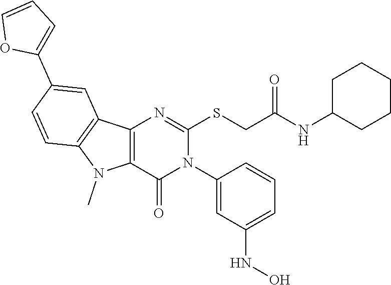 [SMILES: Cn1c2ccc(-c3ccco3)cc2c2nc(SCC(=O)NC3CCCCC3)n(-c3cccc(NO)c3)c(=O)c21] |
| 2G016 | 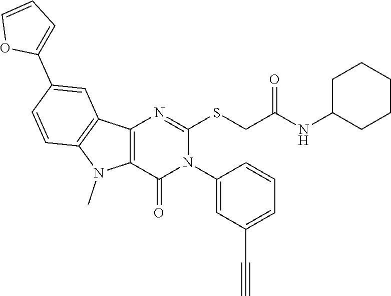 [SMILES: C#Cc1cccc(-n2c(SCC(=O)NC3CCCCC3)nc3c4cc(-c5ccco5)ccc4n(C)c3c2=O)c1] |
| 2G010 | 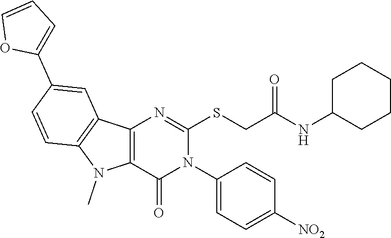 [SMILES: Cn1c2ccc(-c3ccco3)cc2c2nc(SCC(=O)NC3CCCCC3)n(-c3ccc([N+](=O)[O-])cc3)c(=O)c21] |
| 2G002 | 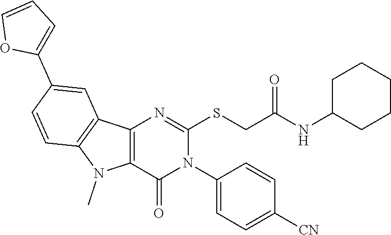 [SMILES: Cn1c2ccc(-c3ccco3)cc2c2nc(SCC(=O)NC3CCCCC3)n(-c3ccc(C#N)cc3)c(=O)c21] |
| 2G019 | 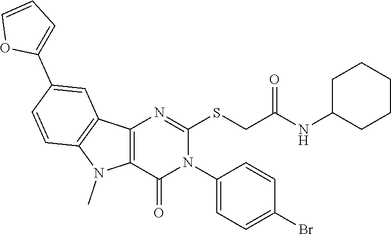 [SMILES: Cn1c2ccc(-c3ccco3)cc2c2nc(SCC(=O)NC3CCCCC3)n(-c3ccc(Br)cc3)c(=O)c21] |
| 2G036a | 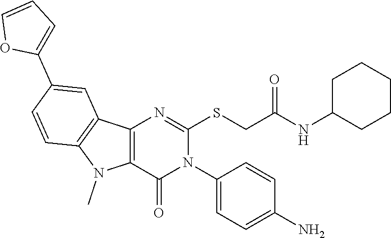 [SMILES: Cn1c2ccc(-c3ccco3)cc2c2nc(SCC(=O)NC3CCCCC3)n(-c3ccc(N)cc3)c(=O)c21] |
